(19) United States
(12) Patent Application Publication
(10) Pub. No.: US 2025/0275346 A1
(43) Pub. Date: Aug. 28, 2025

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Hanill LEE, Suwon-si, Gyeonggi-do (KR); Yoonman LEE, Suwon-si, Gyeonggi-do (KR); Youngkyoung JO, Suwon-si, Gyeonggi-do (KR); Seonyeong GWAK, Suwon-si, Gyeonggi-do (KR); Suheon IM, Suwon-si, Gyeonggi-do (KR); Seungjae LEE, Suwon-si, Gyeonggi-do (KR); Minseok SEO, Suwon-si, Gyeonggi-do (KR); Wook KIM, Suwon-si, Gyeonggi-do (KR); Sung-Hyun JUNG, Suwon-si, Gyeonggi-do (KR); Hyung Sun KIM, Suwon-si, Gyeonggi-do (KR); Dongkyu RYU, Suwon-si, Gyeonggi-do (KR); Chang Ju SHIN, Suwon-si, Gyeonggi-do (KR)

(21) Appl. No.: 18/704,994

(22) PCT Filed: Jan. 17, 2023
(86) PCT No.: PCT/KR2023/000767

§ 371 Date: Apr. 26, 2024

(30) Foreign Application Priority Data
Jan. 17, 2022 (KR) 10-2022-0006763

(51) Int. Cl. H10K 50/11; C07D 251/24; C07D 307/91; C07D 403/10; H10K 85/60

(52) U.S. Cl. CPC H10K 50/11; C07D 251/24; C07D 307/91; C07D 403/10; H10K 85/622; H10K 85/633; H10K 85/636; H10K 85/654; H10K 85/6572; H10K 85/6574; H10K 85/6576

(57) ABSTRACT

Disclosed are a compound for an organic optoelectronic device represented by Chemical Formula 1, an organic optoelectronic device including the same, and a display device.

Details for Chemical Formula 1 are the same as described in the specification.

### TECHNICAL FIELD

A compound for an organic optoelectronic device, a composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device that can implement a low-driving, high-efficiency, and long life-span organic optoelectronic device.

Another embodiment provides a composition for an organic optoelectronic device that can implement a highly efficient and long-life organic optoelectronic device.

Another embodiment provides an organic optoelectronic device including the compound.

Another embodiment provides a display device including the organic optoelectronic device.

### Technical Solution

According to an embodiment, a compound for an organic optoelectronic device represented by Chemical Formula 1 is provided.

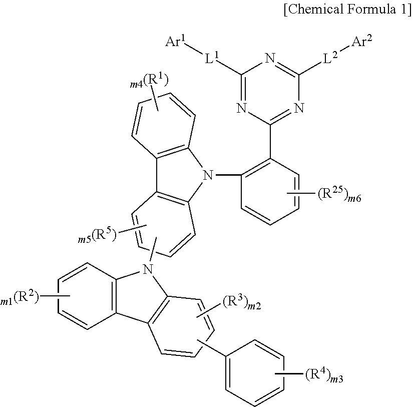

In Chemical Formula 1,
Ar¹ and Ar² are each independently a substituted or unsubstituted C6 to C30 aryl group or a substituted or unsubstituted C2 to C30 heterocyclic group,
L¹ and L² are each independently a single bond, a substituted or unsubstituted C6 to C30 arylene group, or a substituted or unsubstituted C2 to C30 heteroarylene group,
R¹ to R³ and R⁵ are each independently hydrogen, deuterium, a cyano group, or a substituted or unsubstituted C6 to C30 aryl group,
R⁴ is hydrogen, deuterium, a cyano group, a substituted or unsubstituted C1 to C10 alkyl group, or a substituted or unsubstituted C6 to C12 aryl group,
R²⁵ is hydrogen or deuterium,
m1, m4, and m6 are each independently one of integers of 1 to 4,
m2 and m5 are each independently one of integers of 1 to 3, and
m3 is one of integers of 1 to 5.

According to another embodiment, a composition for an organic optoelectronic device includes a first compound and a second compound.

The first compound may be as described above, and the second compound may be a compound for an organic optoelectronic device represented by Chemical Formula 2; or a compound for organic optoelectronic devices represented by a combination of Chemical Formula 3 and Chemical Formula 4.

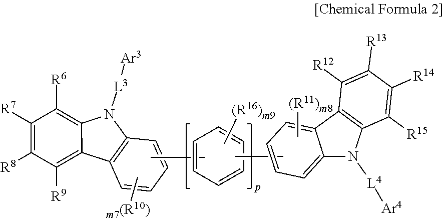

In Chemical Formula 2,
Ar³ and Ar⁴ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁶ to R¹⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m7 and m8 are each independently one of integers of 1 to 3,
m9 is one of integers of 1 to 4, and
p is one of integers of 0 to 2;

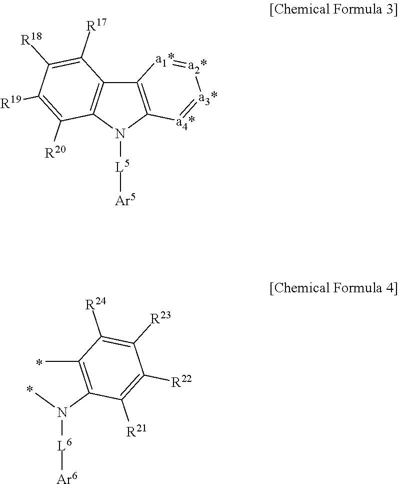

wherein, in Chemical Formulas 3 and 4,
Ar⁵ and Ar⁶ are each independently a substituted or unsubstituted C6 to C20 aryl group or a substituted or unsubstituted C2 to C30 heterocyclic group,
two adjacent ones among a₁* to a₄* in Chemical Formula 3 are each a linking carbon (C) linked to * in Chemical Formula 4,
the remaining two not linked to * in Chemical Formula 4, among a₁* to a₄* in Chemical Formula 3, are each independently C-L^{a}-R^{a},
L^{a}, L⁵, and L⁶ are each independently a single bond or a substituted or unsubstituted C6 to C20 arylene group, and
R^{a} and R¹⁷ to R²⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

According to another embodiment, an organic optoelectronic device includes an anode and a cathode facing each other, and at least one organic layer between the anode and the cathode, and the organic layer includes the compound for an organic optoelectronic device or the composition for an organic optoelectronic device.

According to another embodiment, a display device including the organic optoelectronic device is provided.

### Advantageous Effects

An organic optoelectronic device having low driving, high efficiency, and long life-span may be realized.

### DESCRIPTION OF THE DRAWINGS

FIG. 1 is a cross-sectional view showing an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C₅ alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for an organic optoelectronic device according to an embodiment is represented by Chemical Formula 1.

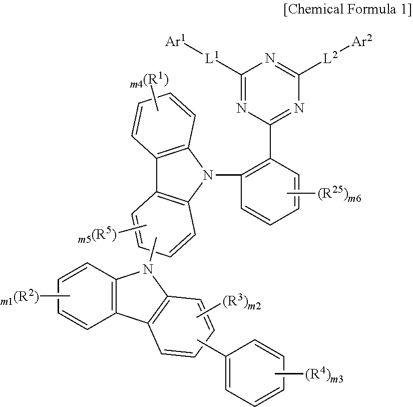

In Chemical Formula 1,
Ar¹ and Ar² are each independently a substituted or unsubstituted C6 to C30 aryl group or a substituted or unsubstituted C2 to C30 heterocyclic group,
L¹ and L² are each independently a single bond, a substituted or unsubstituted C6 to C30 arylene group, or a substituted or unsubstituted C2 to C30 heteroarylene group,
R¹ to R³ and R⁵ are each independently hydrogen, deuterium, a cyano group, or a substituted or unsubstituted C6 to C30 aryl group,
R⁴ is hydrogen, deuterium, a cyano group, a substituted or unsubstituted C1 to C10 alkyl group, or a substituted or unsubstituted C6 to C12 aryl group,
R²⁵ is hydrogen or deuterium,
m1, m4, and m6 are each independently one of integers of 1 to 4,
m2 and m5 are each independently one of integers of 1 to 3, and
m3 is one of integers of 1 to 5.

The compound represented by Chemical Formula 1 has a structure in which carbazole is linked to a triazine by ortho-phenylene and the carbazole is further substituted with phenyl-substituted 9-carbazole and two carbazole groups in a triazine with strong electronic characteristics are designed to have a small ΔEst, thereby enabling rapid energy transfer using a small ΔEst, to provide high efficiency characteristics, especially when applied as a phosphorescent host.

In addition, by further substituting the 9-carbazole with phenyl, the side reaction path in the excited state is reduced, thereby increasing the life-span, especially when applied as a phosphorescent host.

Moreover, the compound represented by Chemical Formula 1 has a structure with low molecular symmetry and steric hindrance, and thus degradation and decomposition can be minimized by reducing the deposition temperature, and thus life-span characteristics can be further improved.

Chemical Formula 1 may be represented by any one of Chemical Formula 1-1 to Chemical Formula 1-4.

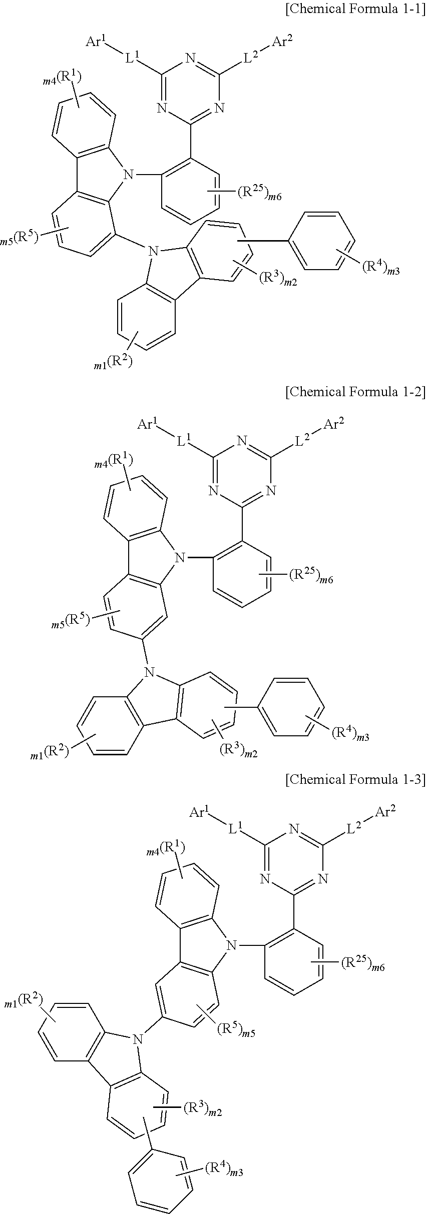

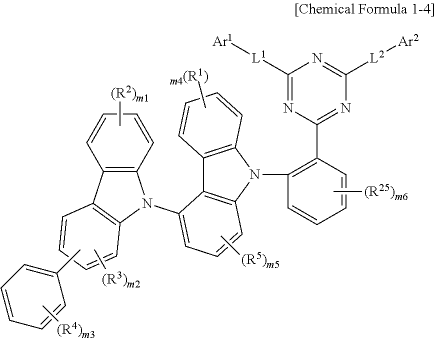

In Chemical Formula 1-1 to Chemical Formula 1-4,
the definitions of Ar¹, Ar², R¹ to R⁵, R²⁵, L¹, L², and m1 to m6 are as described above.

Chemical Formula 1-1 may be represented by any one of Chemical Formula 1-1A, Chemical Formula 1-1B, Chemical Formula 1-1C, and Chemical Formula 1-1D.

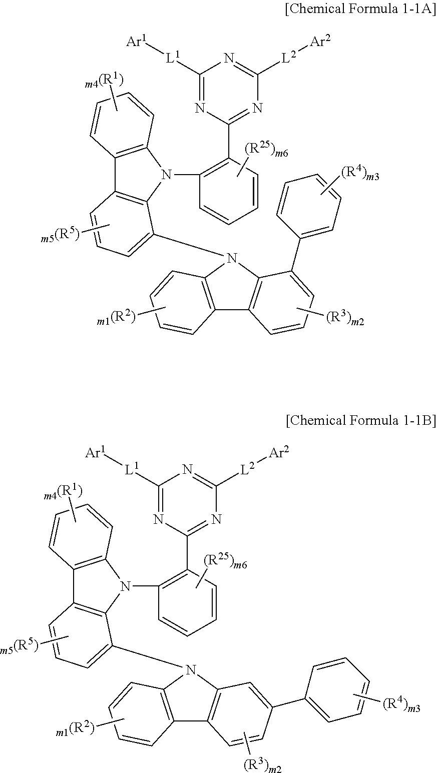

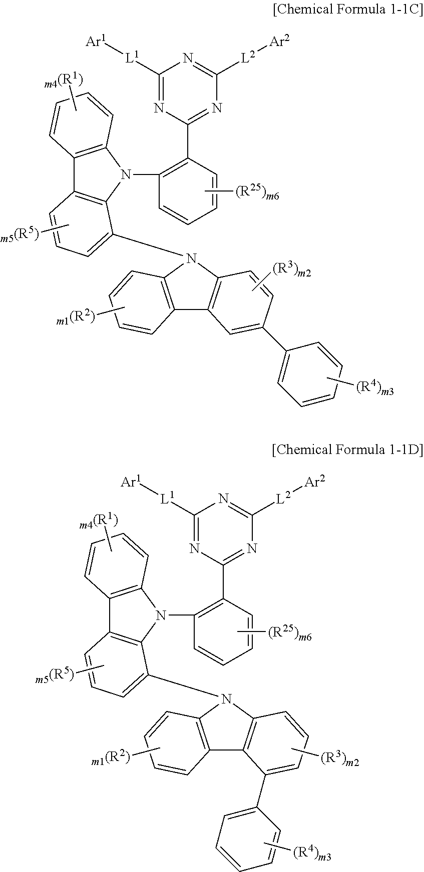

In Chemical Formula 1-1A, Chemical Formula 1-1B, Chemical Formula 1-1C, and Chemical Formula 1-1D, the definitions of Ar¹, Ar², R¹ to R⁵, R²¹, L¹, L², and m1 to m6 are the same as described above.

Chemical Formula 1-2 may be represented by any one of Chemical Formula 1-2A, Chemical Formula 1-2B, Chemical Formula 1-2C, and Chemical Formula 1-2D.

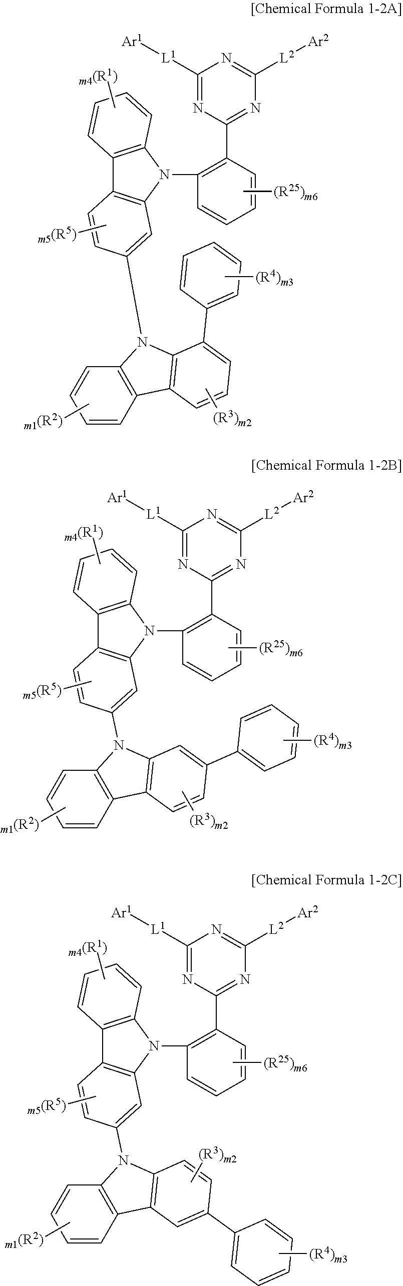

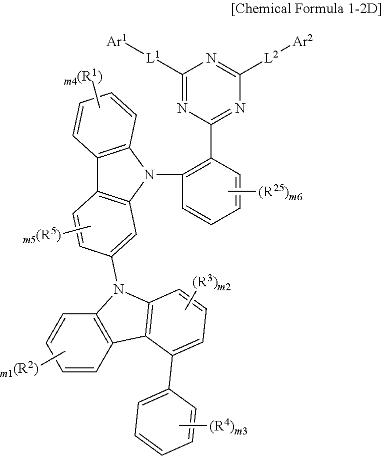

In Chemical Formula 1-2A, Chemical Formula 1-2B, Chemical Formula 1-2C, and Chemical Formula 1-2D, the definitions of Ar¹, Ar², R¹ to R⁵, R²¹, L¹, L², and m1 to m6 are the same as described above.

Chemical Formula 1-3 may be represented by any one of Chemical Formula 1-3A, Chemical Formula 1-3B, Chemical Formula 1-3C, and Chemical Formula 1-3D.

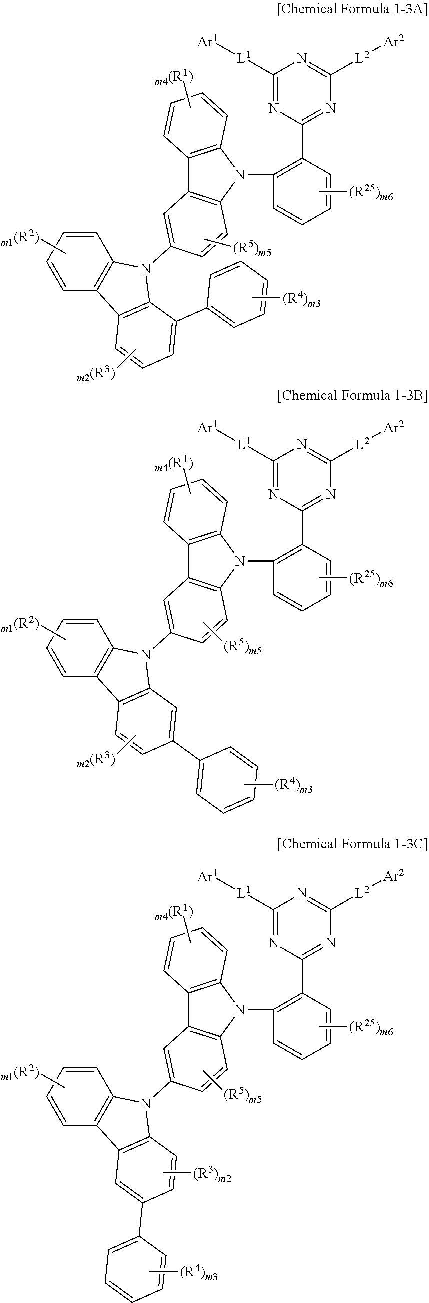

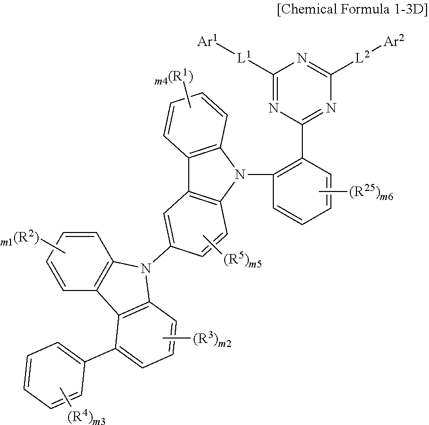

In Chemical Formula 1-3A, Chemical Formula 1-3B, Chemical Formula 1-3C, and Chemical Formula 1-3D,
the definitions of Ar¹, Ar², R¹ to R⁵, R²¹, L¹, L², and m1 to m6 are the same as described above.

Chemical Formula 1-4 may be represented by any one of Chemical Formula 1-4A, Chemical Formula 1-4B, Chemical Formula 1-4C, and Chemical Formula 1-4D.

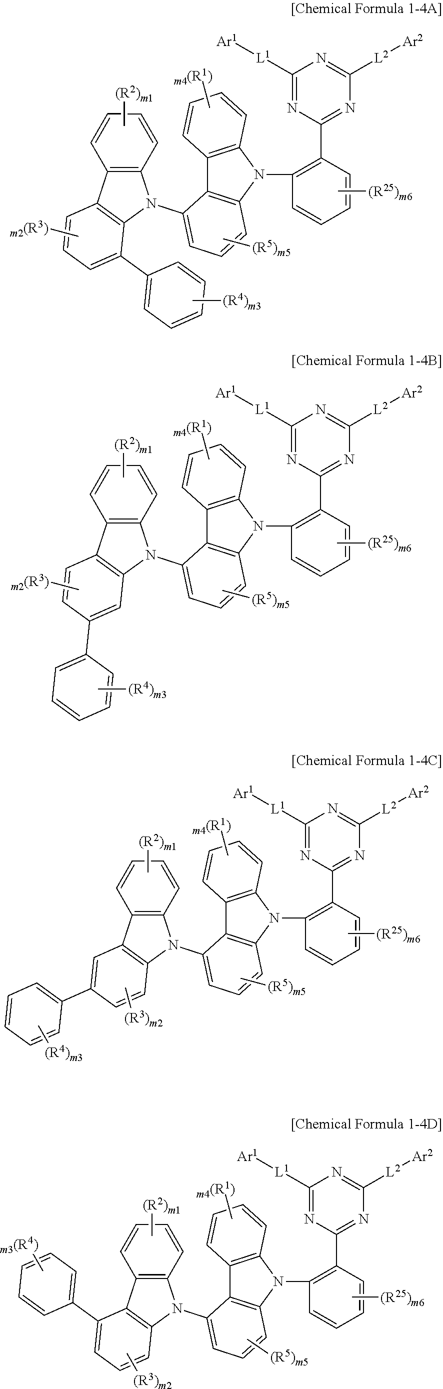

In Chemical Formula 1-4A, Chemical Formula 1-4B, Chemical Formula 1-4C, and Chemical Formula 1-4D,
the definitions of Ar¹, Ar², R¹ to R⁵, R²¹, L¹, L², and m1 to m6 are the same as described above.

As an example, Chemical Formula 1 may be represented by Chemical Formula 1-2.

As a specific example, Chemical Formula 1 may be represented by Chemical Formula 1-2B or Chemical Formula 1-2C.

R¹ to R³ may each independently be hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

For example, R¹ to R³ and R⁵ may each independently be hydrogen, deuterium, a cyano group, a substituted or unsubstituted phenyl group, or a substituted or unsubstituted biphenyl group.

R⁴ may be hydrogen, deuterium, a cyano group, or a substituted or unsubstituted phenyl group.

Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, or a substituted or unsubstituted dibenzothiophenyl group.

For example, Ar¹ and Ar² may each independently be a substituted or unsubstituted phenyl group, a substituted or unsubstituted biphenyl group, a substituted or unsubstituted terphenyl group, a substituted or unsubstituted fluorenyl group, a substituted or unsubstituted dibenzofuranyl group, or a substituted or unsubstituted dibenzothiophenyl group.

As a specific example, *-L¹-Ar¹ and *-L²-Ar² may each independently be selected from the substituents listed in Group I.

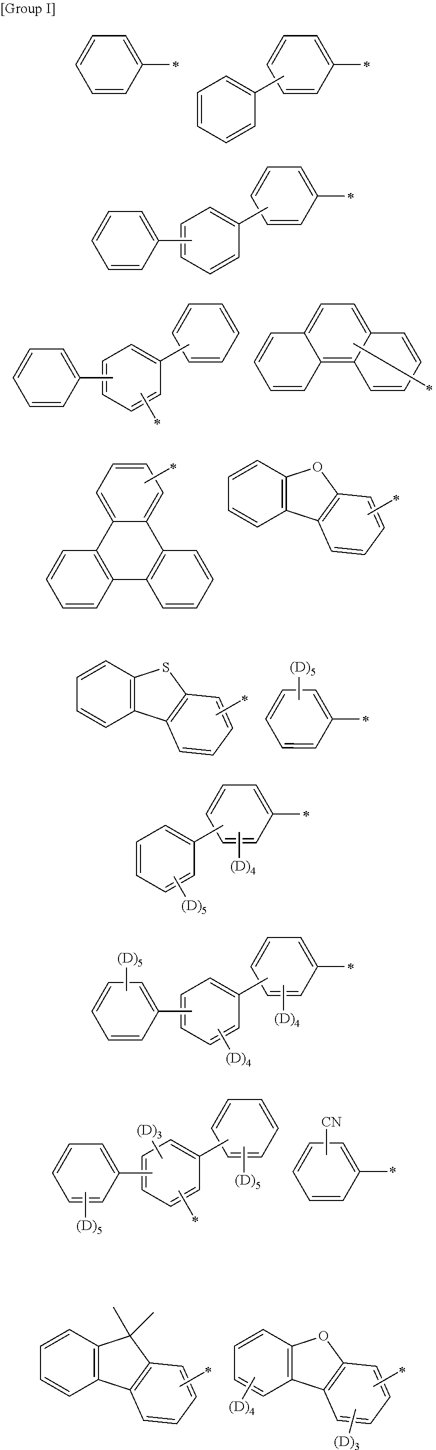

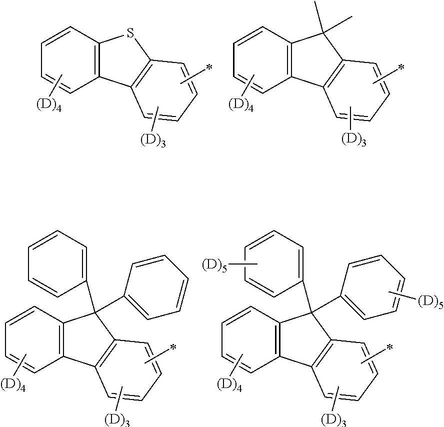

In Group I, * is a linking point.

In the most specific embodiment, the compound for an organic optoelectronic device represented by Chemical Formula 1 may be one selected from the compounds listed in Group 1, but is not limited thereto.

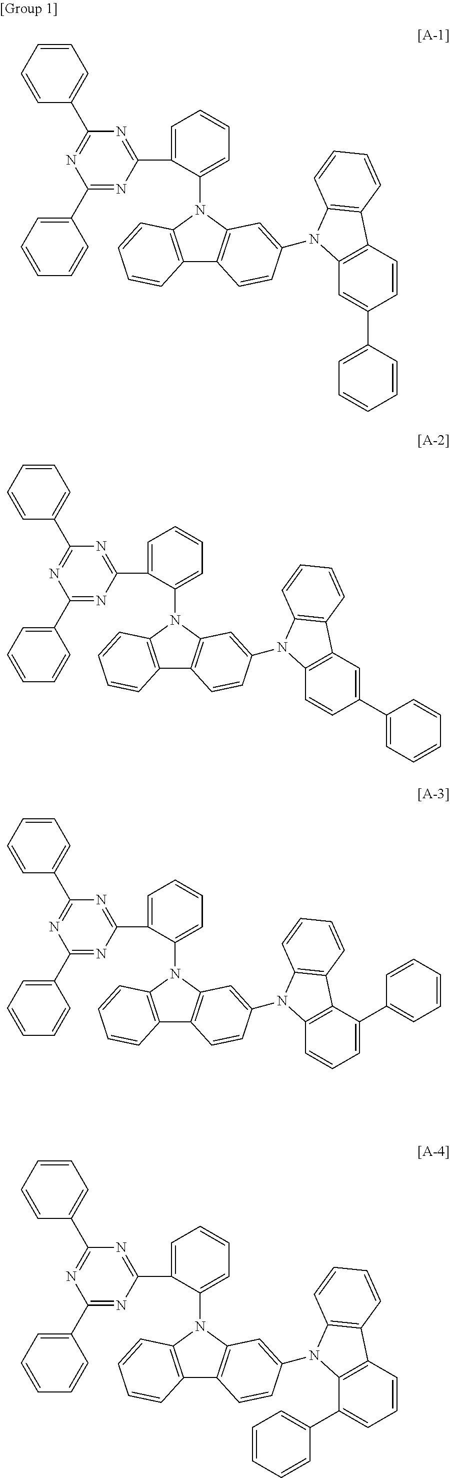 [SMILES: c1ccc(-c2ccc3c4ccccc4n(-c4ccc5c6ccccc6n(-c6ccccc6-c6nc(-c7ccccc7)nc(-c7ccccc7)n6)c5c4)c3c2)cc1 ; c1ccc(-c2ccc3c(c2)c2ccccc2n3-c2ccc3c4ccccc4n(-c4ccccc4-c4nc(-c5ccccc5)nc(-c5ccccc5)n4)c3c2)cc1 ; c1ccc(-c2nc(-c3ccccc3)nc(-c3ccccc3-n3c4ccccc4c4ccc(-n5c6ccccc6c6c(-c7ccccc7)cccc65)cc43)n2)cc1 ; c1ccc(-c2nc(-c3ccccc3)nc(-c3ccccc3-n3c4ccccc4c4ccc(-n5c6ccccc6c6cccc(-c7ccccc7)c65)cc43)n2)cc1]

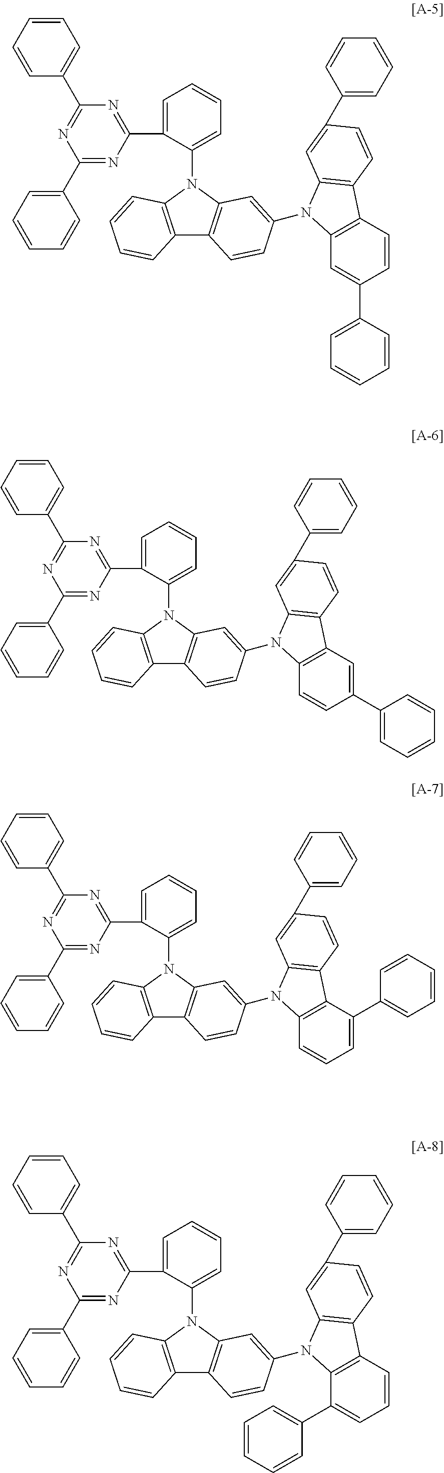 [SMILES: c1ccc(-c2ccc3c4ccc(-c5ccccc5)cc4n(-c4ccc5c6ccccc6n(-c6ccccc6-c6nc(-c7ccccc7)nc(-c7ccccc7)n6)c5c4)c3c2)cc1 ; c1ccc(-c2ccc3c(c2)c2ccc(-c4ccccc4)cc2n3-c2ccc3c4ccccc4n(-c4ccccc4-c4nc(-c5ccccc5)nc(-c5ccccc5)n4)c3c2)cc1 ; c1ccc(-c2ccc3c4c(-c5ccccc5)cccc4n(-c4ccc5c6ccccc6n(-c6ccccc6-c6nc(-c7ccccc7)nc(-c7ccccc7)n6)c5c4)c3c2)cc1 ; c1ccc(-c2ccc3c4cccc(-c5ccccc5)c4n(-c4ccc5c6ccccc6n(-c6ccccc6-c6nc(-c7ccccc7)nc(-c7ccccc7)n6)c5c4)c3c2)cc1]

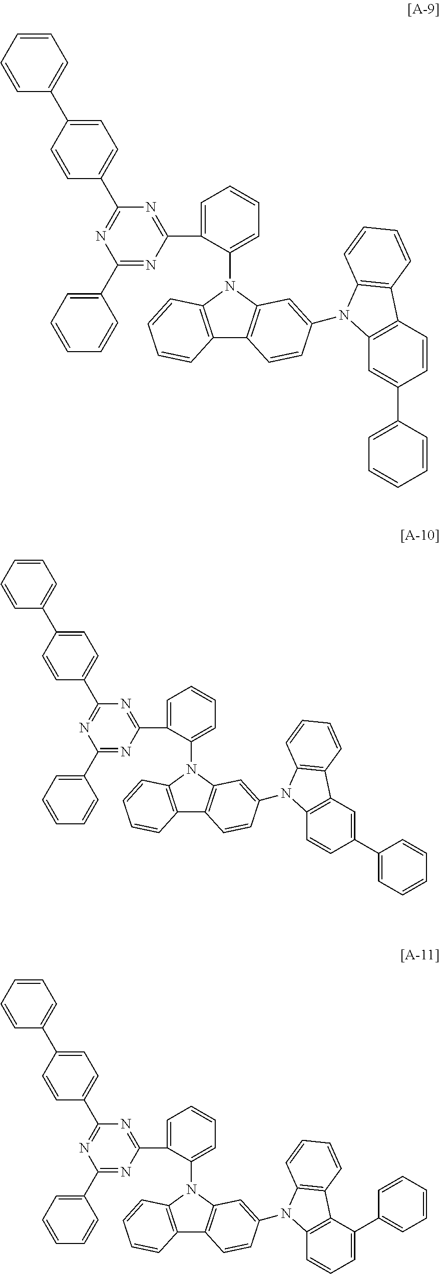 [SMILES: c1ccc(-c2ccc(-c3nc(-c4ccccc4)nc(-c4ccccc4-n4c5ccccc5c5ccc(-n6c7ccccc7c7ccc(-c8ccccc8)cc76)cc54)n3)cc2)cc1 ; c1ccc(-c2ccc(-c3nc(-c4ccccc4)nc(-c4ccccc4-n4c5ccccc5c5ccc(-n6c7ccccc7c7cc(-c8ccccc8)ccc76)cc54)n3)cc2)cc1 ; c1ccc(-c2ccc(-c3nc(-c4ccccc4)nc(-c4ccccc4-n4c5ccccc5c5ccc(-n6c7ccccc7c7c(-c8ccccc8)cccc76)cc54)n3)cc2)cc1]

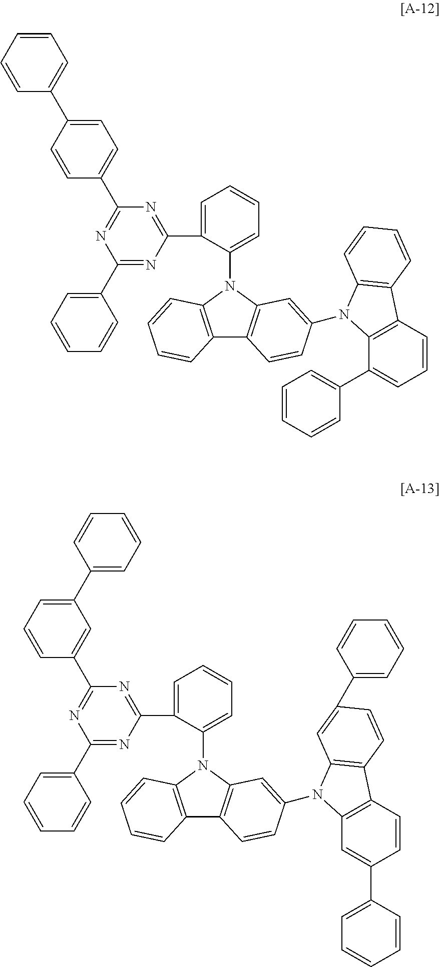 [SMILES: c1ccc(-c2ccc(-c3nc(-c4ccccc4)nc(-c4ccccc4-n4c5ccccc5c5ccc(-n6c7ccccc7c7cccc(-c8ccccc8)c76)cc54)n3)cc2)cc1 ; c1ccc(-c2cccc(-c3nc(-c4ccccc4)nc(-c4ccccc4-n4c5ccccc5c5ccc(-n6c7cc(-c8ccccc8)ccc7c7ccc(-c8ccccc8)cc76)cc54)n3)c2)cc1]

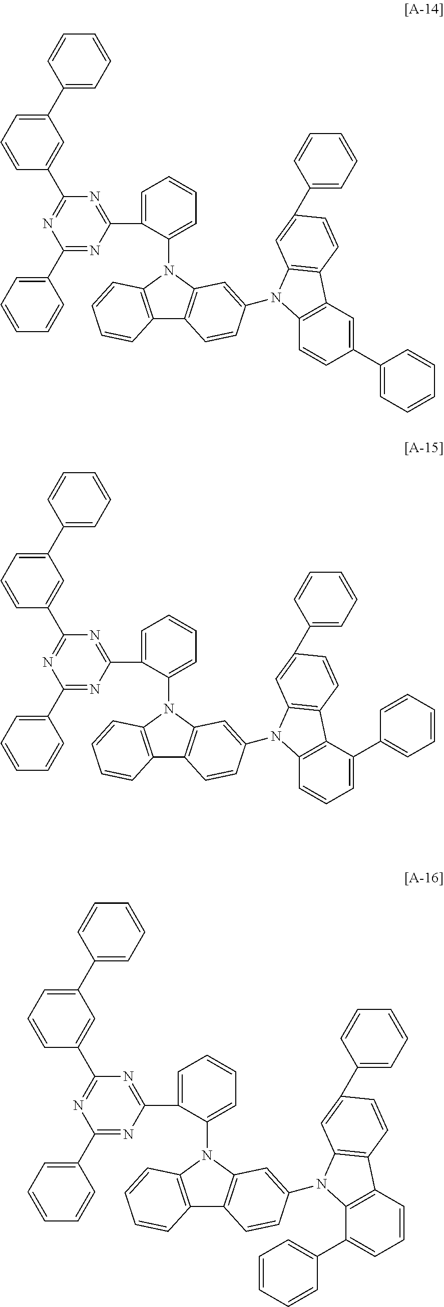 [SMILES: c1ccc(-c2cccc(-c3nc(-c4ccccc4)nc(-c4ccccc4-n4c5ccccc5c5ccc(-n6c7ccc(-c8ccccc8)cc7c7ccc(-c8ccccc8)cc76)cc54)n3)c2)cc1 ; c1ccc(-c2cccc(-c3nc(-c4ccccc4)nc(-c4ccccc4-n4c5ccccc5c5ccc(-n6c7cc(-c8ccccc8)ccc7c7c(-c8ccccc8)cccc76)cc54)n3)c2)cc1 ; c1ccc(-c2cccc(-c3nc(-c4ccccc4)nc(-c4ccccc4-n4c5ccccc5c5ccc(-n6c7cc(-c8ccccc8)ccc7c7cccc(-c8ccccc8)c76)cc54)n3)c2)cc1]

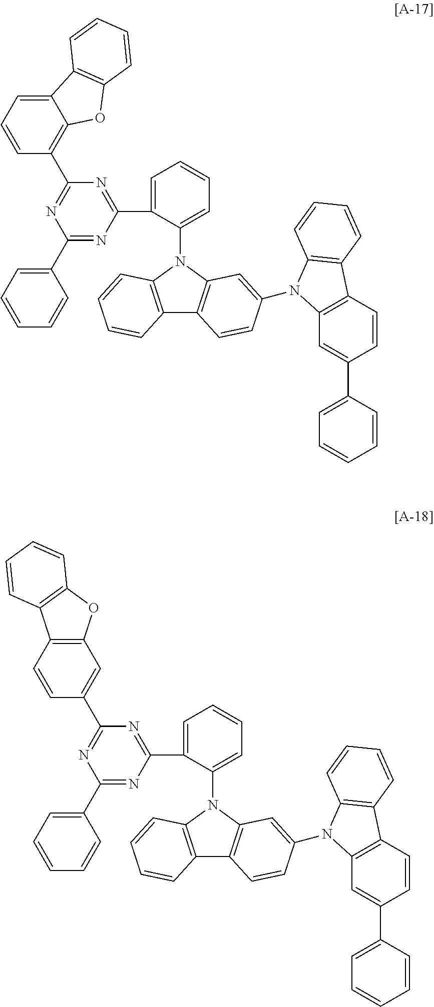 [SMILES: c1ccc(-c2ccc3c4ccccc4n(-c4ccc5c6ccccc6n(-c6ccccc6-c6nc(-c7ccccc7)nc(-c7cccc8c7oc7ccccc78)n6)c5c4)c3c2)cc1 ; c1ccc(-c2ccc3c4ccccc4n(-c4ccc5c6ccccc6n(-c6ccccc6-c6nc(-c7ccccc7)nc(-c7ccc8c(c7)oc7ccccc78)n6)c5c4)c3c2)cc1]

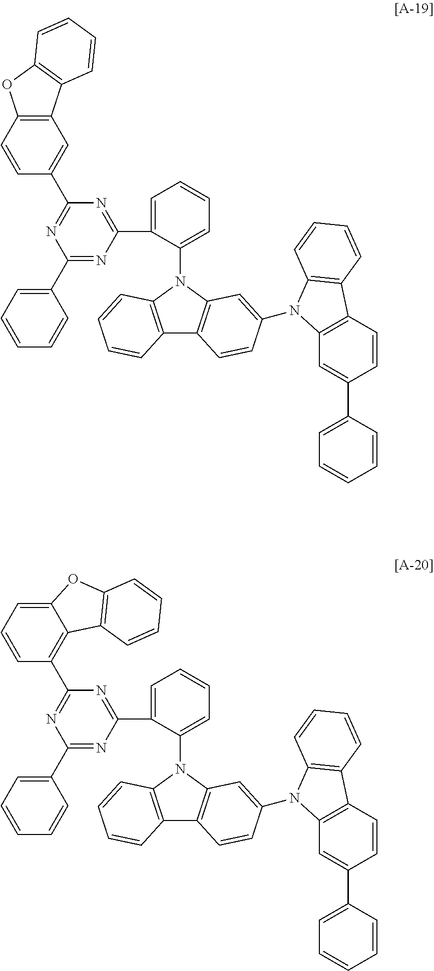 [SMILES: c1ccc(-c2ccc3c4ccccc4n(-c4ccc5c6ccccc6n(-c6ccccc6-c6nc(-c7ccccc7)nc(-c7ccc8oc9ccccc9c8c7)n6)c5c4)c3c2)cc1 ; c1ccc(-c2ccc3c4ccccc4n(-c4ccc5c6ccccc6n(-c6ccccc6-c6nc(-c7ccccc7)nc(-c7cccc8oc9ccccc9c78)n6)c5c4)c3c2)cc1]

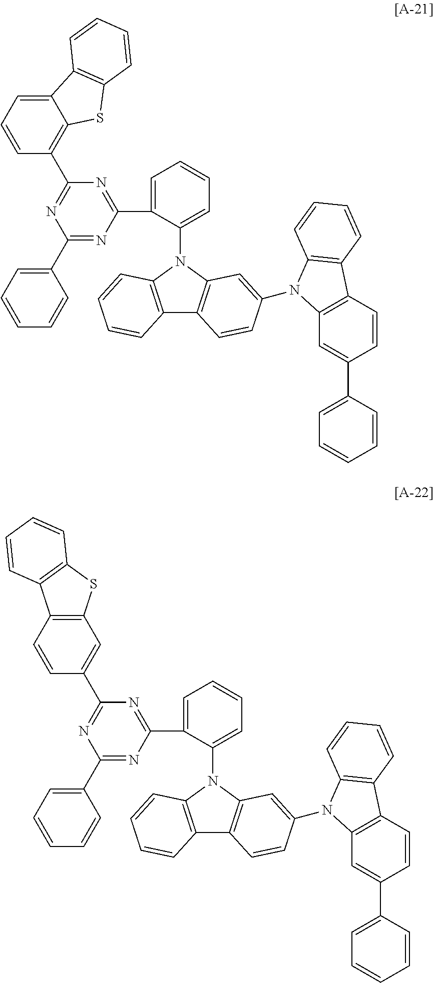 [SMILES: c1ccc(-c2ccc3c4ccccc4n(-c4ccc5c6ccccc6n(-c6ccccc6-c6nc(-c7ccccc7)nc(-c7cccc8c7sc7ccccc78)n6)c5c4)c3c2)cc1 ; c1ccc(-c2ccc3c4ccccc4n(-c4ccc5c6ccccc6n(-c6ccccc6-c6nc(-c7ccccc7)nc(-c7ccc8c(c7)sc7ccccc78)n6)c5c4)c3c2)cc1]

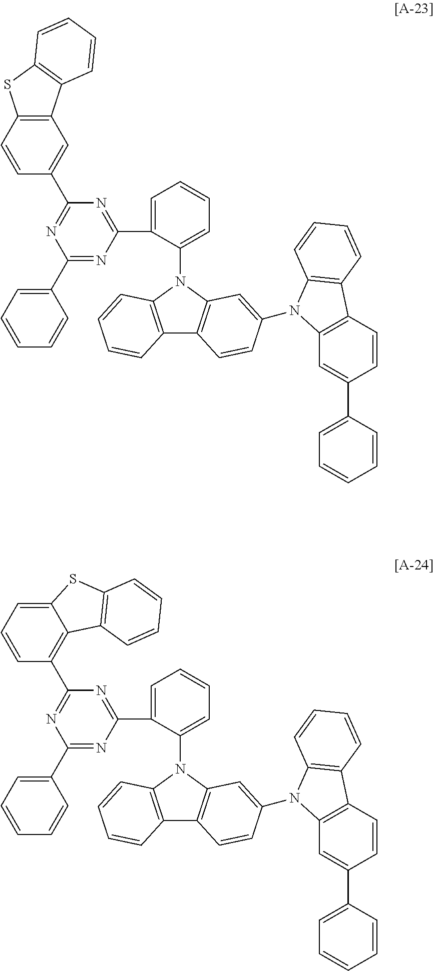 [SMILES: c1ccc(-c2ccc3c4ccccc4n(-c4ccc5c6ccccc6n(-c6ccccc6-c6nc(-c7ccccc7)nc(-c7ccc8sc9ccccc9c8c7)n6)c5c4)c3c2)cc1 ; c1ccc(-c2ccc3c4ccccc4n(-c4ccc5c6ccccc6n(-c6ccccc6-c6nc(-c7ccccc7)nc(-c7cccc8sc9ccccc9c78)n6)c5c4)c3c2)cc1]

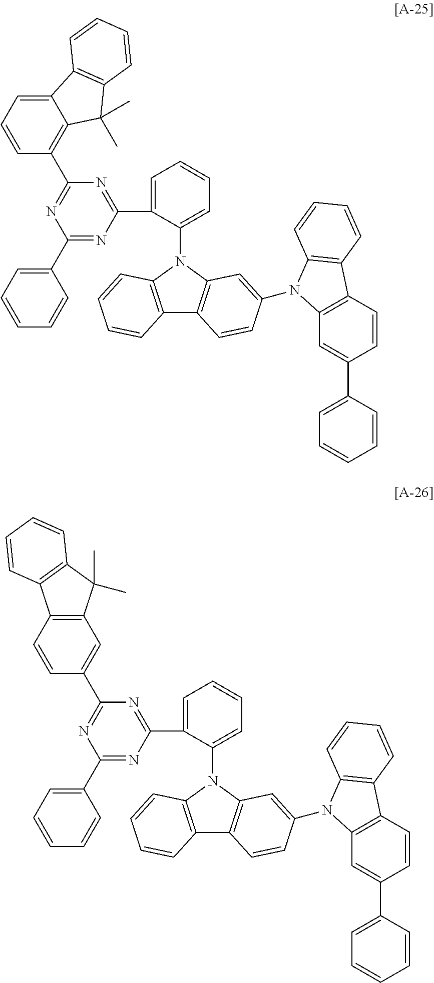 [SMILES: CC1(C)c2ccccc2-c2cccc(-c3nc(-c4ccccc4)nc(-c4ccccc4-n4c5ccccc5c5ccc(-n6c7ccccc7c7ccc(-c8ccccc8)cc76)cc54)n3)c21 ; CC1(C)c2ccccc2-c2ccc(-c3nc(-c4ccccc4)nc(-c4ccccc4-n4c5ccccc5c5ccc(-n6c7ccccc7c7ccc(-c8ccccc8)cc76)cc54)n3)cc21]

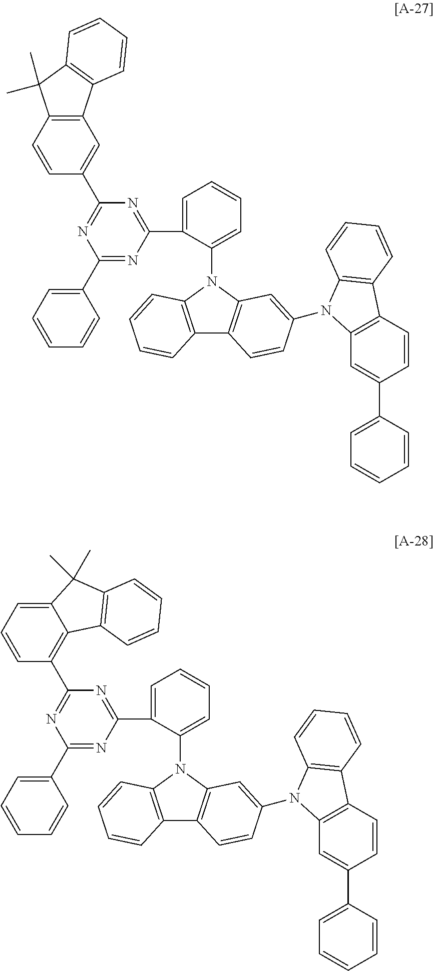 [SMILES: CC1(C)c2ccccc2-c2cc(-c3nc(-c4ccccc4)nc(-c4ccccc4-n4c5ccccc5c5ccc(-n6c7ccccc7c7ccc(-c8ccccc8)cc76)cc54)n3)ccc21 ; CC1(C)c2ccccc2-c2c(-c3nc(-c4ccccc4)nc(-c4ccccc4-n4c5ccccc5c5ccc(-n6c7ccccc7c7ccc(-c8ccccc8)cc76)cc54)n3)cccc21]

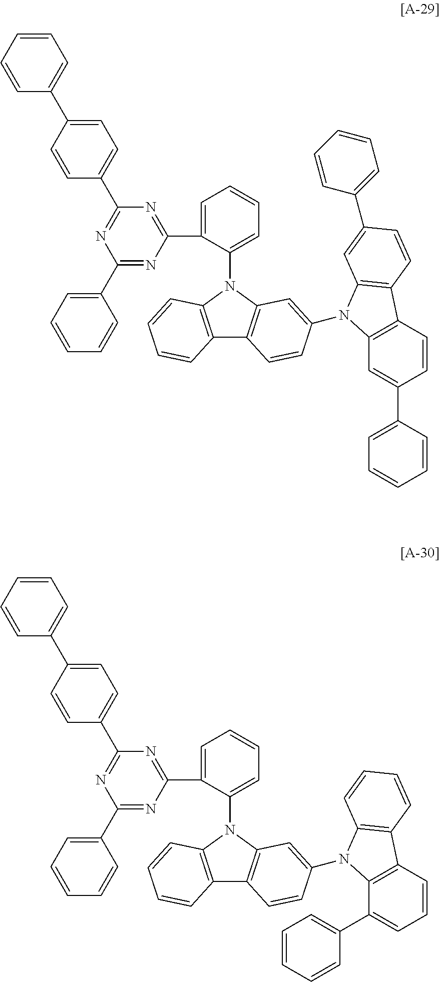 [SMILES: c1ccc(-c2ccc(-c3nc(-c4ccccc4)nc(-c4ccccc4-n4c5ccccc5c5ccc(-n6c7cc(-c8ccccc8)ccc7c7ccc(-c8ccccc8)cc76)cc54)n3)cc2)cc1 ; c1ccc(-c2ccc(-c3nc(-c4ccccc4)nc(-c4ccccc4-n4c5ccccc5c5ccc(-n6c7ccccc7c7cccc(-c8ccccc8)c76)cc54)n3)cc2)cc1]

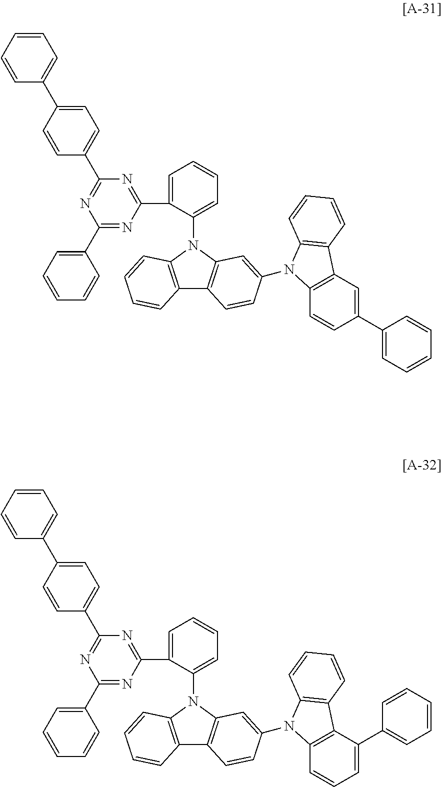 [SMILES: c1ccc(-c2ccc(-c3nc(-c4ccccc4)nc(-c4ccccc4-n4c5ccccc5c5ccc(-n6c7ccccc7c7cc(-c8ccccc8)ccc76)cc54)n3)cc2)cc1 ; c1ccc(-c2ccc(-c3nc(-c4ccccc4)nc(-c4ccccc4-n4c5ccccc5c5ccc(-n6c7ccccc7c7c(-c8ccccc8)cccc76)cc54)n3)cc2)cc1]

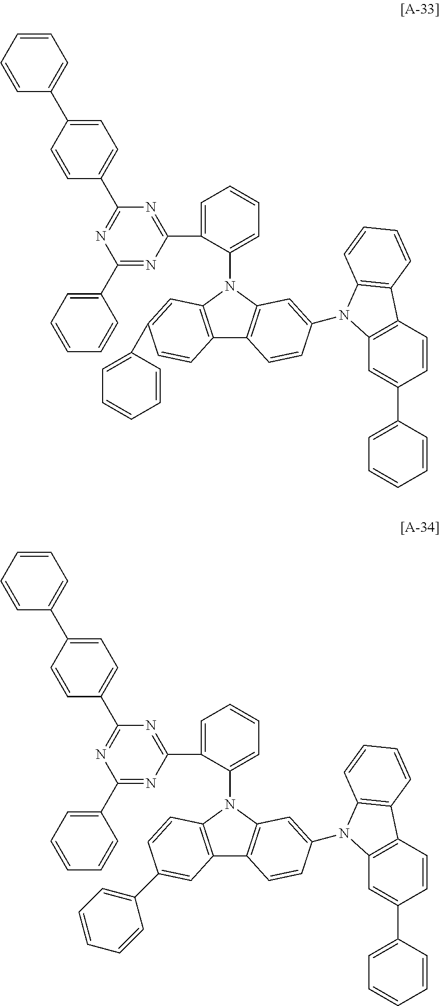 [SMILES: c1ccc(-c2ccc(-c3nc(-c4ccccc4)nc(-c4ccccc4-n4c5cc(-c6ccccc6)ccc5c5ccc(-n6c7ccccc7c7ccc(-c8ccccc8)cc76)cc54)n3)cc2)cc1 ; c1ccc(-c2ccc(-c3nc(-c4ccccc4)nc(-c4ccccc4-n4c5ccc(-c6ccccc6)cc5c5ccc(-n6c7ccccc7c7ccc(-c8ccccc8)cc76)cc54)n3)cc2)cc1]

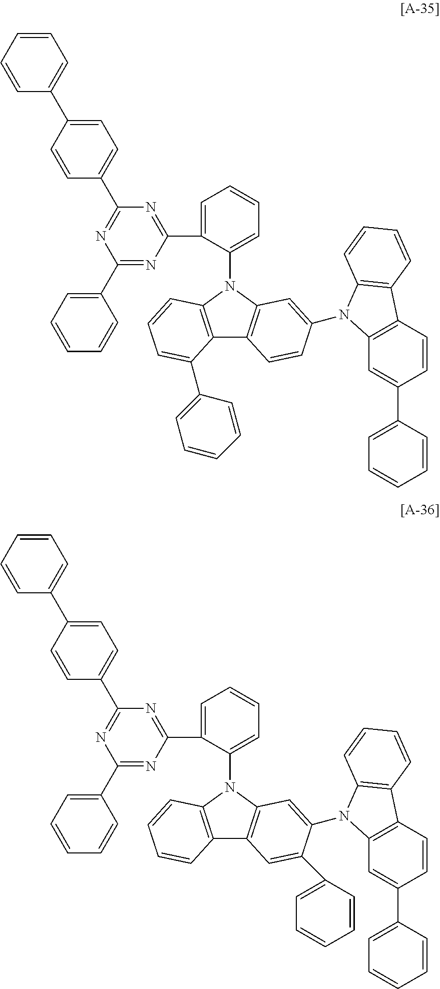 [SMILES: c1ccc(-c2ccc(-c3nc(-c4ccccc4)nc(-c4ccccc4-n4c5cc(-n6c7ccccc7c7ccc(-c8ccccc8)cc76)ccc5c5c(-c6ccccc6)cccc54)n3)cc2)cc1 ; c1ccc(-c2ccc(-c3nc(-c4ccccc4)nc(-c4ccccc4-n4c5ccccc5c5cc(-c6ccccc6)c(-n6c7ccccc7c7ccc(-c8ccccc8)cc76)cc54)n3)cc2)cc1]

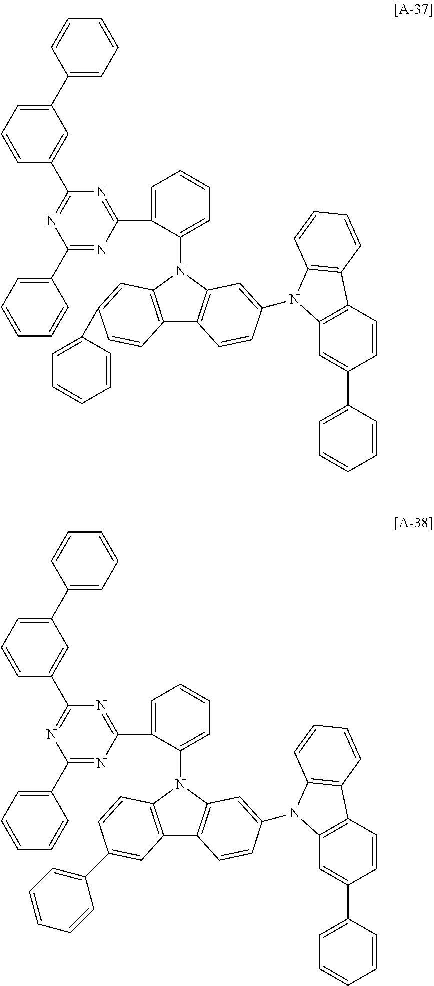 [SMILES: c1ccc(-c2cccc(-c3nc(-c4ccccc4)nc(-c4ccccc4-n4c5cc(-c6ccccc6)ccc5c5ccc(-n6c7ccccc7c7ccc(-c8ccccc8)cc76)cc54)n3)c2)cc1 ; c1ccc(-c2cccc(-c3nc(-c4ccccc4)nc(-c4ccccc4-n4c5ccc(-c6ccccc6)cc5c5ccc(-n6c7ccccc7c7ccc(-c8ccccc8)cc76)cc54)n3)c2)cc1]

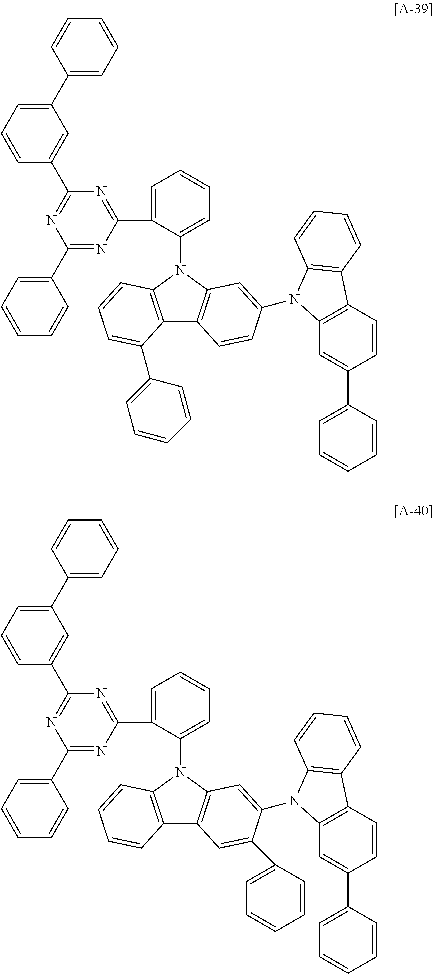 [SMILES: c1ccc(-c2cccc(-c3nc(-c4ccccc4)nc(-c4ccccc4-n4c5cc(-n6c7ccccc7c7ccc(-c8ccccc8)cc76)ccc5c5c(-c6ccccc6)cccc54)n3)c2)cc1 ; c1ccc(-c2cccc(-c3nc(-c4ccccc4)nc(-c4ccccc4-n4c5ccccc5c5cc(-c6ccccc6)c(-n6c7ccccc7c7ccc(-c8ccccc8)cc76)cc54)n3)c2)cc1]

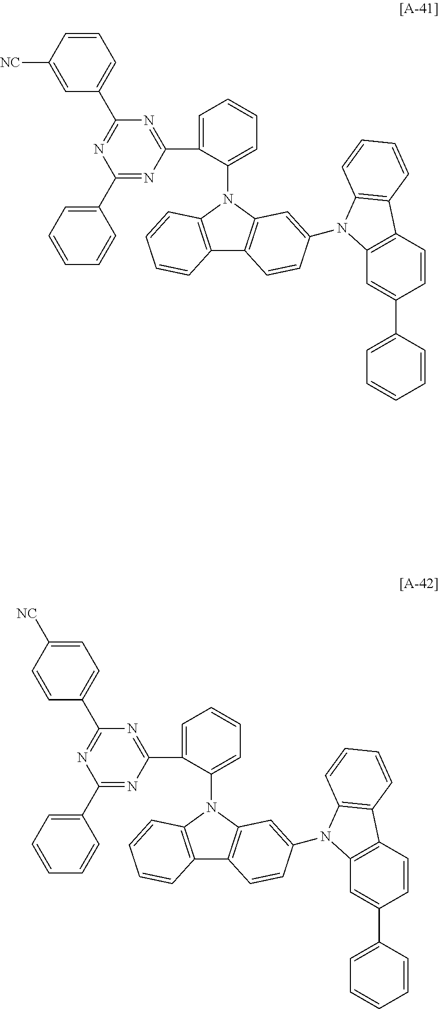 [SMILES: N#Cc1cccc(-c2nc(-c3ccccc3)nc(-c3ccccc3-n3c4ccccc4c4ccc(-n5c6ccccc6c6ccc(-c7ccccc7)cc65)cc43)n2)c1 ; N#Cc1ccc(-c2nc(-c3ccccc3)nc(-c3ccccc3-n3c4ccccc4c4ccc(-n5c6ccccc6c6ccc(-c7ccccc7)cc65)cc43)n2)cc1]

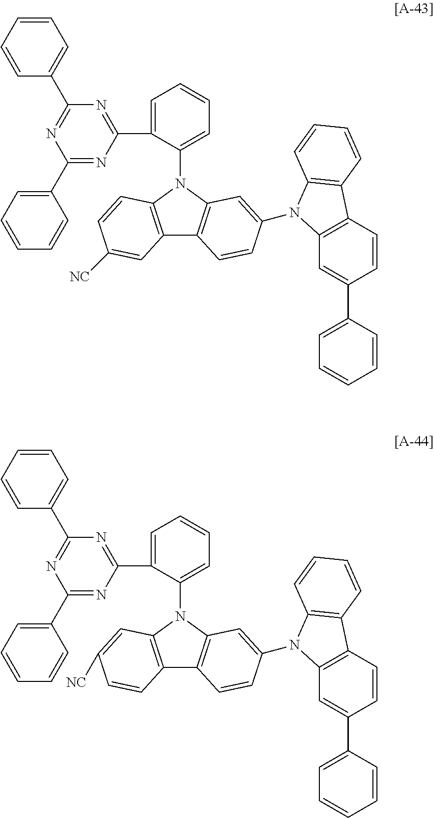 [SMILES: N#Cc1ccc2c(c1)c1ccc(-n3c4ccccc4c4ccc(-c5ccccc5)cc43)cc1n2-c1ccccc1-c1nc(-c2ccccc2)nc(-c2ccccc2)n1 ; N#Cc1ccc2c3ccc(-n4c5ccccc5c5ccc(-c6ccccc6)cc54)cc3n(-c3ccccc3-c3nc(-c4ccccc4)nc(-c4ccccc4)n3)c2c1]

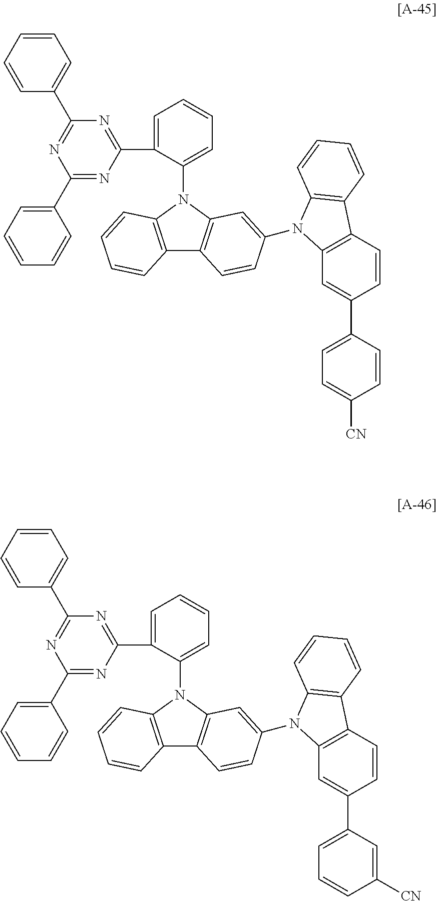 [SMILES: N#Cc1ccc(-c2ccc3c4ccccc4n(-c4ccc5c6ccccc6n(-c6ccccc6-c6nc(-c7ccccc7)nc(-c7ccccc7)n6)c5c4)c3c2)cc1 ; N#Cc1cccc(-c2ccc3c4ccccc4n(-c4ccc5c6ccccc6n(-c6ccccc6-c6nc(-c7ccccc7)nc(-c7ccccc7)n6)c5c4)c3c2)c1]

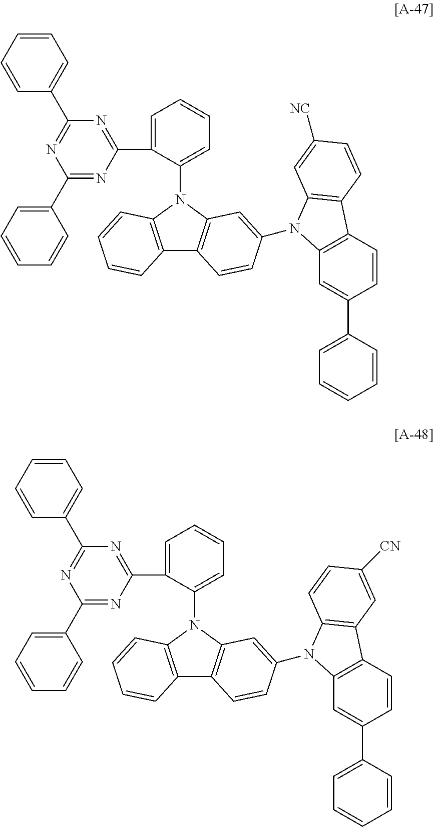 [SMILES: N#Cc1ccc2c3ccc(-c4ccccc4)cc3n(-c3ccc4c5ccccc5n(-c5ccccc5-c5nc(-c6ccccc6)nc(-c6ccccc6)n5)c4c3)c2c1 ; N#Cc1ccc2c(c1)c1ccc(-c3ccccc3)cc1n2-c1ccc2c3ccccc3n(-c3ccccc3-c3nc(-c4ccccc4)nc(-c4ccccc4)n3)c2c1]

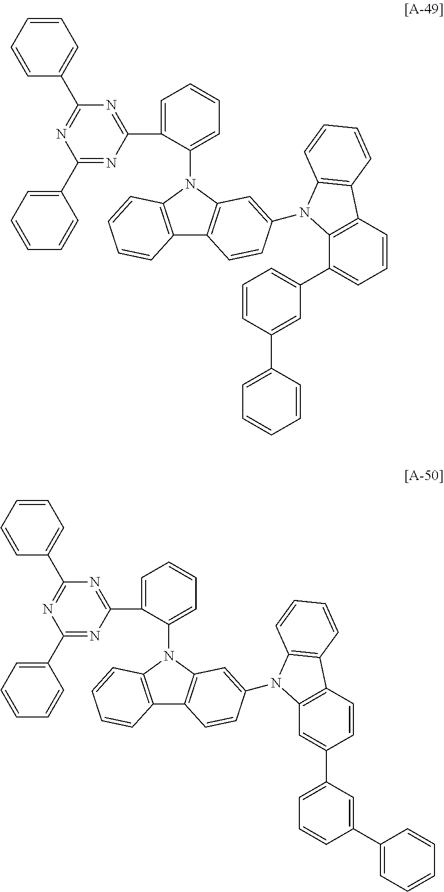 [SMILES: c1ccc(-c2cccc(-c3cccc4c5ccccc5n(-c5ccc6c7ccccc7n(-c7ccccc7-c7nc(-c8ccccc8)nc(-c8ccccc8)n7)c6c5)c34)c2)cc1 ; c1ccc(-c2cccc(-c3ccc4c5ccccc5n(-c5ccc6c7ccccc7n(-c7ccccc7-c7nc(-c8ccccc8)nc(-c8ccccc8)n7)c6c5)c4c3)c2)cc1]

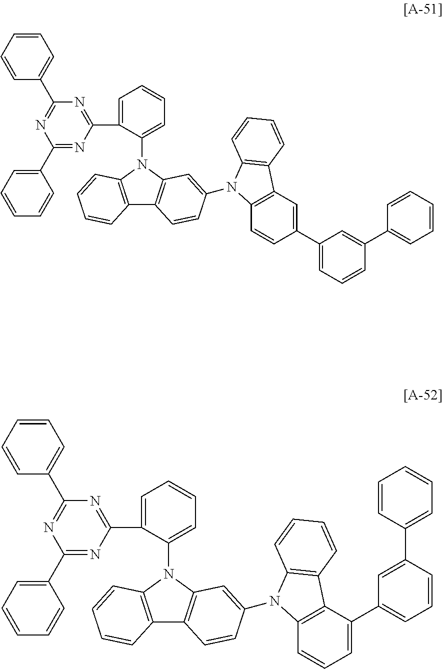 [SMILES: c1ccc(-c2cccc(-c3ccc4c(c3)c3ccccc3n4-c3ccc4c5ccccc5n(-c5ccccc5-c5nc(-c6ccccc6)nc(-c6ccccc6)n5)c4c3)c2)cc1 ; c1ccc(-c2cccc(-c3cccc4c3c3ccccc3n4-c3ccc4c5ccccc5n(-c5ccccc5-c5nc(-c6ccccc6)nc(-c6ccccc6)n5)c4c3)c2)cc1]

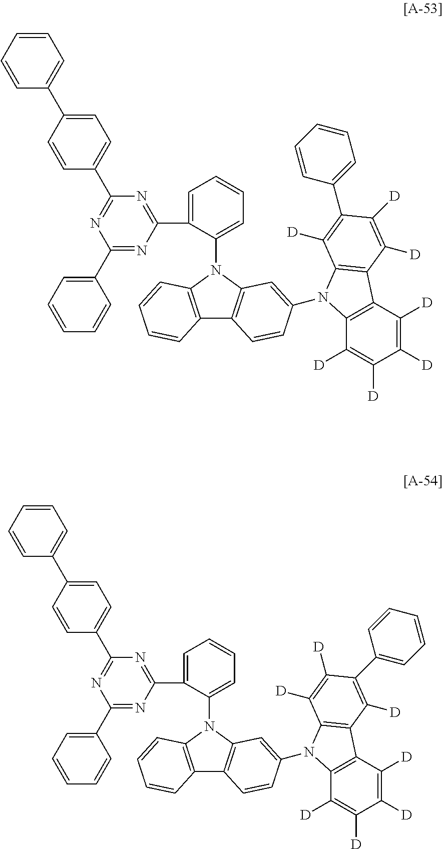 [SMILES: [2H]c1c([2H])c([2H])c2c(c1[2H])c1c([2H])c([2H])c(-c3ccccc3)c([2H])c1n2-c1ccc2c3ccccc3n(-c3ccccc3-c3nc(-c4ccccc4)nc(-c4ccc(-c5ccccc5)cc4)n3)c2c1 ; [2H]c1c([2H])c([2H])c2c(c1[2H])c1c([2H])c(-c3ccccc3)c([2H])c([2H])c1n2-c1ccc2c3ccccc3n(-c3ccccc3-c3nc(-c4ccccc4)nc(-c4ccc(-c5ccccc5)cc4)n3)c2c1]

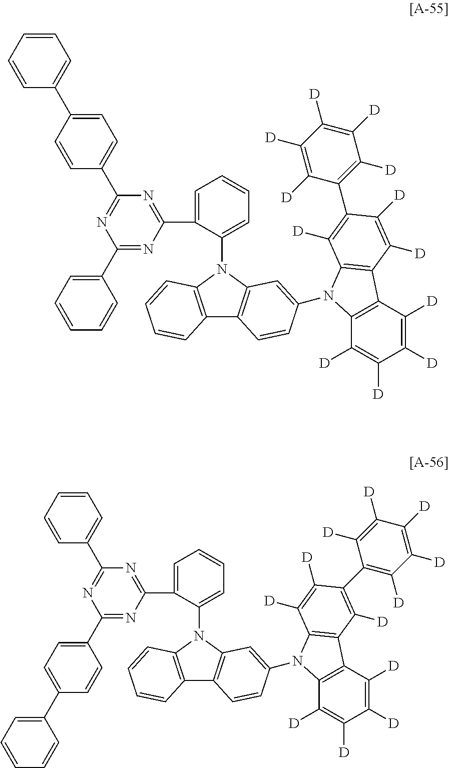 [SMILES: [2H]c1c([2H])c([2H])c(-c2c([2H])c([2H])c3c4c([2H])c([2H])c([2H])c([2H])c4n(-c4ccc5c6ccccc6n(-c6ccccc6-c6nc(-c7ccccc7)nc(-c7ccc(-c8ccccc8)cc7)n6)c5c4)c3c2[2H])c([2H])c1[2H] ; [2H]c1c([2H])c([2H])c(-c2c([2H])c([2H])c3c(c2[2H])c2c([2H])c([2H])c([2H])c([2H])c2n3-c2ccc3c4ccccc4n(-c4ccccc4-c4nc(-c5ccccc5)nc(-c5ccc(-c6ccccc6)cc5)n4)c3c2)c([2H])c1[2H]]

A composition for an organic optoelectronic device according to another embodiment includes a first compound and a second compound, wherein the first compound may be the compound for an organic optoelectronic device described above, and the second compound may be a compound for an organic optoelectronic device represented by Chemical Formula 2; or a compound for organic optoelectronic devices represented by a combination of Chemical Formula 3 and Chemical Formula 4.

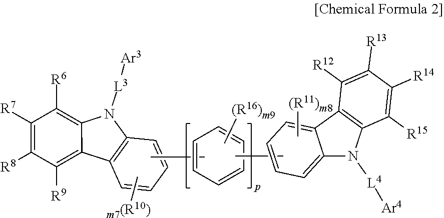

In Chemical Formula 2,
Ar³ and Ar⁴ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁶ to R¹⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m7 and m8 are each independently one of integers of 1 to 3,
m9 is one of integers of 1 to 4, and
p is one of integers of 0 to 2;

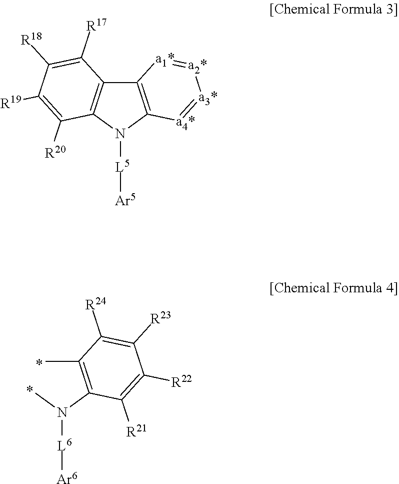

wherein, in Chemical Formulas 3 and 4,
Ar⁵ and Ar⁶ are each independently a substituted or unsubstituted C6 to C20 aryl group or a substituted or unsubstituted C₂ to C30 heterocyclic group,
two adjacent ones among a₁* to a₄* in Chemical Formula 3 are each a linking carbon (C) linked to * in Chemical Formula 4,
the remaining two not linked to * in Chemical Formula 4, among a₁* to a₄* in Chemical Formula 3, are each independently C-L^{a}-R^{a},
L^{a}, L⁵, and L⁶ are each independently a single bond or a substituted or unsubstituted C6 to C20 arylene group, and
R^{a} and R¹⁷ to R²⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

The second compound may be used together with the first compound in the light emitting layer to increase charge mobility and stability, thereby improving luminous efficiency and life-span characteristics.

For example, Ar³ and Ar⁴ in Chemical Formula 2 may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,
L³ and L⁴ in Chemical Formula 2 may each independently be a single bond, a substituted or unsubstituted phenylene group, or a substituted or unsubstituted biphenylene group,
R⁶ to R¹⁰ in Chemical Formula 2 may each independently be hydrogen, deuterium, or a substituted or unsubstituted C6 to C12 aryl group, and
p may be 0 or 1.

For example, “substituted” in Chemical Formula 2 refers to replacement of at least one hydrogen by deuterium, a C1 to C4 alkyl group, a C6 to C18 aryl group, or a C2 to C30 heteroaryl group.

In a specific embodiment of the present invention, Chemical Formula 2 may be represented by any one of Chemical Formula 2-1 to Chemical Formula 2-15.

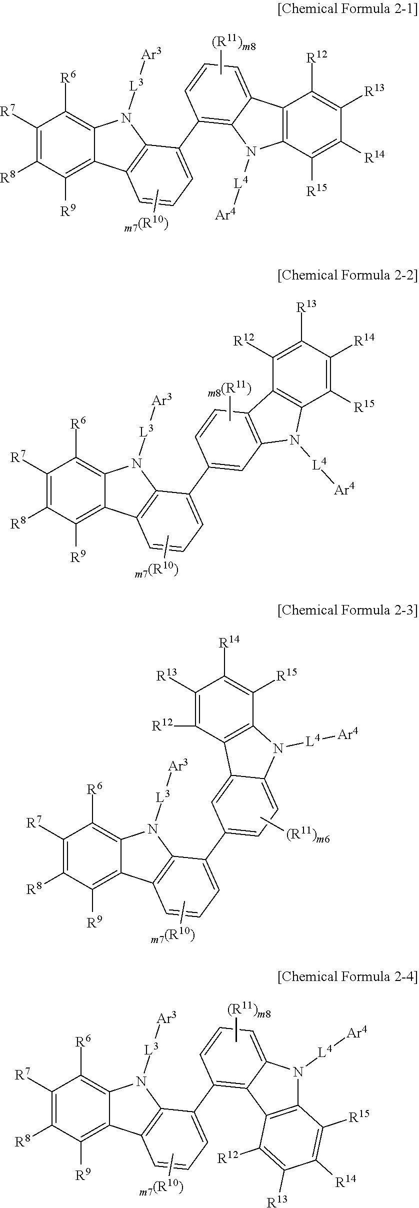

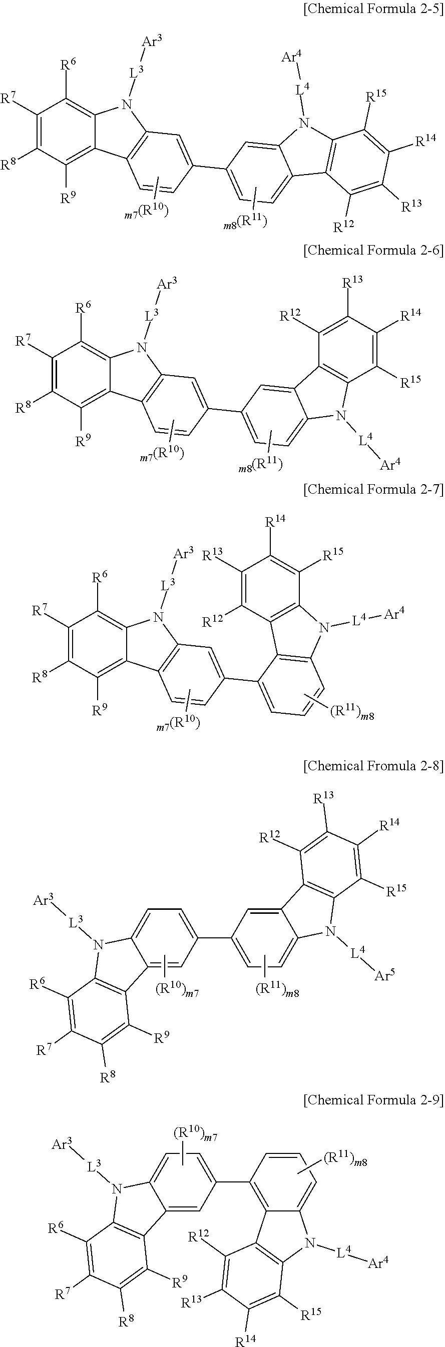

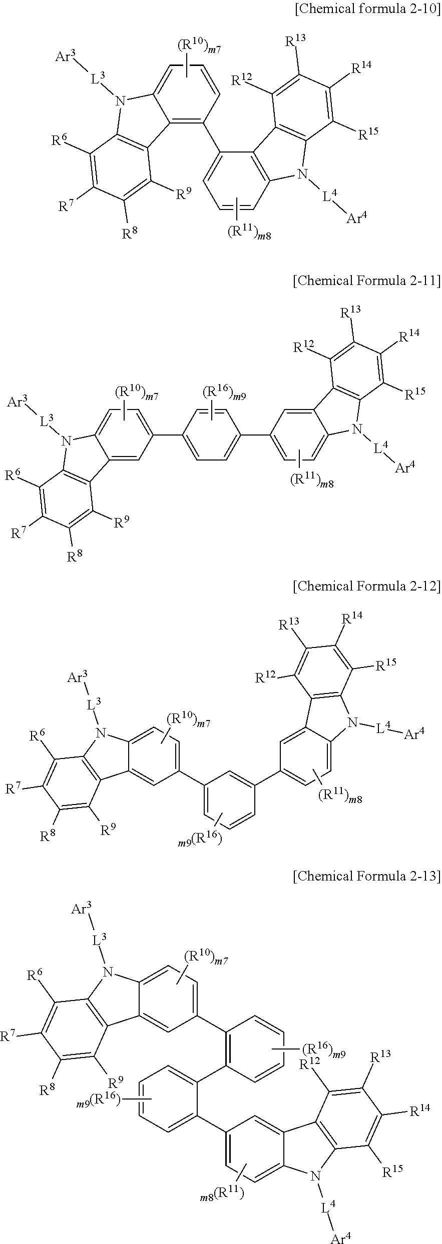

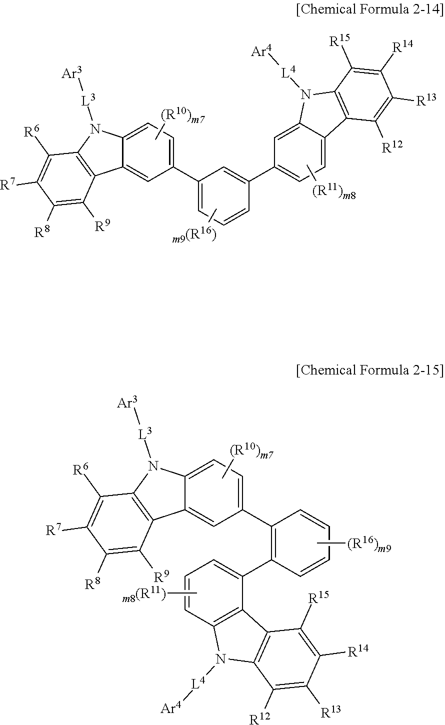

In Chemical Formula 2-1 to Chemical Formula 2-15, R⁶ to R¹⁶ may each independently be hydrogen, deuterium, or a substituted or unsubstituted C6 to C12 aryl group, and *-L³-Ar³ and *-L⁴-Ar⁴ may each independently be one of the substituents listed in Group II.

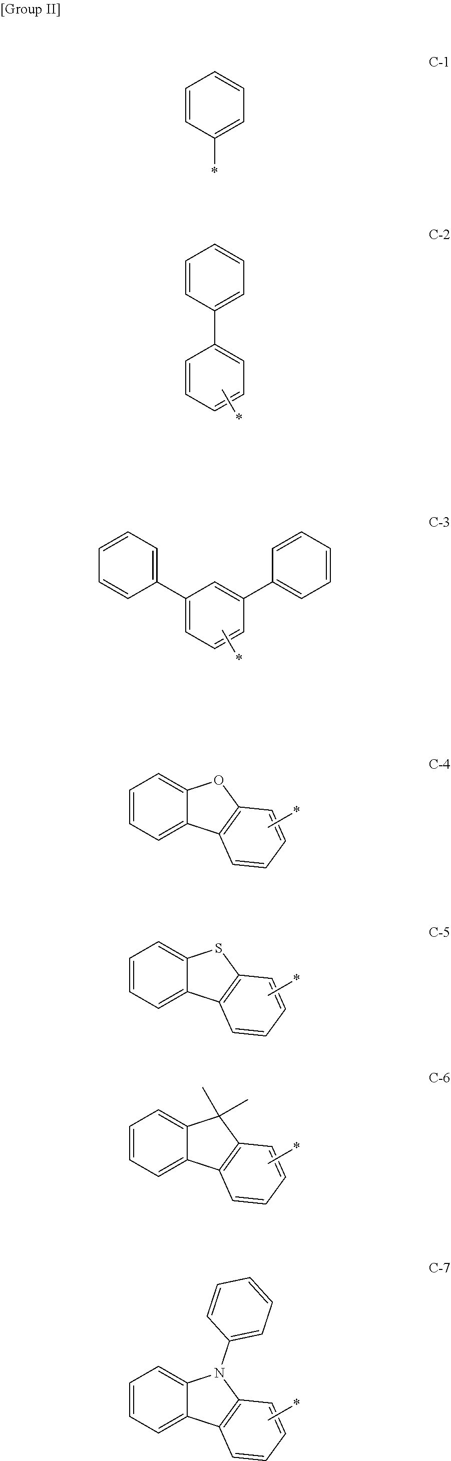

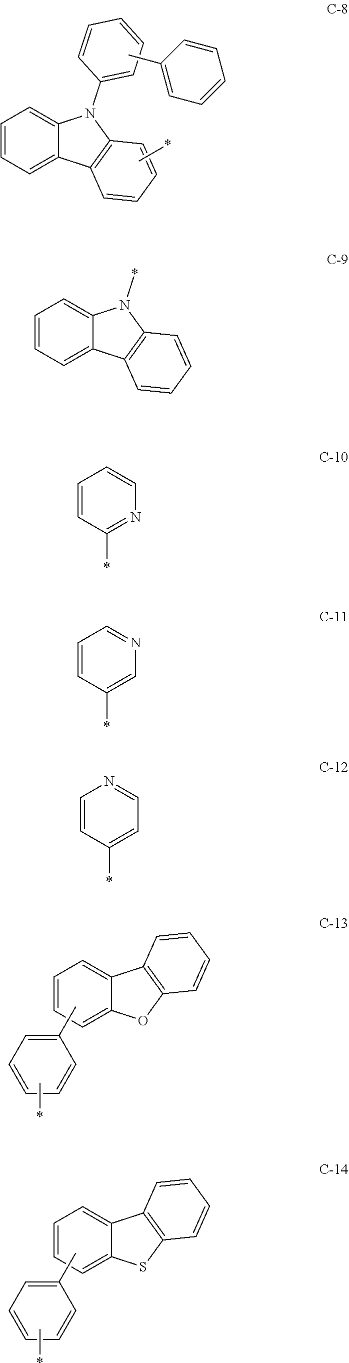

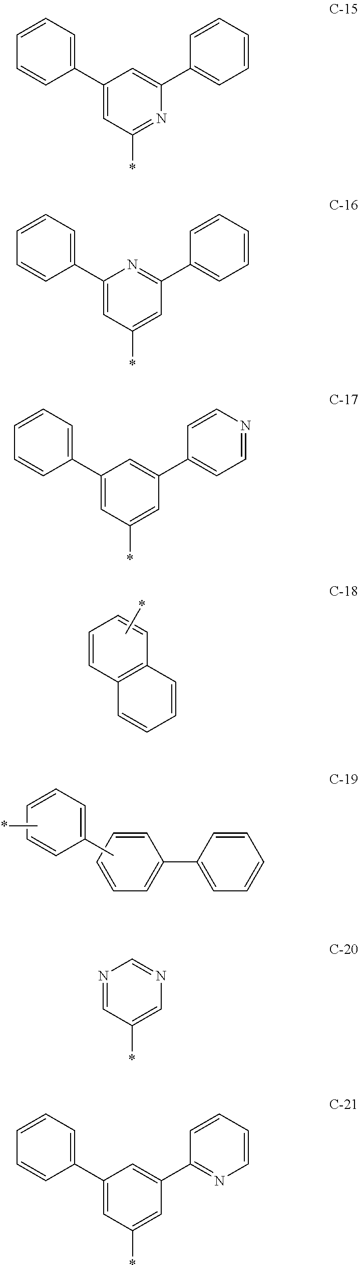

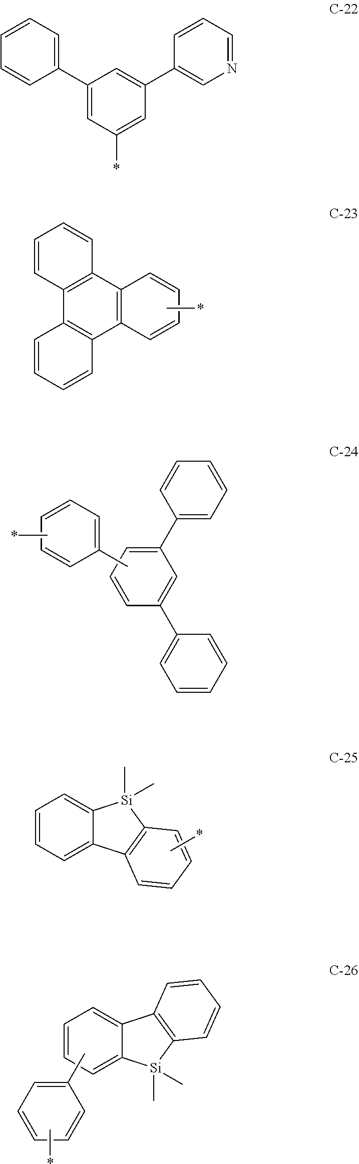

In Group II, the substituent may be unsubstituted or substituted with an additional substituent, and * is a linking point.

The additional substituent may be deuterium, a C1 to C5 alkyl group, a C6 to C18 aryl group, or a C2 to C20 heterocyclic group.

In an embodiment, Chemical Formula 2 may be represented by Chemical Formula 2-8.

In addition, *-L³-Ar³ and *-L⁴-Ar⁴ of Chemical Formula 2-8 may each independently be selected from Group II, and may be for example any one of C-1, C-2, C-3, C-4, C-7, C-8, and C-9.

As an example, the second compound represented by the combination of Chemical Formula 3 and Chemical Formula 4 may be represented by any of Chemical Formula 3A, Chemical Formula 3B, Chemical Formula 3C, Chemical Formula 3D and Chemical Formula 3E.

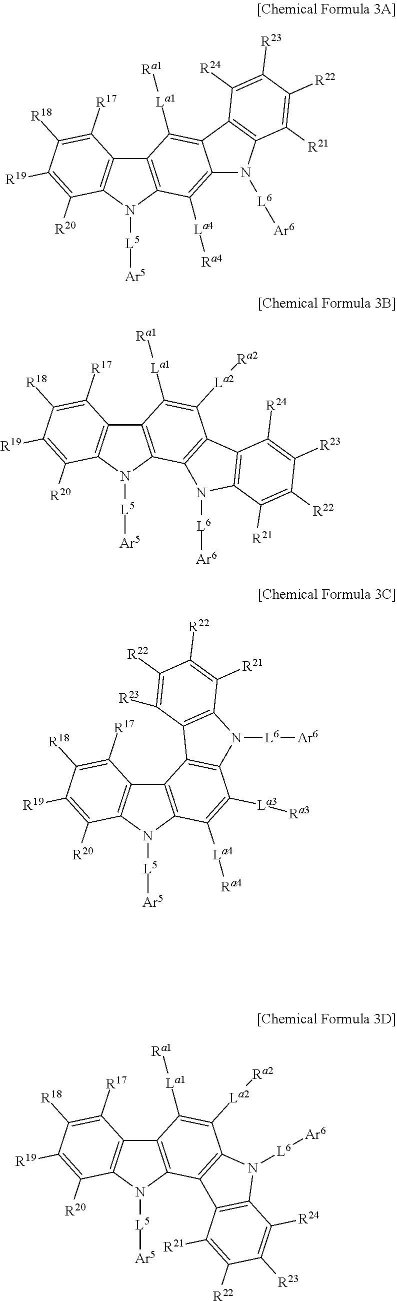

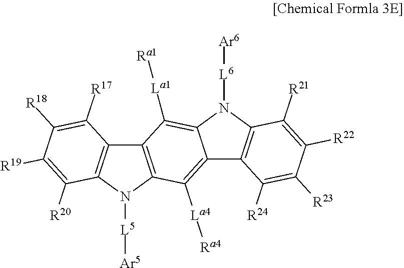

In Chemical Formula 3A to Chemical Formula 3E, Ar⁵, Ar⁶, L³, L⁴, and R¹⁷ to R²⁴ are the same as described above,
L^{a1} to L^{a4} are the same as the definitions of L⁵ and L⁶ described above, and
R^{a1} to R^{a4} are the same as the definitions of R¹⁷ to R²⁴ described above.

For example, in Chemical Formulas 3 and 4, Ar⁵ and Ar⁶ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and
R^{a1} to R^{a4} and R¹⁷ to R²⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In a specific embodiment of the present invention, *-L⁵-Ar⁵ and *-L⁶-Ar⁶ in Chemical Formulas 3 and 4 may each independently be selected from the substituents listed in Group II.

In an embodiment, R^{a1} to R^{a4} and R¹⁷ to R²⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, R^{a1} to R^{a4} and R¹⁷ to R²⁴ may each independently be hydrogen, deuterium, a cyano group, or a substituted or unsubstituted phenyl group,
in a specific embodiment, R^{a1} to R^{a4}, and R¹⁷ to R²⁴ may each independently be hydrogen, deuterium or a phenyl group.

In a specific embodiment of the present invention, the second compound may be represented by Chemical Formula 2-8, wherein in Chemical Formula 2-8, Ar³ and Ar⁴ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L³ and L⁴ may each independently be a single bond, or a substituted or unsubstituted C6 to C20 arylene group, and R⁶ to R¹⁵ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In another specific embodiment of the present invention, the second compound may be represented by Chemical Formula 3C, wherein in Chemical Formula 3C, L^{a3} and L^{a4} may be a single bond, L⁵ and L⁶ may each independently be a single bond or a substituted or unsubstituted C6 to C12 arylene group, R¹⁷ to R²⁴, R^{a3}, and R^{a4} may each independently be hydrogen, deuterium, or a phenyl group, and Ar⁵ and Ar⁶ may each independently be a substituted or unsubstituted phenyl group, a substituted or unsubstituted biphenyl group, or a substituted or unsubstituted terphenyl group.

For example, the second compound may be one selected from the compounds listed in Group 2, but is not limited thereto.

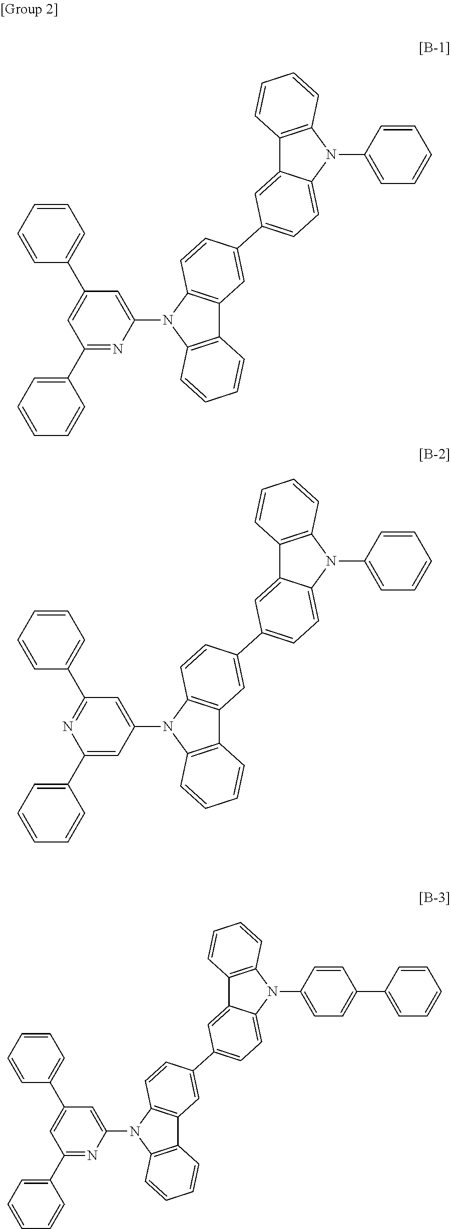 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

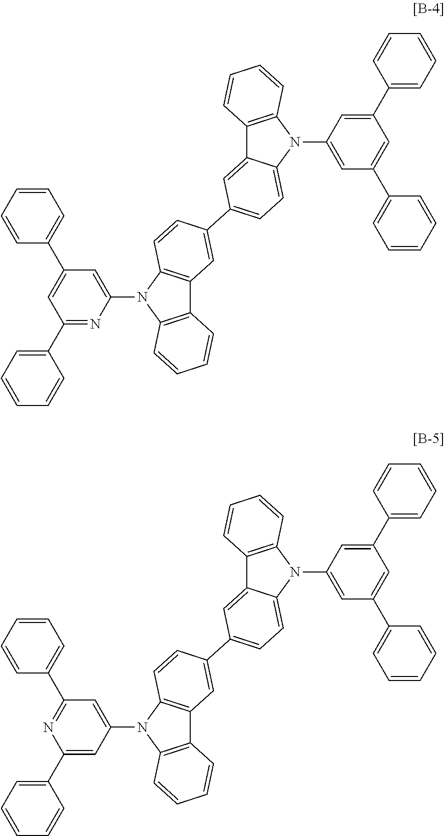 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]

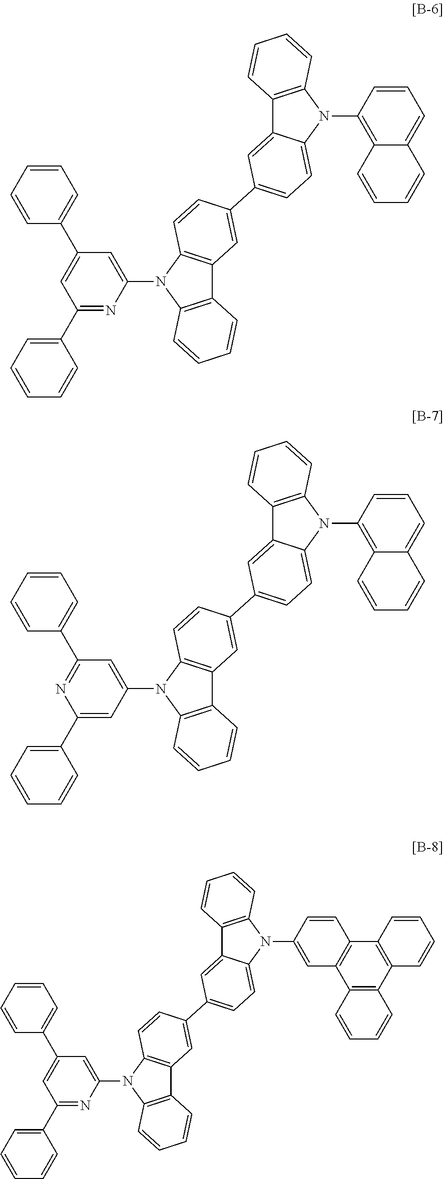 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

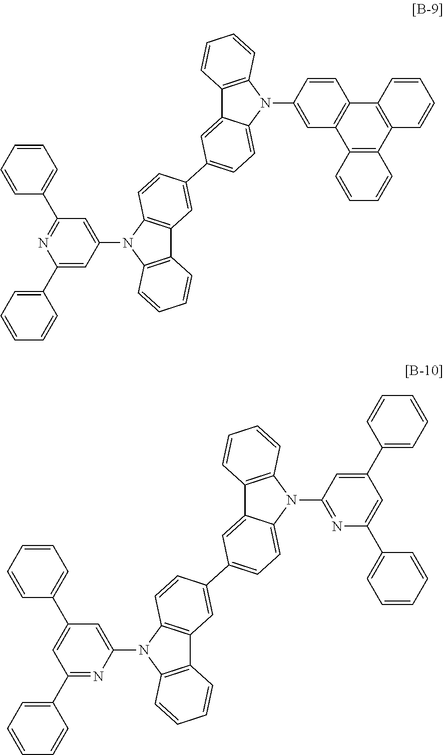 [SMILES: c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1]

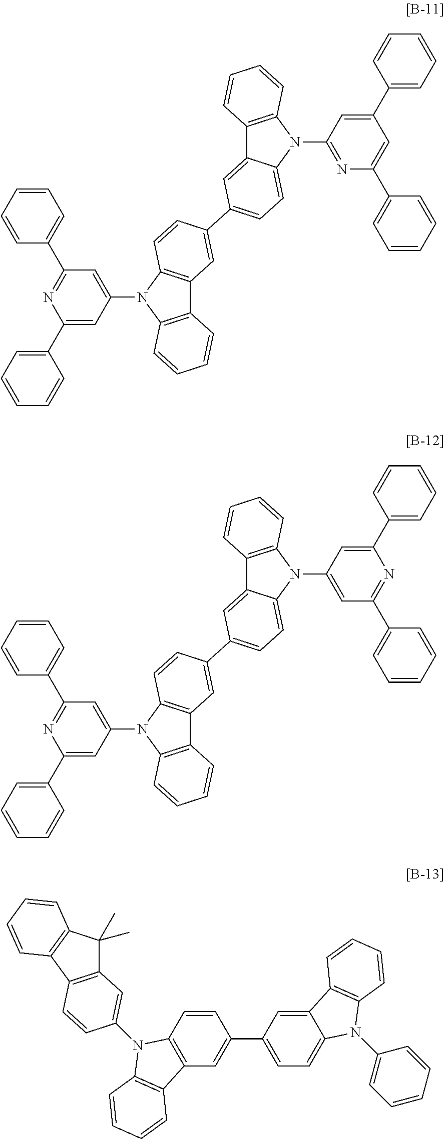 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1 ; CC1(C)c2ccccc2-c2ccc(-n3c4ccccc4c4cc(-c5ccc6c(c5)c5ccccc5n6-c5ccccc5)ccc43)cc21]

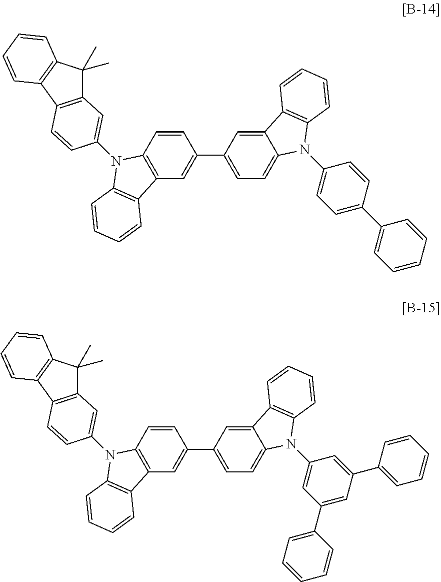 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

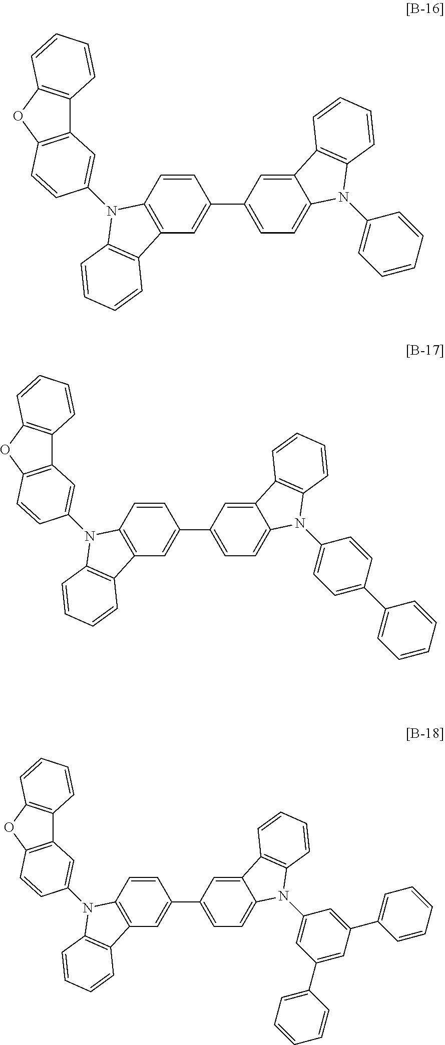 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

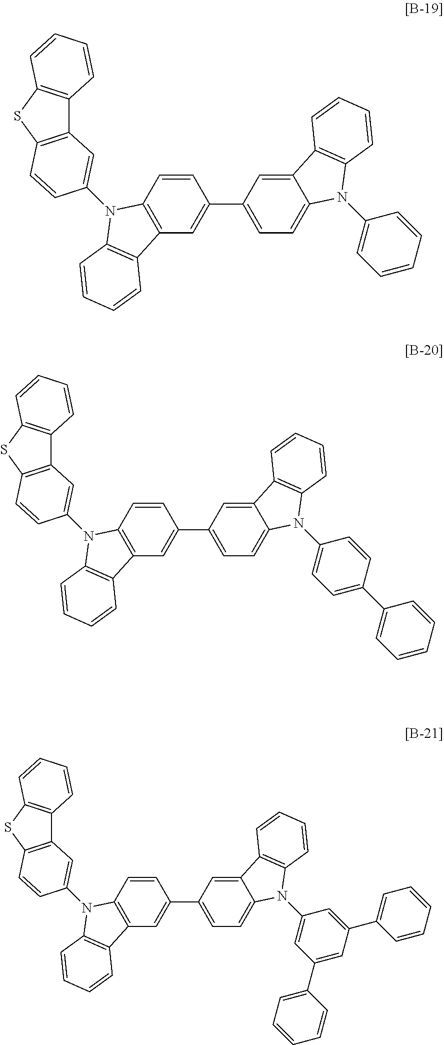 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

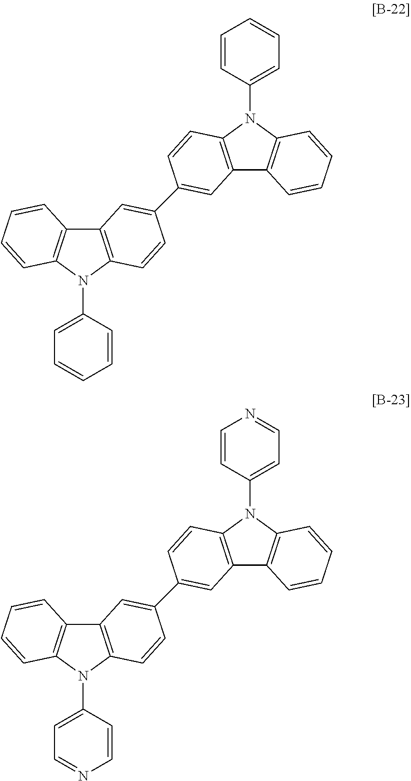 [SMILES: c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1]

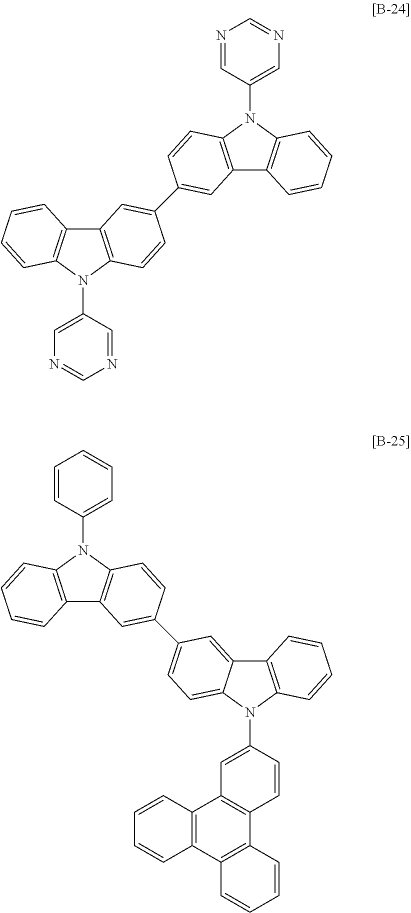 [SMILES: c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1]

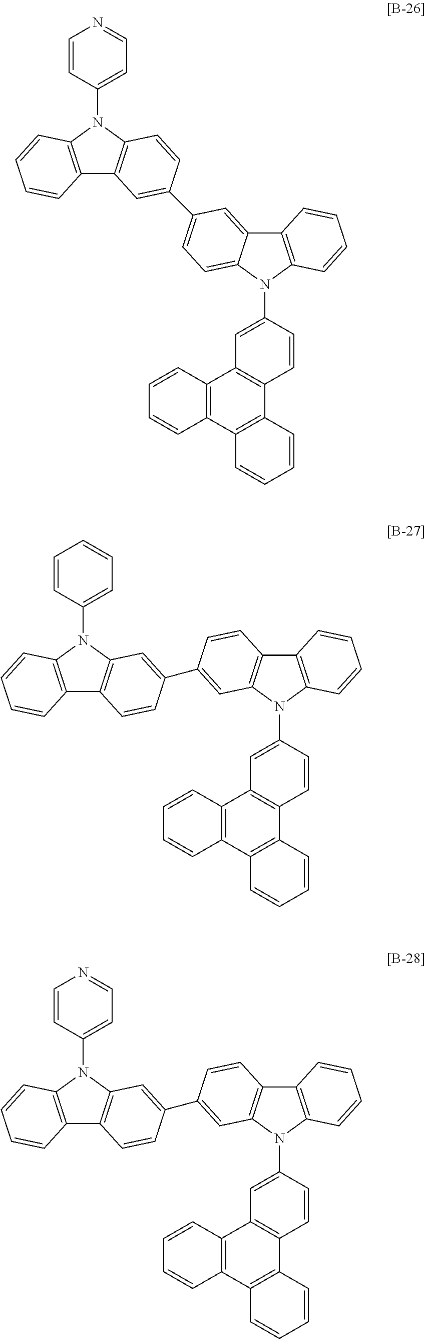 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21]

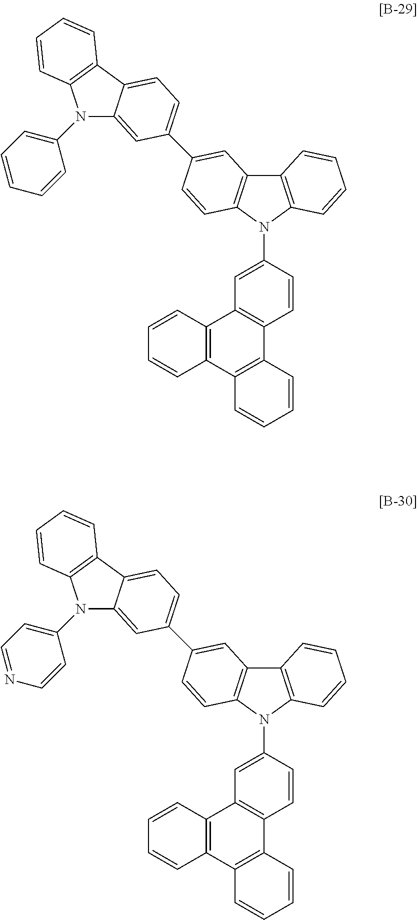 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

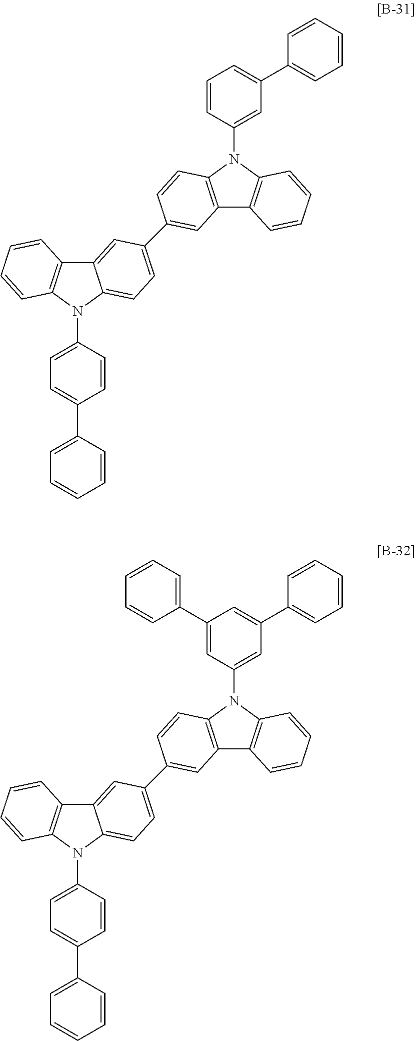 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

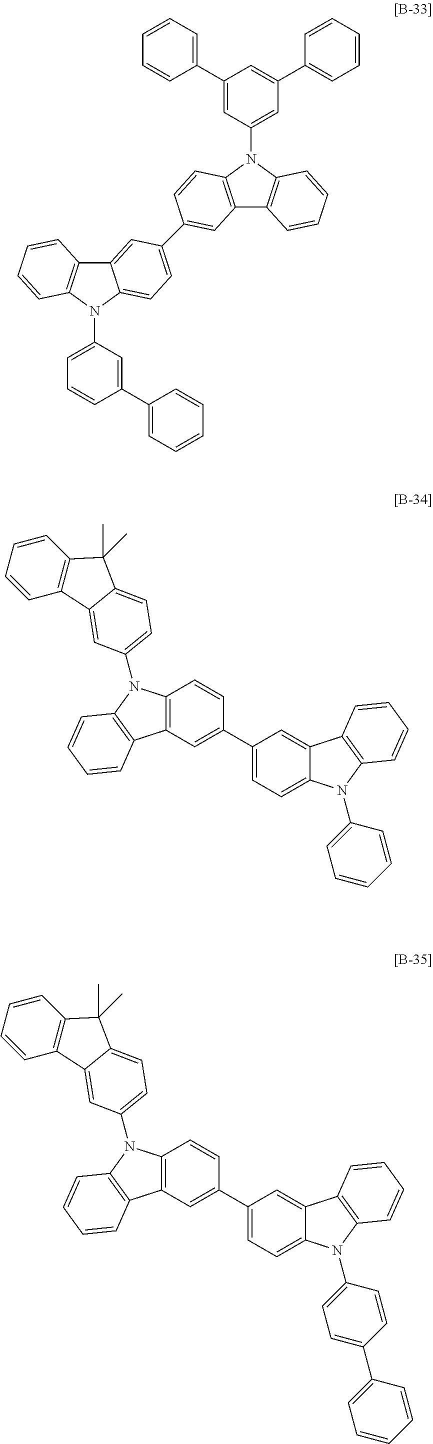 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

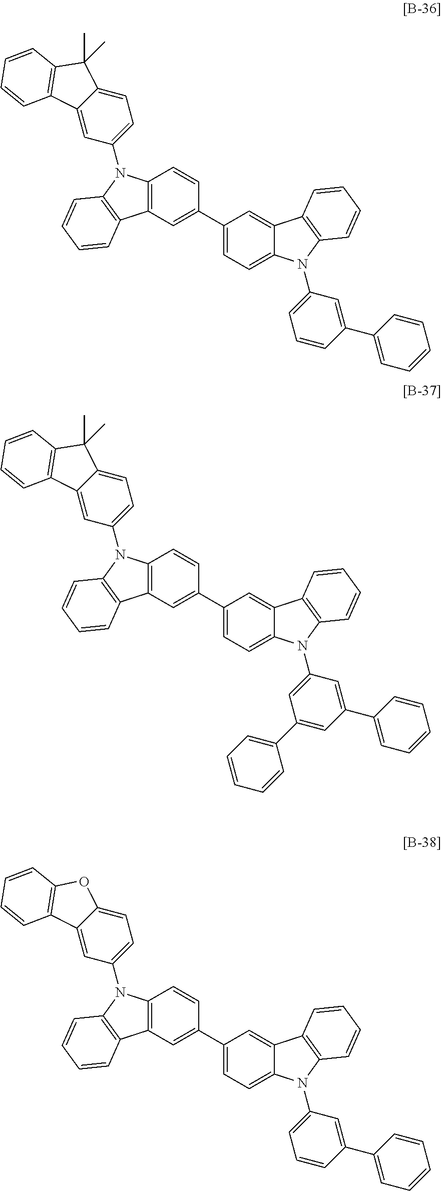 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

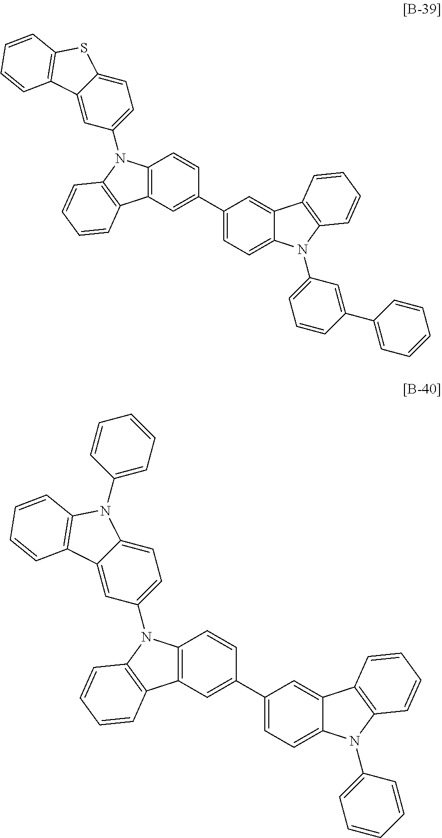 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

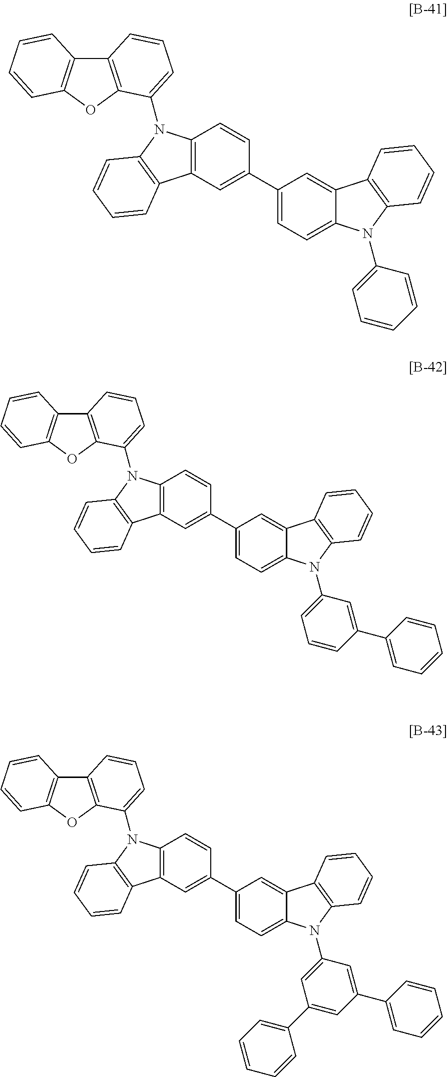 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

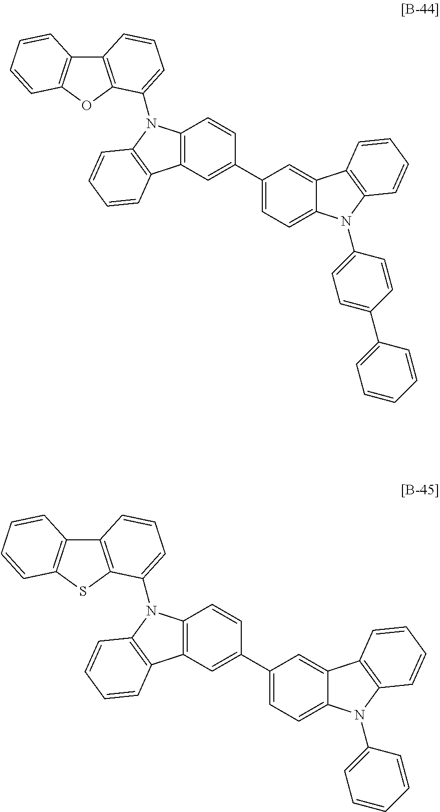 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1]

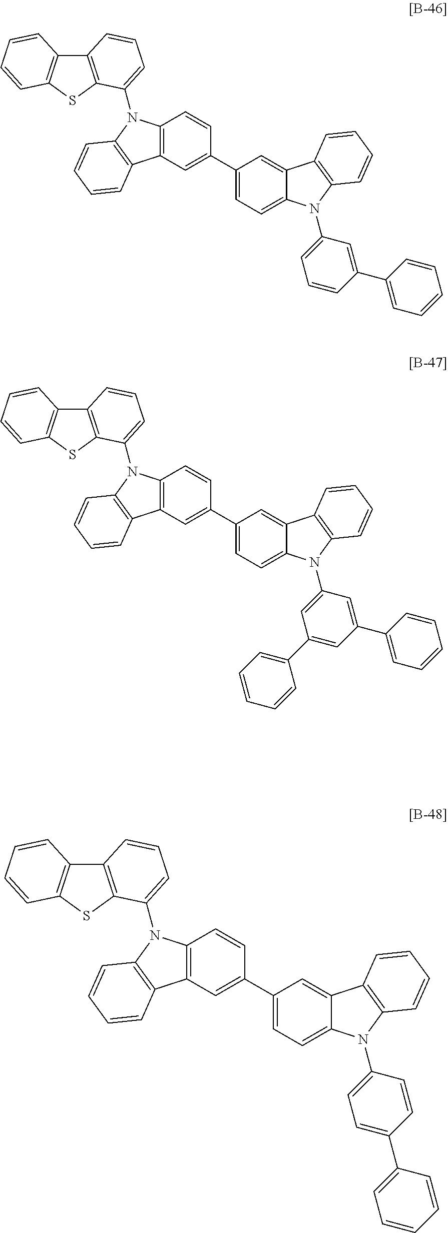 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

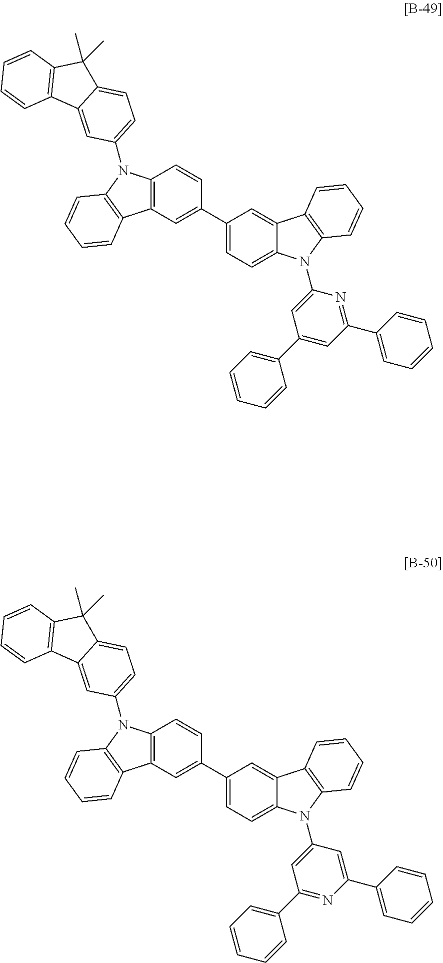 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

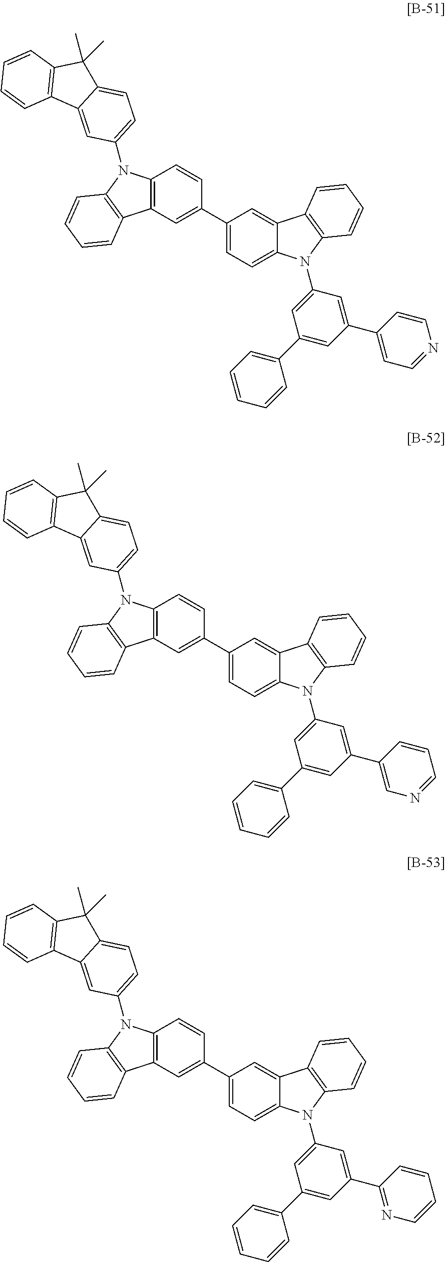 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

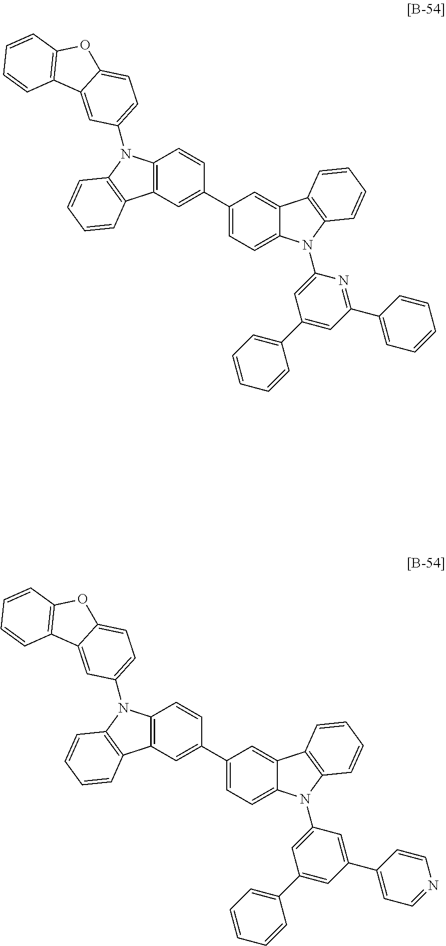 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1]

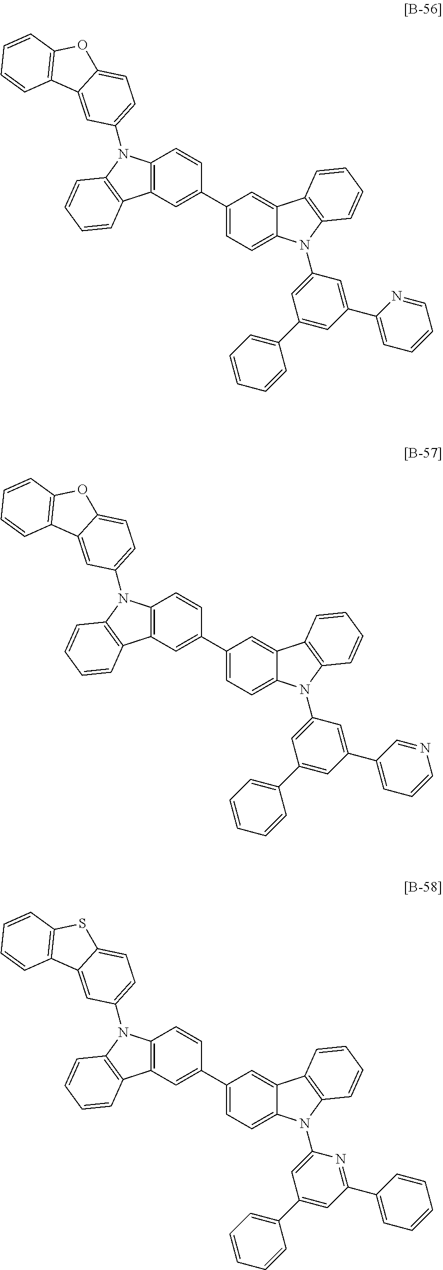 [SMILES: c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1]

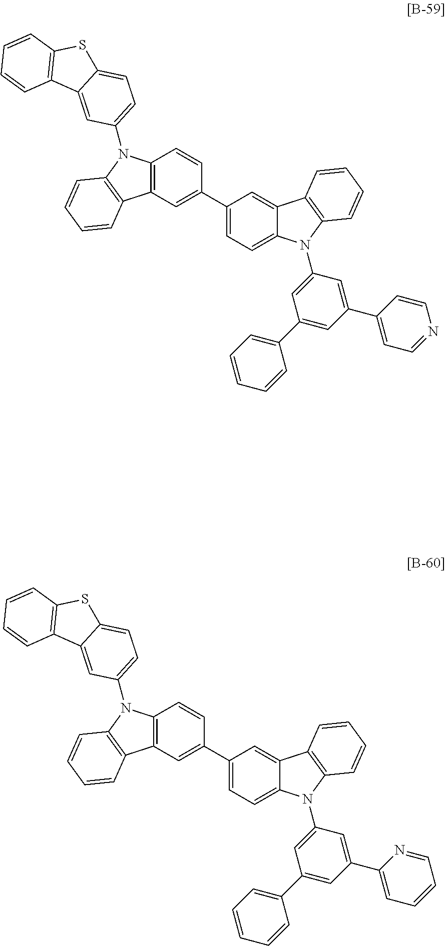 [SMILES: c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

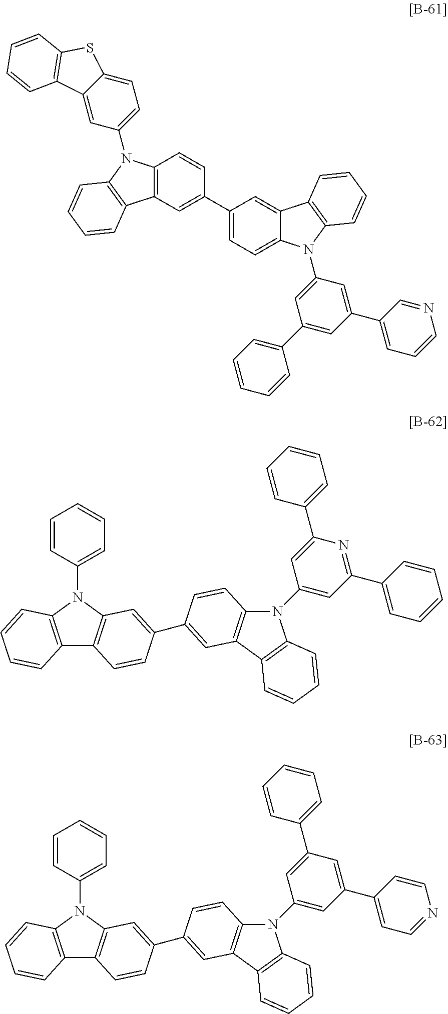 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1]

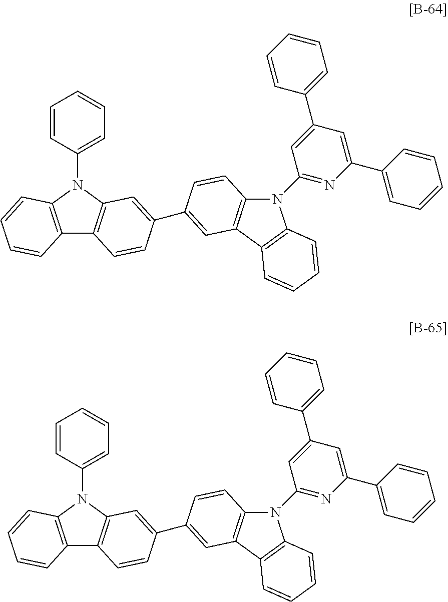 [SMILES: c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

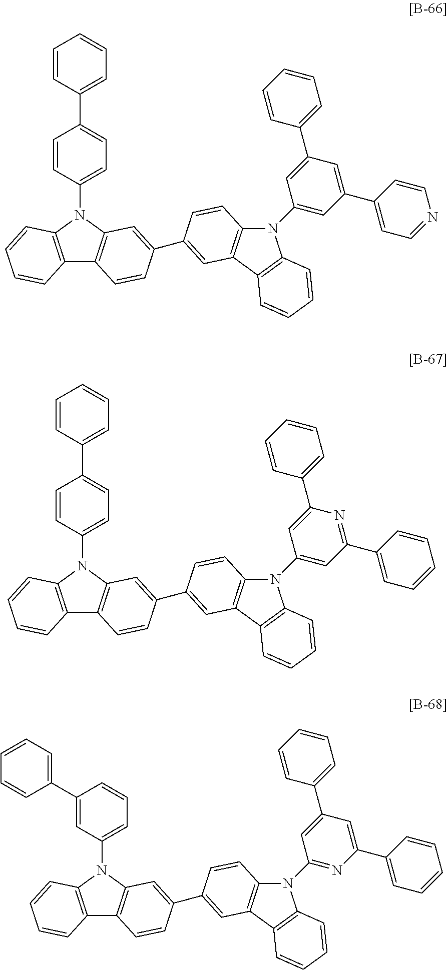 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

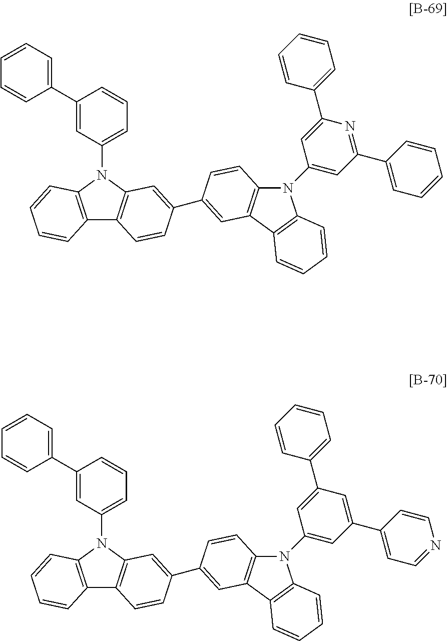 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1]

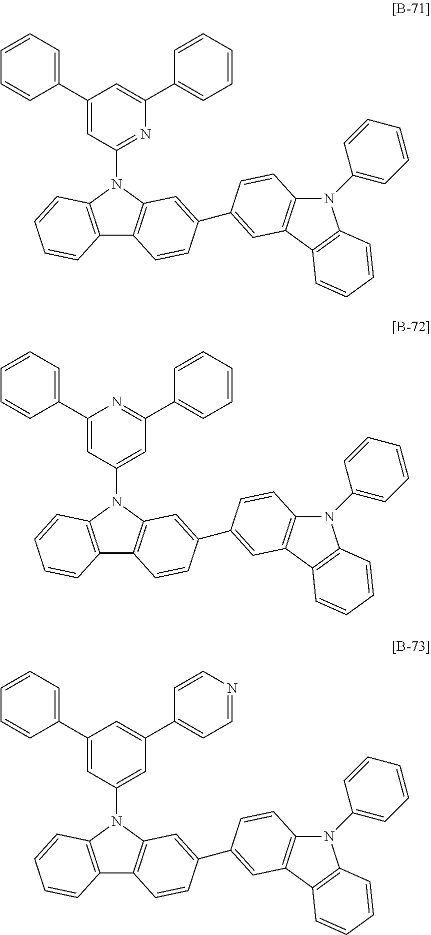 [SMILES: c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1]

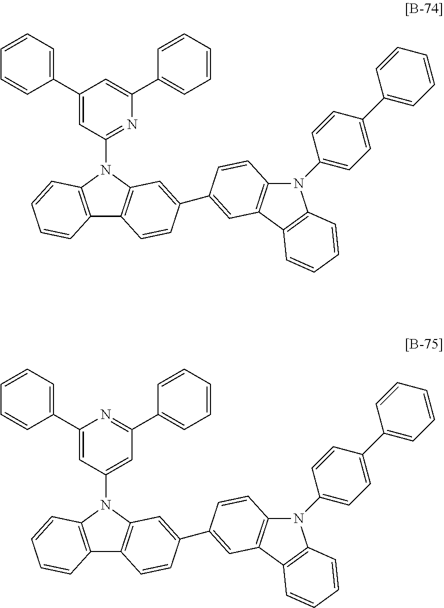 [SMILES: c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

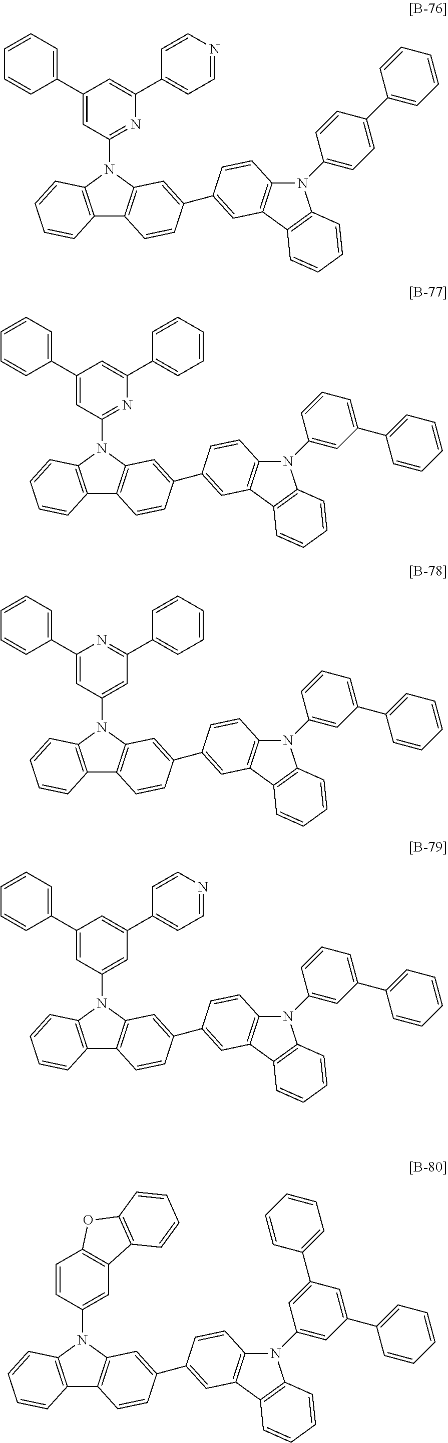 [SMILES: c1ccc(-c2ccc(-n3c4ccccc4c4cc(-c5ccc6c7ccccc7n(-c7cc(-c8ccccc8)cc(-c8ccncc8)n7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

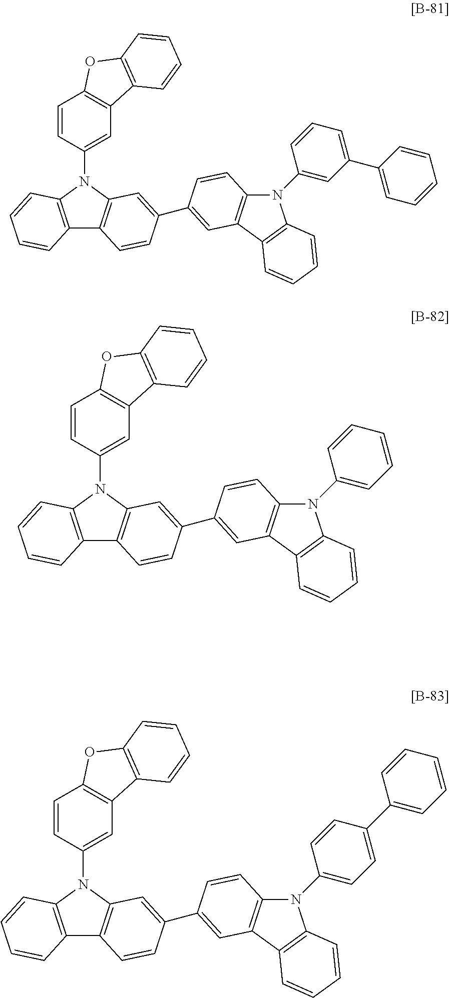 [SMILES: c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

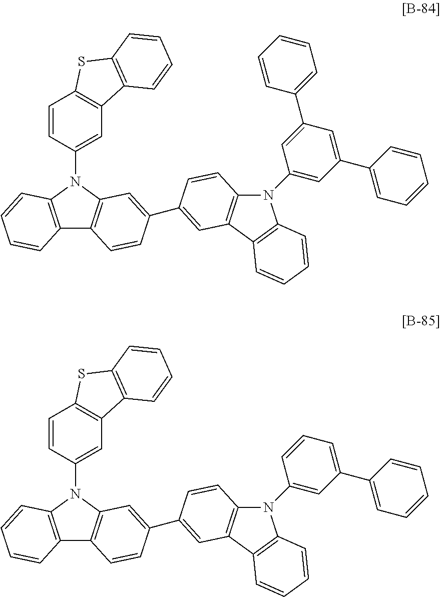 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1]

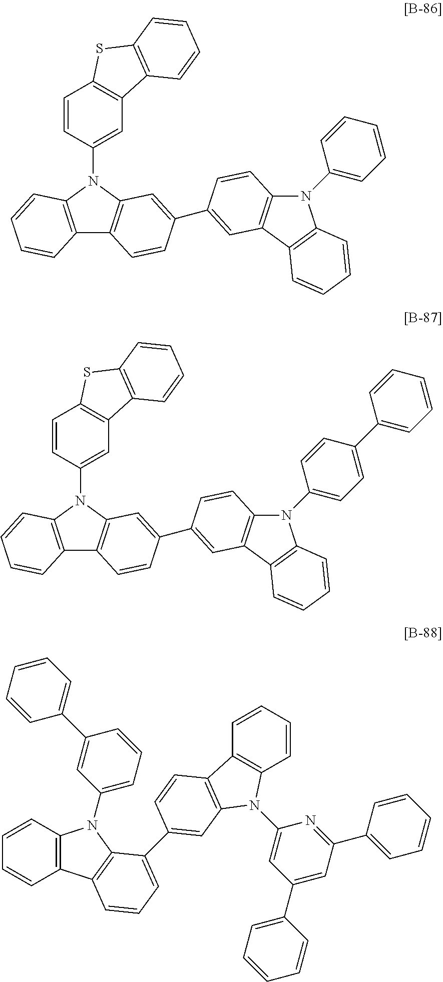 [SMILES: c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1]

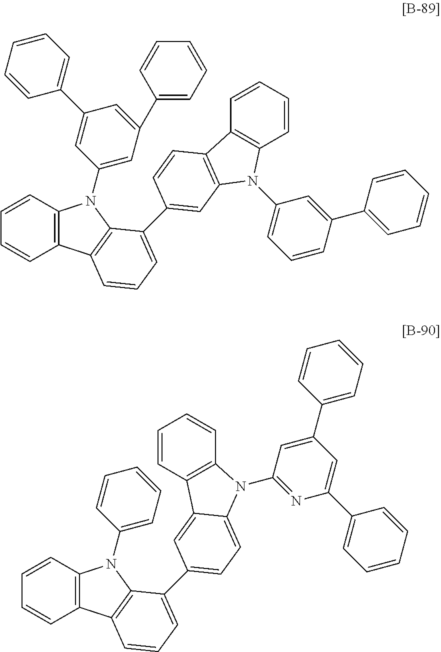 [SMILES: c1ccc(-c2cccc(-n3c4ccccc4c4ccc(-c5cccc6c7ccccc7n(-c7cc(-c8ccccc8)cc(-c8ccccc8)c7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

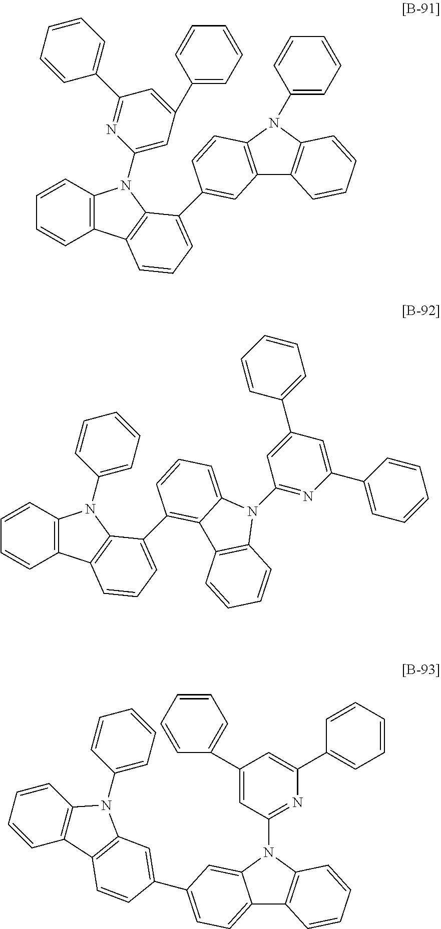 [SMILES: c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(-n3c4ccccc4c4ccc(-c5ccc6c7ccccc7n(-c7ccccc7)c6c5)cc43)c2)cc1]

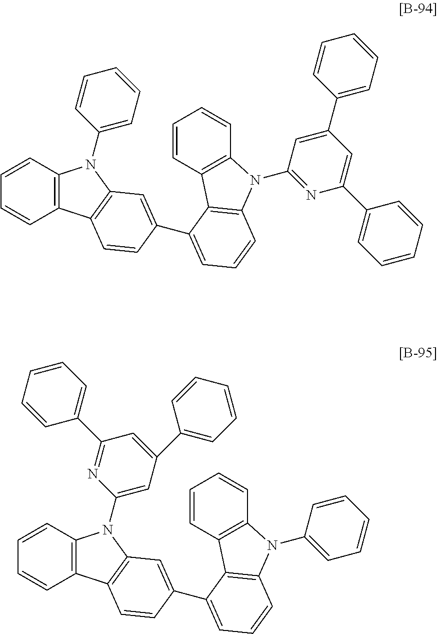 [SMILES: c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1]

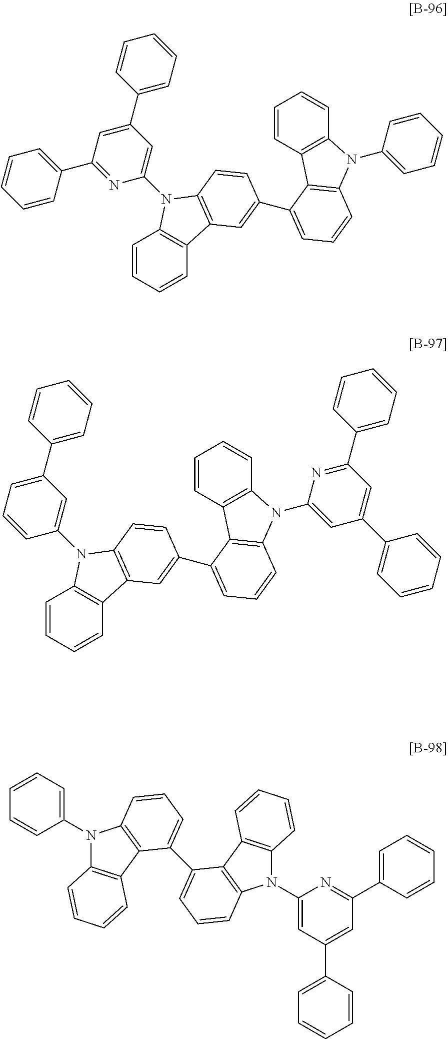 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

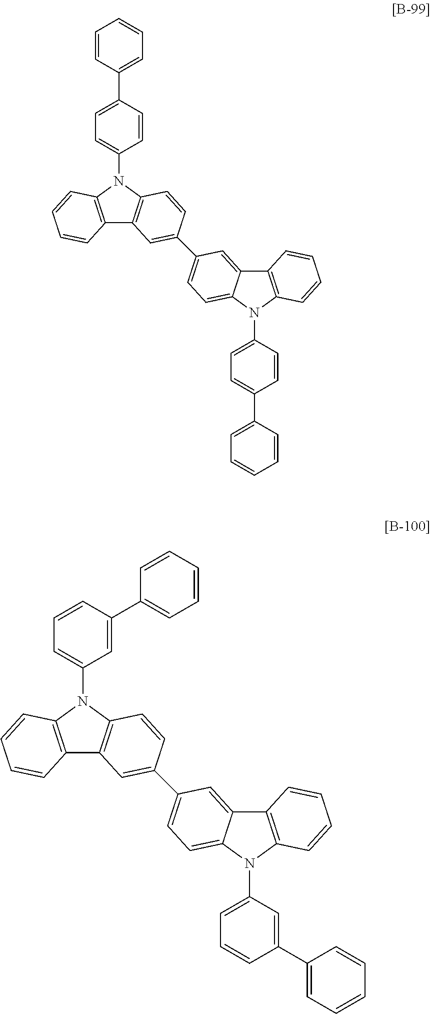 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

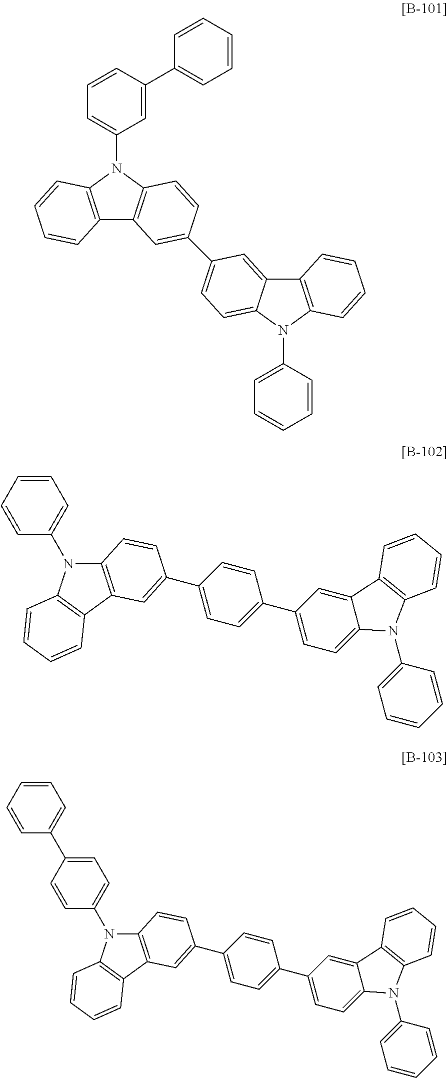 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

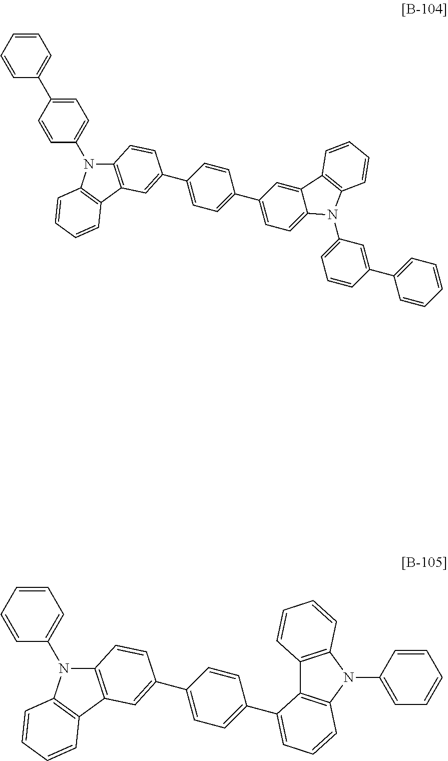 [SMILES: c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4ccc(-c5cccc6c5c5ccccc5n6-c5ccccc5)cc4)ccc32)cc1]

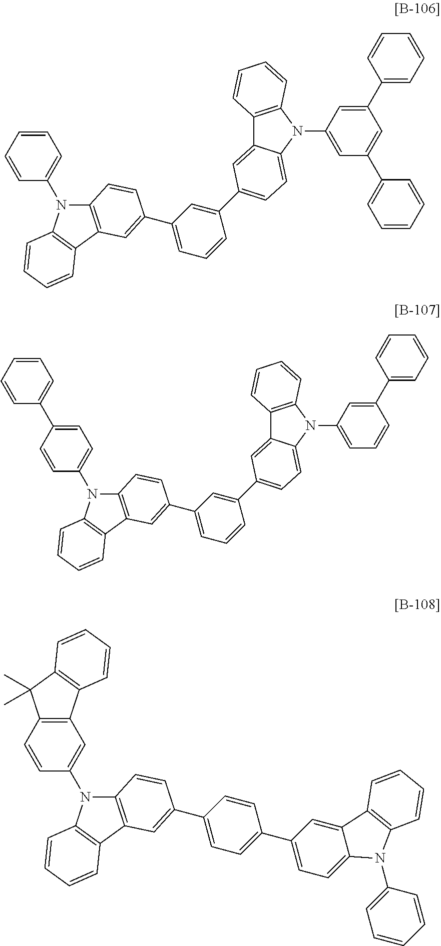 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21]

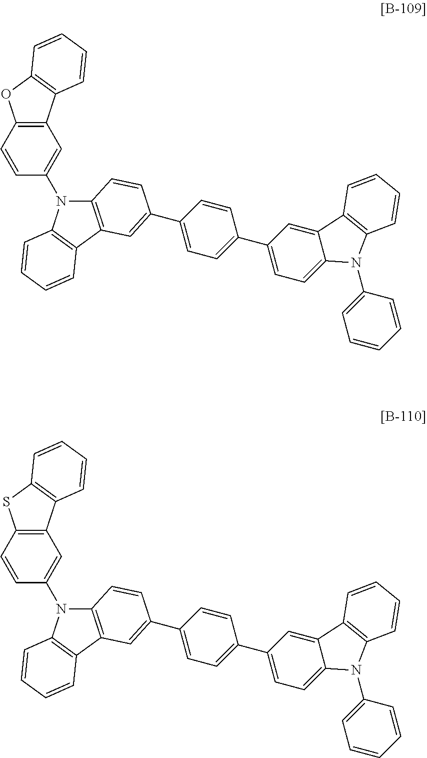 [SMILES: c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1]

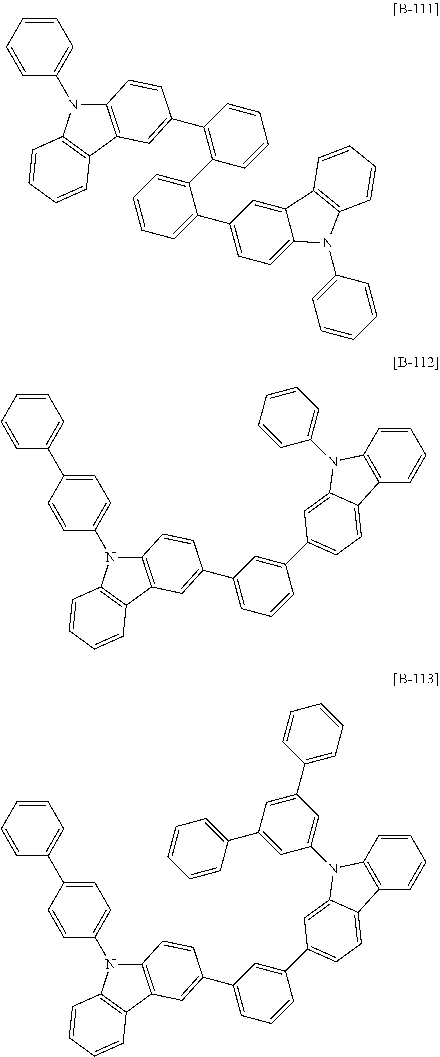 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

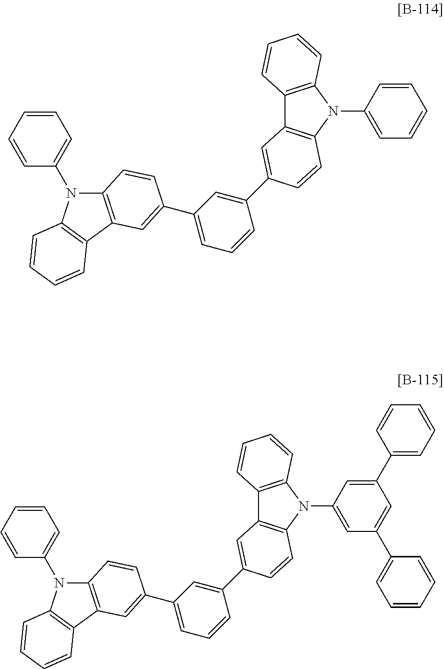 [SMILES: c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

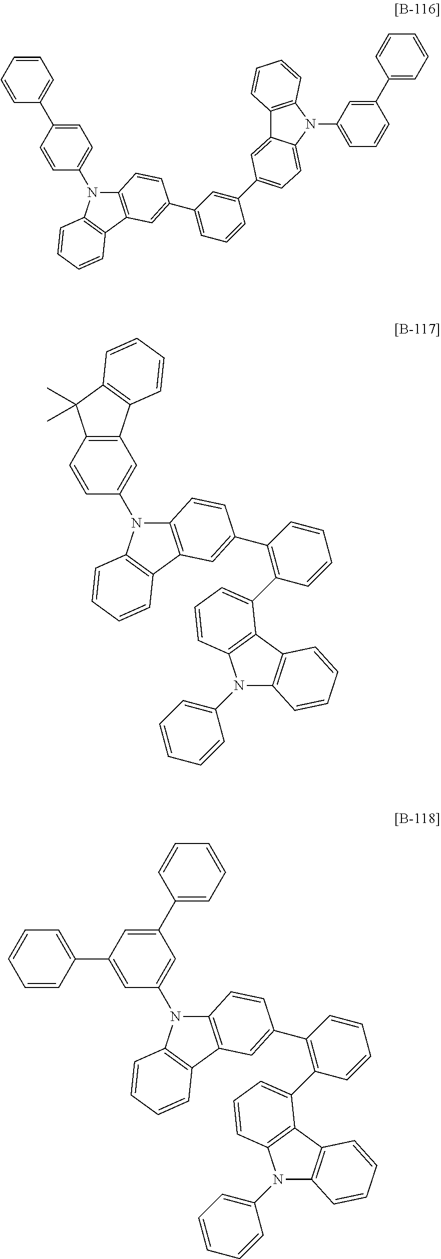 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

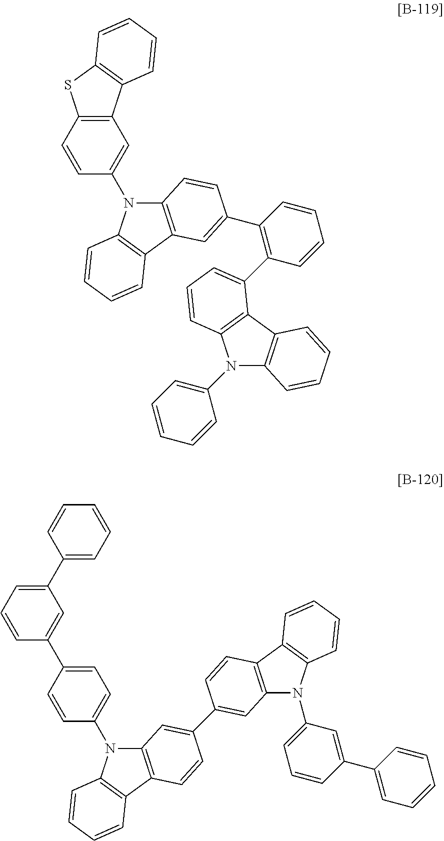 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

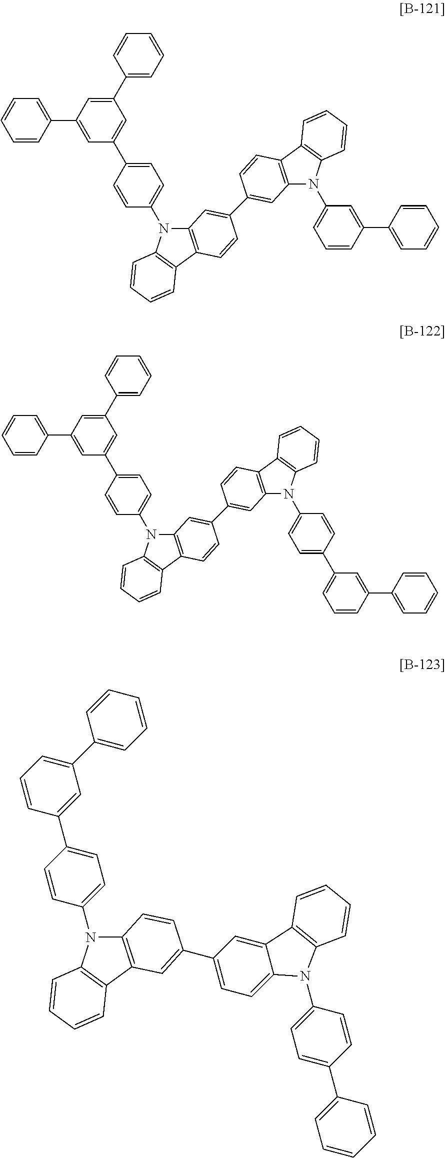 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5ccc(-c6ccc7c8ccccc8n(-c8ccc(-c9cc(-c%10ccccc%10)cc(-c%10ccccc%10)c9)cc8)c7c6)cc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

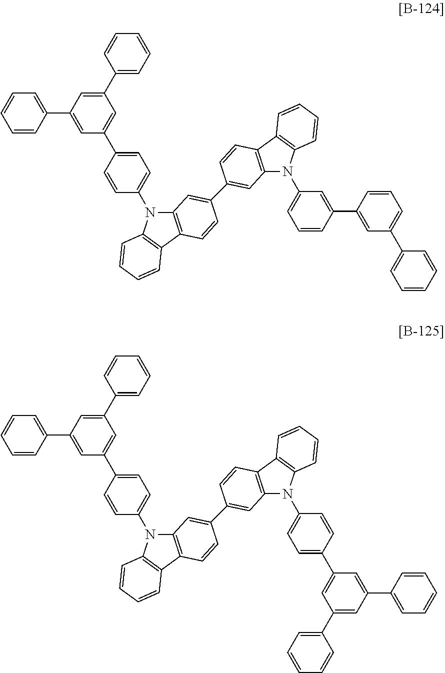 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5ccc(-c6ccc7c8ccccc8n(-c8ccc(-c9cc(-c%10ccccc%10)cc(-c%10ccccc%10)c9)cc8)c7c6)cc54)cc3)c2)cc1]

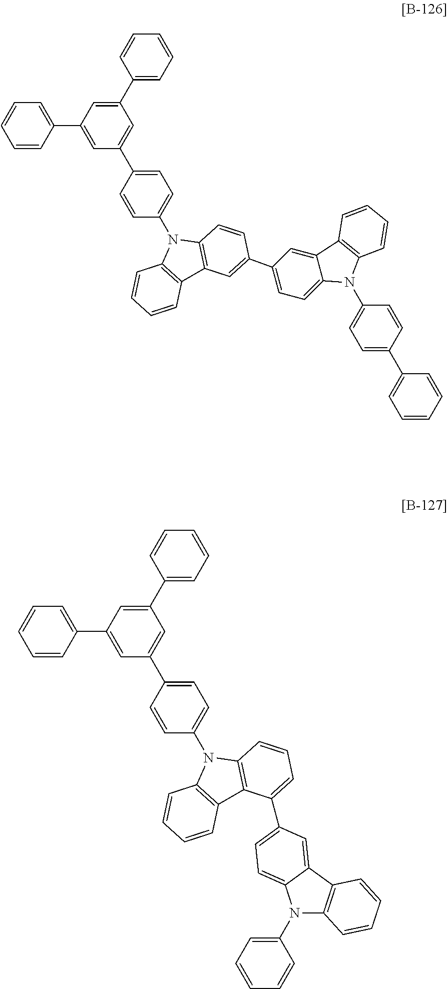 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

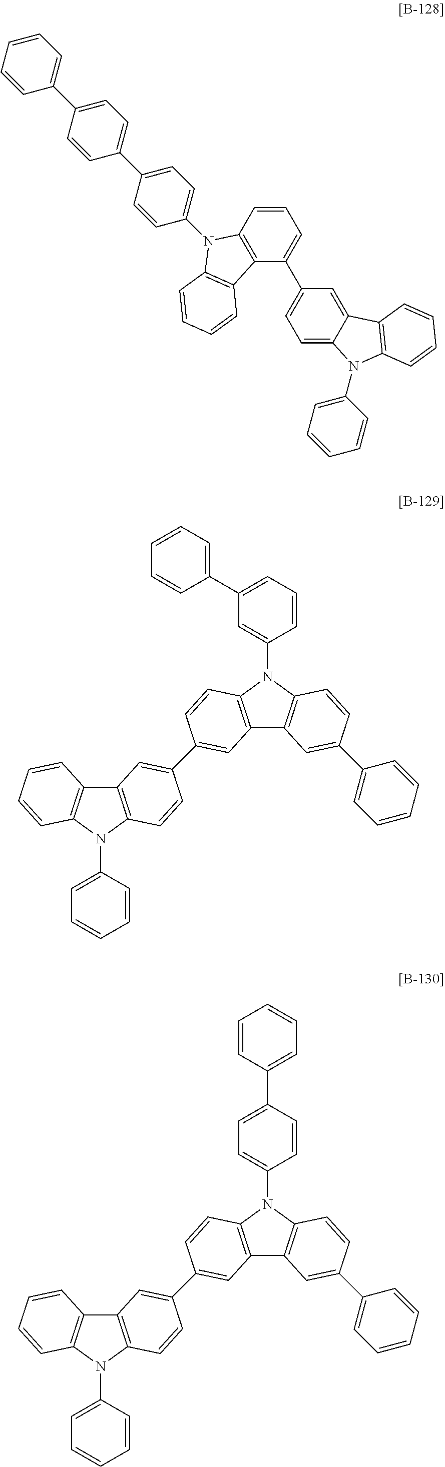 [SMILES: c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

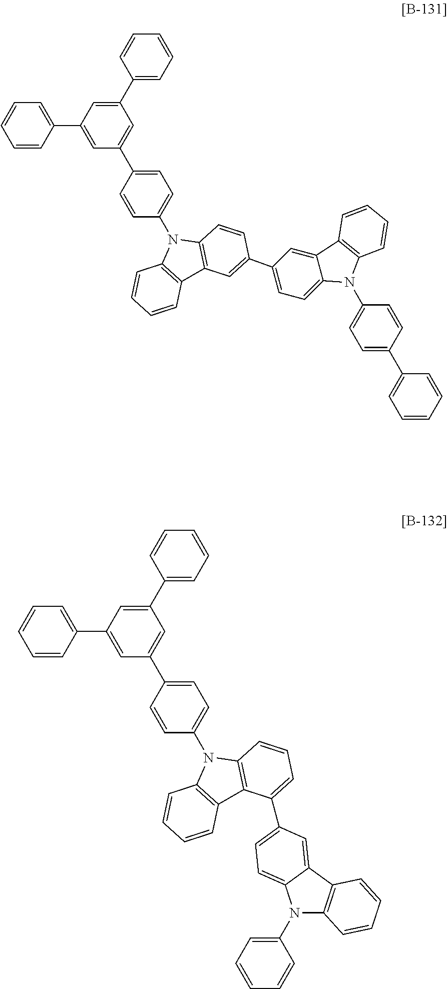 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

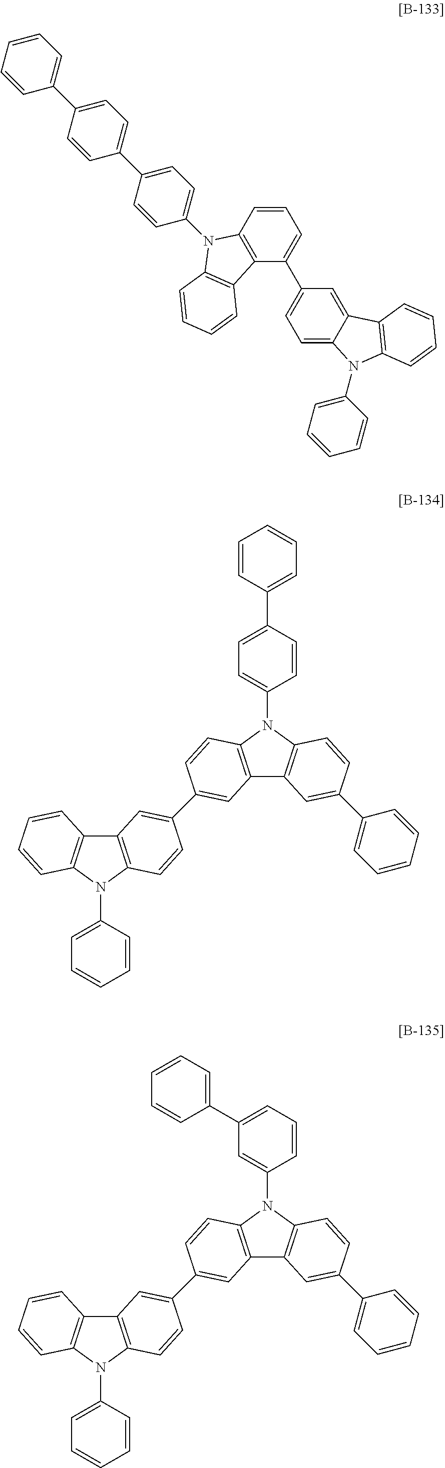 [SMILES: c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

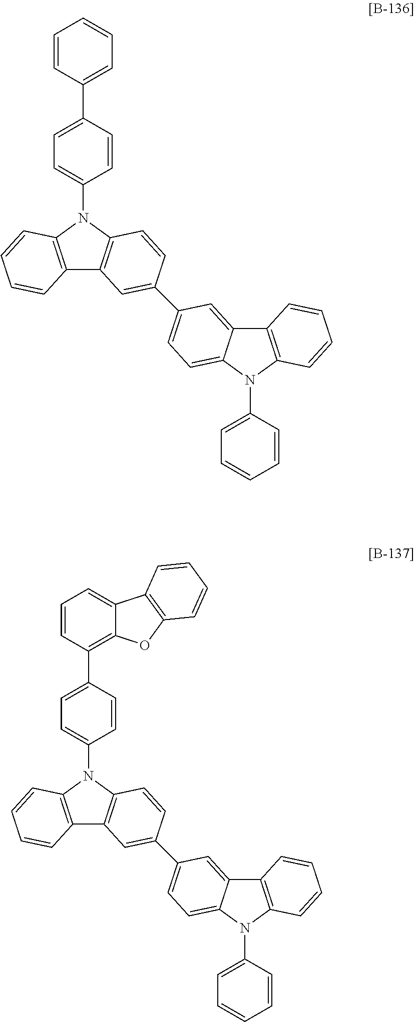 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

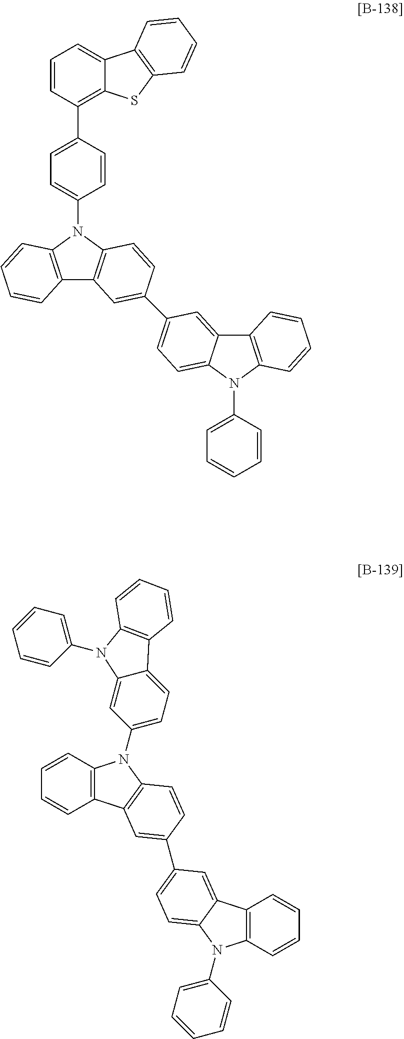 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

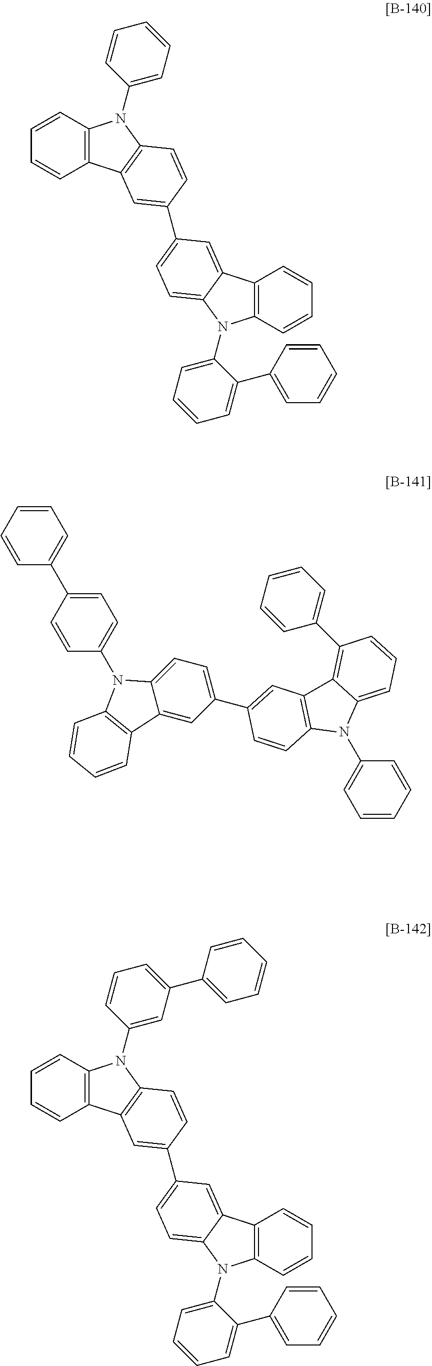

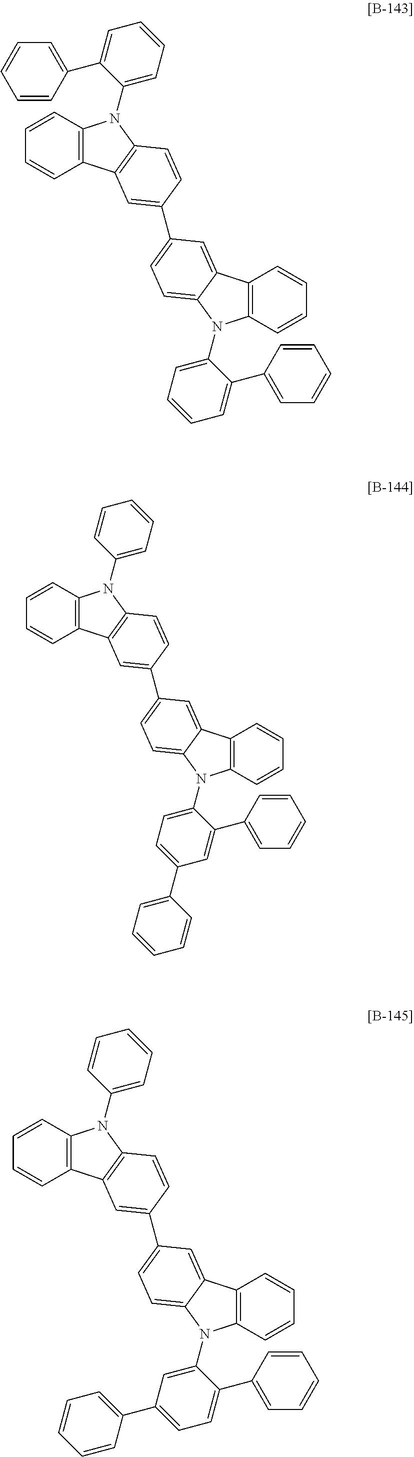

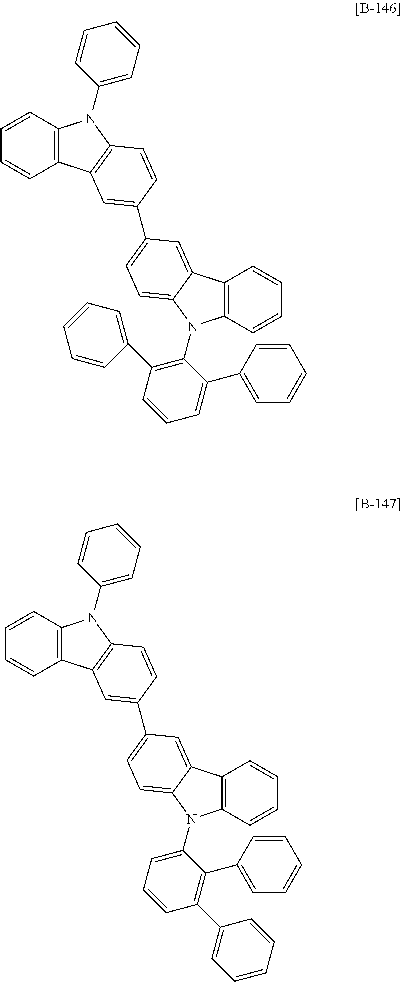

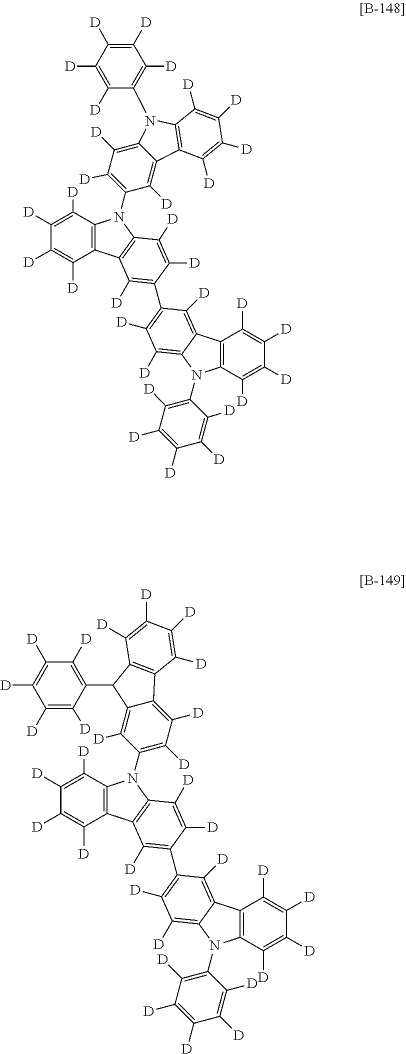

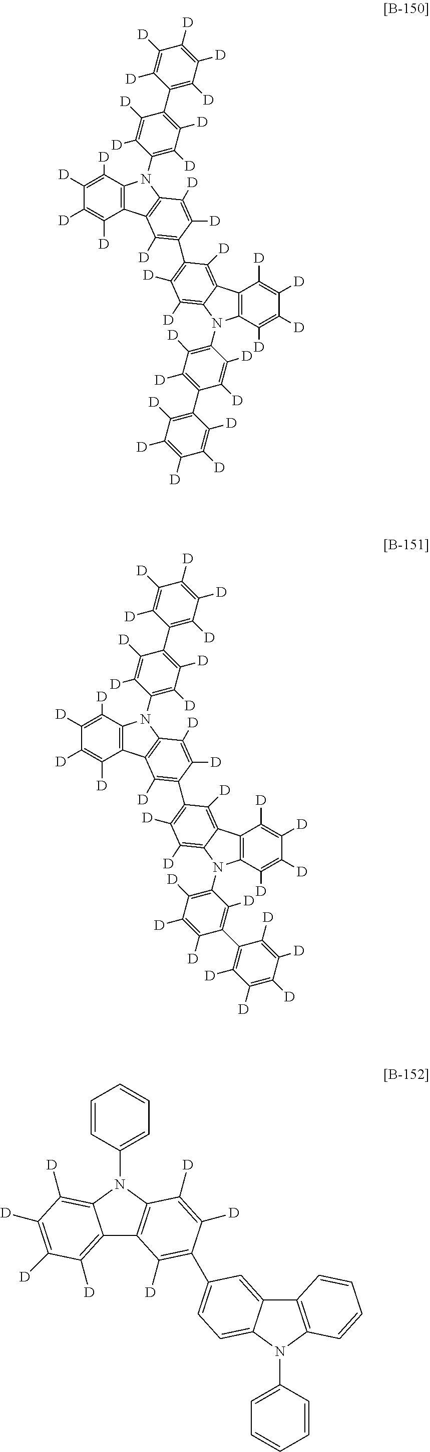

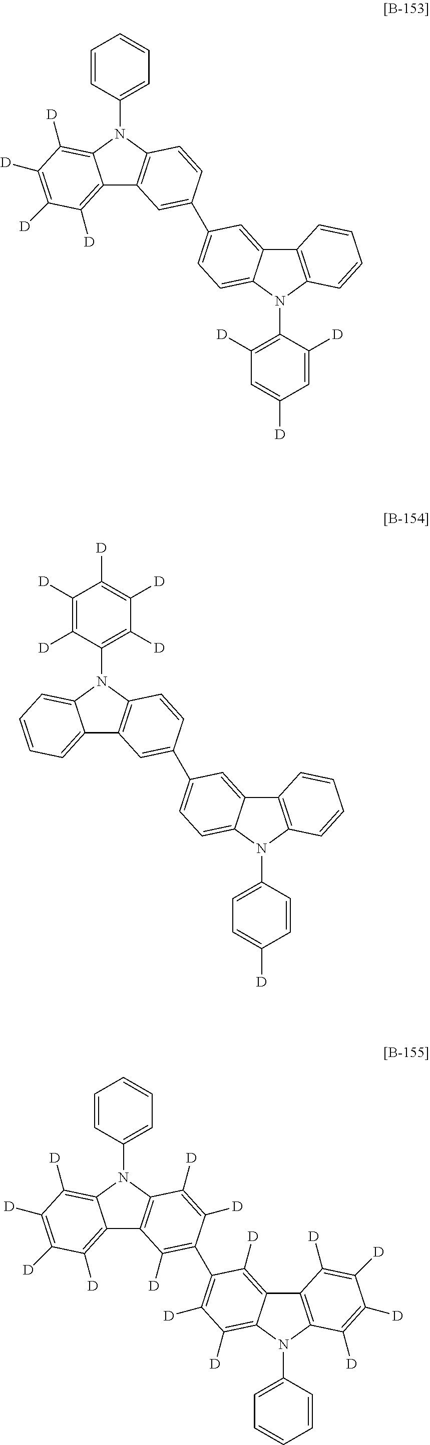 [SMILES: [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

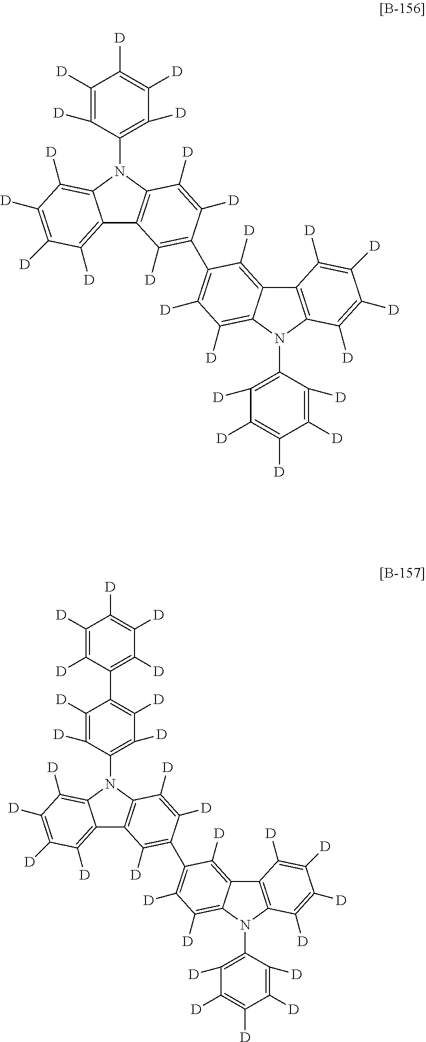 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

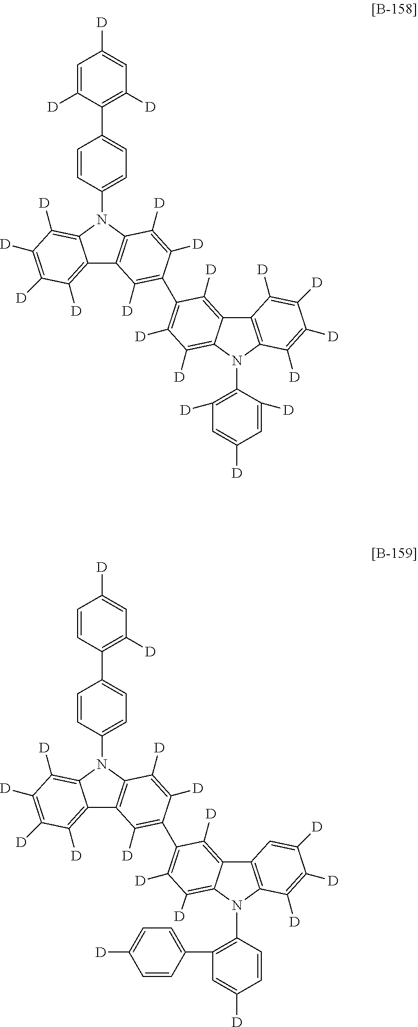 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2cc([2H])ccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

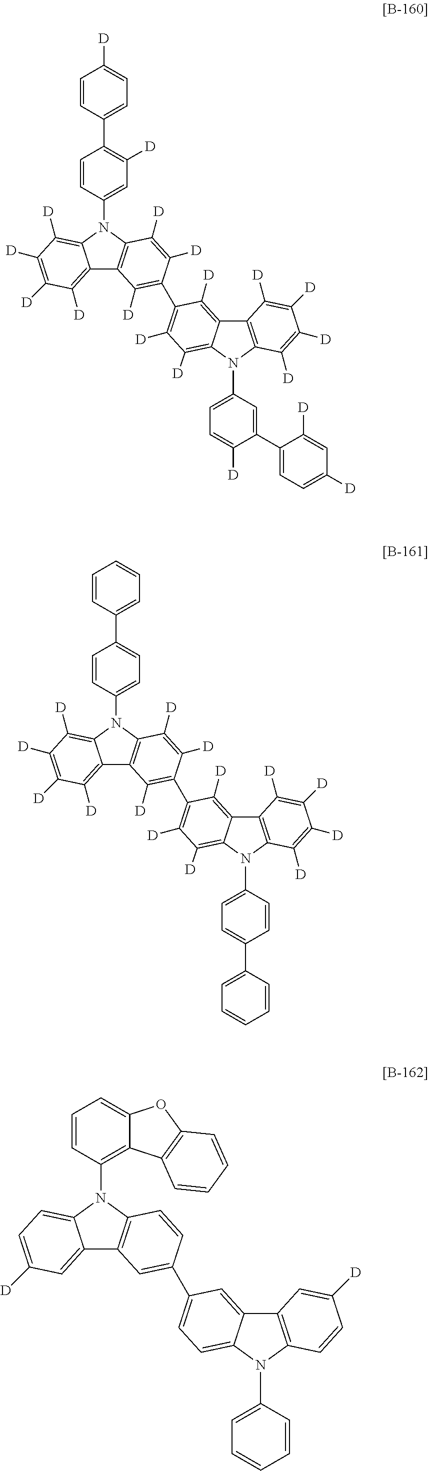 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1]

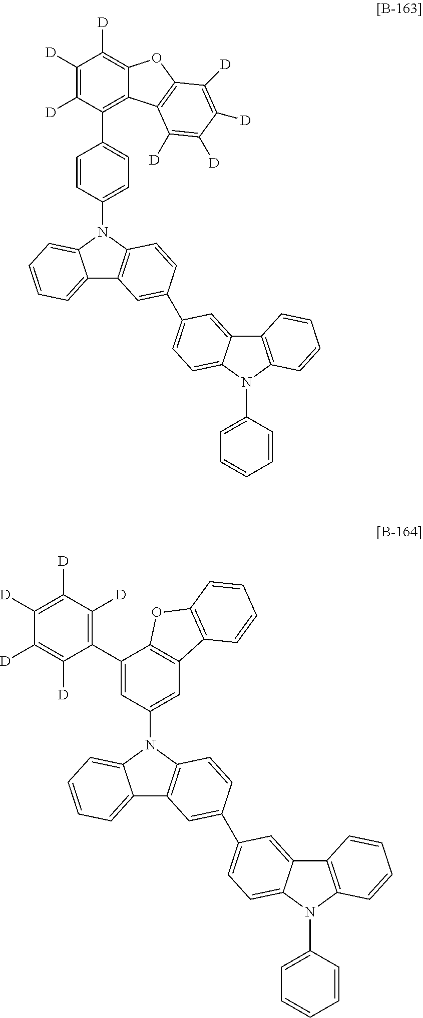 [SMILES: [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

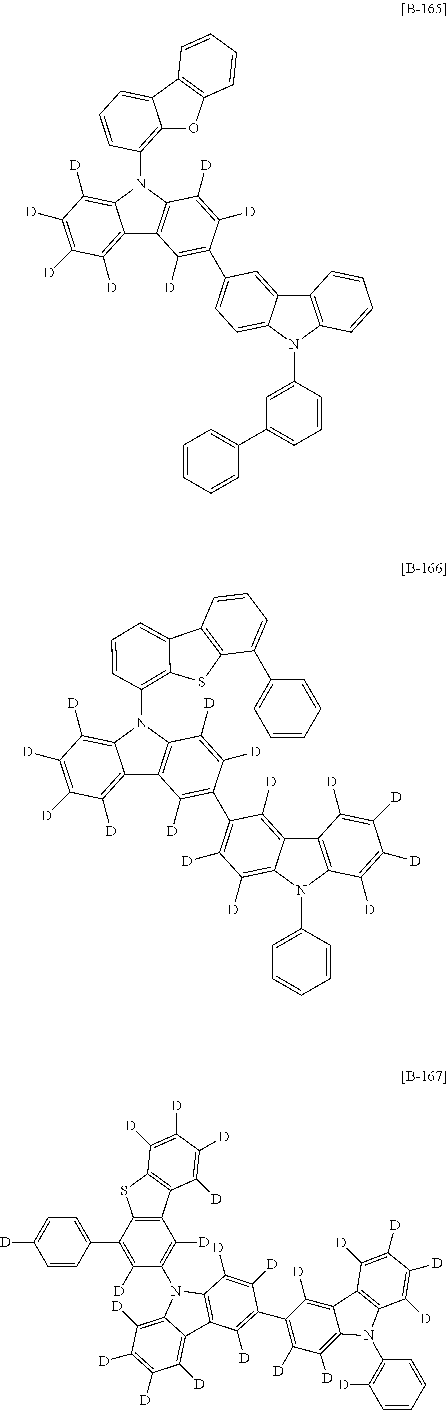

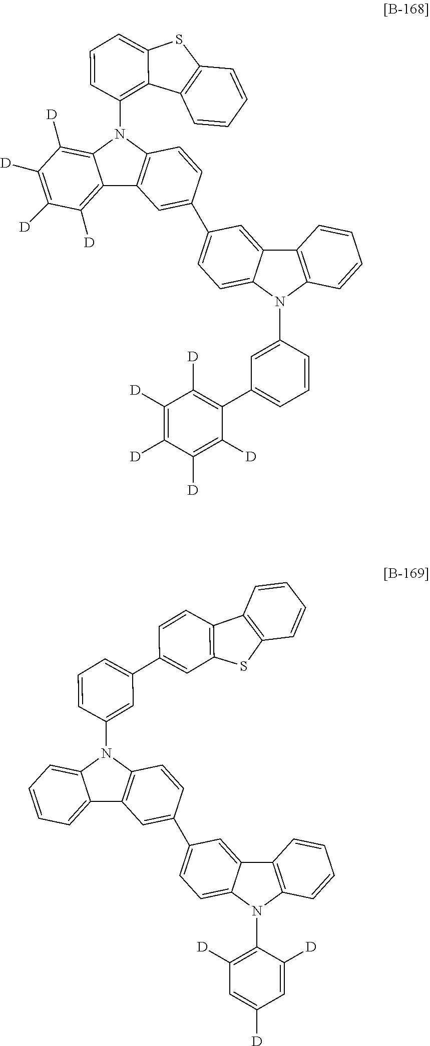 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

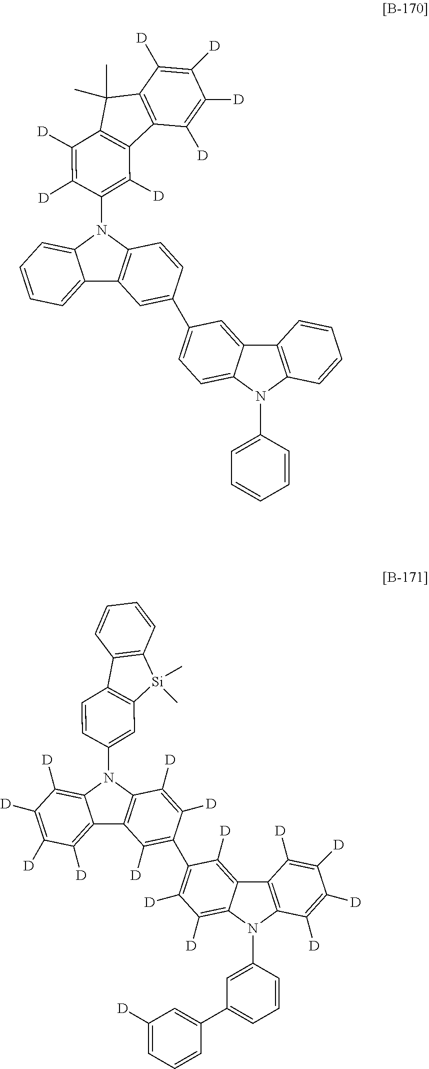 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

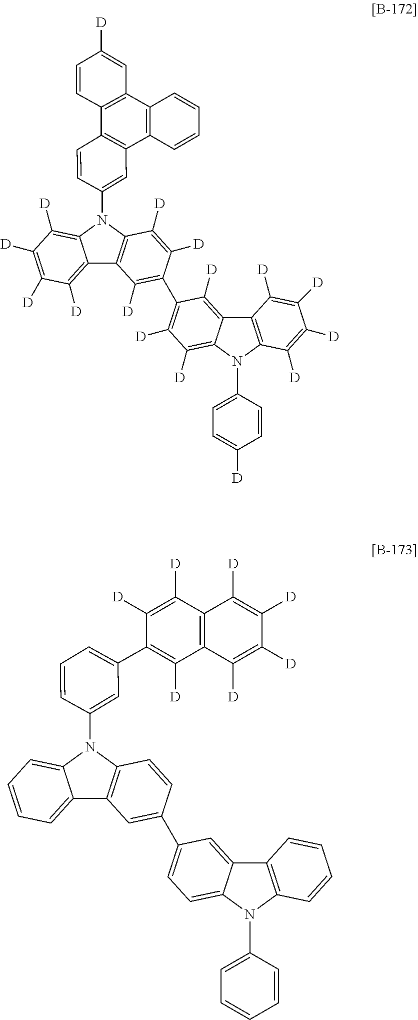 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

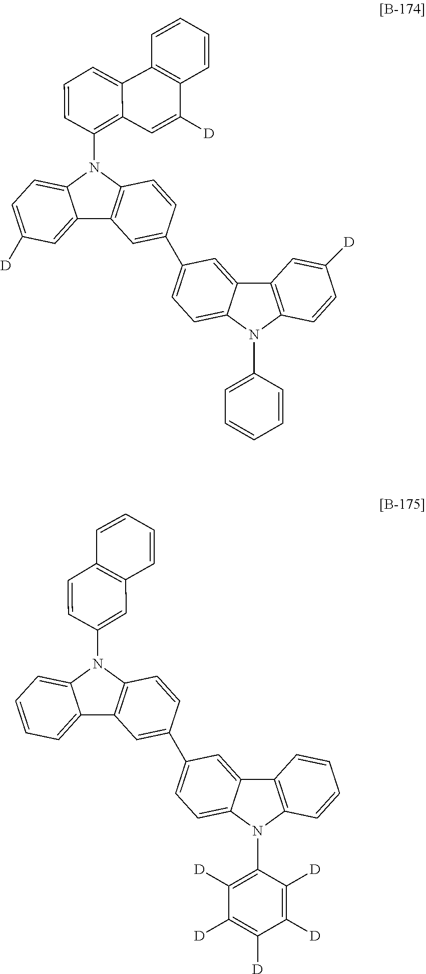 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

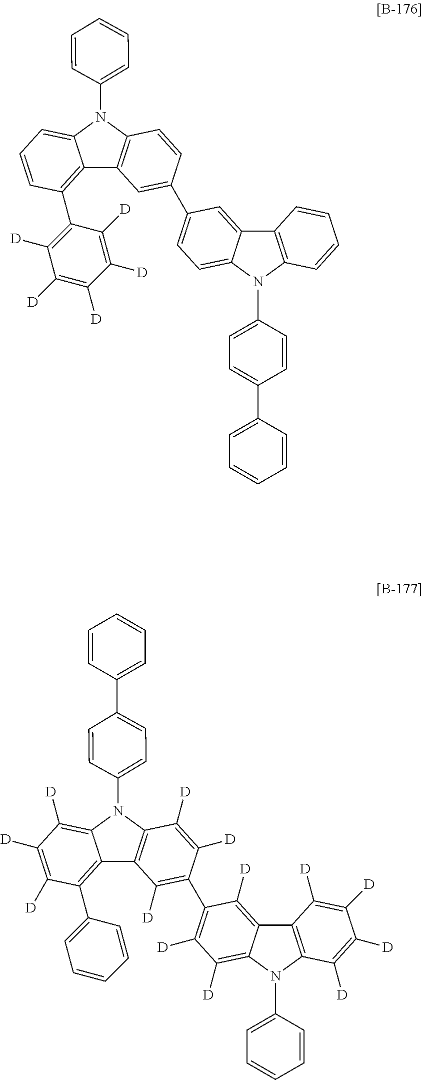 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

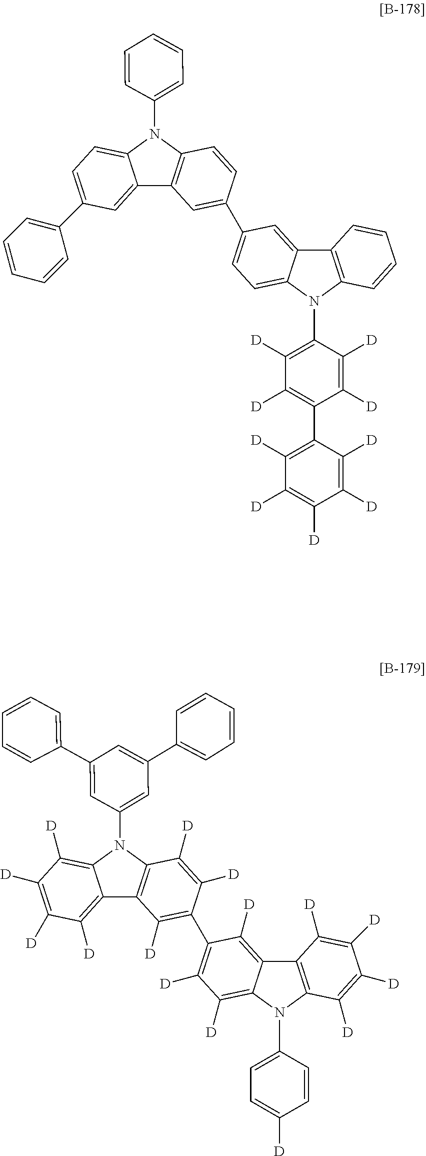 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

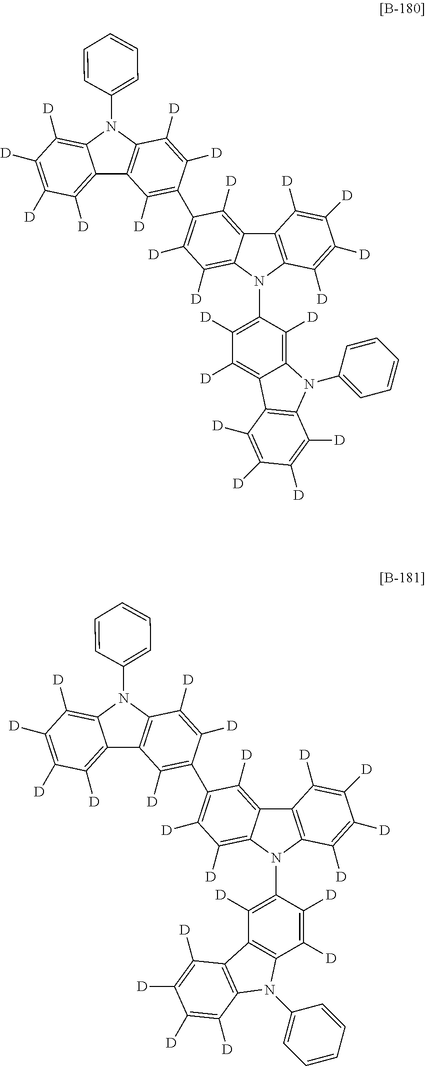 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

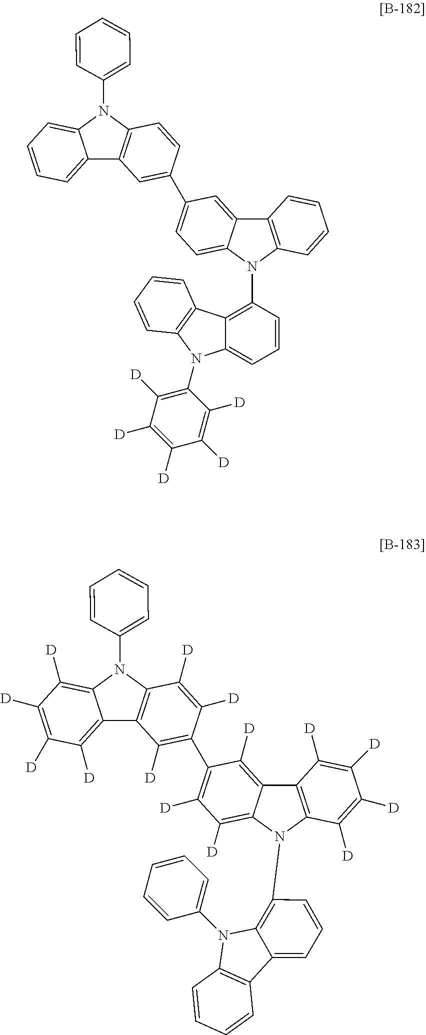 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

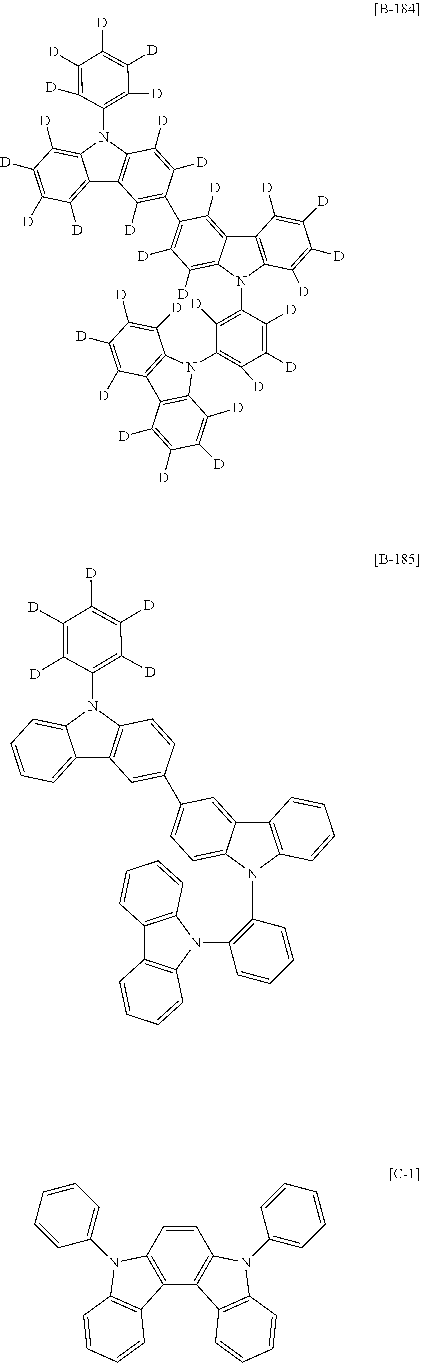 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H] ; c1ccc(-n2c3ccccc3c3c4c5ccccc5n(-c5ccccc5)c4ccc32)cc1]

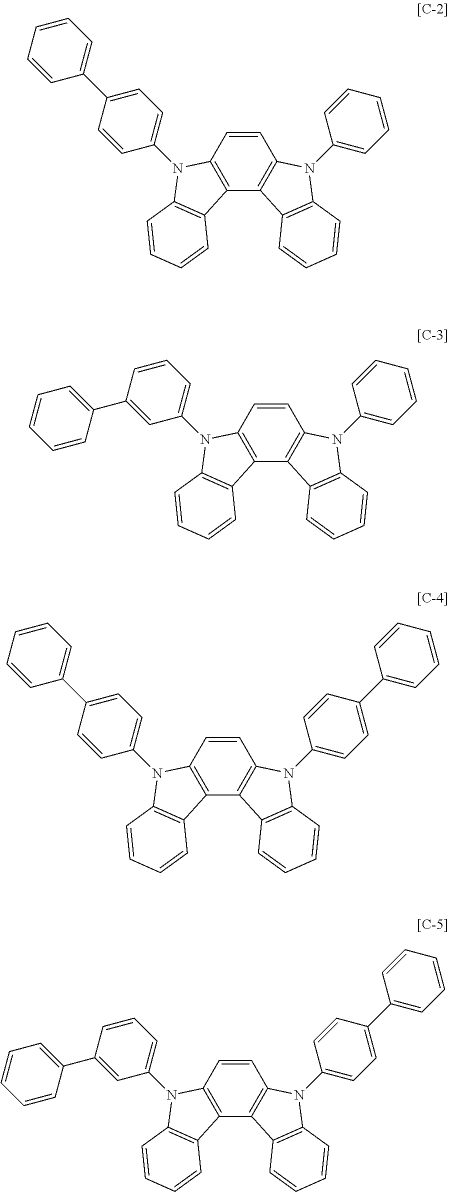 [SMILES: c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1]

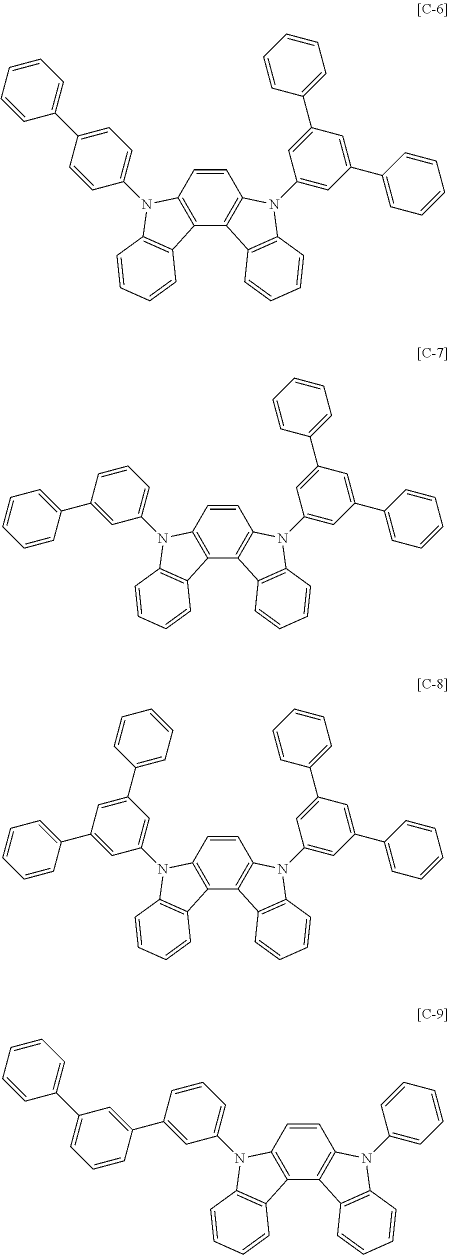 [SMILES: c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1]

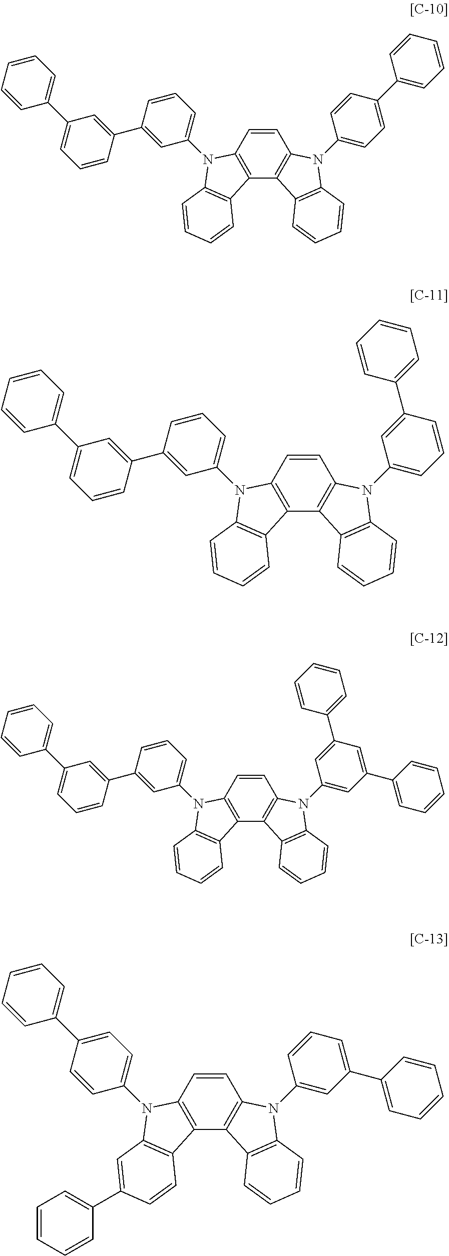 [SMILES: c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1 ; c1ccc(-c2ccc(-n3c4cc(-c5ccccc5)ccc4c4c5c6ccccc6n(-c6cccc(-c7ccccc7)c6)c5ccc43)cc2)cc1]

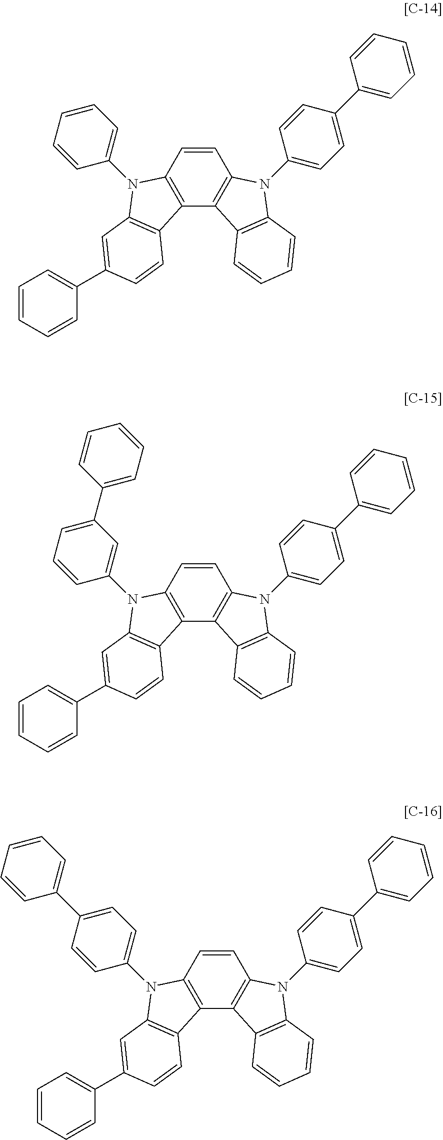 [SMILES: c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

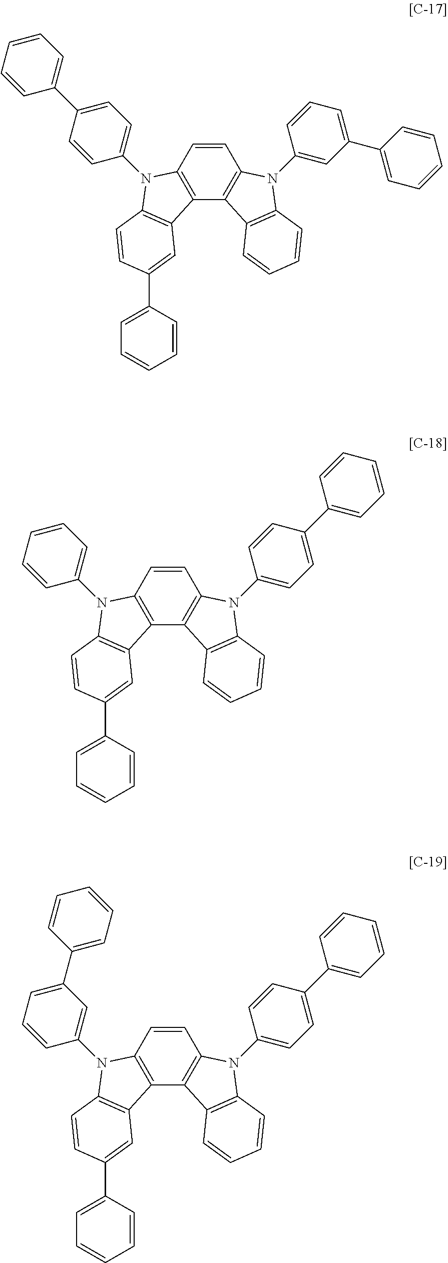 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

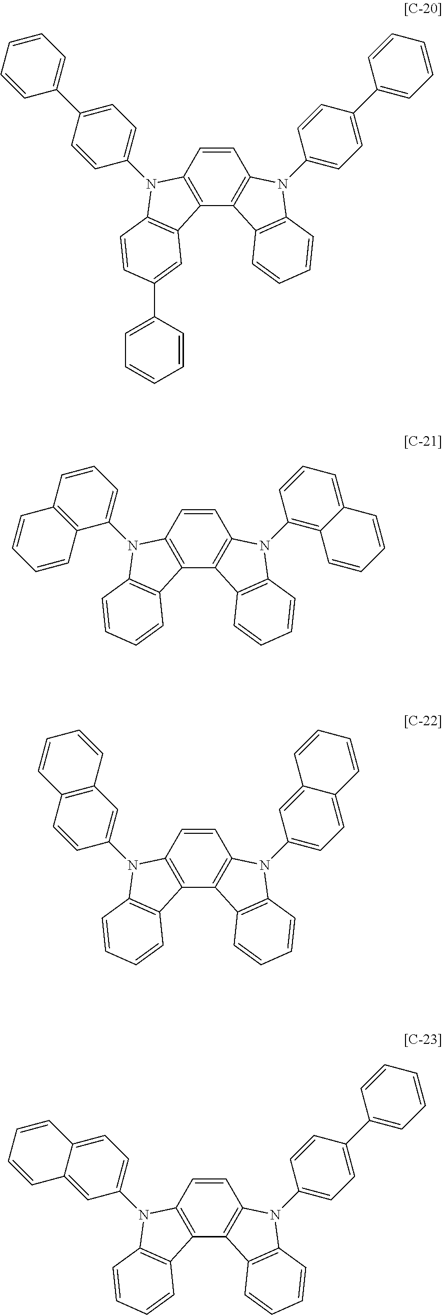 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1]

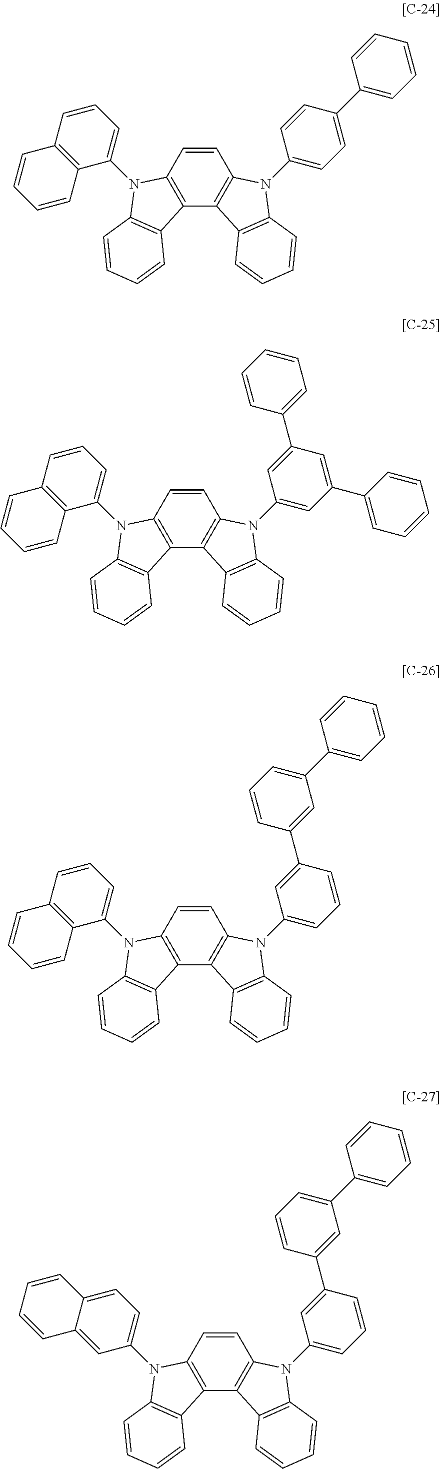 [SMILES: c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

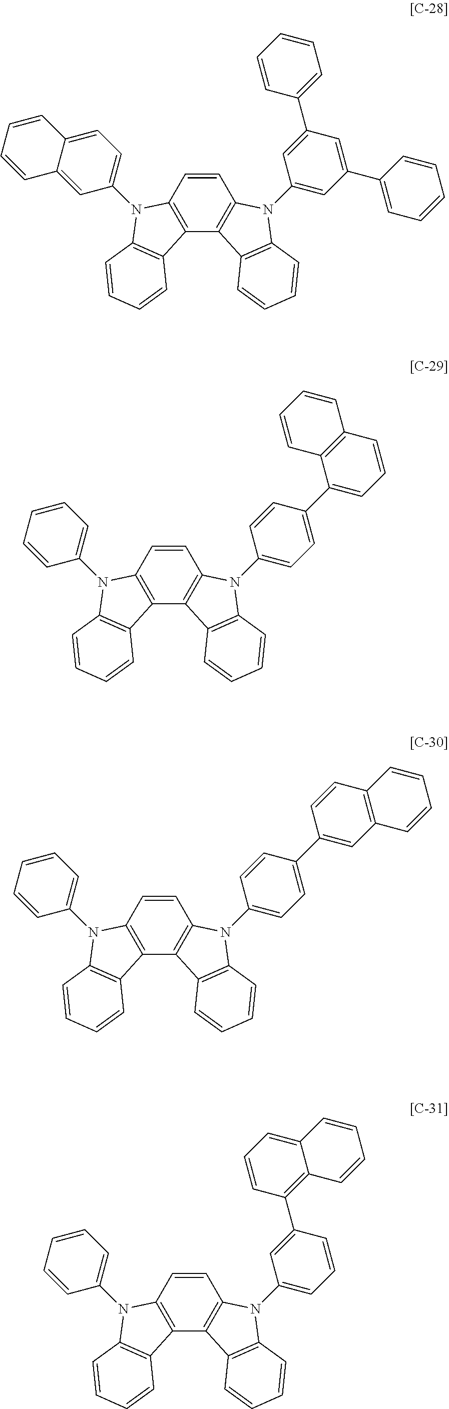 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

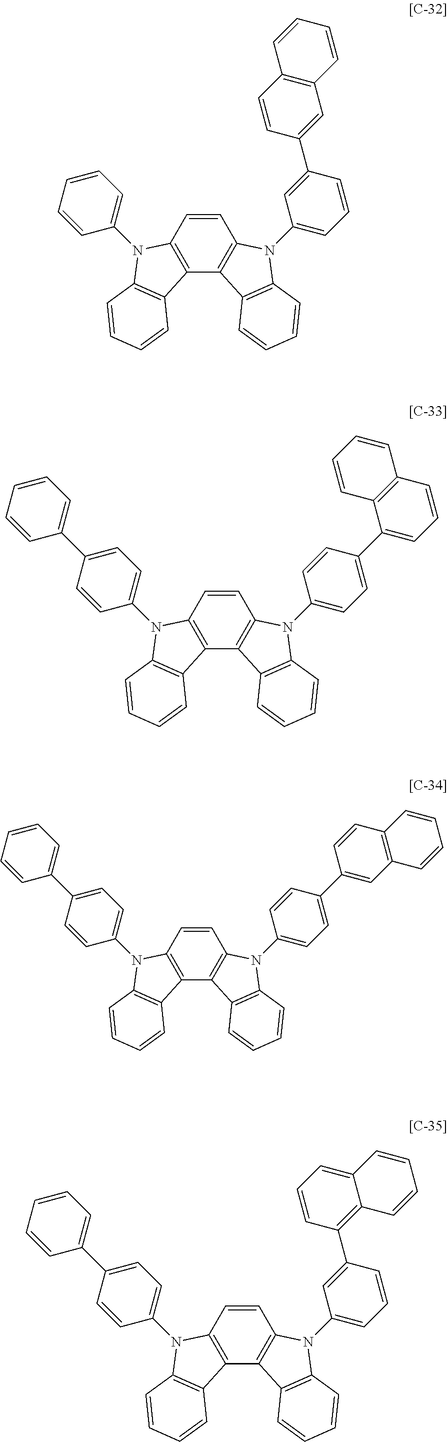 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

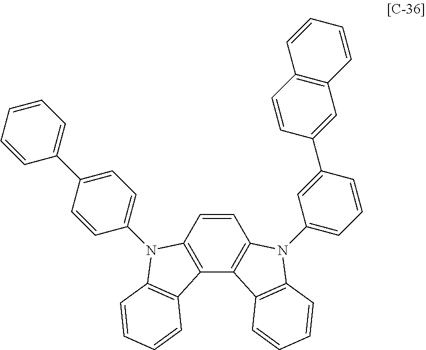 [SMILES: c1ccc(-c2ccc(-n3c4ccccc4c4c5c6ccccc6n(-c6cccc(-c7ccc8ccccc8c7)c6)c5ccc43)cc2)cc1]

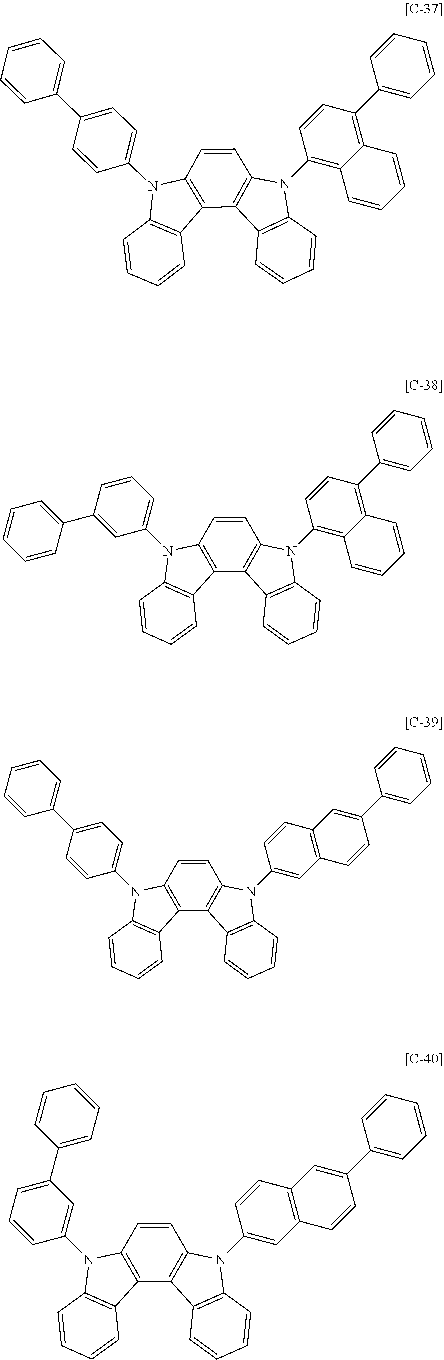 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

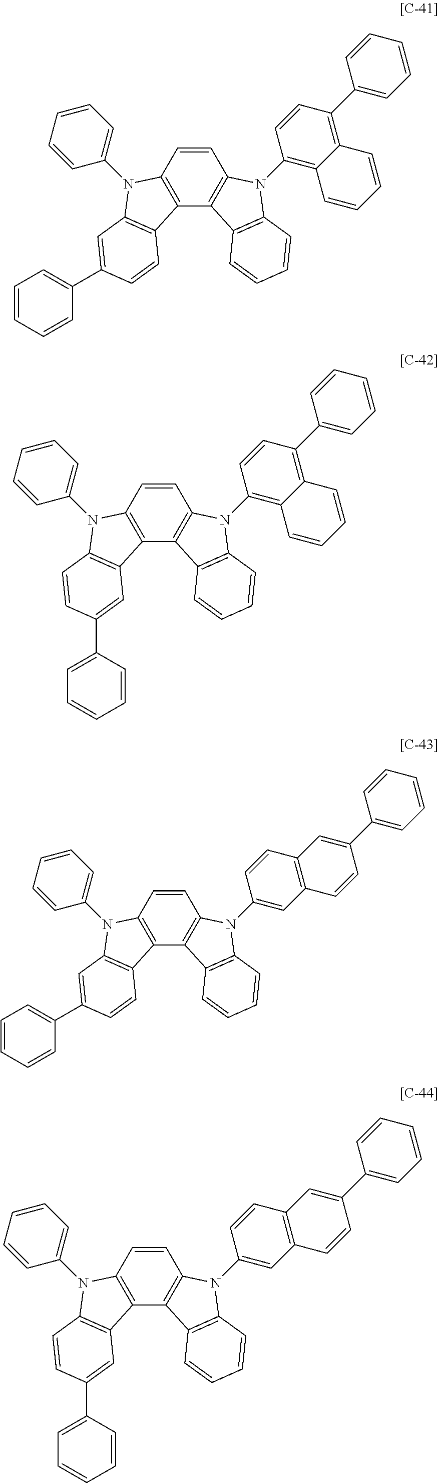 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

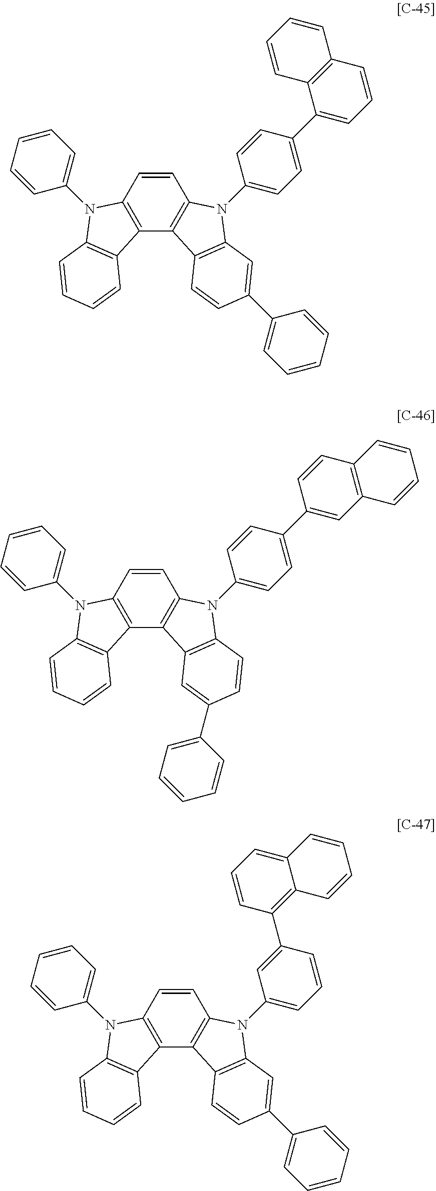 [SMILES: c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1]

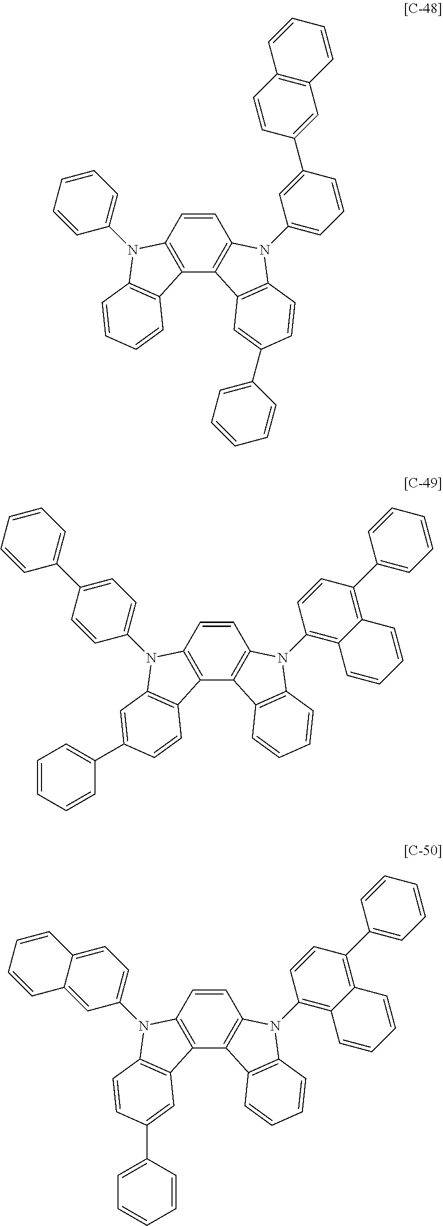 [SMILES: c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1]

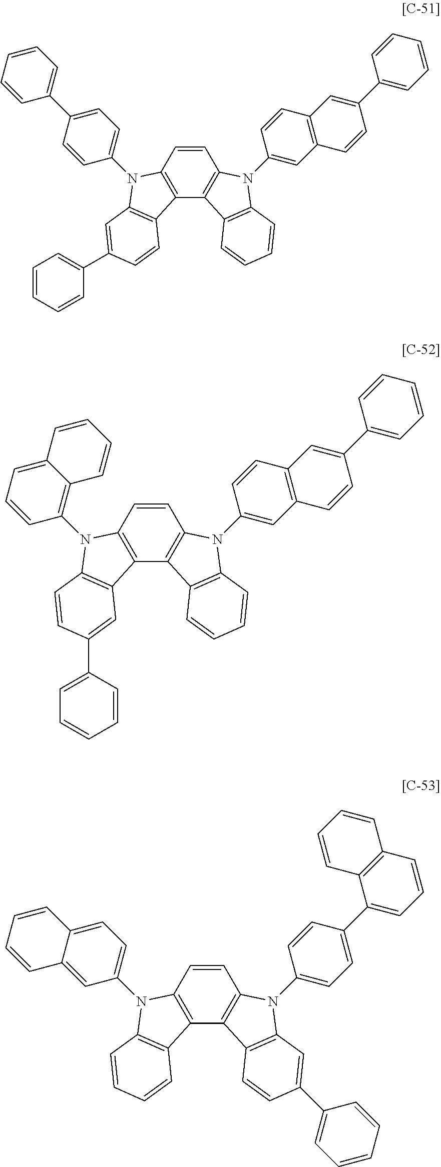 [SMILES: c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1 ; c1ccc(-c2ccc3c4c5c6ccccc6n(-c6ccc7ccccc7c6)c5ccc4n(-c4ccc(-c5cccc6ccccc56)cc4)c3c2)cc1]

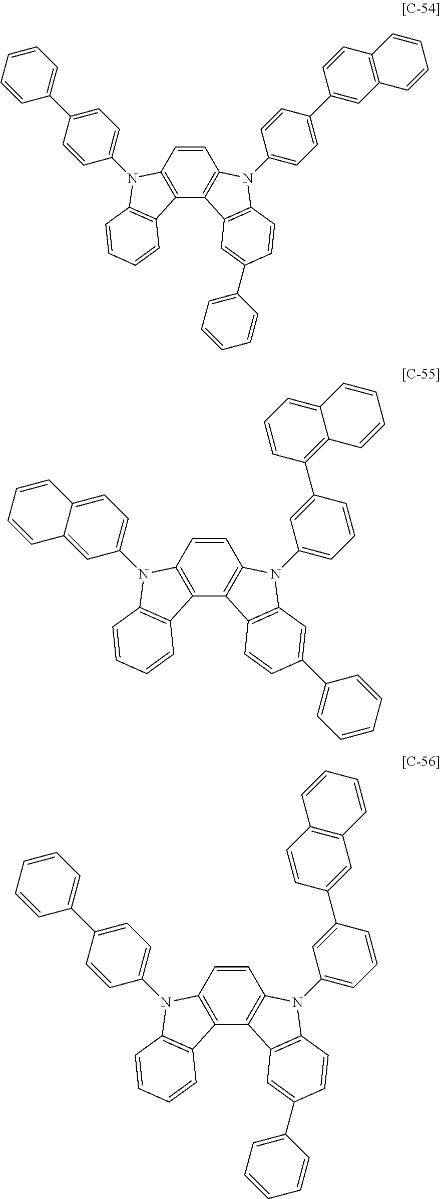

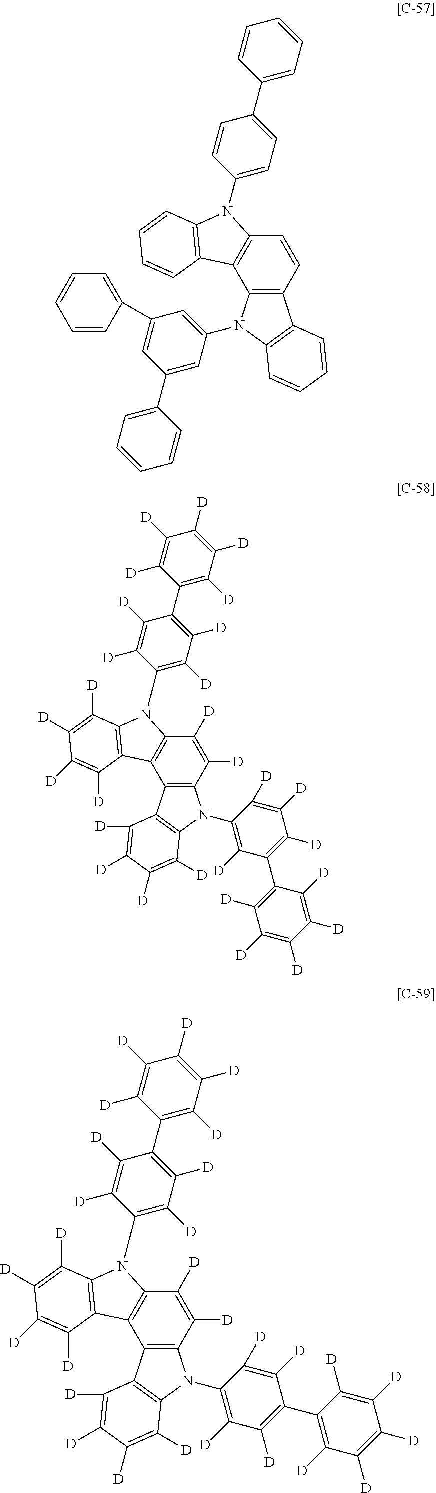

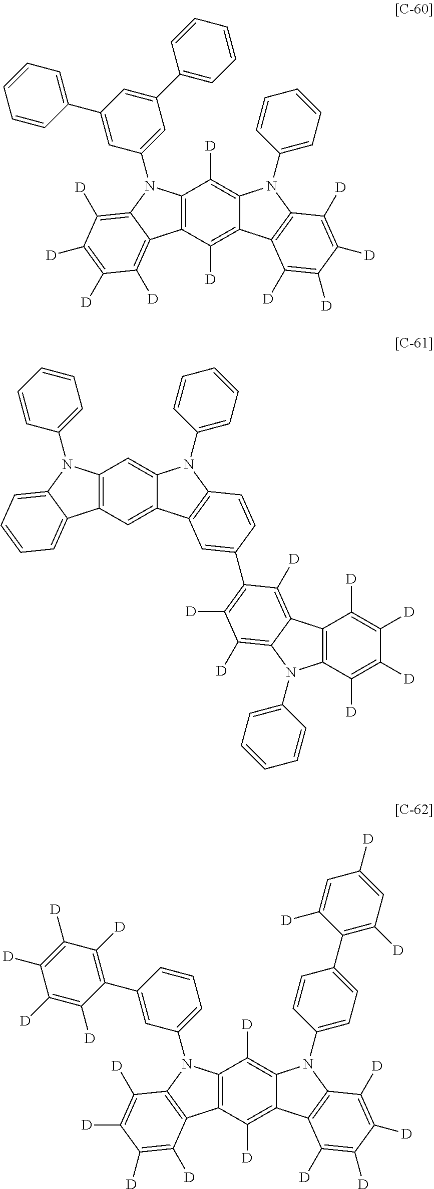 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

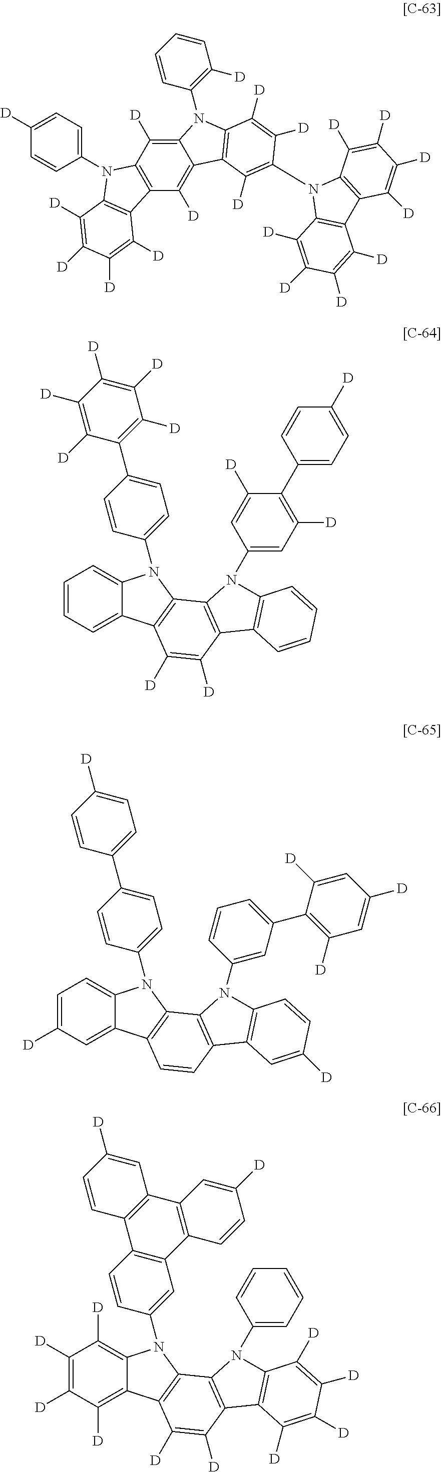

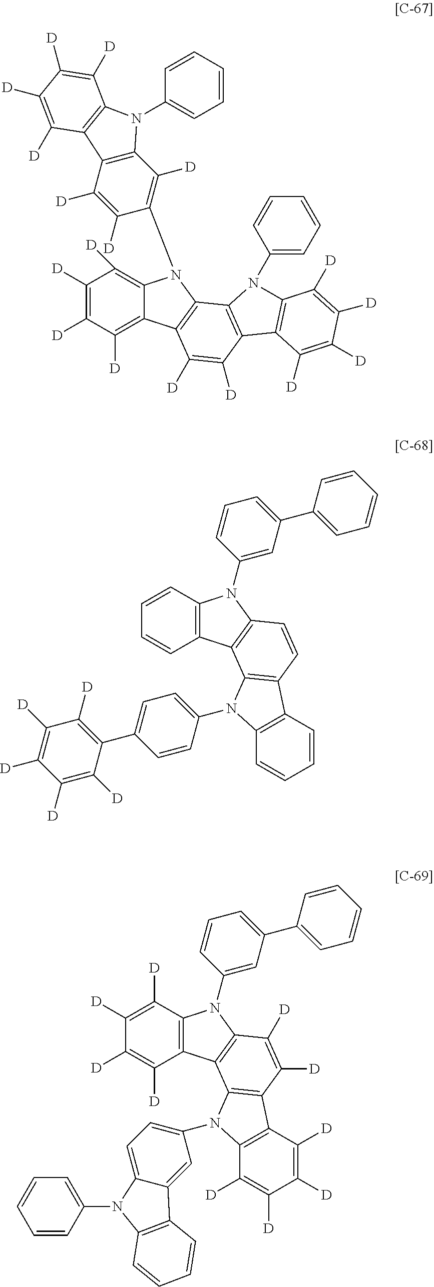 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1]

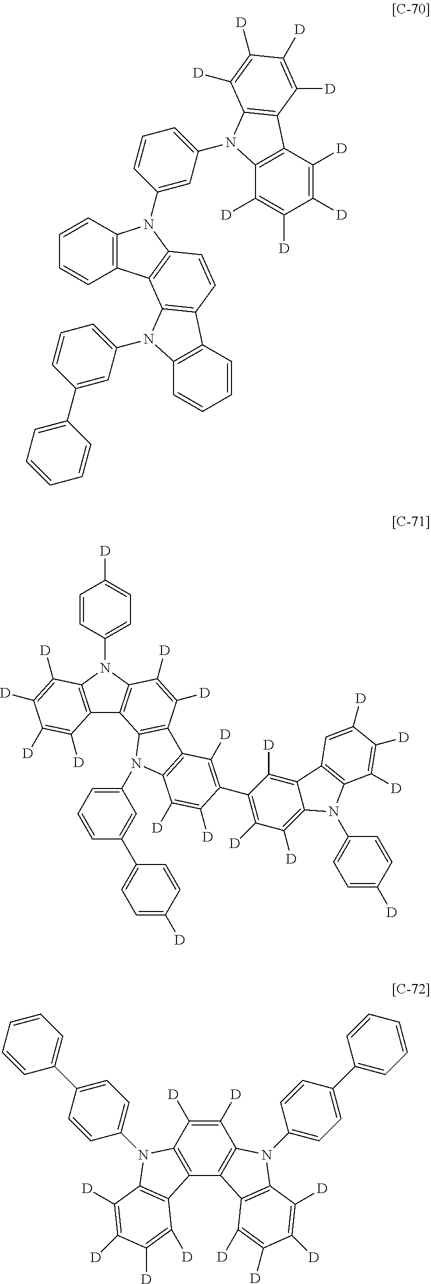 [SMILES: [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1 ; [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

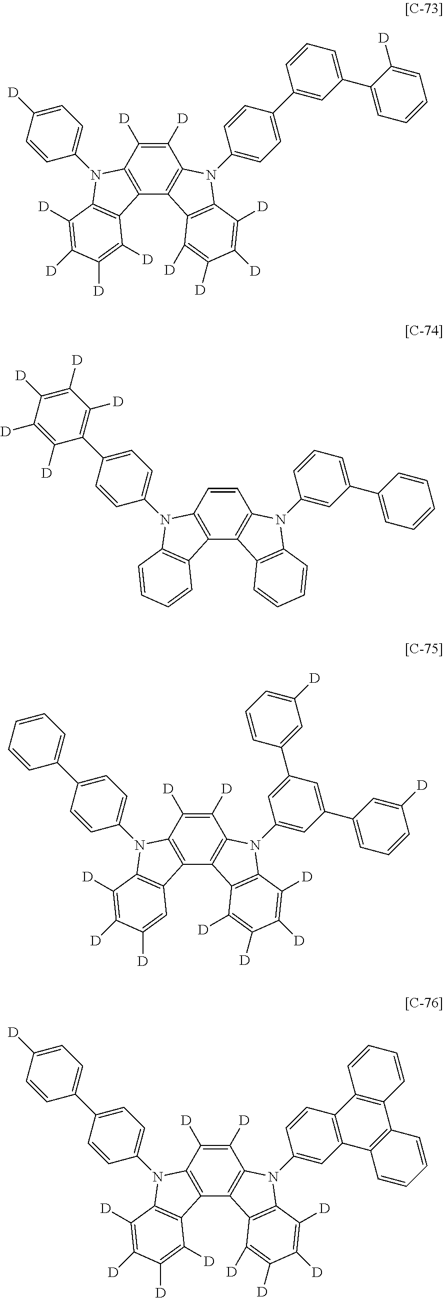

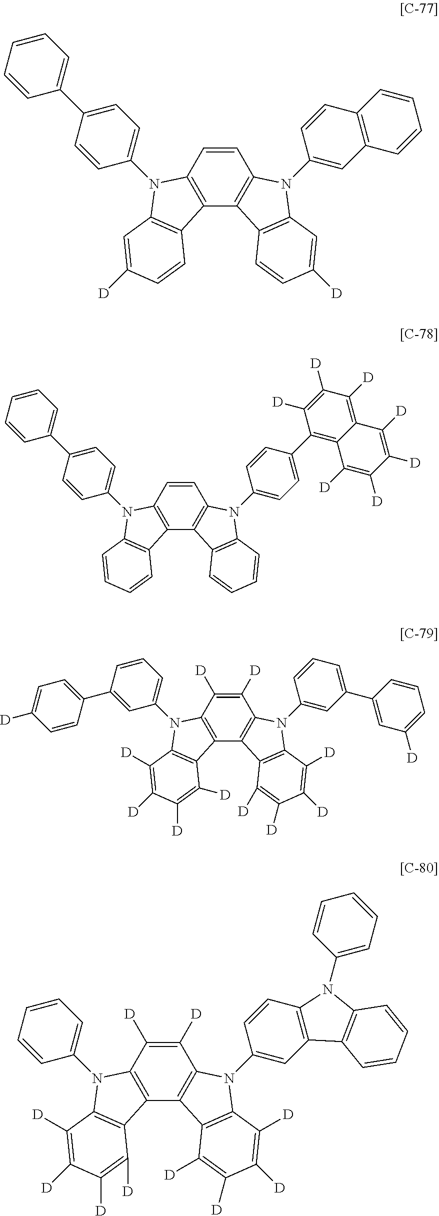 [SMILES: [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H] ; [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1 ; [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1]

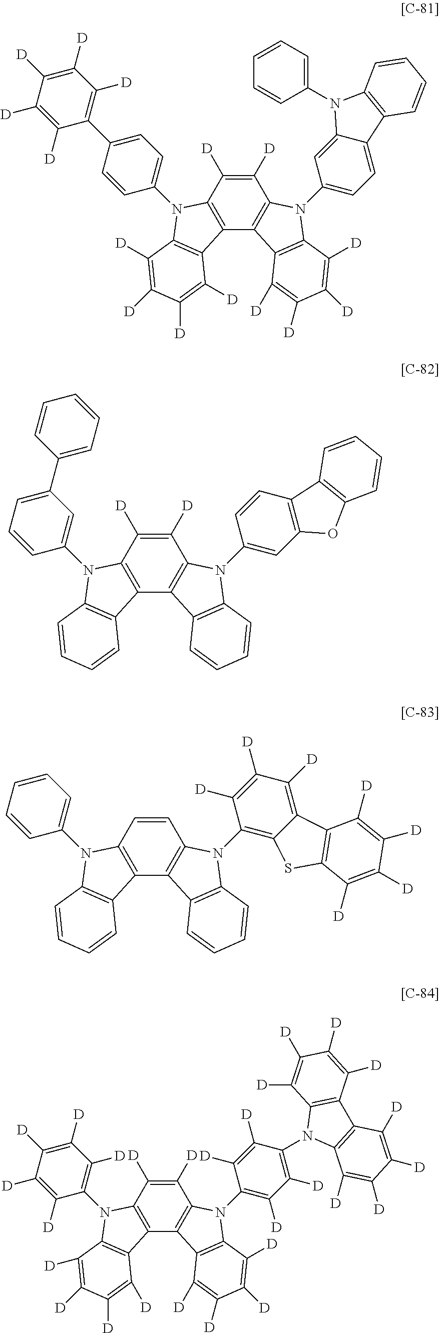

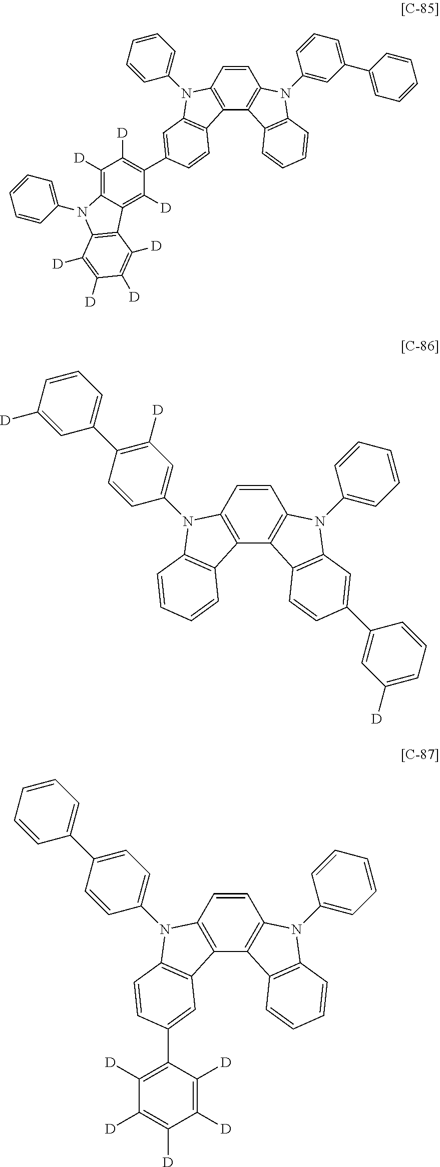 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

The first compound and the second compound may be included in a weight ratio of, for 10 example, 1:99 to 99:1. By being included in the above range, efficiency and life-span can be improved by implementing bipolar characteristics by adjusting the appropriate weight ratio using the electron transport capability of the first compound and the hole transport capability of the second compound. Within the above range, they may be included in a weight ratio of, for example, about 10:90 to 90:10, about 20:80 to 80:20, for example about 20:80 to about 70:30, about 20:80 to about 60:40, and about 20:80 to about 50:50. As a specific example, they may be included in a weight ratio of 20:80, 30:70, or 40:60.

In addition to the aforementioned first compound and second compound described above, one or more compounds may be further included.

For example, the aforementioned compound for an organic optoelectronic device or composition for an organic optoelectronic device may further include a dopant.

The dopant may be, for example, a phosphorescent dopant, such as a red, green, or blue phosphorescent dopant, and may be, for example, a red or green phosphorescent dopant.

The dopant is a material mixed with the compound for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L⁷MX [Chemical Formula Z]

In Chemical Formula Z, M is a metal, and L⁷ and X are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L⁷ and X may be, for example a bidentate ligand.

Examples of the ligands represented by L⁷ and X may be selected from the Chemical Formulas listed in Group A, but are not limited thereto.

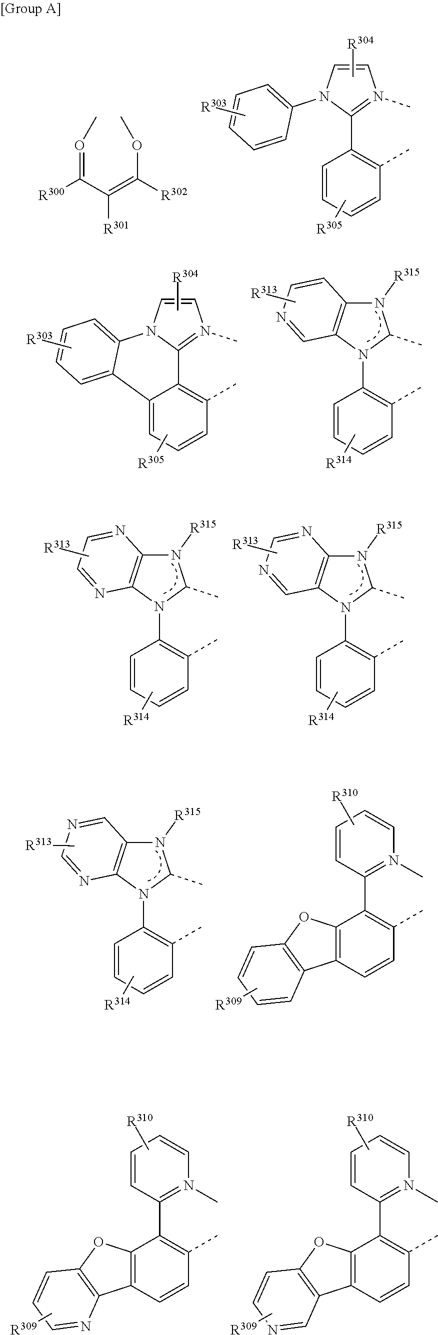

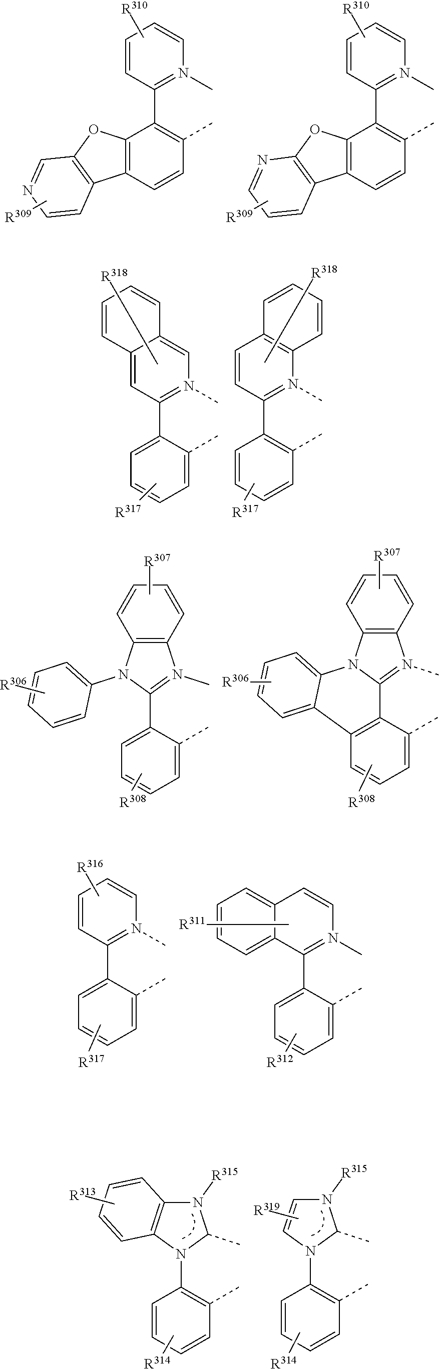

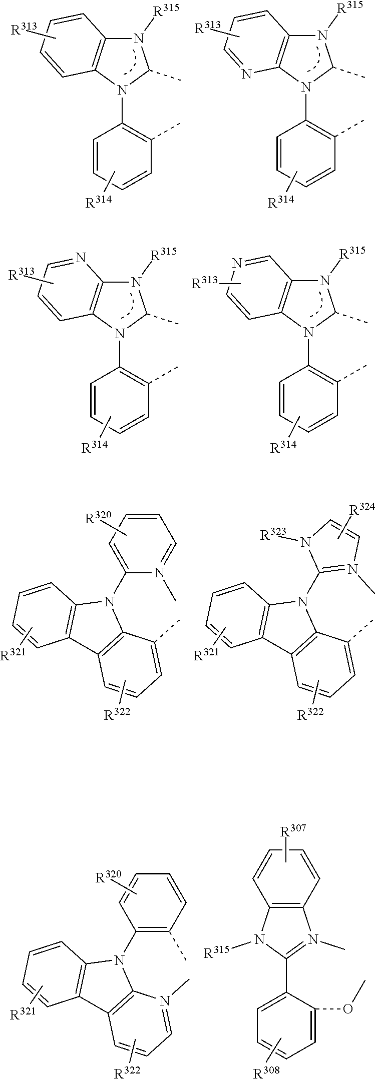

In Group A,
R³⁰⁰ to R³⁰² are each independently hydrogen, deuterium a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

For example, a dopant represented by Chemical Formula V may be included.

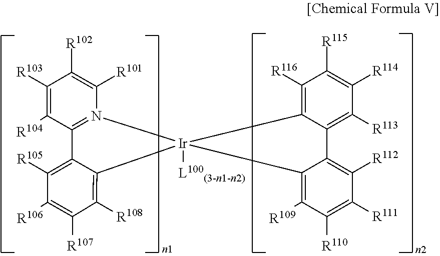

In Chemical Formula V,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴
R¹³² to R¹³⁴ are each independently a C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3,

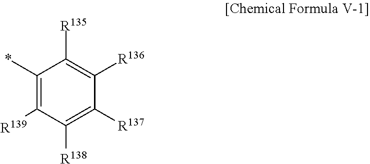

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴ and
* means a portion linked to a carbon atom.

For example, the dopant represented by Chemical Formula Z-1 may be included.

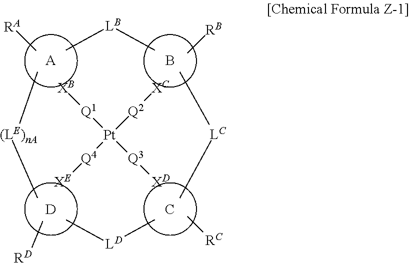

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof; and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, arylalkyl group, an alkoxy group, aryloxy group, an amino group, a silyl group, an alkenyl group, a cycloalkenyl group, a heteroalkenyl group, an alkynyl group, aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by Chemical Formula VI.

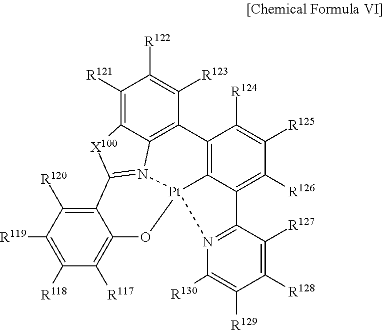

In Chemical Formula VI,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴
R¹³² to R¹³⁴ are each independently a C1 to C6 alkyl group, and
at least one of R¹¹⁷ to R¹³¹ is —SiR¹³²R¹³³R¹³⁴ or a tert-butyl group.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

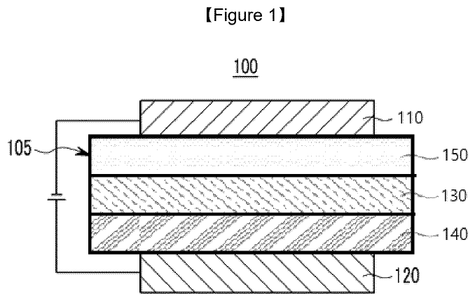

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca, but is not limited thereto.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including a dopant may be, for example, a red or green light emitting composition.

The light emitting layer 130 may include, for example, the aforementioned compound for organic optoelectronic devices or composition for organic optoelectronic devices, respectively, as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

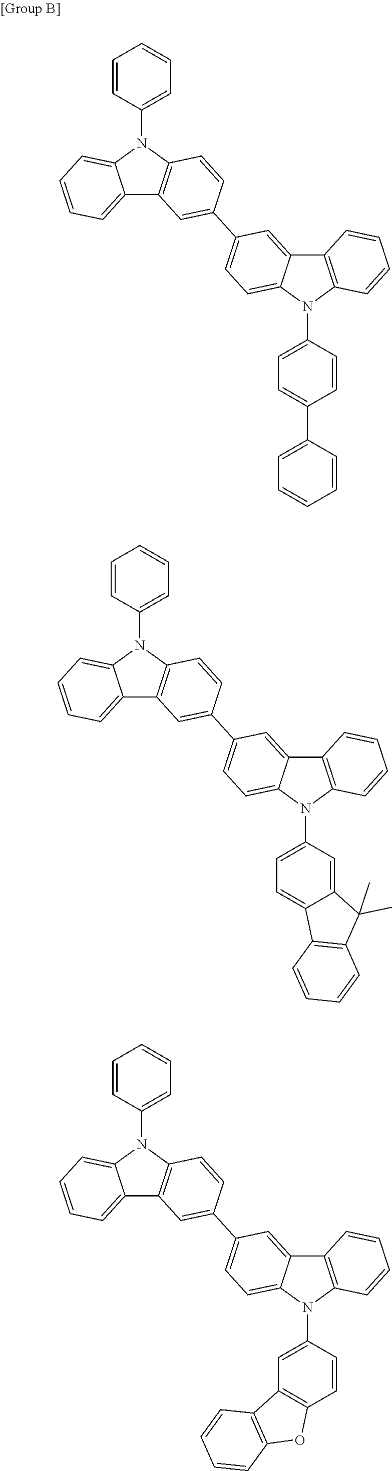 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

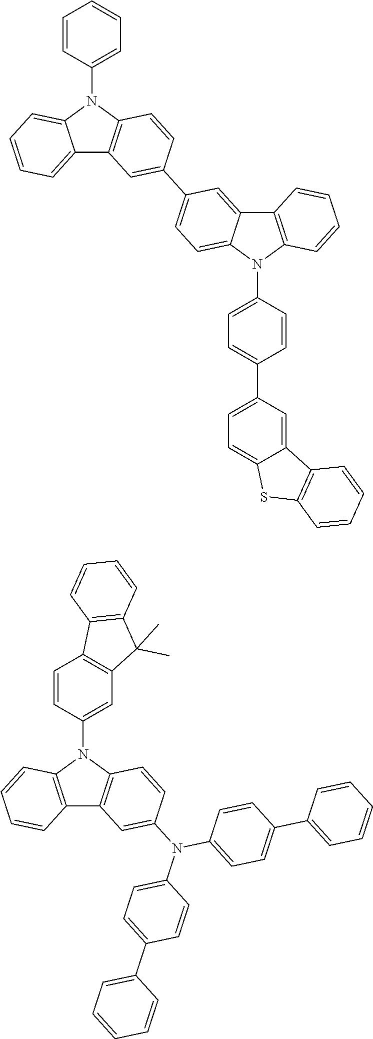 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

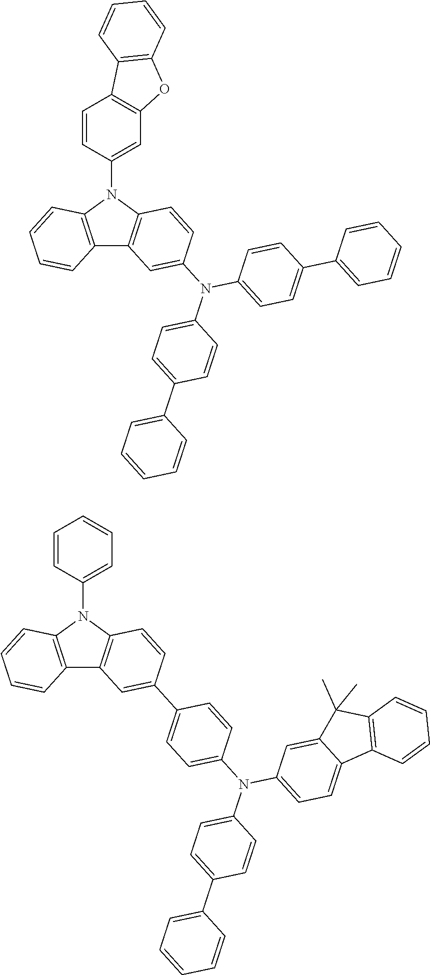 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

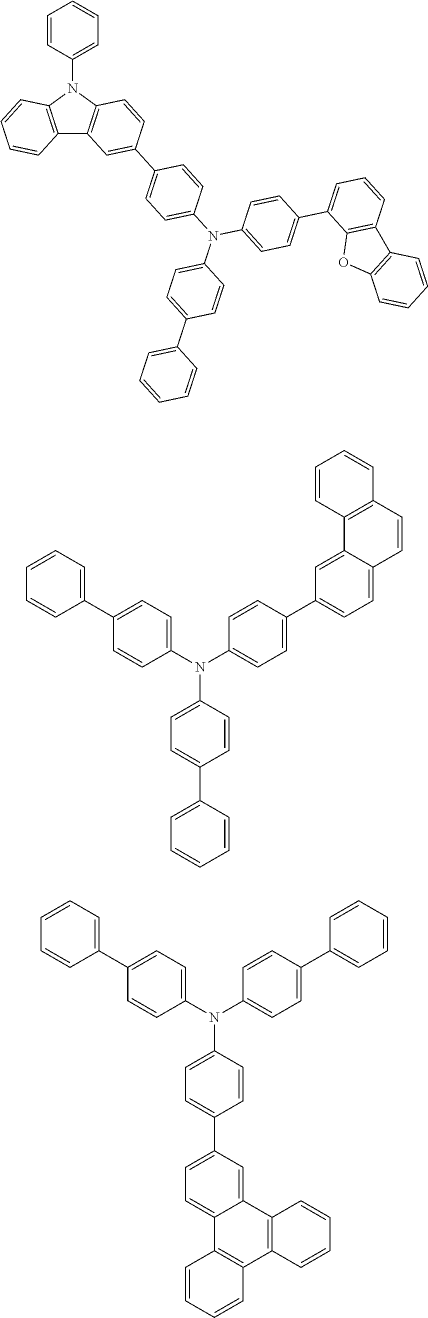 [SMILES: c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1]

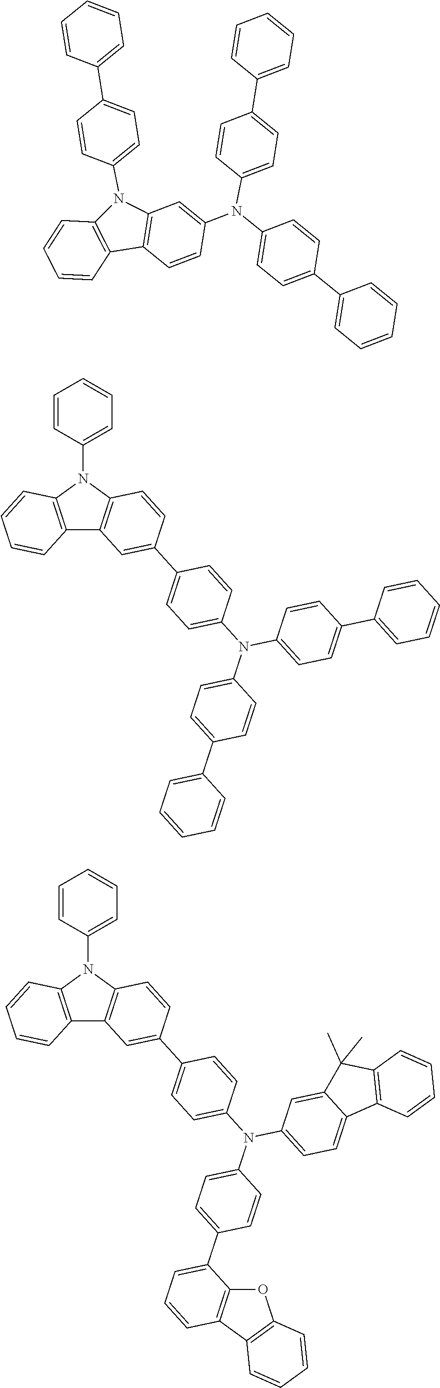 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

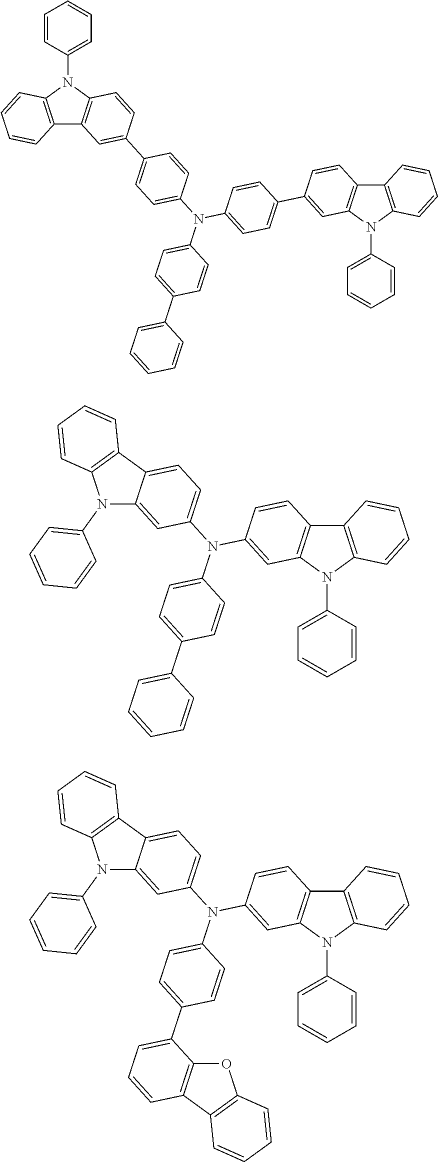 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

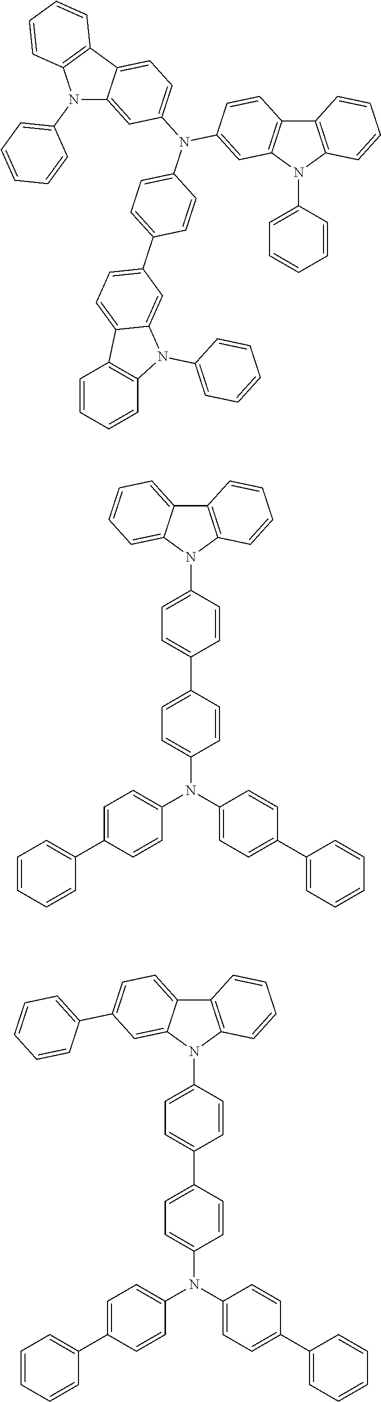 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

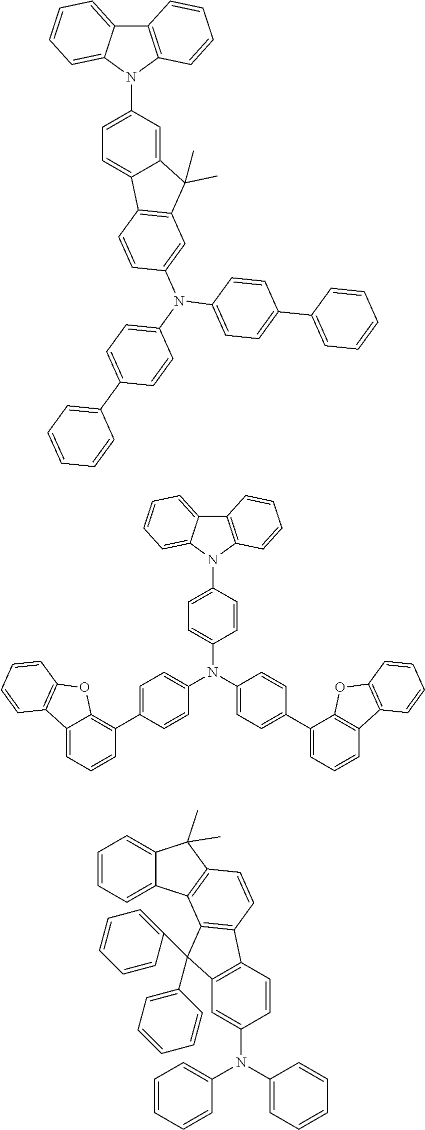 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

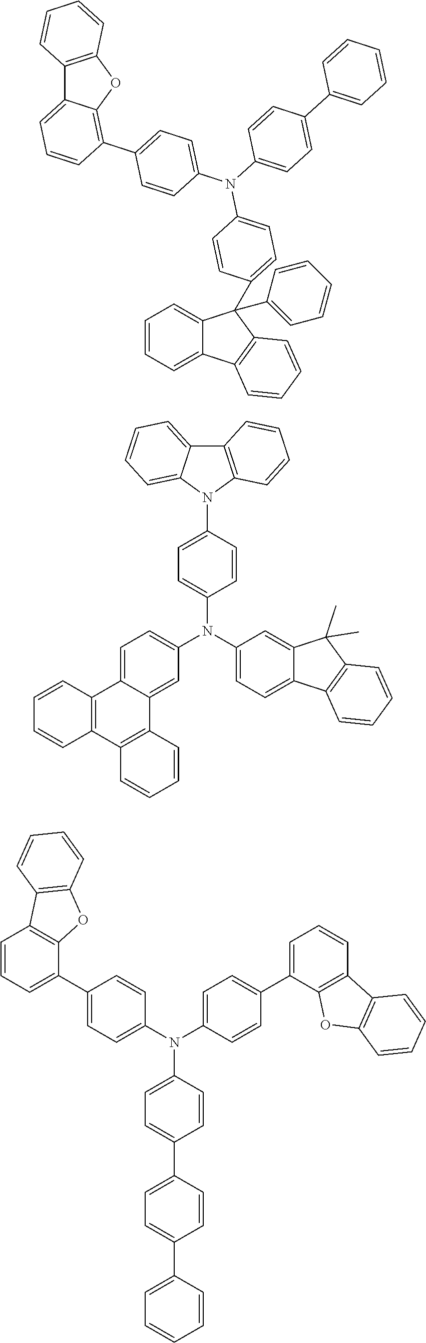

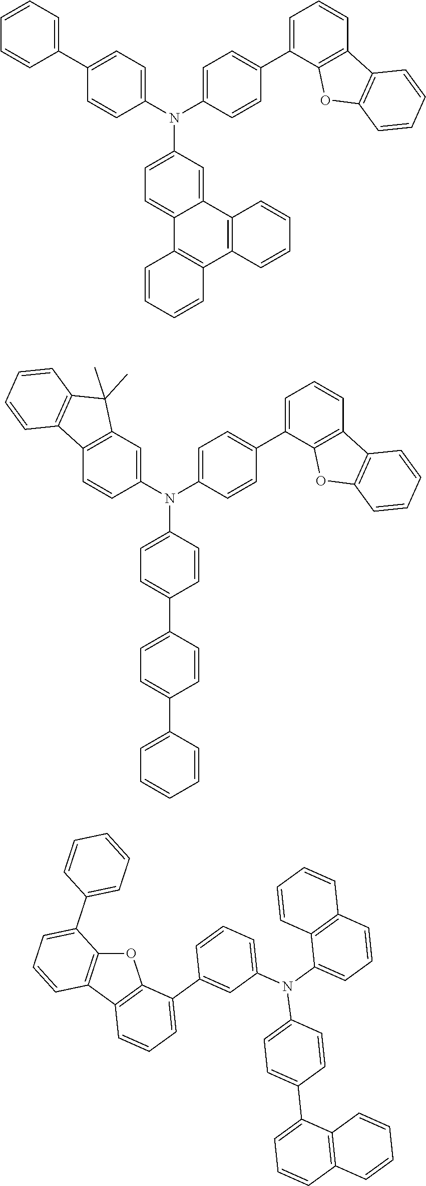 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

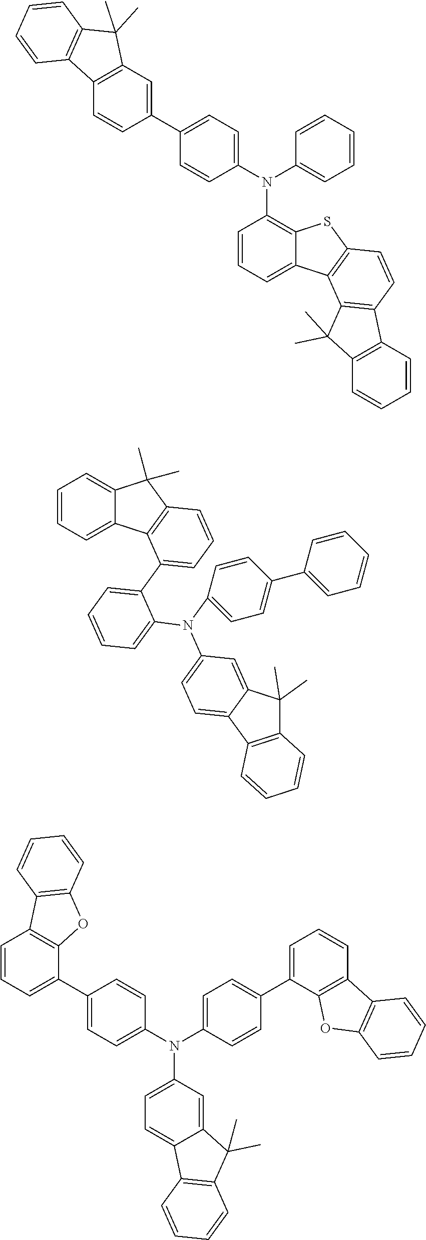 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

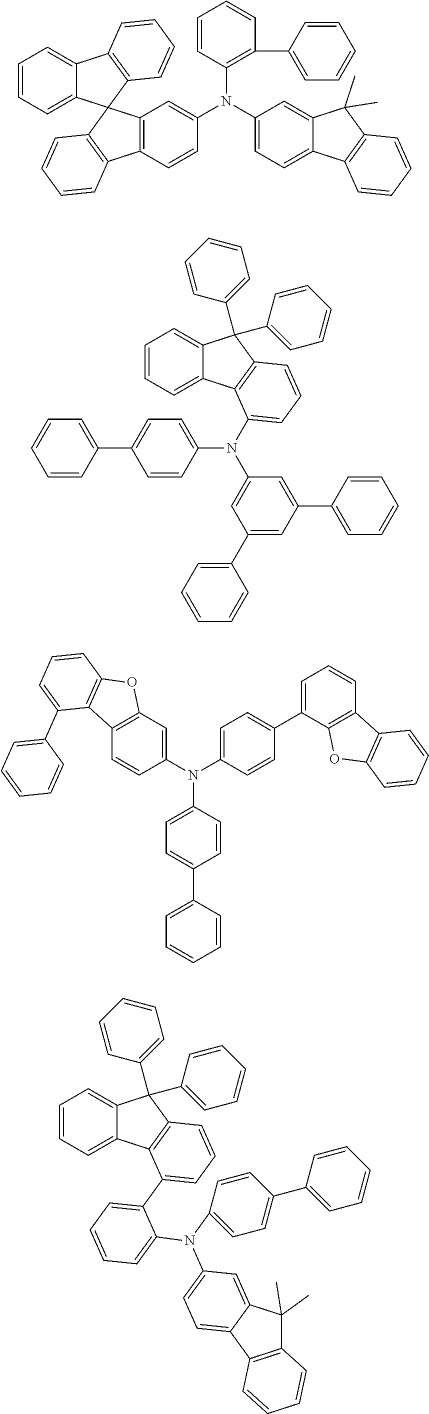 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

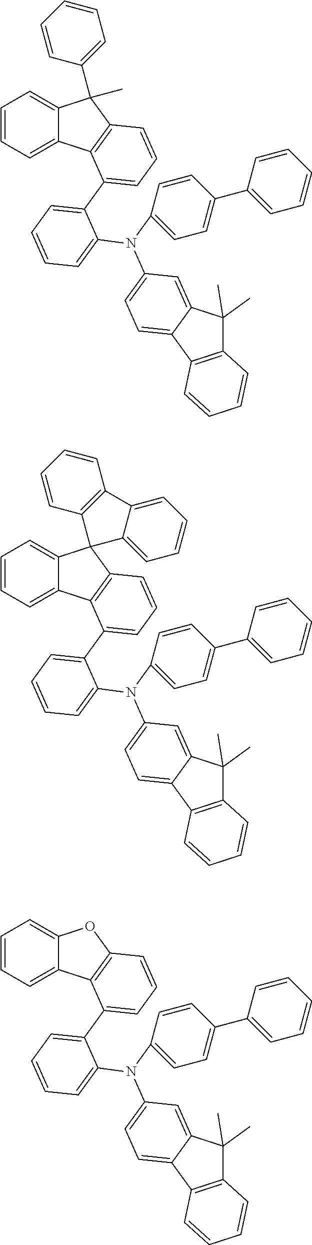 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

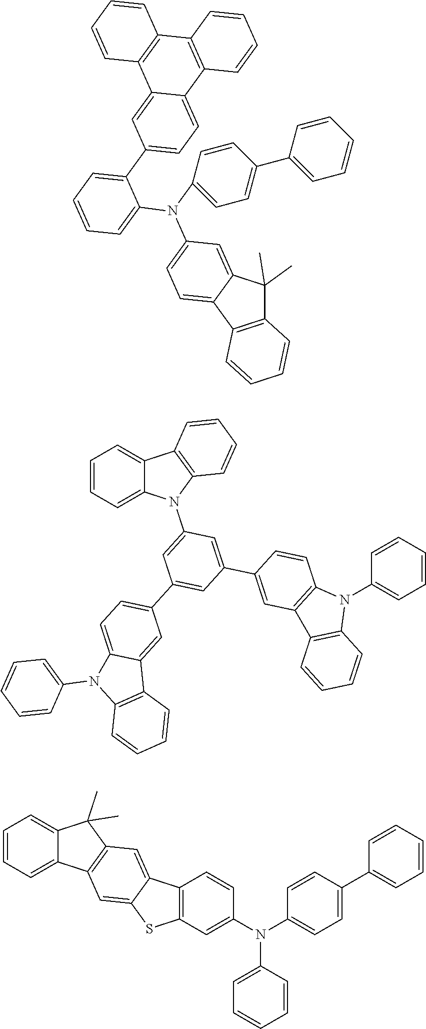 [SMILES: CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21]

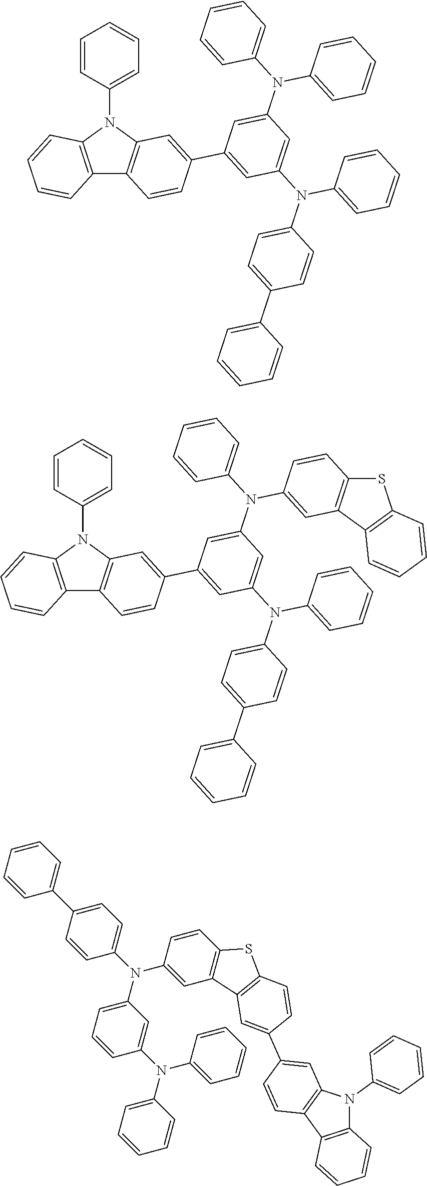

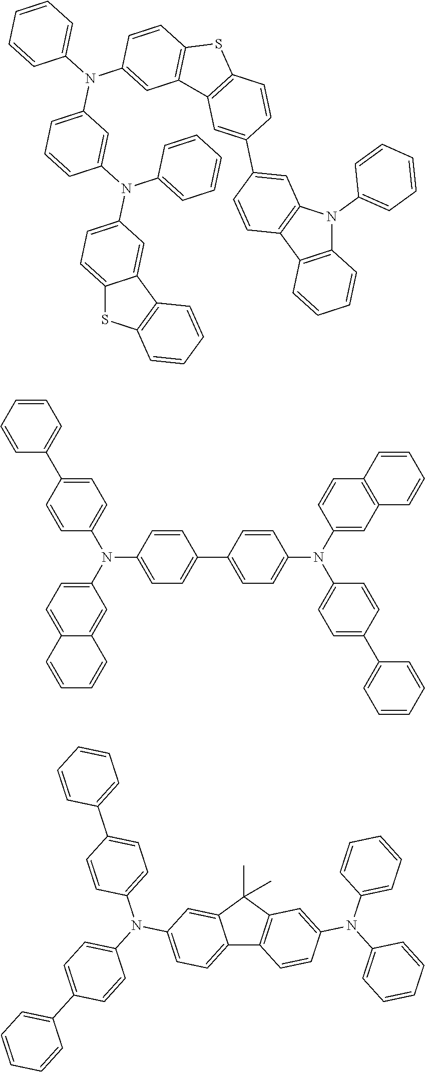 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

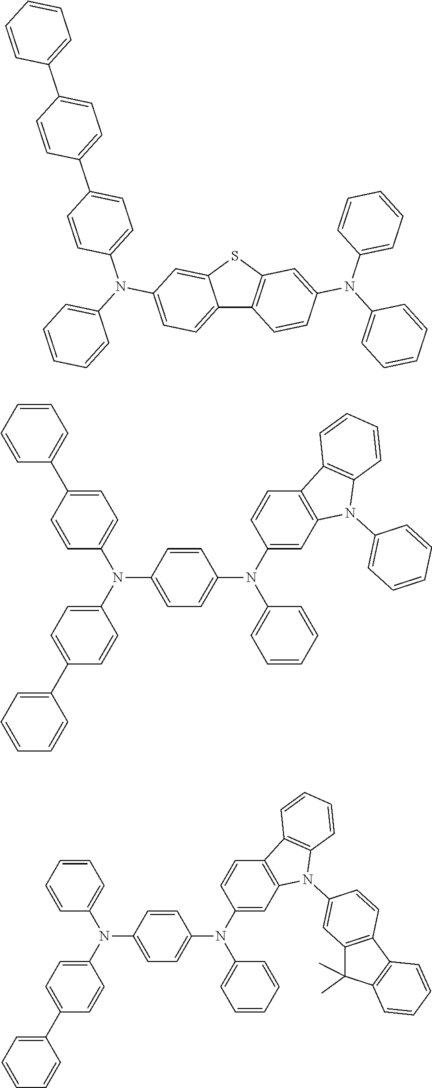

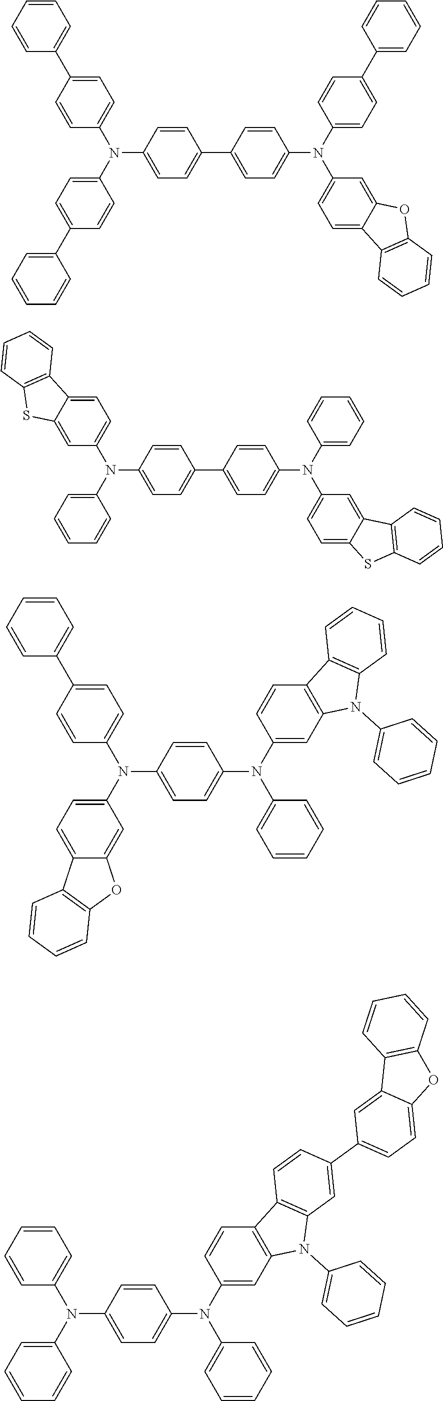

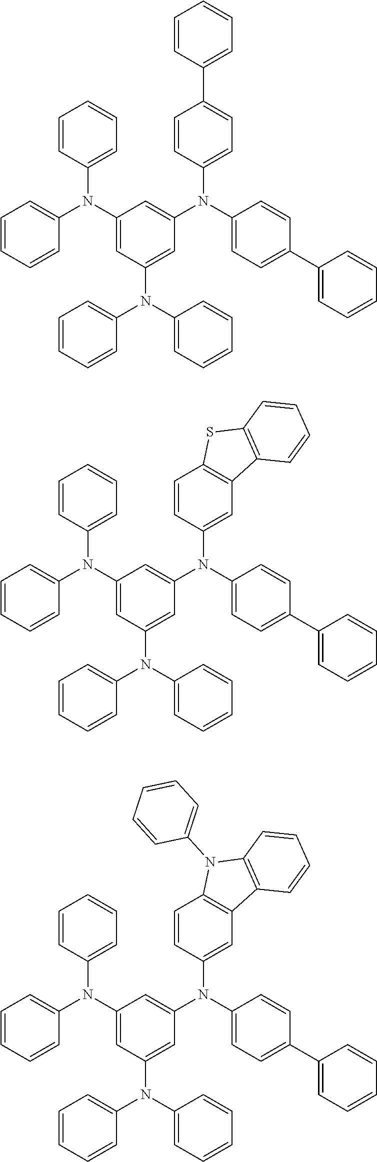 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

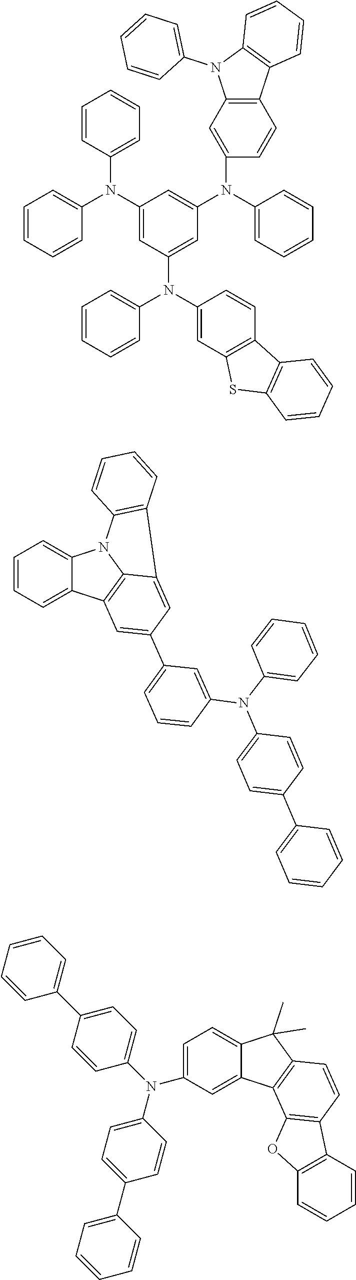 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

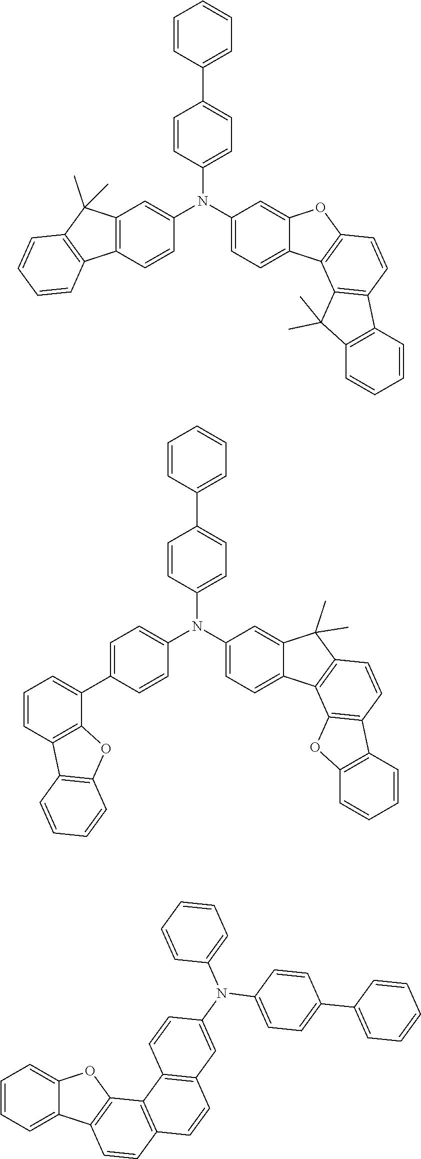 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1]

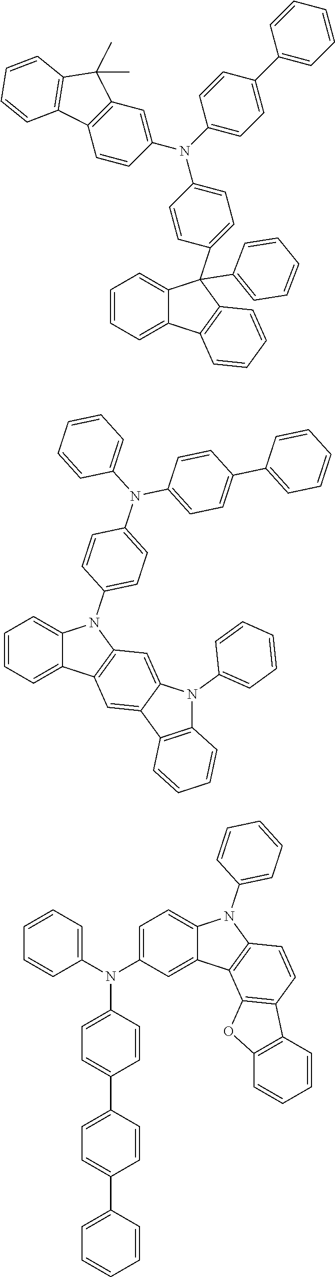 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

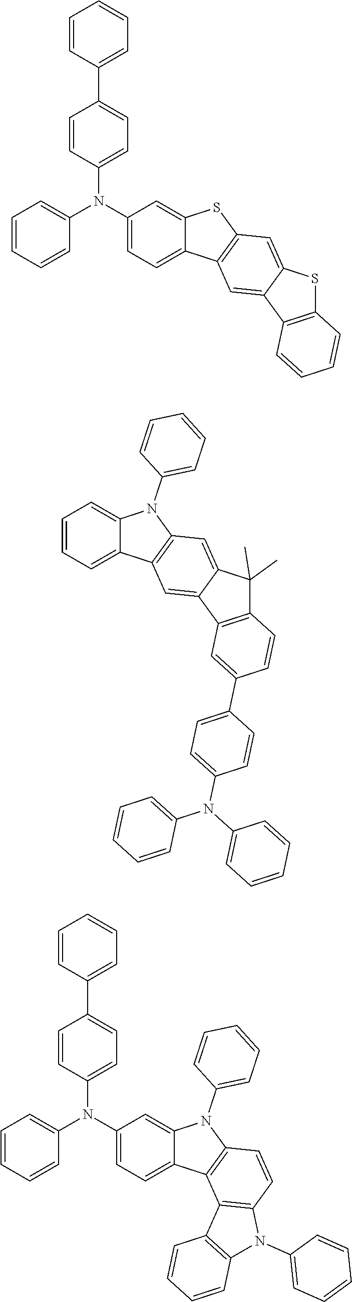 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

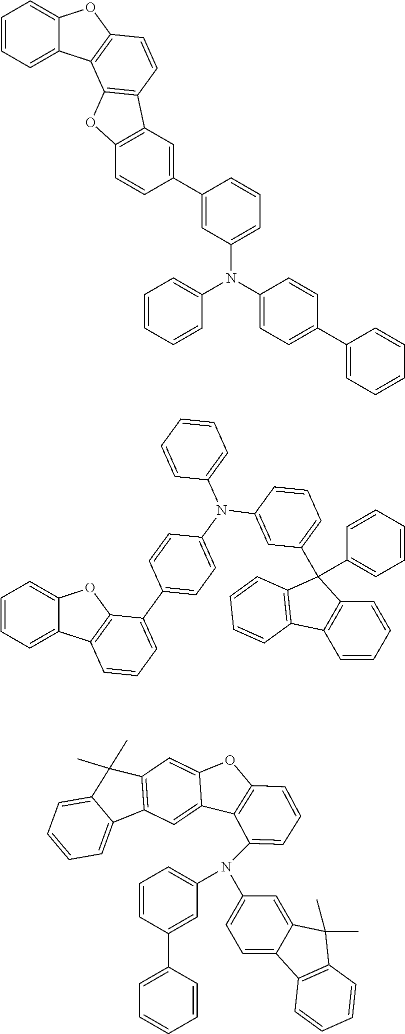 [SMILES: c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21]

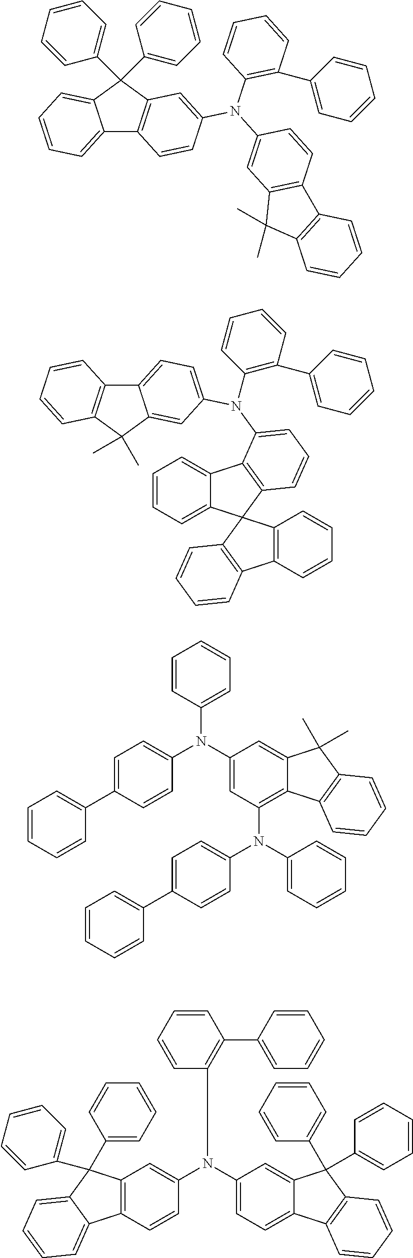

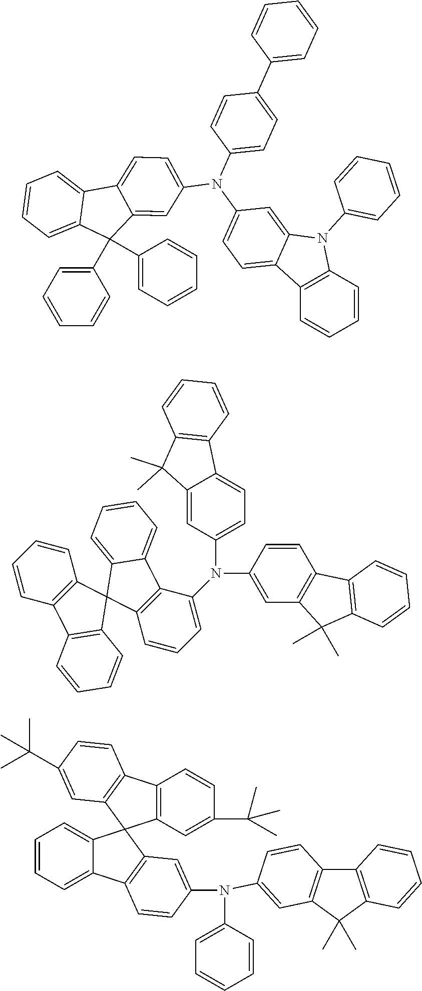 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

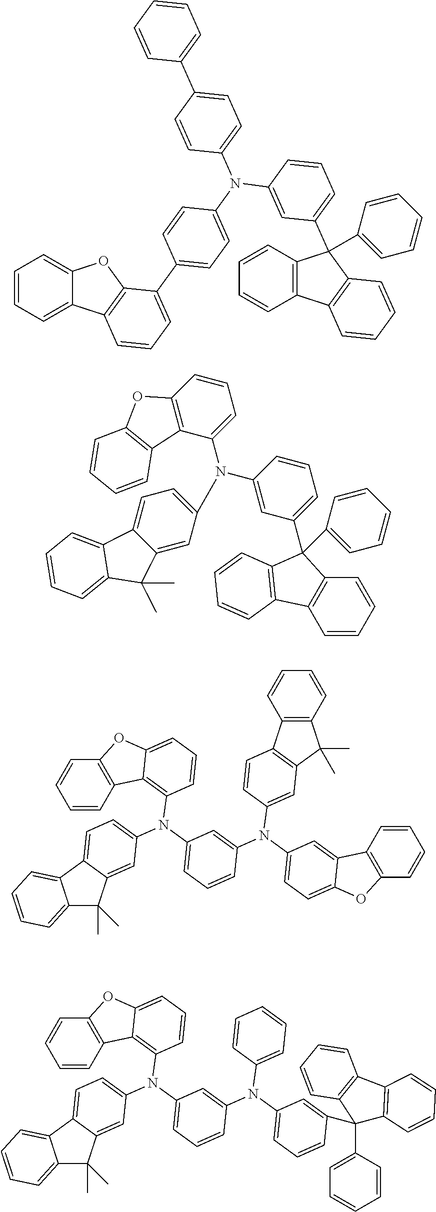

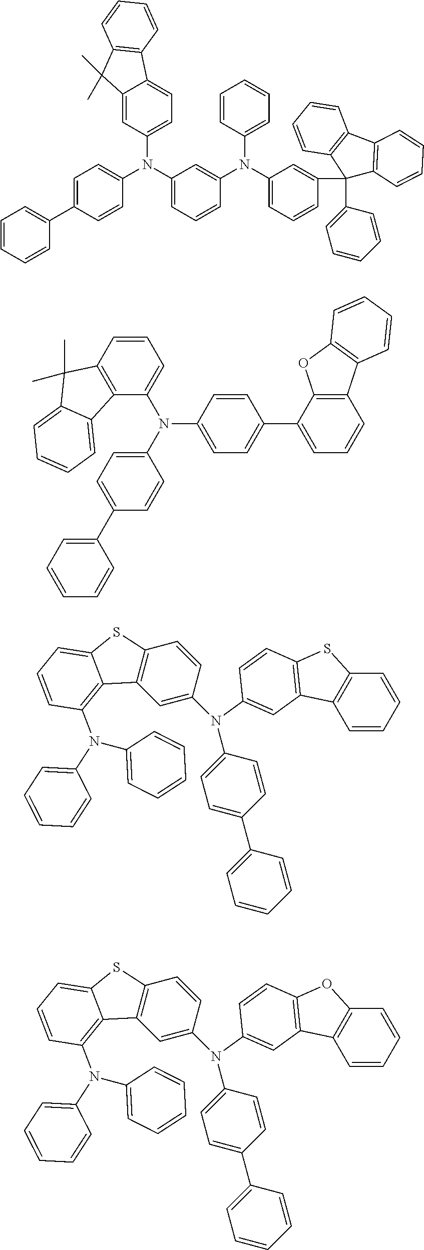

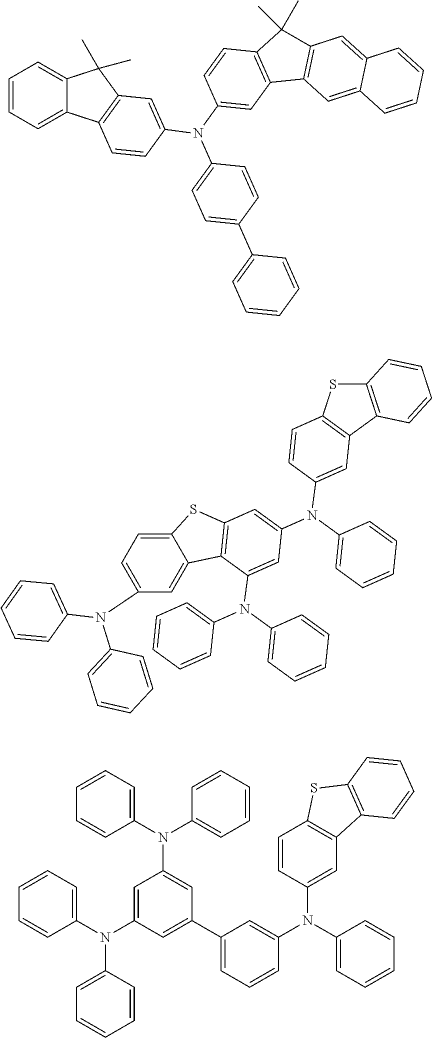 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1]

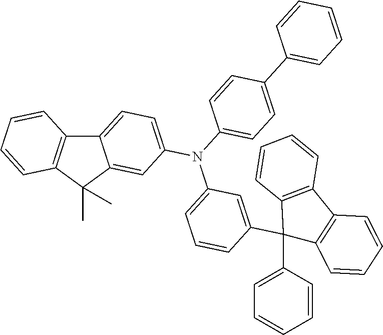 [SMILES: CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

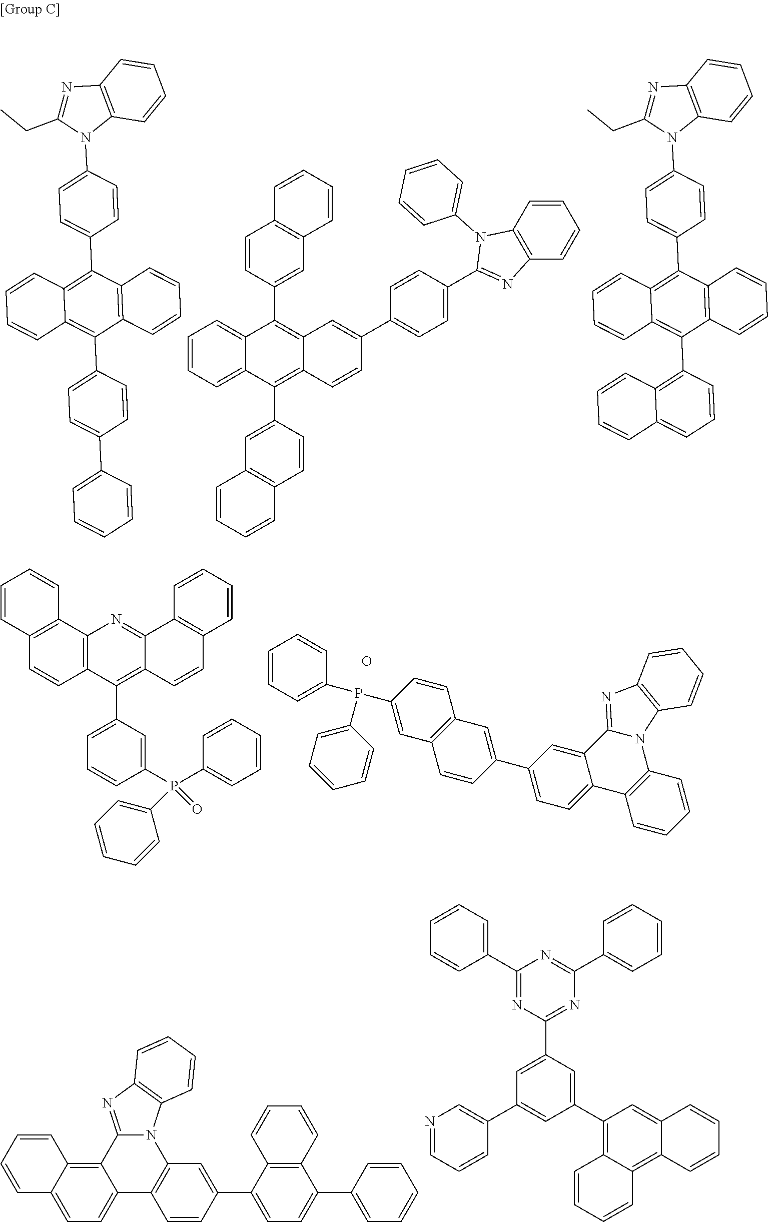

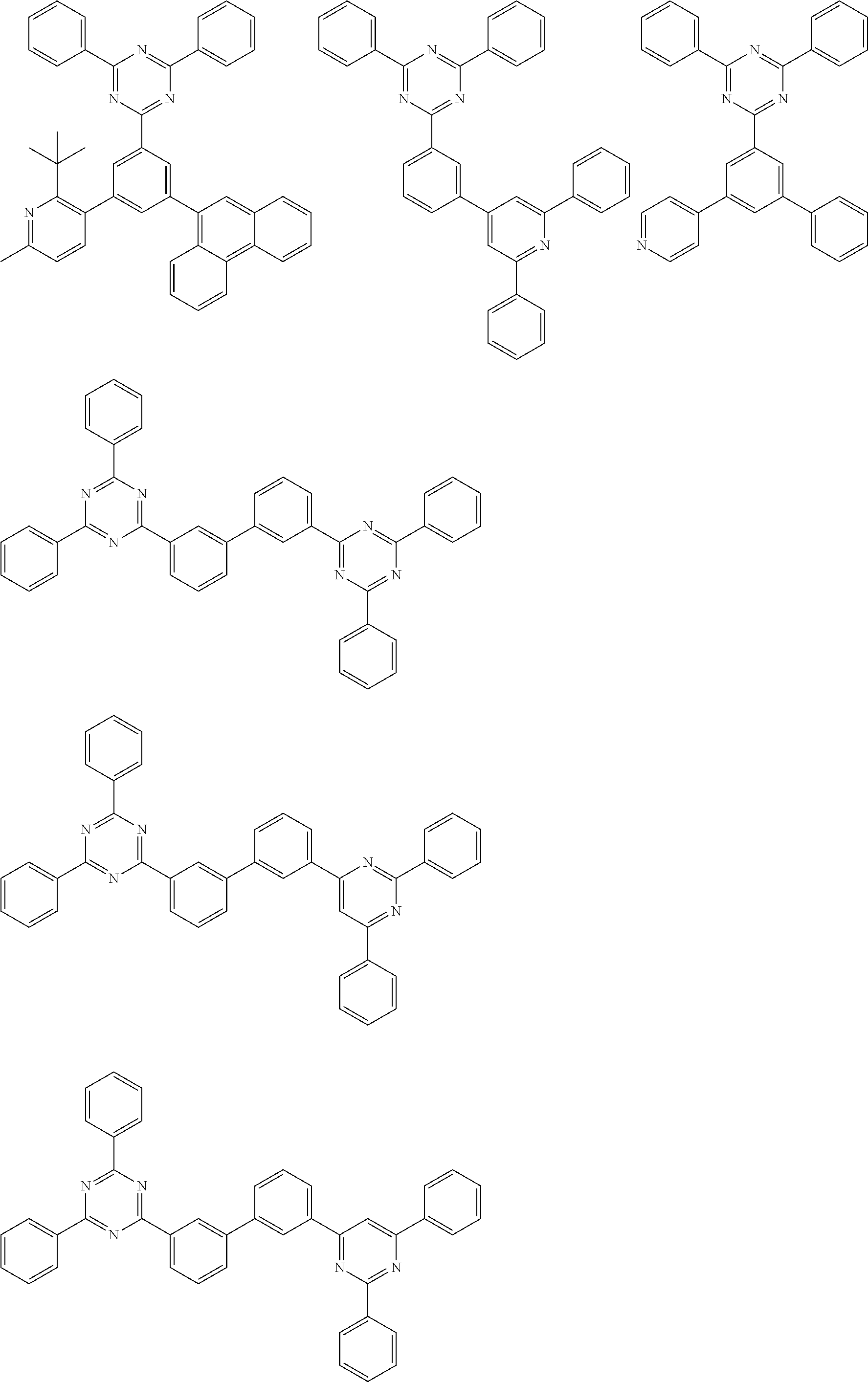

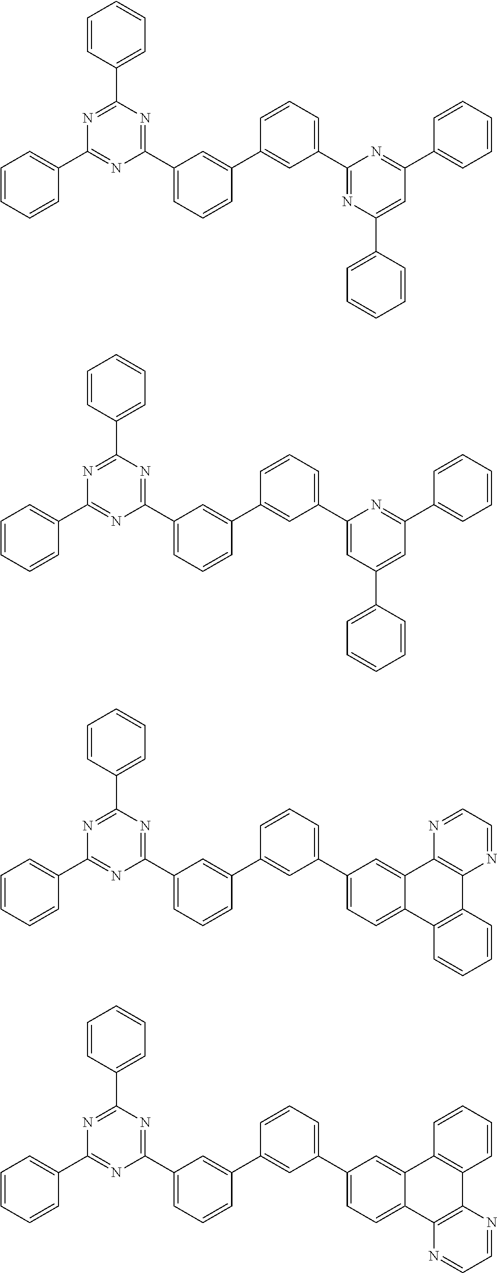 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

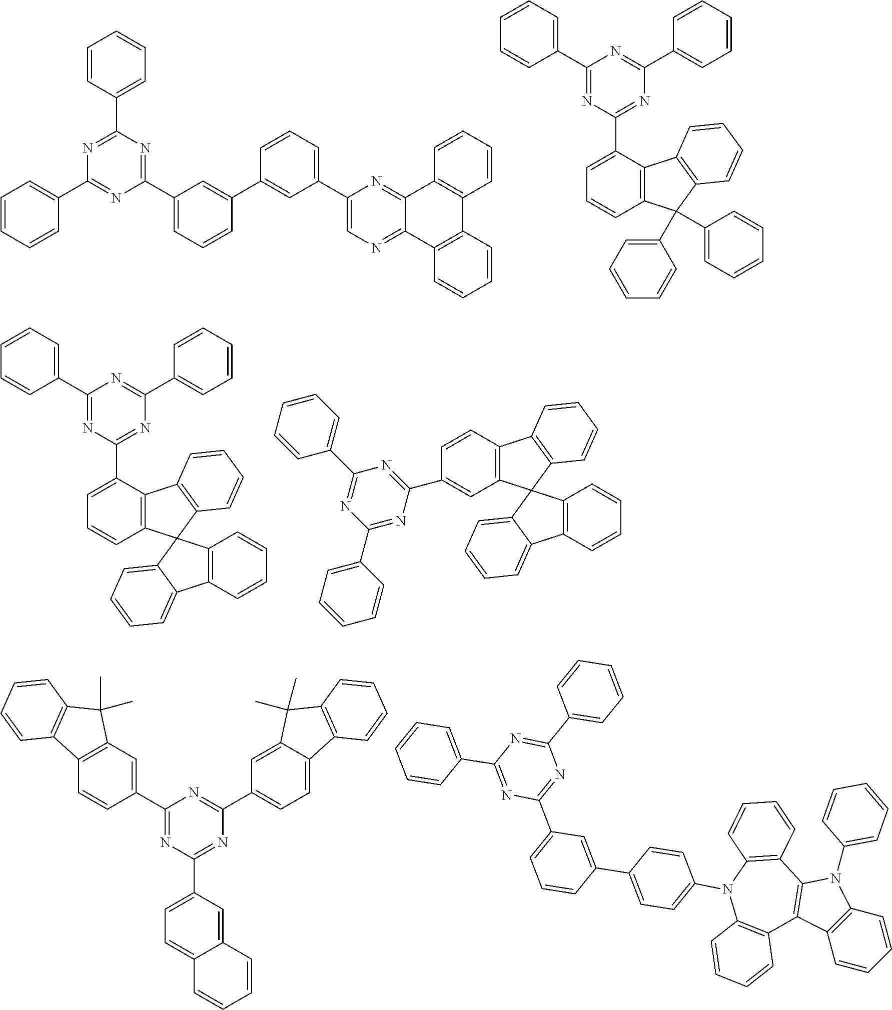

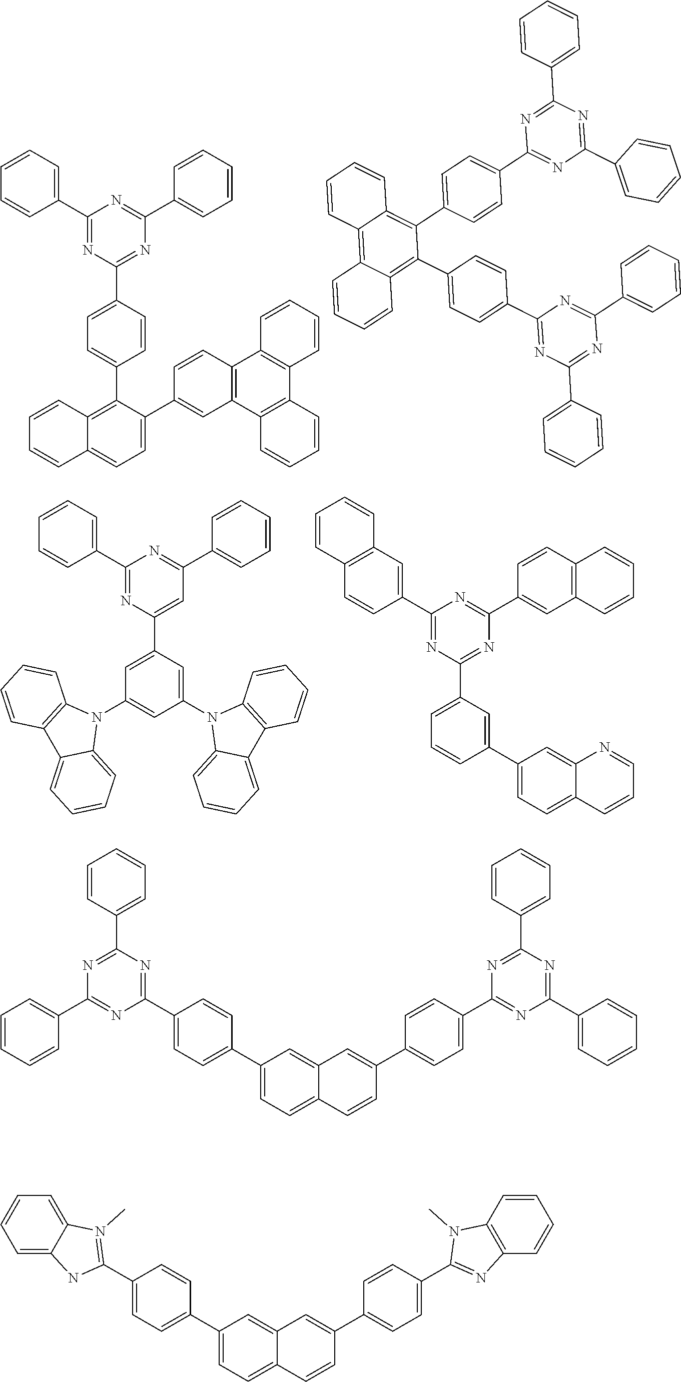

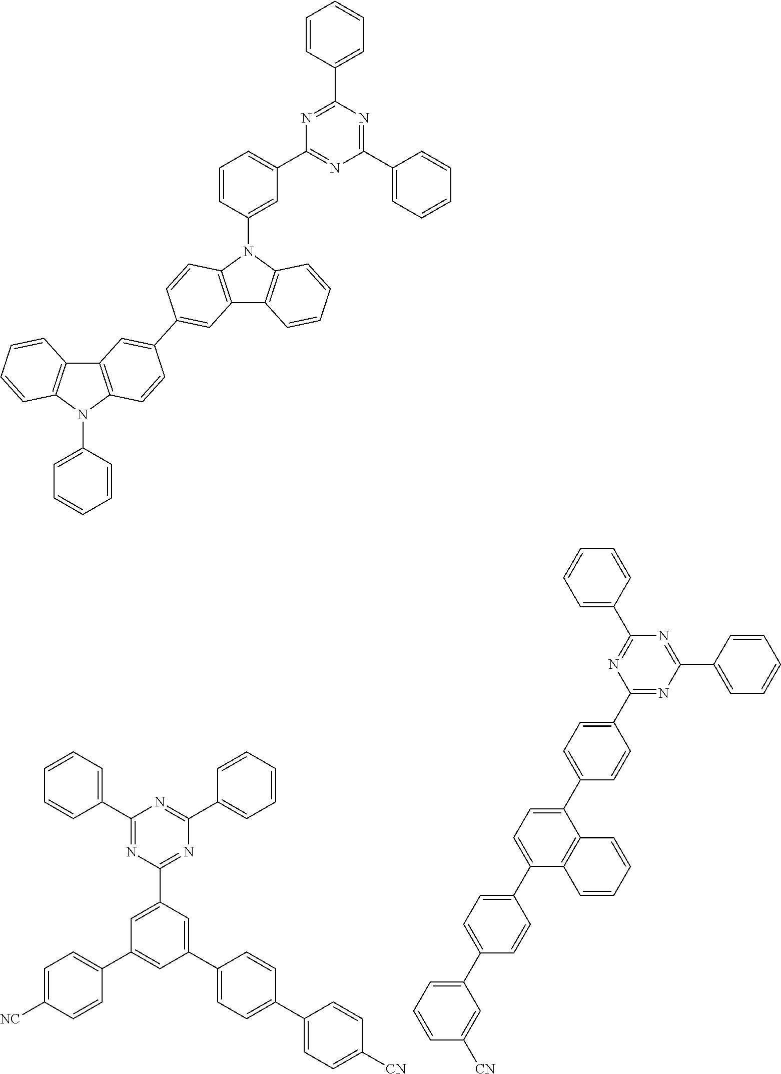 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1]

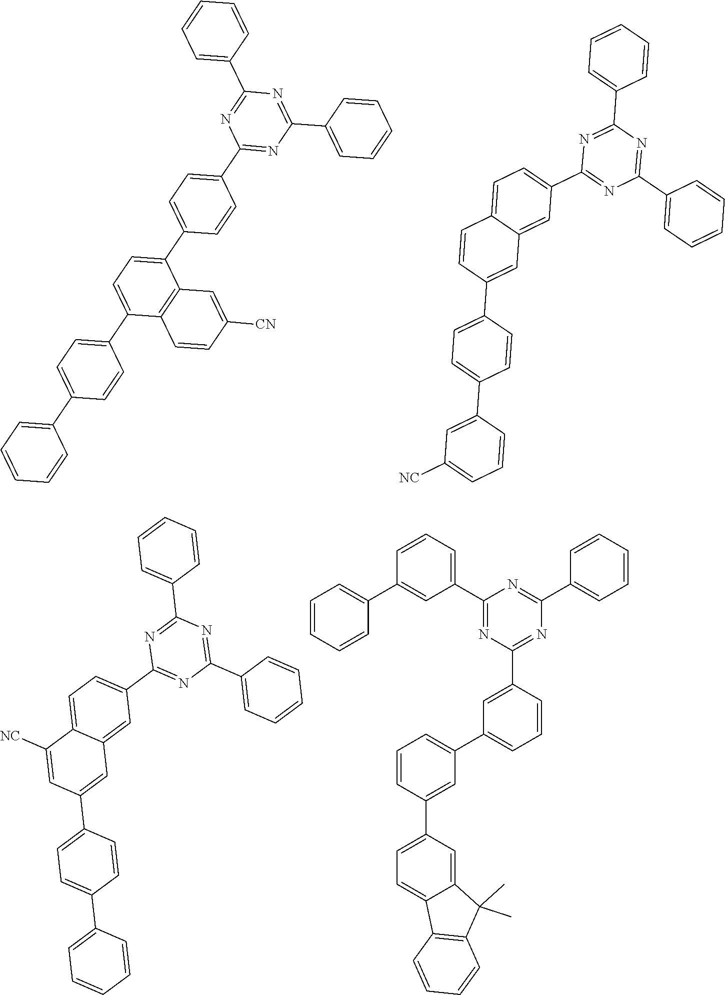

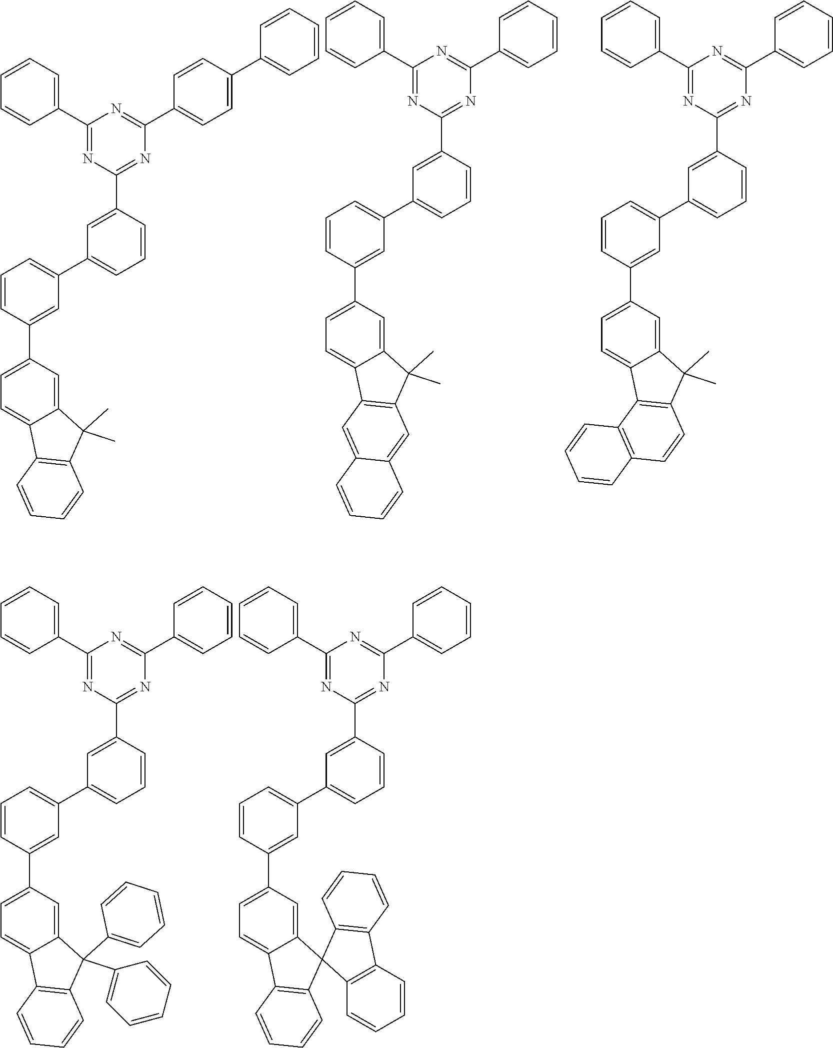

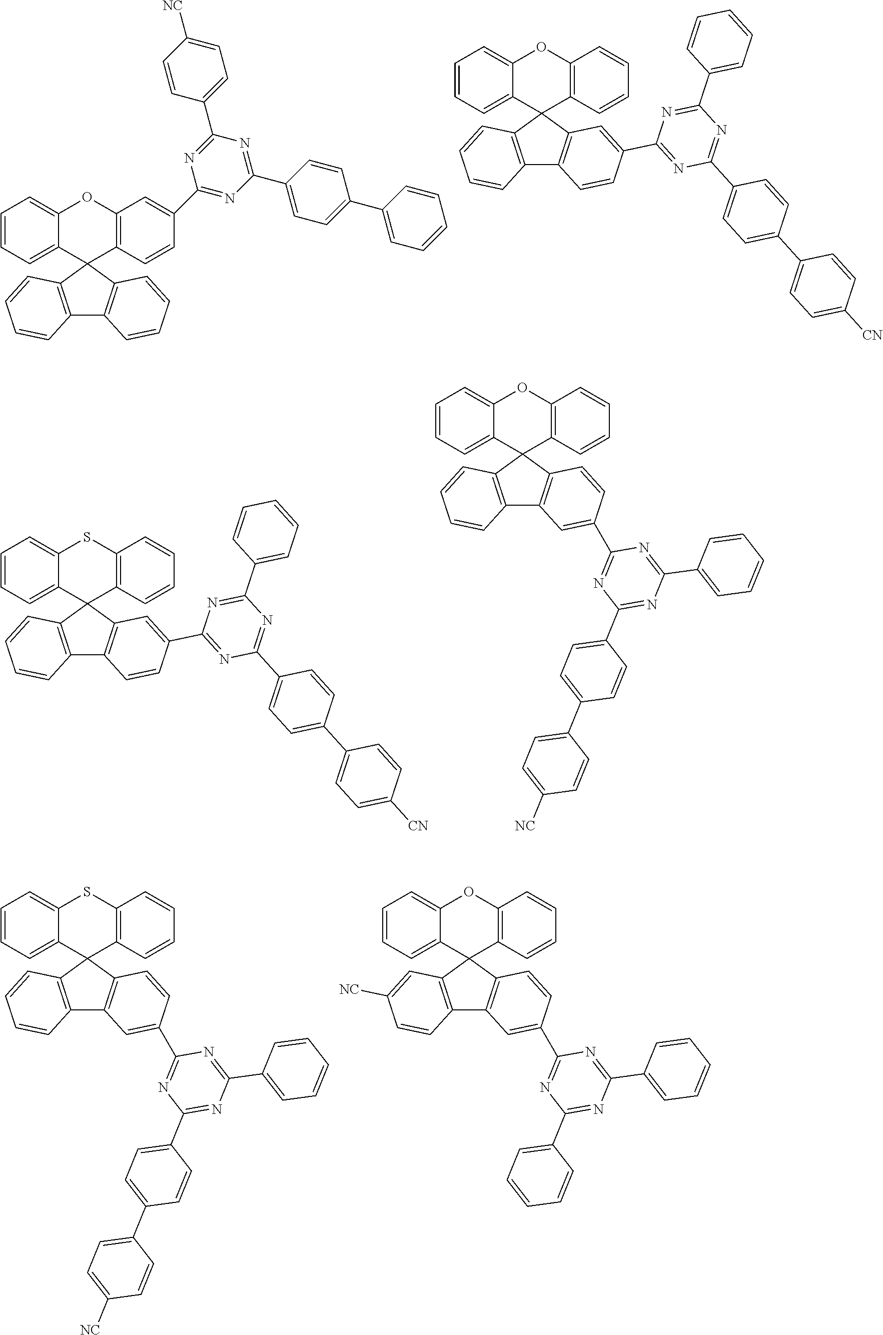

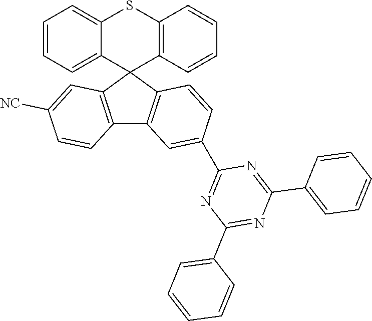 [SMILES: N#Cc1ccc2c(c1)C1(c3ccccc3Sc3ccccc31)c1ccc(-c3nc(-c4ccccc4)nc(-c4ccccc4)n3)cc1-2]

An embodiment of the present invention may provide an organic light emitting diode including the light emitting layer as the organic layer.

Another embodiment of the present invention may provide an organic light emitting diode including a light emitting layer and a hole transport region as the organic layer.

Another embodiment of the present invention may provide an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

In another embodiment of the present invention, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### DETAILED DESCRIPTION OF THE EMBODIMENTS

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there in no particular comment or were synthesized by known methods.

### (Preparation of Compound for Organic Optoelectronic Device)

The compounds presented as a more specific example of the compound of the present invention were synthesized through the following steps.

### Synthesis Example 1: Synthesis of Intermediate I-1

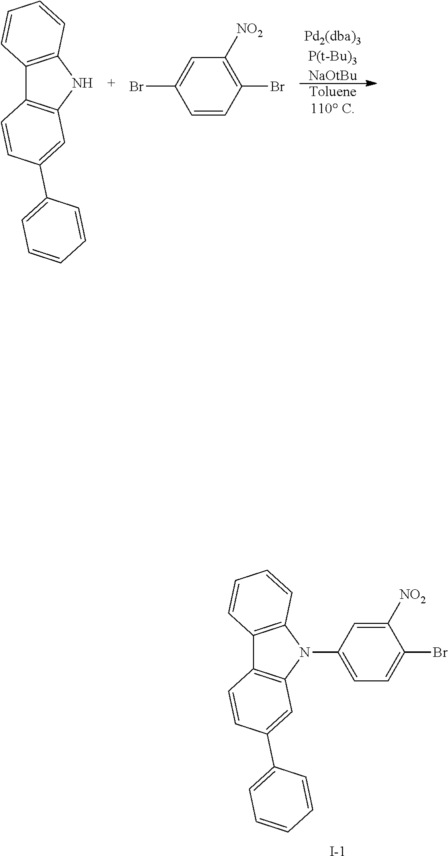 [SMILES: c1ccc(-c2ccc3c(c2)[nH]c2ccccc23)cc1 ; O=[N+]([O-])c1cc(Br)ccc1Br ; "Pd2(dba)3P(t-Bu)3NaOtBu" ; "Toluene110° C." ; O=[N+]([O-])c1cc(-n2c3ccccc3c3ccc(-c4ccccc4)cc32)ccc1Br ; [I-]]

In a nitrogen environment, after dissolving 2-phenyl-9H-carbazole (50 g, 206 mmol) in 0.5 L of toluene, 1,4-dibromo-2-nitrobenzene (57.7 g, 206 mmol), tris(diphenylideneacetone)dipalladium (0) (1.89 g, 2.06 mmol), tris-tert butylphosphine (2.08 g, 10.3 mmol), and sodium tert-butoxide (23.8 g, 247 mmol) were sequentially added thereto and then, heated under reflux at 110° C. for 8 hours. When a reaction was completed, after adding water to the reaction solution, an extract was obtained therefrom with dichloromethane (DCM), treated with magnesium sulfate anhydrous to remove moisture, filtered, and concentrated under a reduced pressure. The obtained residue was separated and purified through flash column chromatography to obtain Intermediate I-1 (66.7 g, 73%).

HRMS (70 eV, EI+): m/z calcd for C24H15BrN2O2: 442.0317, found: 442.

Elemental Analysis: C, 65%; H, 3%

### Synthesis Example 2: Synthesis of Intermediate I-2

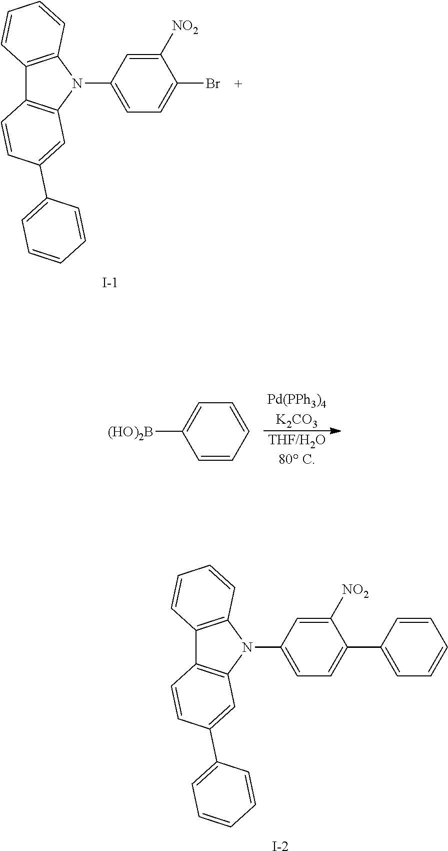

In the nitrogen environment, Intermediate I-1 (65 g, 147 mmol) was dissolved in 0.4 L of tetrahydrofuran (THF), and phenyl boronic acid (19.7 g, 161 mmol) and tetrakis(triphenylphosphine)palladium (3.40 g, 2.94 mmol) were added thereto and then, stirred. Subsequently, potassium carbonate (50.8 g, 368 mmol) saturated in water was added thereto and then, refluxed by heating at 80° C. for 12 hours. When a reaction was completed, after adding water to the reaction solution, an extract was obtained therefrom with dichloromethane (DCM), treated with magnesium sulfate anhydrous to remove moisture, filtered, and concentrated under a reduced pressure. The obtained residue was separated and purified through flash column chromatography to obtain Intermediate I-2 (55.0 g, 85%).

HRMS (70 eV, EI+): m/z calcd for C30H20N2O2: 440.1525, found: 440.

Elemental Analysis: C, 82%; H, 5%

### Synthesis Example 3: Synthesis of Intermediate I-3

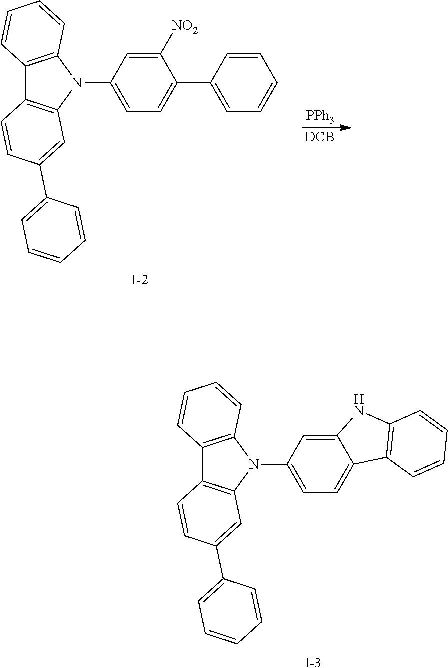

In the nitrogen environment, Intermediate I-2 (50 g, 114 mmol) was dissolved in 0.3 L of dichlorobenzene (DCB), and triphenylphosphine (59.8 g, 228 mmol) was added thereto and then, refluxed by heating at 200° C. for 3 hours. When a reaction was completed, after adding water to the reaction solution, an extract was obtained therefrom with dichloromethane (DCM), treated with magnesium sulfate anhydrous to remove moisture, filtered, and concentrated under a reduced pressure. The obtained residue was separated and purified through flash column chromatography to obtain Intermediate I-3 (44.2 g, 95%).

HRMS (70 eV, EI+): m/z calcd for C30H20N2: 408.1626, found: 408.

Elemental Analysis: C, 88%; H, 5%

### Synthesis Example 4: Synthesis of Intermediate I-4

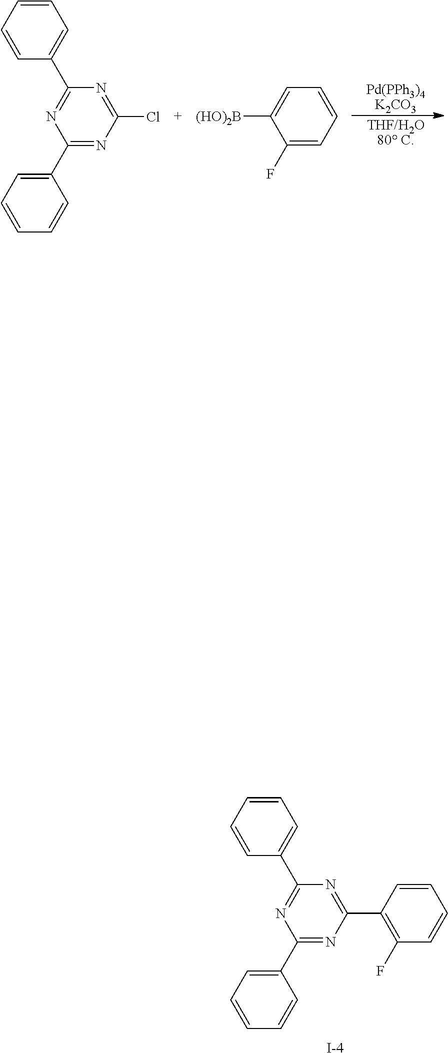

Intermediate I-4 (60.0 g, 98%) was obtained by using 2-chloro-4,6-diphenyl-1,3,5-triazine (50 g, 187 mmol) and 2-fluorophenylboronic acid (67.3 g, 205 mmol) in the same manner as in Synthesis Example 2.

HRMS (70 eV, EI+): m/z calcd for C21H14FN3: 327.1172, found: 327.

Elemental Analysis: C, 77%; H, 4%

### Synthesis Example 5: Synthesis of Compound A-1

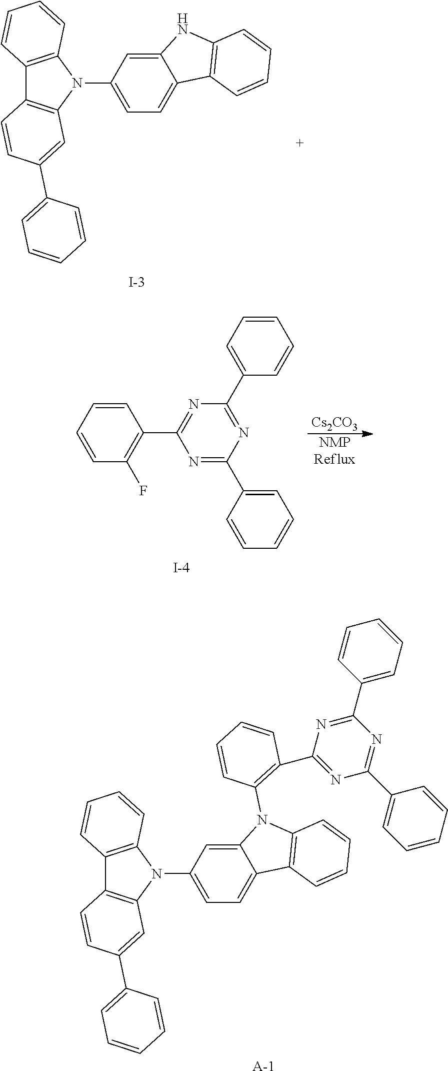

In the nitrogen environment, Intermediate I-3 (10 g, 24.5 mmol) was dissolved in 0.1 L of N-methyl-2-pyrrolidone (NMP), and intermediate I-4 (8.01 g, 24.5 mmol) and cesium carbonate (16.0 g, 49 mmol) were added thereto and then, refluxed by heating for 18 hours. When a reaction was completed, after distilling off the solvent therefrom and adding water to the reaction solution, an extract was obtained therefrom with dichloromethane (DCM), treated with magnesium sulfate anhydrous to remove moisture, filtered, and concentrated under a reduced pressure. The obtained residue was separated and purified through flash column chromatography to obtain Compound A-1 (13.3 g, 76%).

HRMS (70 eV, EI+): m/z calcd for C51H33N5: 715.2736, found: 715.

Elemental Analysis: C, 86%; H, 5%

### Synthesis Example 6: Synthesis of Intermediate I-5

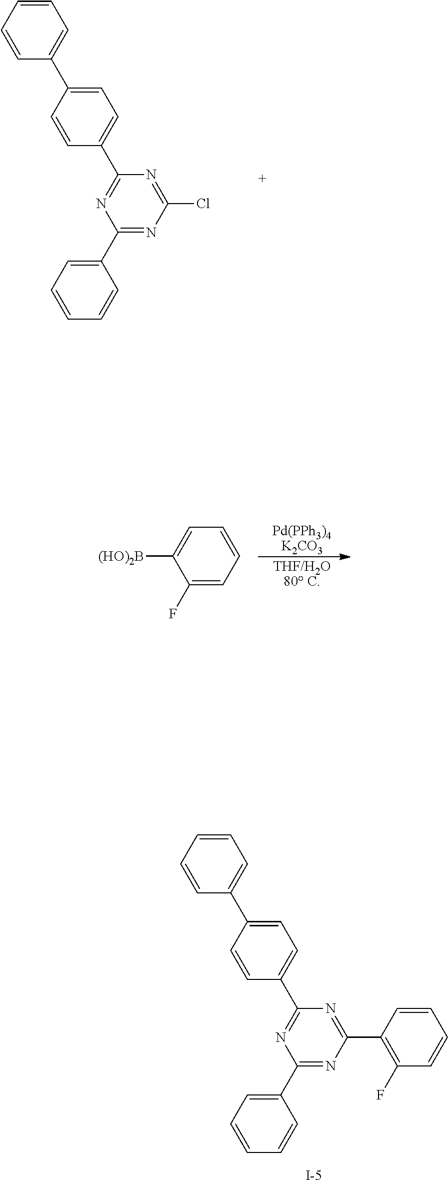

Intermediate I-5 (55.6 g, 95%) was obtained by using 2-(biphenyl-4-yl)-4-chloro-6-phenyl-1,3,5-triazine (50 g, 145 mmol) and 2-fluorophenylboronic acid (22.4 g, 160 mmol) in the same manner as in Synthesis Example 2.

HRMS (70 eV, EI+): m/z calcd for C27H18FN3: 403.1485, found: 403.

Elemental Analysis: C, 80%; H, 5%

### Synthesis Example 7: Synthesis of Compound A-9

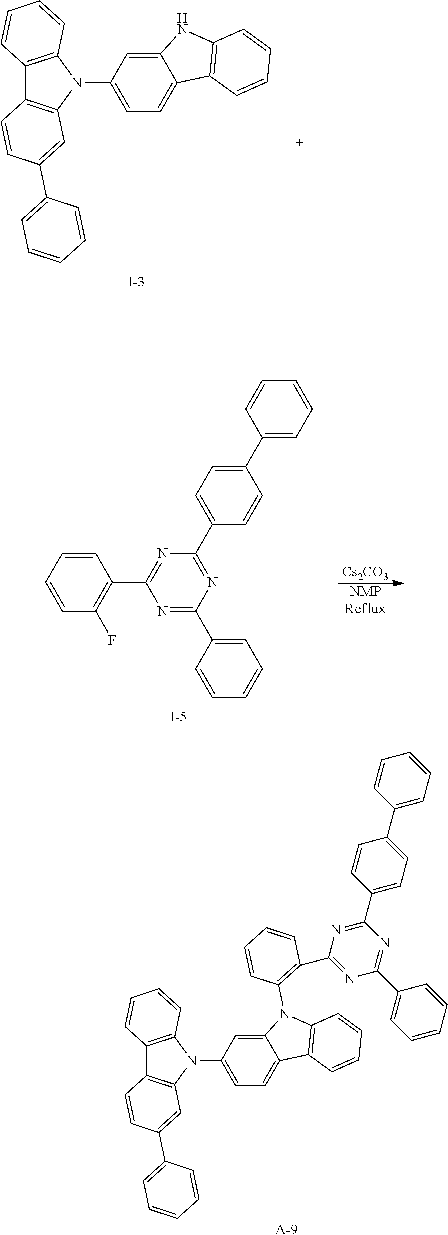

Compound A-9 (16.3 g, 84%) was obtained by using Intermediate I-3 (10 g, 24.5 mmol) and Intermediate I-5 (9.88 g, 24.5 mmol) in the same manner as in Synthesis Example 5.

HRMS (70 eV, ET+): m/z calcd for C57H37N5: 791.3049, found: 791.

Elemental Analysis: C, 86%; H, 5%

### Synthesis Example 8: Synthesis of Intermediate I-6

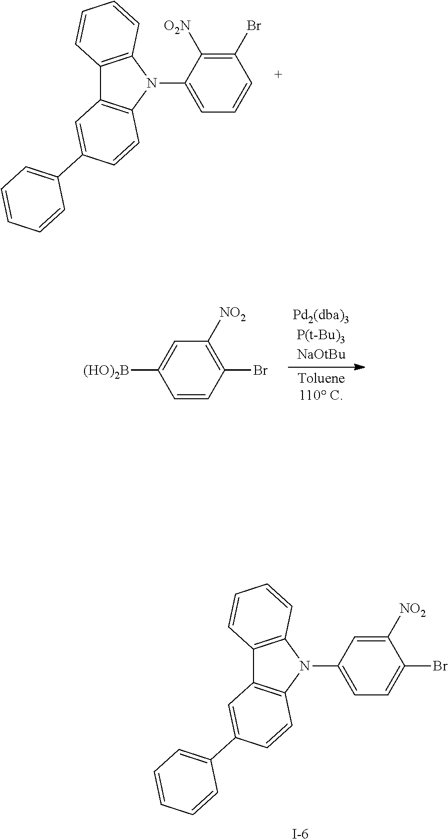

Intermediate I-6 (66.7 g, 73%) was obtained by using 3-phenyl-9H-carbazole (50 g, 206 mmol) and 1,4-dibromo-2-nitrobenzene (63.5 g, 226 mmol) in the same manner as in Synthesis Example 1.

HRMS (70 eV, EI+): m/z calcd for C24H₁₅BrN2O2: 442.0317, found: 442.

Elemental Analysis: C, 65%; H, 3%

### Synthesis Example 9: Synthesis of Intermediate I-7

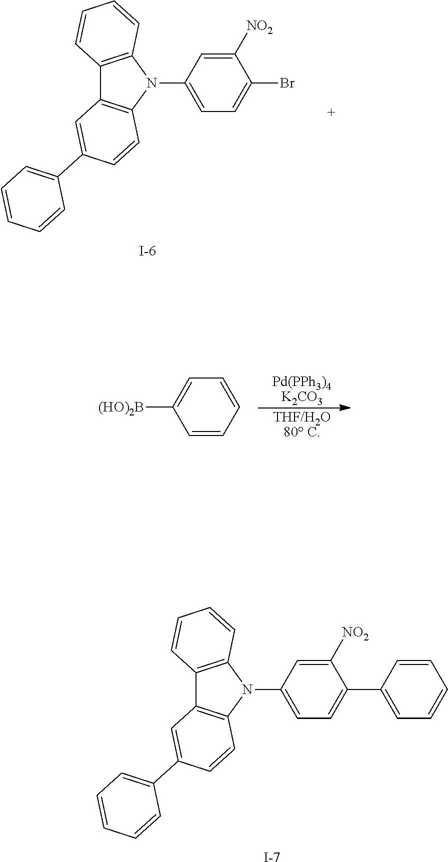

Intermediate I-7 (55.7 g, 86%) was obtained by using Intermediate I-6 (65 g, 147 mmol) and phenylboronic acid (21.5 g, 176 mmol) in the same manner as in Synthesis Example 2.

HRMS (70 eV, EI+): m/z calcd for C30H20N2O2: 440.1525, found: 440.

Elemental Analysis: C, 82%; H, 5%

### Synthesis Example 10: Synthesis of Intermediate I-8

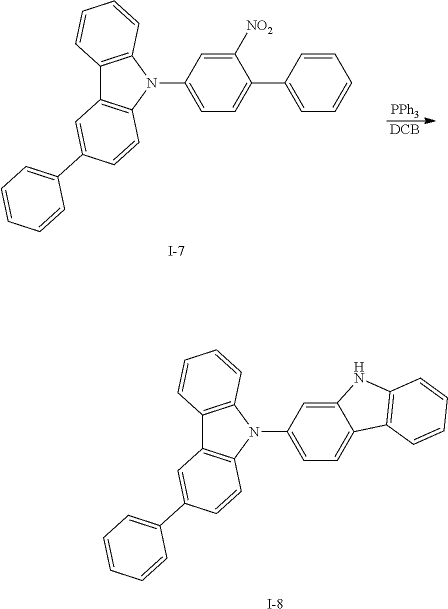

Intermediate I-8 (44.2 g, 90%) was obtained by using Intermediate I-7 (53 g, 120 mmol) in the same manner as in Synthesis Example 3.

HRMS (70 eV, EI+): m/z calcd for C30H20N2: 408.1626, found: 408.

Elemental Analysis: C, 88%; H, 5%

### Synthesis Example 11: Synthesis of Compound A-10

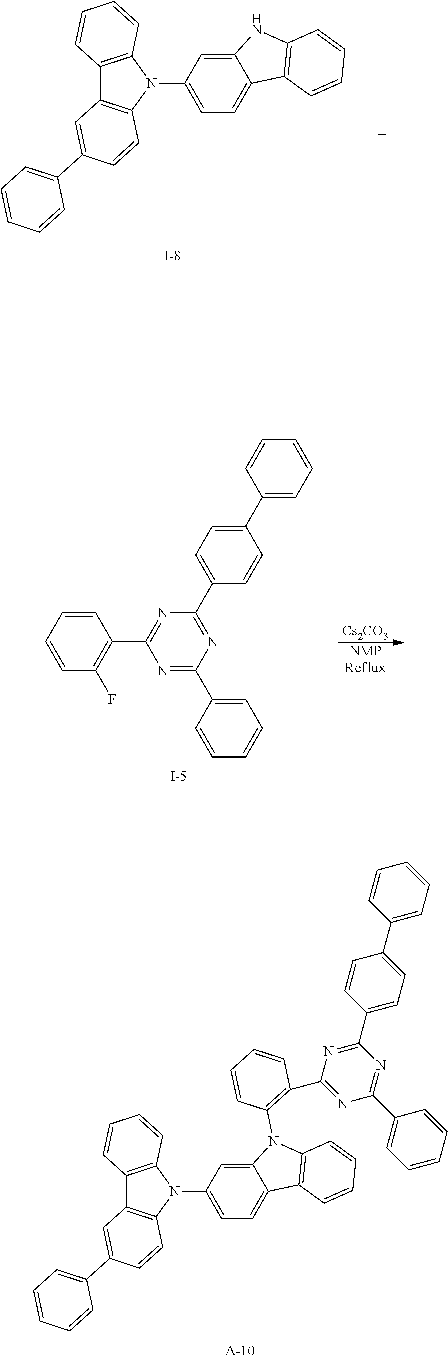

Compound A-10 (14.6 g, 75%) was obtained by using Intermediate I-8 (10 g, 24.5 mmol) and Intermediate I-5 (9.88 g, 24.5 mmol) in the same manner as in Synthesis Example 5.

HRMS (70 eV, EI+): m/z calcd for C57H37N5: 791.3049, found: 791.

Elemental Analysis: C, 86%; H, 5%

### Synthesis Example 12: Synthesis of Compound I-9

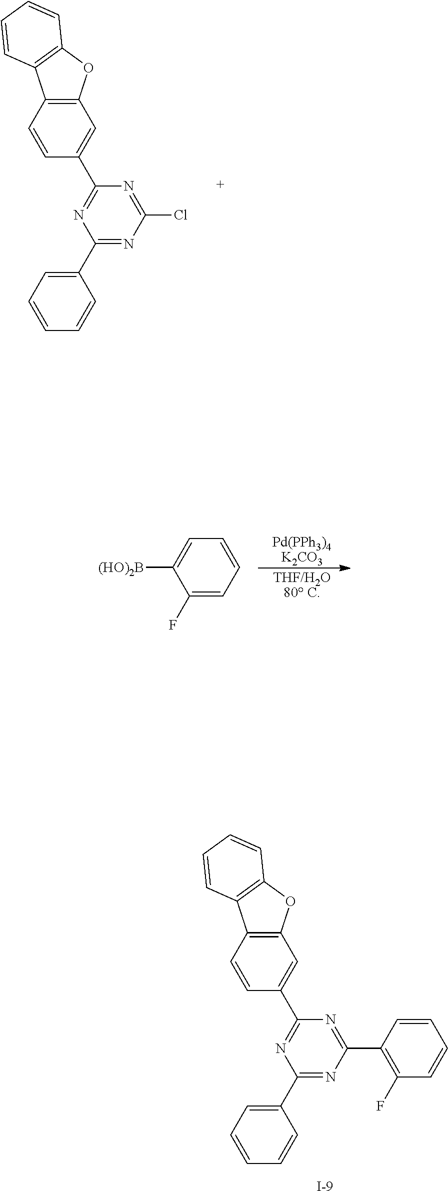

Intermediate I-9 (50.8 g, 87%) was obtained by using 2-chloro-4-(dibenzofuran-3-yl)-6-phenyl-1,3,5-triazine (50 g, 140 mmol) purchased from P&H Tech Co., Ltd. (http://www.phtech.co.kr/) and 2-fluorophenylboronic acid (21.5 g, 154 mmol) in the same manner as in Synthesis Example 2.

HRMS (70 eV, EI+): m/z calcd for C27H16FN3O: 417.1277, found: 417.

Elemental Analysis: C, 78%; H, 4%

### Synthesis Example 13: Synthesis of Compound A-18

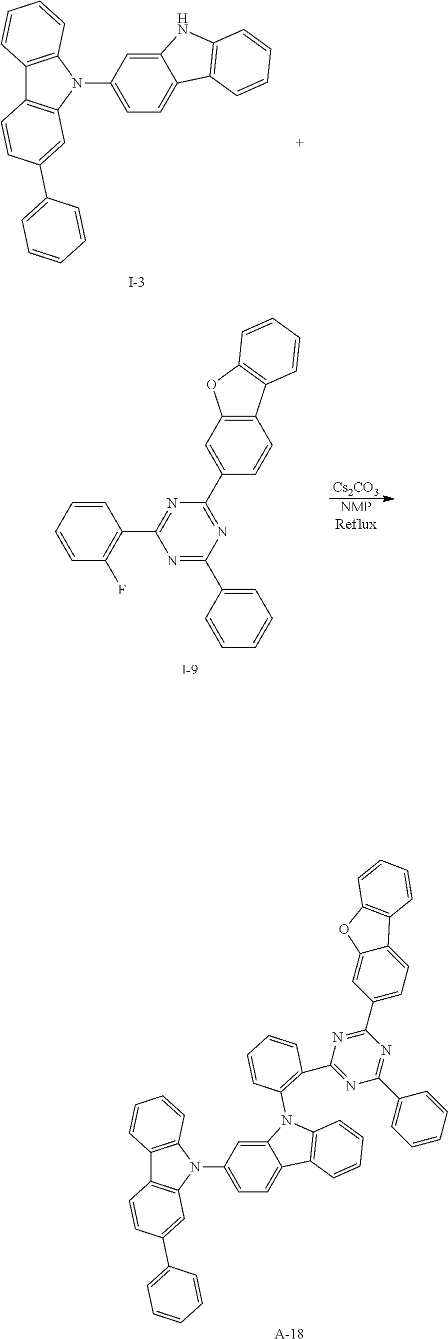

Compound A-18 (15.4 g, 78%) was obtained by using Intermediate I-3 (10 g, 24.5 mmol) and Intermediate I-9 (10.2 g, 24.5 mmol) in the same manner as in Synthesis Example 5.

HRMS (70 eV, EI+): m/z calcd for C57H35N5O: 805.2842, found: 805.

Elemental Analysis: C, 85%; H, 5%

### Synthesis Example 14: Synthesis of Intermediate I-10

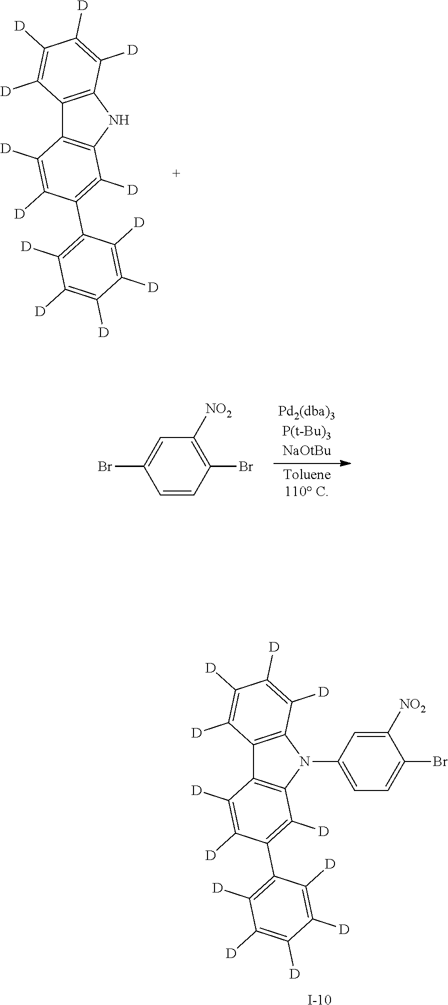

Intermediate I-10 (58.9 g, 66%) was obtained by using deuterium substituted 2-phenyl-9H-carbazole (50 g, 196 mmol) purchased from GemChem, Inc. (http://www.ytgemchem.com) and 1,4-dibromo-2-nitrobenzene (60.5 g, 216 mmol) in the same manner as in Synthesis Example 1.

HRMS (70 eV, EI+): m/z calcd for C24H3D12BrN202: 454.1070, found: 454.

Elemental Analysis: C, 63%; H, 6%

### Synthesis Example 15: Synthesis of Intermediate I-11

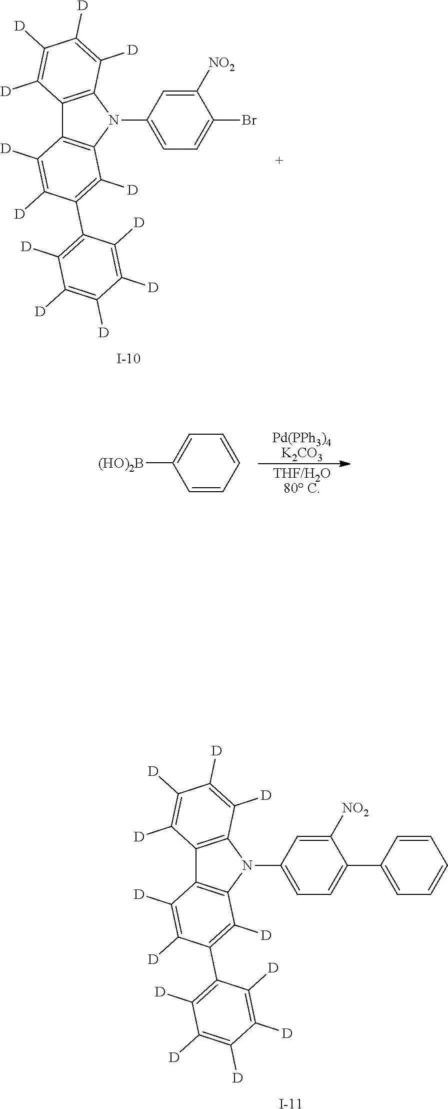

Intermediate I-11 (50.9 g, 93%) was obtained by using Intermediate T-10 (55 g, 121 mmol) and phenylboronic acid (17.7 g, 145 mmol) in the same manner as in Synthesis Example 2.

HRMS (70 eV, EI+): m/z calcd for C30H8D12N202: 452.2278, found: 452.

Elemental Analysis: C, 80%; H, 7%

### Synthesis Example 16: Synthesis of Intermediate I-12

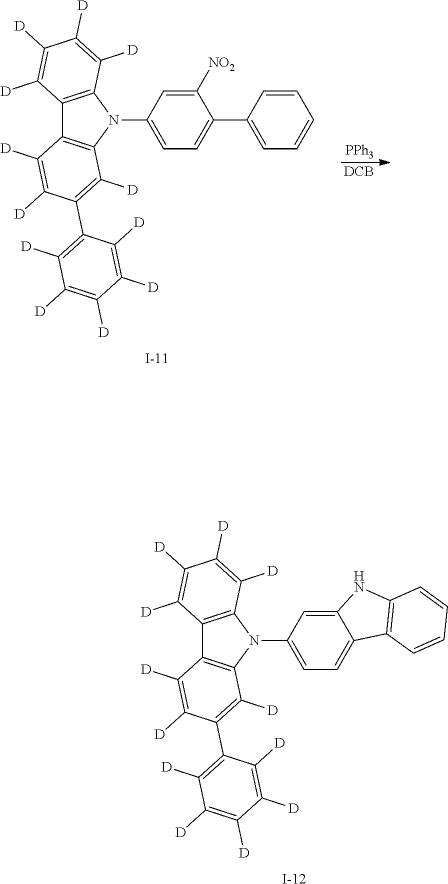

Intermediate I-12 (26.8 g, 64%) was obtained by using Intermediate I-11 (45 g, 99.4 mmol) in the same manner as in Synthesis Example 3.

HRMS (70 eV, EI+): m/z calcd for C30H8D12N2: 420.2380, found: 420.

Elemental Analysis: C, 86%; H, 8%

### Synthesis Example 17: Synthesis of Compound A-55

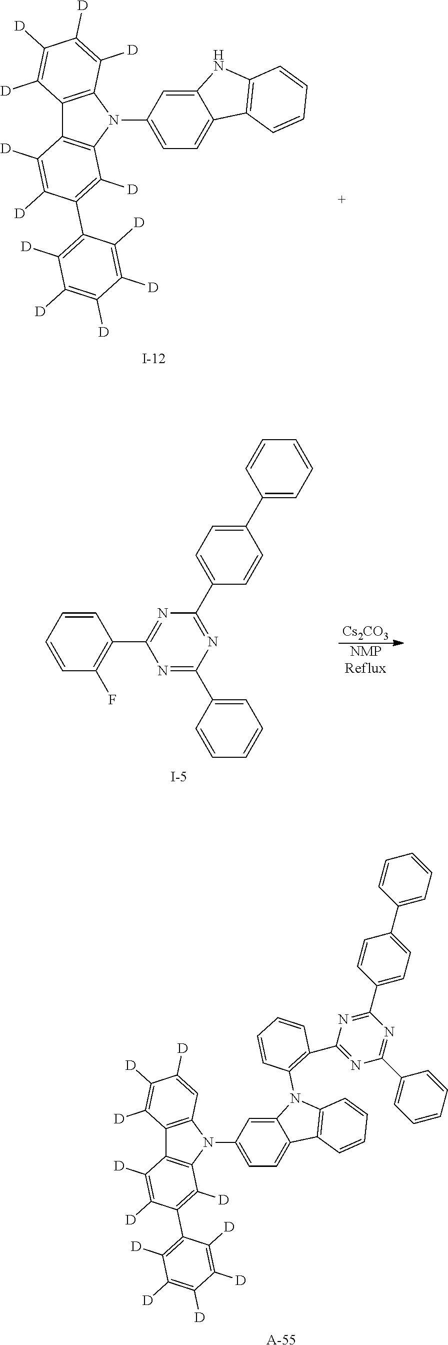

Compound A-55 (11.3 g, 59%) was obtained by using Intermediate I-12 (10 g, 23.8 mmol) and Intermediate I-5 (9.60 g, 23.8 mmol) in the same manner as in Synthesis Example 5.

HRMS (70 eV, EI+): m/z calcd for C57H25D12N5: 803.3802, found: 803.

Elemental Analysis: C, 85%; H, 6%

### Synthesis Example 18: Synthesis of Intermediate I-13

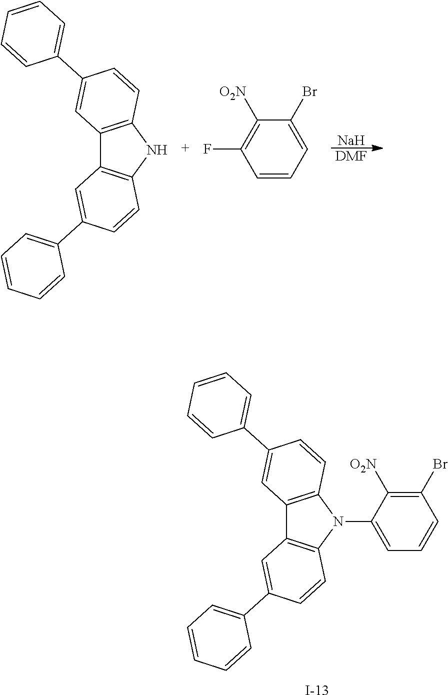

In the nitrogen environment, 3,6-diphenyl-9H-carbazole (50 g, 157 mmol) purchased from Tokyo Chemical Industry Co., Ltd. was dissolved in 0.5 L of dimethylformamide (DMF), and sodium hydride (4.5 g, 188 mmol) was added thereto and then, stirred at 0° C. After 1 hour, 1-bromo-3-fluoro-2-nitrobenzene (41.4 g, 188 mmol) purchased from Tokyo Chemical Industry Co., Ltd. was added thereto and then, stirred for 1 hour. When a reaction was completed, after adding water to the reaction solution at 0° C., an extract was obtained therefrom with dichloromethane (DCM), treated with magnesium sulfate anhydrous to remove moisture, filtered, and concentrated under a reduced pressure. The obtained residue was separated and purified through flash column chromatography to obtain Intermediate I-13 (66.9 g, 82%).

HRMS (70 eV, EI+): m/z calcd for C30H19BrN2O2: 518.0630, found: 518.

Elemental Analysis: C, 69%; H, 4%

### Synthesis Example 19: Synthesis of Intermediate I-14

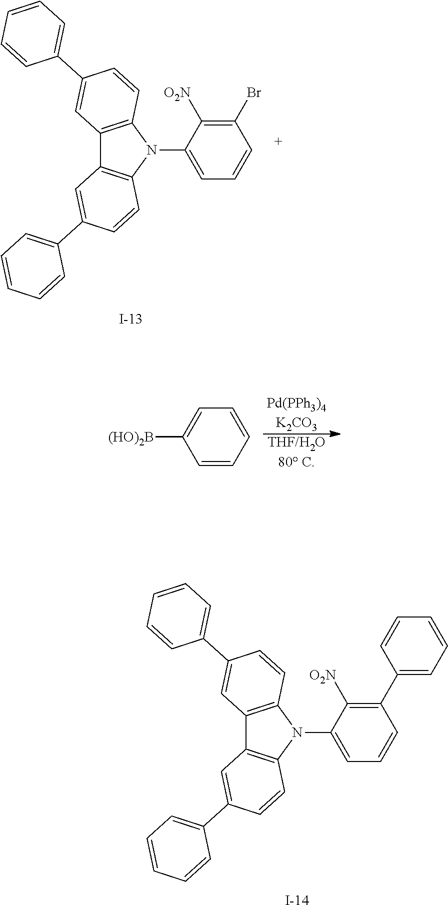

Intermediate I-14 (58.1 g, 90%) was obtained by using Intermediate I-13 (65 g, 125 mmol) and phenylboronic acid (16.8 g, 138 mmol) in the same manner as in Synthesis Example 2.

HRMS (70 eV, EI+): m/z calcd for C36H24N2O2: 440.1525, found: 440.

Elemental Analysis: C, 84%; H, 5%

### Synthesis Example 20: Synthesis of Intermediate I-15

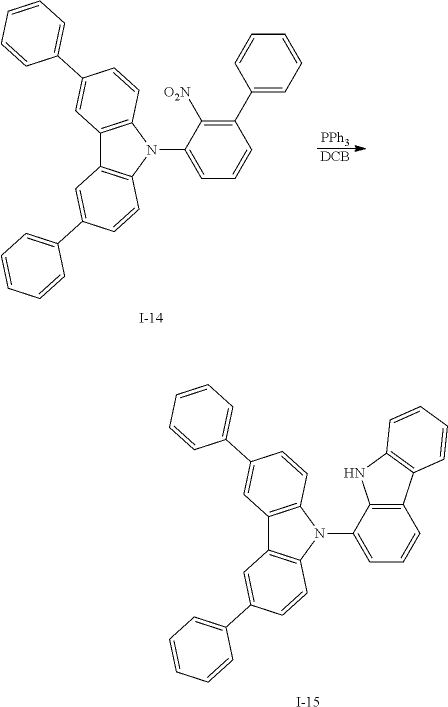

Intermediate I-15 (45.2 g, 88%) was obtained by using Intermediate I-14 (55 g, 106 mmol) in the same manner as in Synthesis Example 3.

HRMS (70 eV, EI+): m/z calcd for C36H24N2: 484.1939, found: 484.

Elemental Analysis: C, 89%; H, 5%

### Synthesis Example 21: Synthesis of Compound R-1

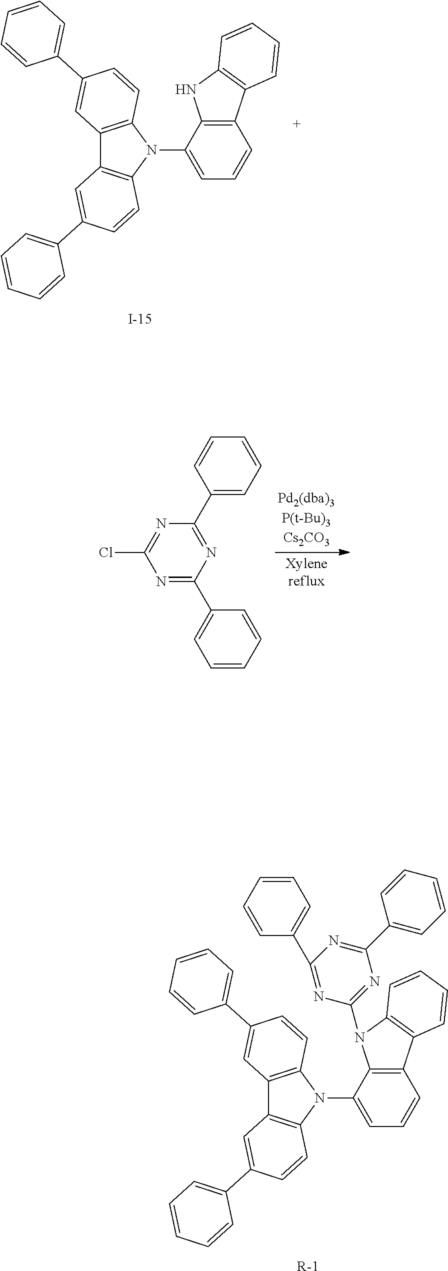

In the nitrogen environment, Intermediate I-15 (10 g, 20.6 mmol) was dissolved in 0.1 L of xylene, and 2-chloro-4,6-diphenyl-1,3,5-triazine (6.63 g, 24.8 mmol), tris(diphenylideneacetone)dipalladium (0) (0.57 g, 0.62 mmol), tris-tert butylphosphine (0.50 g, 2.47 mmol), and cesium carbonate (8.08 g, 24.8 mmol) were sequentially added thereto and then, refluxed by heating at 130° C. for 15 hours. When a reaction was completed, after adding water to the reaction solution, an extract was obtained therefrom with dichloromethane (DCM), treated with magnesium sulfate anhydrous to remove moisture, filtered, and concentrated under a reduced pressure. The obtained residue was separated and purified through flash column chromatography to obtain Comparative Example R-1 (9.29 g, 63%).

HRMS (70 eV, EI+): m/z calcd for C51H33N5: 715.2736, found: 715.

Elemental Analysis: C, 86%; H, 5%

### Synthesis Example 22: Synthesis of Compound R-2

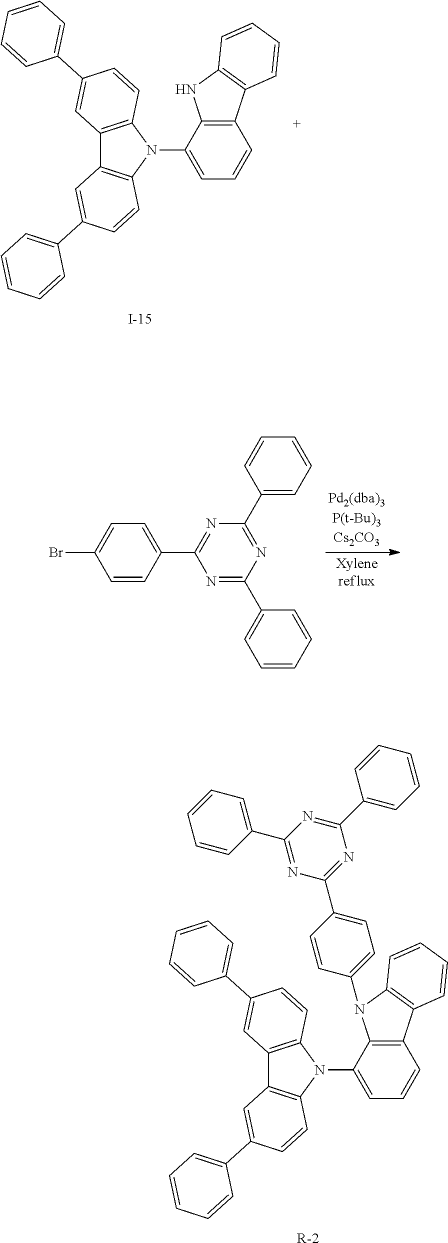

Comparative Example R-2 (8.85 g, 60%) was obtained by using Intermediate I-15 (10 g, 20.6 mmol) and 2-(4-bromophenyl)-4,6-diphenyl-1,3,5-triazine (9.61 g, 24.8 mmol) in the same manner as in Synthesis Example 21.

HRMS (70 eV, EI+): m/z calcd for C57H37N5: 791.3049, found: 791.

Elemental Analysis: C, 86%; H, 5%

### Synthesis Example 23: Synthesis of Intermediate I-16

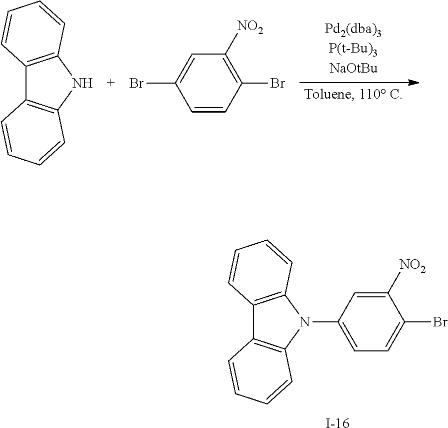

Intermediate I-16 (82.3 g, 75%) was obtained by using 9H-carbazole (50 g, 299 mmol) and 1,4-dibromo-2-nitrobenzene (101 g, 359 mmol) in the same manner as in Synthesis Example 1.

HRMS (70 eV, EI+): m/z calcd for C18H11BrN2O2: 366.0004, found: 336.

Elemental Analysis: C, 59%; H, 3%

### Synthesis Example 24: Synthesis of Intermediate I-17

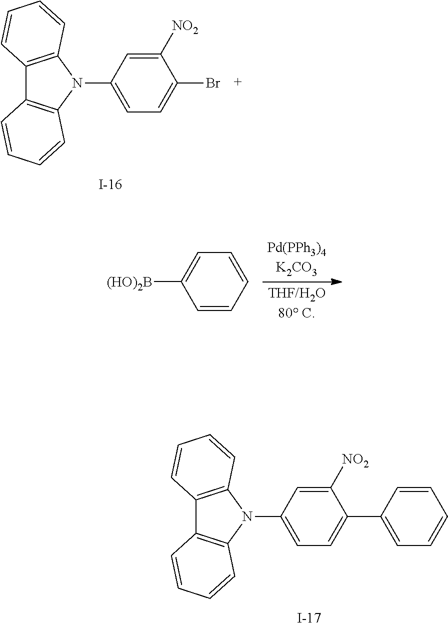

Intermediate I-17 (79.4 g, 81%) was obtained by using Intermediate I-16 (80 g, 218 mmol) and phenylboronic acid (95.3 g, 261 mmol) in the same manner as in Synthesis Example 2.

HRMS (70 eV, EI+): m/z calcd for C24H16N2O2: 364.1212, found: 364.

Elemental Analysis: C, 79%; H, 4%

### Synthesis Example 25: Synthesis of Intermediate I-18

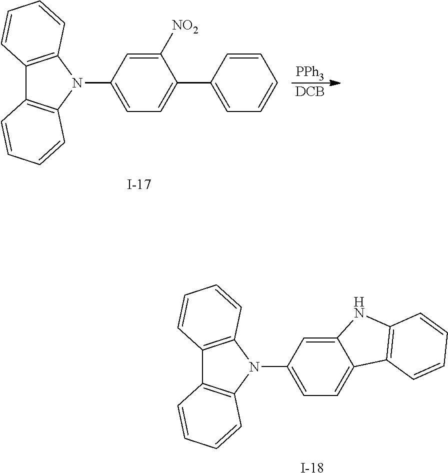

Intermediate I-18 (57.5 g, 84%) was obtained by using Intermediate I-17 (75 g, 256 mmol) in the same manner as in Synthesis Example 3.

HRMS (70 eV, EI+): m/z calcd for C24H16N2: 332.1313, found: 332.

Elemental Analysis: C, 87%; H, 5%

### Synthesis Example 26: Synthesis of Intermediate I-19

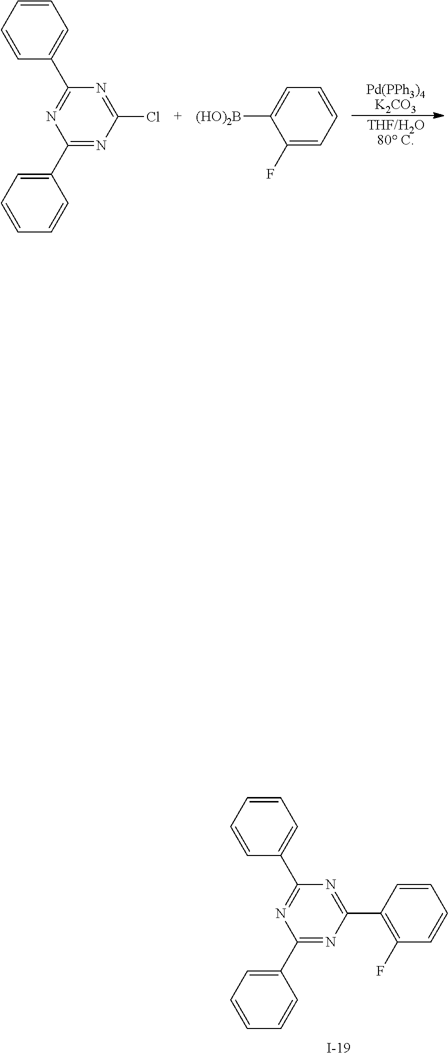

Intermediate I-19 (55.7 g, 91%) was obtained by using 2-chloro-4,6-diphenyl-1,3,5-triazine (50 g, 187 mmol) and 2-fluorophenylboronic acid (28.7 g, 205 mmol) in the same manner as in Synthesis Example 2.

HRMS (70 eV, EI+): m/z calcd for C21H14FN3: 327.1172, found: 327.

Elemental Analysis: C, 77%; H, 4%

### Synthesis Example 27: Synthesis of Compound R-3

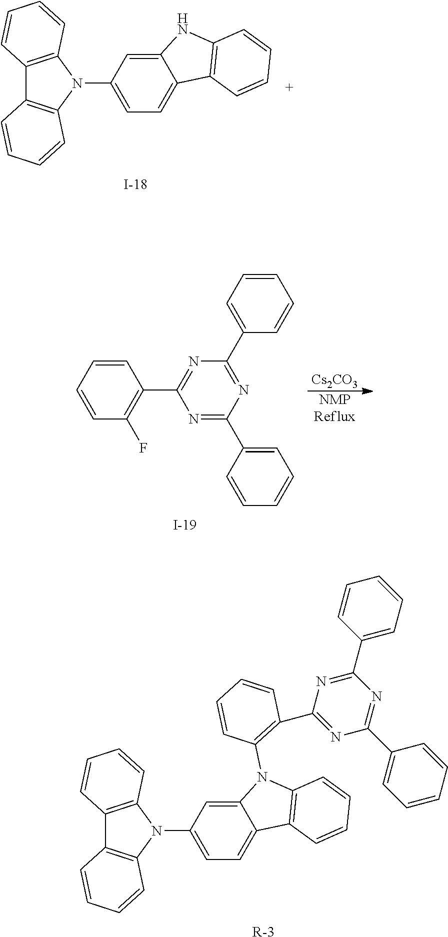

Compound R-3 (13.9 g, 72%) was obtained by using Intermediate I-18 (10 g, 30.1 mmol) and Intermediate I-19 (10.8 g, 33.1 mmol) in the same manner as in Synthesis Example 5.

HRMS (70 eV, EI+): m/z calcd for C45H29N5: 639.2423, found: 639.

Elemental Analysis: C, 84%; H, 5%

### Synthesis Example 28: Synthesis of Intermediate I-20

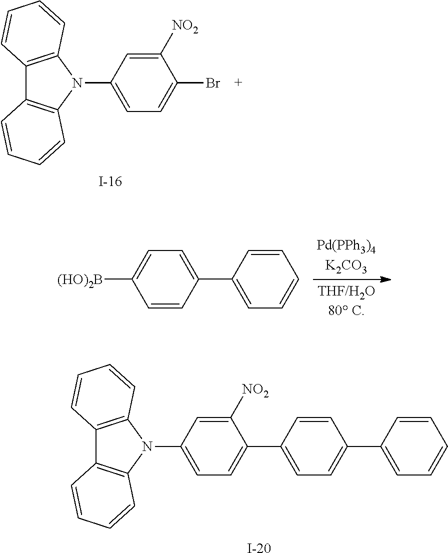

Intermediate I-20 (29.9 g, 83%) was obtained by using Intermediate I-16 (30 g, 81.7 mmol) and biphenyl-4-ylboronic acid (19.4 g, 98.0 mmol) in the same manner as in Synthesis Example 2.

HRMS (70 eV, EI+): m/z calcd for C30H20N2O2: 440.1525, found: 440.

Elemental Analysis: C, 82%; H, 5%

### Synthesis Example 29: Synthesis of Intermediate I-21

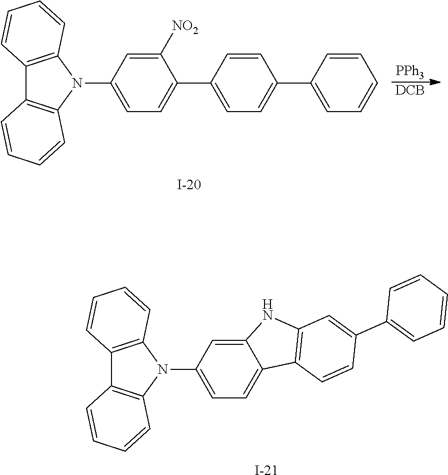

Intermediate I-21 (20.8 g, 80%) was obtained by using Intermediate I-20 (28 g, 63.6 mmol) in the same manner as in Synthesis Example 3.

HRMS (70 eV, EI+): m/z calcd for C30H20N2: 408.1626, found: 408.

Elemental Analysis: C, 85%; H, 5%

### Synthesis Example 30: Synthesis of Compound R-4

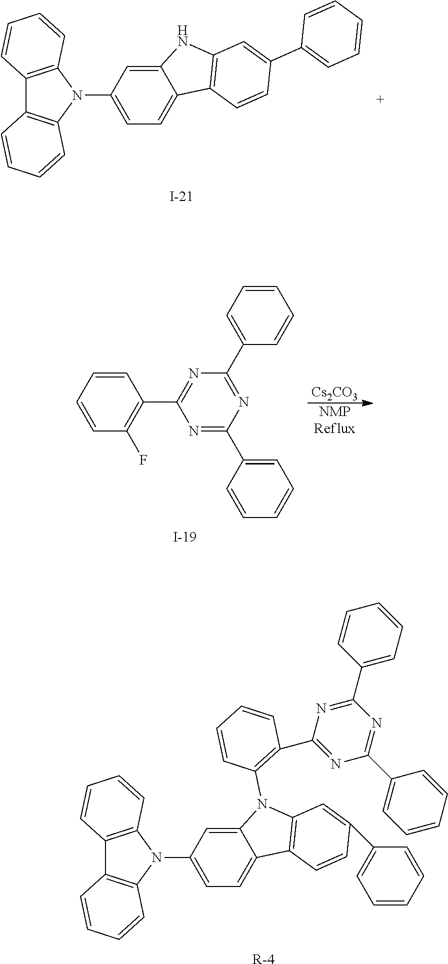

Compound R-4 (12.3 g, 70%) was obtained by using Intermediate I-21 (10 g, 24.5 mmol) and Intermediate I-19 (9.62 g, 29.4 mmol) in the same manner as in Synthesis Example 5.

HRMS (70 eV, EI+): m/z calcd for C51H33N5: 715.2736, found: 715.

Elemental Analysis: C, 86%; H, 5%

### Synthesis Example 31: Synthesis of Compound B-136

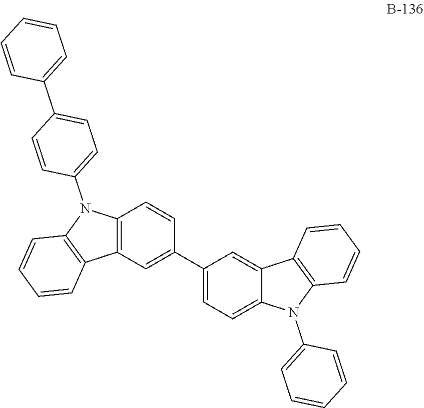 [SMILES: c1ccc(-c2ccc(-n3c4ccccc4c4cc(-c5ccc6c(c5)c5ccccc5n6-c5ccccc5)ccc43)cc2)cc1]

Compound B-136 was synthesized by referring to the synthesis method of patent EP3034581.

HRMS (70 eV, EI+): m/z calcd for C42H28N2: 560.2252, found: 560.

Elemental Analysis: C, 90%; H, 5%

### Synthesis Example 32: Synthesis of Compound B-99

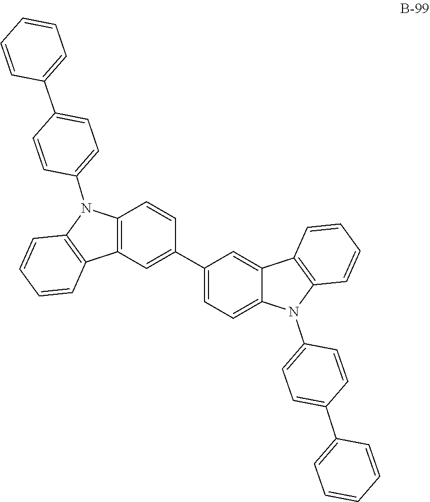 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

Compound B-99 was synthesized by referring to the synthesis method of patent KR10-2019-0000597.

HRMS (70 eV, EI+): m/z calcd for C48H32N2: 636.2565, found: 636.

Elemental Analysis: C, 91%; H, 5%

### Synthesis Example 33: Synthesis of Compound B-31

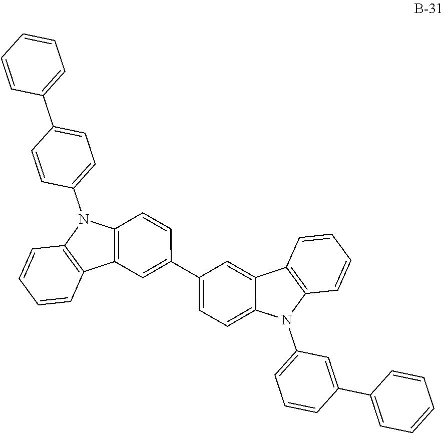 [SMILES: c1ccc(-c2ccc(-n3c4ccccc4c4cc(-c5ccc6c(c5)c5ccccc5n6-c5cccc(-c6ccccc6)c5)ccc43)cc2)cc1]

Compound B-31 was synthesized by referring to the synthesis method of patent EP2947071.

HRMS (70 eV, EI+): m/z calcd for C48H32N2: 636.2565, found: 636.

Elemental Analysis: C, 91%; H, 5%

### Synthesis Example 34: Synthesis of Compound C-4

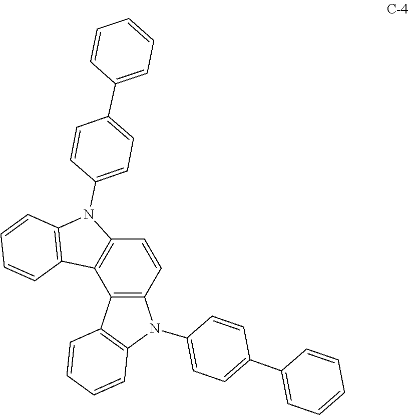 [SMILES: c1ccc(-c2ccc(-n3c4ccccc4c4c5c6ccccc6n(-c6ccc(-c7ccccc7)cc6)c5ccc43)cc2)cc1]

Compound C-4 was synthesized by referring to the synthesis method of patent KR2031300.

HRMS (70 eV, EI+): m/z calcd for C42H28N2: 560.2252, found: 560.

Elemental Analysis: C, 90%; H, 5%

### Synthesis Example 35: Synthesis of Compound C-6

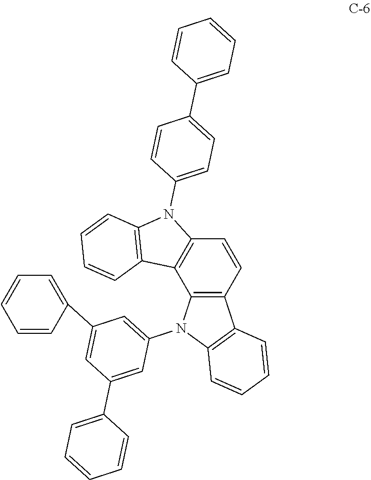 [SMILES: c1ccc(-c2ccc(-n3c4ccccc4c4c3ccc3c5ccccc5n(-c5cc(-c6ccccc6)cc(-c6ccccc6)c5)c34)cc2)cc1]

Compound C-6 was synthesized by referring to the synthesis method of patent WO2018-095391.

HRMS (70 eV, EI+): m/z calcd for C48H32N2: 636.2565, found: 636.

Elemental Analysis: C, 91%; H, 5%

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. Compound B was deposited on the hole transport layer to a thickness of 350 Å to form a hole transport auxiliary layer, Compound A-1 synthesized in Synthesis Example 5 was used as a host on the hole transport auxiliary layer, and 7 wt % of PhGD was used as a dopant by vacuum deposition to form a 400 Å-thick light emitting layer and the ratios thereof are described separately in the following Examples and Comparative Examples. Subsequently, Compound C was deposited on the emitting layer to a thickness of 50 Å to form an electron transport auxiliary layer, and Compound D and Liq were simultaneously vacuum deposited in a weight ratio of 1:1 to form a 300 Å-thick electron transport layer. An organic light emitting device was manufactured by sequentially vacuum depositing 15 Å of LiQ and 1,200 Å of Al on the electron transport layer to form a cathode.

ITO/Compound A (3% NDP-9 doping, 100 Å)/Compound A (1350 Å)/Compound B (350 Å)/EML [Compound A-1: PhGD (7 wt %)](400 Å)/Compound C (50 Å)/Compound D: LiQ (300 Λ)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′:2′,1″:3″,1′″:3′″,1″″-quinquephenyl]-3″″-yl)-1,3,5-triazine
Compound D: 2-(1,1′-Biphenyl-4-yl)-4-(9,9-diphenylfluoren-4-yl)-6-phenyl-1,3,5-triazine

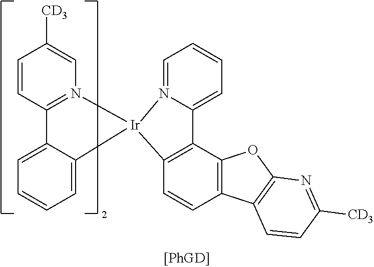

### Examples 2 to 5 and Comparative Examples 1 to 4

Each organic light emitting diode was manufactured in the same manner as in Example 1, except that the composition was changed to the one shown in Table 1.

### Example 6

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. Compound E was deposited on the hole transport layer to a thickness of 350 Å to form a hole transport auxiliary layer. On the hole transport auxiliary layer, Compound A-1 obtained in Synthesis Example 5 and Compound B-136 obtained in Synthesis Example 31 were used simultaneously as a host, and 10 wt % of PhGD was doped as a dopant to form a 400 Å-thick light emitting layer by vacuum deposition. Herein, Compound A-1 and Compound B-136 were used in a weight ratio of 3:7. Subsequently, Compound F was deposited on the emitting layer to a thickness of 50 Å to form an electron transport auxiliary layer, and Compound G and Liq were simultaneously vacuum-deposited in a weight ratio of 1:1 to form an electron transport layer with a thickness of 300 Å. An organic light emitting diode was manufactured by sequentially vacuum depositing 15 Å of LiQ and 1200 Å of Al on the electron transport layer to form a cathode.

ITO/Compound A (3% NDP-9 doping, 100 Å)/Compound A (1350 Å)/Compound E (350 Å)/EML [Compound A-1: Compound B-136:PhGD=27:63:10 wt %)]400 Å/Compound F (50 Å)/Compound G:LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound E: N,N-bis(9,9-dimethyl-9H-fluoren-4-yl)-9,9-spirobi(fluorene)-2-amine
Compound F: 2-[3′-(9,9-Dimethyl-9H-fluoren-2-yl)[1,1′-biphenyl]-3-yl]-4,6-diphenyl-1,3,5-triazine
Compound G: 2-[4-[4-(4′-Cyano-1,1′-biphenyl-4-yl)-1-naphthyl]phenyl]-4,6-diphenyl-1,3,5-triazine

### Examples 7 to 16 and Comparative Examples 5 to 8

Each organic light emitting diode was manufactured in the same manner as in Example 6, except that the composition was changed to the those shown in Table 2.

### Evaluation

The driving voltage, luminous efficiency, and life-span characteristics of the organic light emitting diodes according to Examples 1 to 16 and Comparative Examples 1 to 8 were evaluated.

The specific measurement method is as follows, and the results are as shown in Tables 1 and 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Current efficiency (cd/A) at the same current density (10 mA/cm²) were calculated by using the luminance and current density from (1) and (2) above and voltage.

The luminous efficiency values of Examples 1 to 5 and Comparative Examples 1 to 4 were calculated as relative values based on Comparative Example 1 and are shown in Table 1.

The luminous efficiency values of Examples 6 to 16 and Comparative Examples 5 to 8 were calculated as relative values based on Comparative Example 5 and are shown in Table 2.

### (4) Measurement of Life-Span

While luminance (cd/m²) was maintained at 24000 cd/m², time taken until luminous efficiency (cd/A) was reduced to 97% was measured.

The measured life-spans of Examples 1 to 5 and Comparative Examples 1 to 4 were calculated as relative values based on Comparative Example 1 and are shown in Table 1.

The life-span measurements of Examples 6 to 16 and Comparative Examples 5 to 8 were calculated as relative values based on Comparative Example 5 and are shown in Table 2.

TABLE 1

| | | Driving | | | Life- |
|---|---|---|---|---|---|
| | Compound | voltage | Color | Efficiency | span |
| Nos. | (wt %) | (%) | (EL color) | (%) | (%) |
| Example 1 | A-1 (10) | 98% | Green | 121% | 200% |
| Example 2 | A-9 (10) | 97% | Green | 122% | 250% |
| Example 3 | A-10 (10) | 96% | Green | 113% | 175% |
| Example 4 | A-18 (10) | 94% | Green | 119% | 175% |
| Example 5 | A-55 (10) | 97% | Green | 122% | 375% |
| Comparative | R-1 (10) | 100% | Green | 100% | 100% |
| Example 1 | | | | | |
| Comparative | R-2 (10) | 101% | Green | 93% | 50% |
| Example 2 | | | | | |
| Comparative | R-3 (10) | 100% | Green | 104% | 100% |
| Example 3 | | | | | |
| Comparative | R-4 (10) | 99% | Green | 11% | 125% |
| Example 4 | | | | | |

TABLE 2

| | | Driving | | | Life- |
|---|---|---|---|---|---|
| | Compound | voltage | Color | Efficiency | span |
| Nos. | (weight ratio) | (%) | (EL color) | (%) | (%) |
| Example 6 | A-1/B-136 (3:7) | 96% | Green | 110% | 200% |
| Example 7 | A-9/B-136 (3:7) | 95% | Green | 113% | 225% |
| Example 8 | A-10/B-136 (3:7) | 95% | Green | 112% | 188% |
| Example 9 | A-18/B-136 (3:7) | 93% | Green | 109% | 213% |
| Example 10 | A-55/B-136 (3:7) | 95% | Green | 113% | 250% |
| Example 11 | A-9/B-99 (3:7) | 94% | Green | 114% | 213% |
| Example 12 | A-9/B-31 (3:7) | 98% | Green | 115% | 225% |
| Example 13 | A-9/C-4 (3:7) | 93% | Green | 116% | 175% |
| Example 14 | A-9/C-6 (3:7) | 94% | Green | 119% | 175% |
| Example 15 | A-9/B-136 (4:6) | 93% | Green | 115% | 200% |
| Example 16 | A-9/B-136 (2:8) | 97% | Green | 112% | 213% |
| Comparative | R-1/B-136 (3:7) | 100% | Green | 100% | 100% |
| Example 5 | | | | | |
| Comparative | R-2/B-136 (3:7) | 101% | Green | 105% | 88% |
| Example 6 | | | | | |
| Comparative | R-3/B-136 (3:7) | 99% | Green | 105% | 163% |
| Example 7 | | | | | |
| Comparative | R-4/B-136 (3:7) | 100% | Green | 103% | 170% |
| Example 8 | | | | | |

Referring to Tables 1 and 2, the luminous efficiency and life-span characteristics of the organic light emitting diodes according to Examples 1 to 16 are significantly improved compared to the organic light emitting diodes according to Comparative Examples 1 to 8.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

1. A compound for an organic optoelectronic device, the compound being represented by Chemical Formula 1: 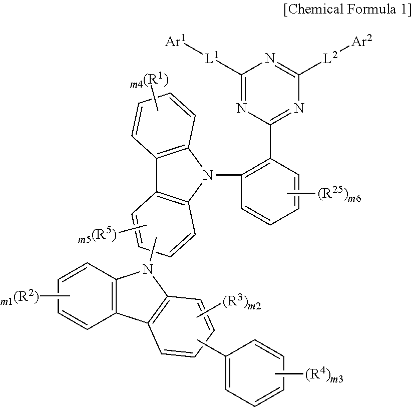
wherein, in Chemical Formula 1,
Ar¹ and Ar² are each independently a substituted or unsubstituted C6 to C30 aryl group or a substituted or unsubstituted C2 to C30 heterocyclic group,
L¹ and L² are each independently a single bond, a substituted or unsubstituted C6 to C30 arylene group, or a substituted or unsubstituted C2 to C30 heteroarylene group,
R¹ to R³ and R⁵ are each independently hydrogen, deuterium, a cyano group, or a substituted or unsubstituted C6 to C30 aryl group,
R⁴ is hydrogen, deuterium, a cyano group, a substituted or unsubstituted C1 to C10 alkyl group, or a substituted or unsubstituted C6 to C12 aryl group,
R²⁵ is hydrogen or deuterium,
m1, m4, and m6 are each independently an integer of 1 to 4,
m2 and m5 are each independently an integer of 1 to 3, and
m3 is an integer of 1 to 5.

2. The compound for an organic optoelectronic device of claim 1, wherein;
the compound is represented by one of Chemical Formula 1-1 to Chemical Formula 1-4: 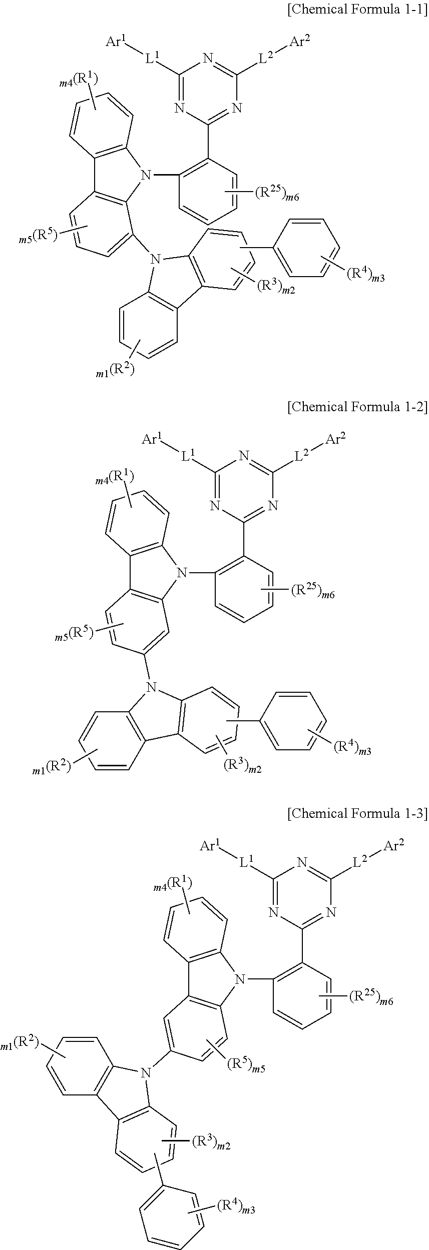 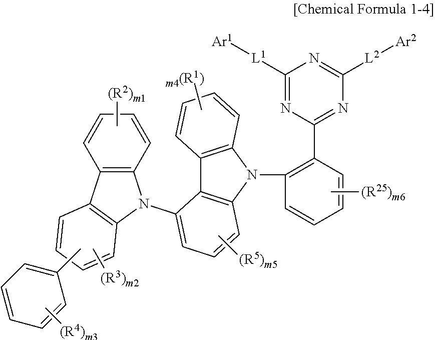
in Chemical Formula 1-1 to Chemical Formula 1-4, Ar¹, Ar², R¹ to R⁵, R²⁵, L¹, L², and m1 to m6 are defined the same as those of Chemical Formula 1.

3. The compound for an organic optoelectronic device of claim 1, wherein:
the compound is represented by Chemical Formula 1-2B or Chemical Formula 1-2C: 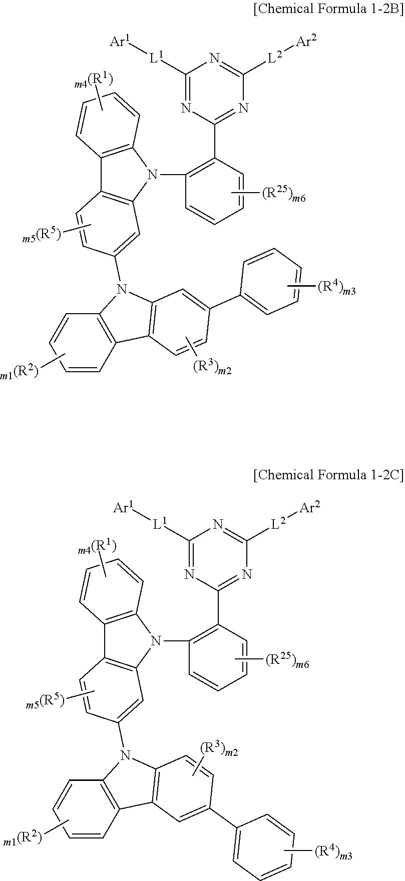
in Chemical Formula 1-2B and Chemical Formula 1-2C, Ar¹, Ar², R¹ to R⁵, R²⁵, L¹, L², and m1 to m6 are defined the same as those of Chemical Formula 1.

4. The compound for an organic optoelectronic device of claim 1, wherein R¹ to R³ are each independently hydrogen, deuterium, a cyano group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

5. The compound for an organic optoelectronic device of claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, or a substituted or unsubstituted dibenzothiophenyl group.

6. The compound for an organic optoelectronic device of claim 1, wherein;
moieties *-L¹-Ar¹ and *-L²-Ar² are each independently a moiety of Group I: 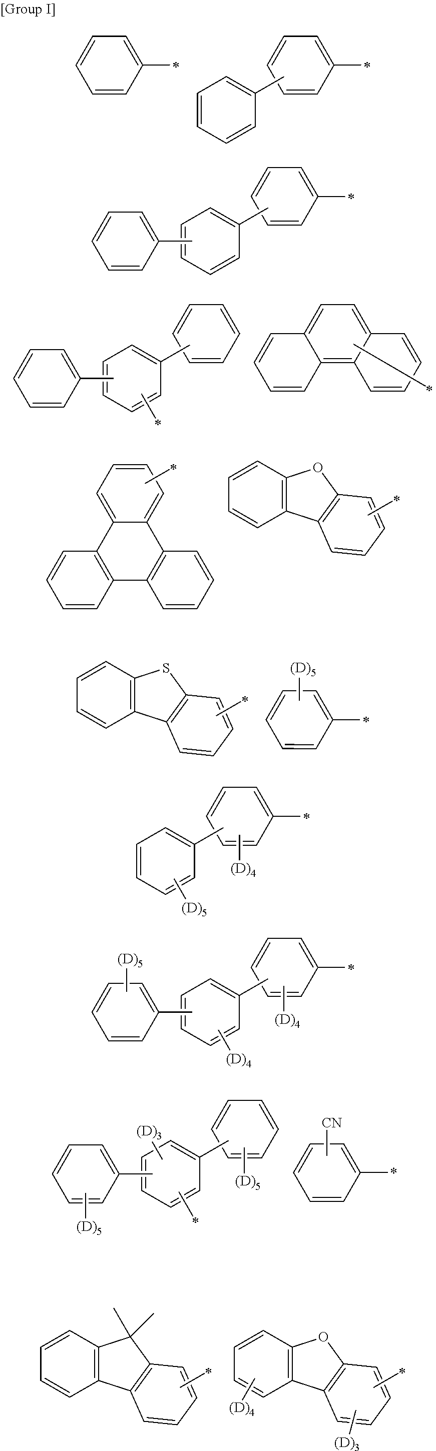 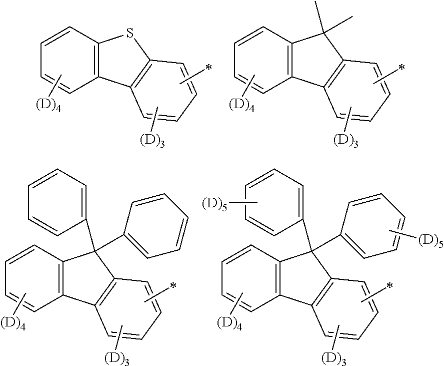
in Group I, * is a linking point.

7. The compound for an organic optoelectronic device of claim 1, wherein the compound is a compound of Group 1:

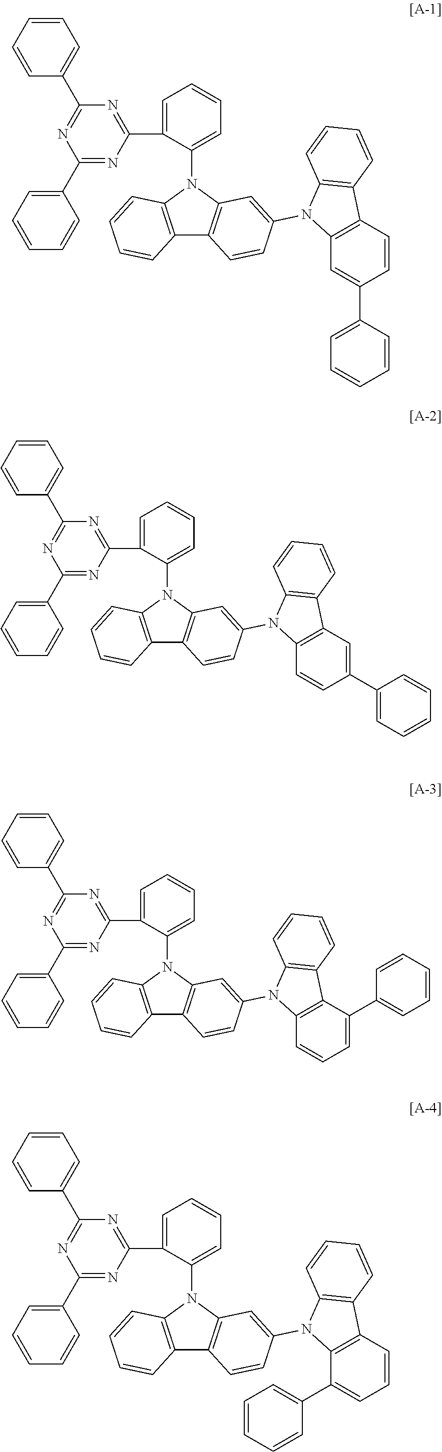 [SMILES: c1ccc(-c2ccc3c4ccccc4n(-c4ccc5c6ccccc6n(-c6ccccc6-c6nc(-c7ccccc7)nc(-c7ccccc7)n6)c5c4)c3c2)cc1 ; c1ccc(-c2ccc3c(c2)c2ccccc2n3-c2ccc3c4ccccc4n(-c4ccccc4-c4nc(-c5ccccc5)nc(-c5ccccc5)n4)c3c2)cc1 ; c1ccc(-c2nc(-c3ccccc3)nc(-c3ccccc3-n3c4ccccc4c4ccc(-n5c6ccccc6c6c(-c7ccccc7)cccc65)cc43)n2)cc1 ; c1ccc(-c2nc(-c3ccccc3)nc(-c3ccccc3-n3c4ccccc4c4ccc(-n5c6ccccc6c6cccc(-c7ccccc7)c65)cc43)n2)cc1]

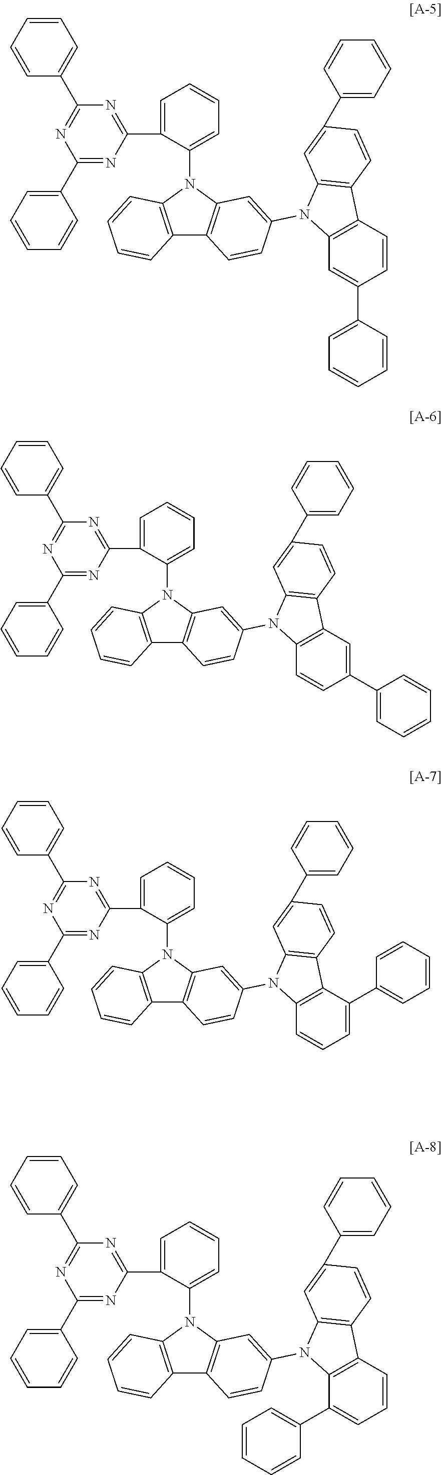 [SMILES: c1ccc(-c2ccc3c4ccc(-c5ccccc5)cc4n(-c4ccc5c6ccccc6n(-c6ccccc6-c6nc(-c7ccccc7)nc(-c7ccccc7)n6)c5c4)c3c2)cc1 ; c1ccc(-c2ccc3c(c2)c2ccc(-c4ccccc4)cc2n3-c2ccc3c4ccccc4n(-c4ccccc4-c4nc(-c5ccccc5)nc(-c5ccccc5)n4)c3c2)cc1 ; c1ccc(-c2ccc3c4c(-c5ccccc5)cccc4n(-c4ccc5c6ccccc6n(-c6ccccc6-c6nc(-c7ccccc7)nc(-c7ccccc7)n6)c5c4)c3c2)cc1 ; c1ccc(-c2ccc3c4cccc(-c5ccccc5)c4n(-c4ccc5c6ccccc6n(-c6ccccc6-c6nc(-c7ccccc7)nc(-c7ccccc7)n6)c5c4)c3c2)cc1]

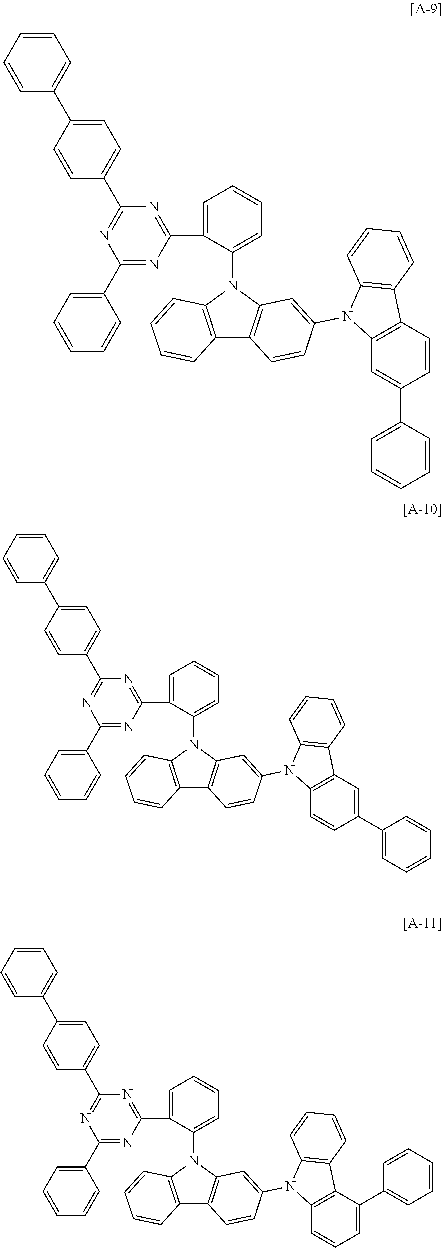 [SMILES: c1ccc(-c2ccc(-c3nc(-c4ccccc4)nc(-c4ccccc4-n4c5ccccc5c5ccc(-n6c7ccccc7c7ccc(-c8ccccc8)cc76)cc54)n3)cc2)cc1 ; c1ccc(-c2ccc(-c3nc(-c4ccccc4)nc(-c4ccccc4-n4c5ccccc5c5ccc(-n6c7ccccc7c7cc(-c8ccccc8)ccc76)cc54)n3)cc2)cc1 ; c1ccc(-c2ccc(-c3nc(-c4ccccc4)nc(-c4ccccc4-n4c5ccccc5c5ccc(-n6c7ccccc7c7c(-c8ccccc8)cccc76)cc54)n3)cc2)cc1]

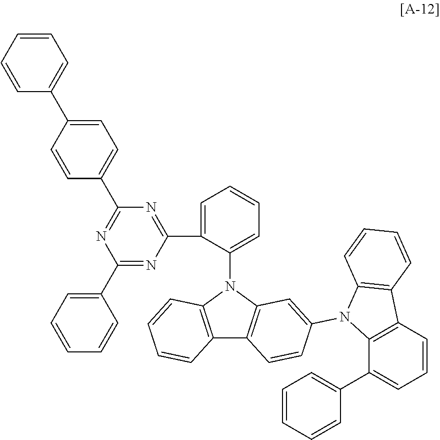 [SMILES: c1ccc(-c2ccc(-c3nc(-c4ccccc4)nc(-c4ccccc4-n4c5ccccc5c5ccc(-n6c7ccccc7c7cccc(-c8ccccc8)c76)cc54)n3)cc2)cc1]

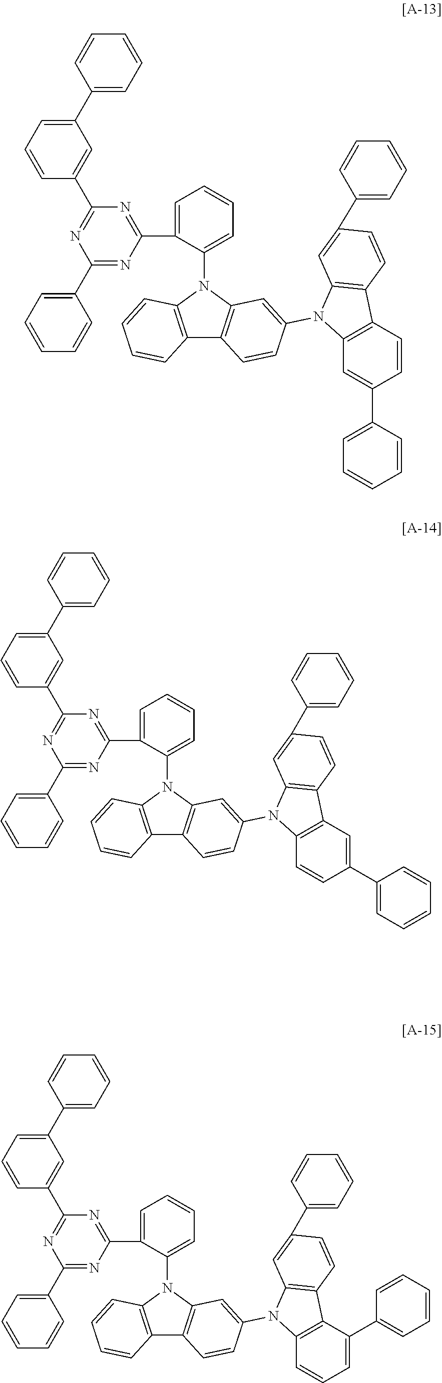 [SMILES: c1ccc(-c2cccc(-c3nc(-c4ccccc4)nc(-c4ccccc4-n4c5ccccc5c5ccc(-n6c7cc(-c8ccccc8)ccc7c7ccc(-c8ccccc8)cc76)cc54)n3)c2)cc1 ; c1ccc(-c2cccc(-c3nc(-c4ccccc4)nc(-c4ccccc4-n4c5ccccc5c5ccc(-n6c7ccc(-c8ccccc8)cc7c7ccc(-c8ccccc8)cc76)cc54)n3)c2)cc1 ; c1ccc(-c2cccc(-c3nc(-c4ccccc4)nc(-c4ccccc4-n4c5ccccc5c5ccc(-n6c7cc(-c8ccccc8)ccc7c7c(-c8ccccc8)cccc76)cc54)n3)c2)cc1]

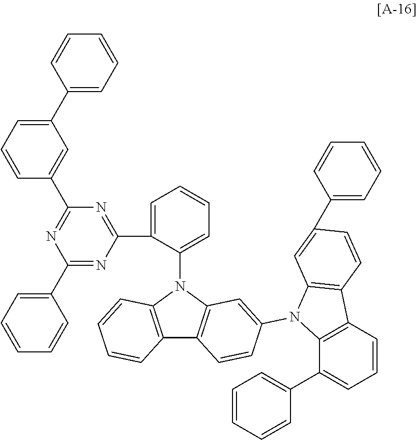 [SMILES: c1ccc(-c2cccc(-c3nc(-c4ccccc4)nc(-c4ccccc4-n4c5ccccc5c5ccc(-n6c7cc(-c8ccccc8)ccc7c7cccc(-c8ccccc8)c76)cc54)n3)c2)cc1]

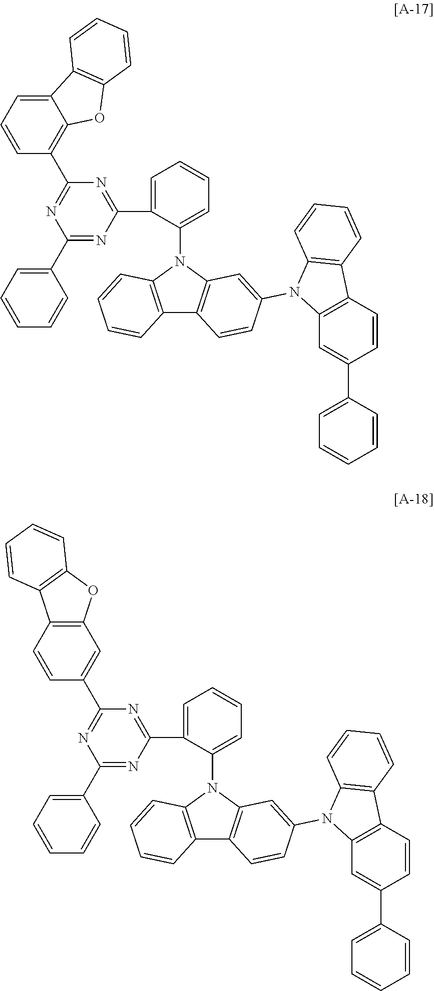 [SMILES: c1ccc(-c2ccc3c4ccccc4n(-c4ccc5c6ccccc6n(-c6ccccc6-c6nc(-c7ccccc7)nc(-c7cccc8c7oc7ccccc78)n6)c5c4)c3c2)cc1 ; c1ccc(-c2ccc3c4ccccc4n(-c4ccc5c6ccccc6n(-c6ccccc6-c6nc(-c7ccccc7)nc(-c7ccc8c(c7)oc7ccccc78)n6)c5c4)c3c2)cc1]

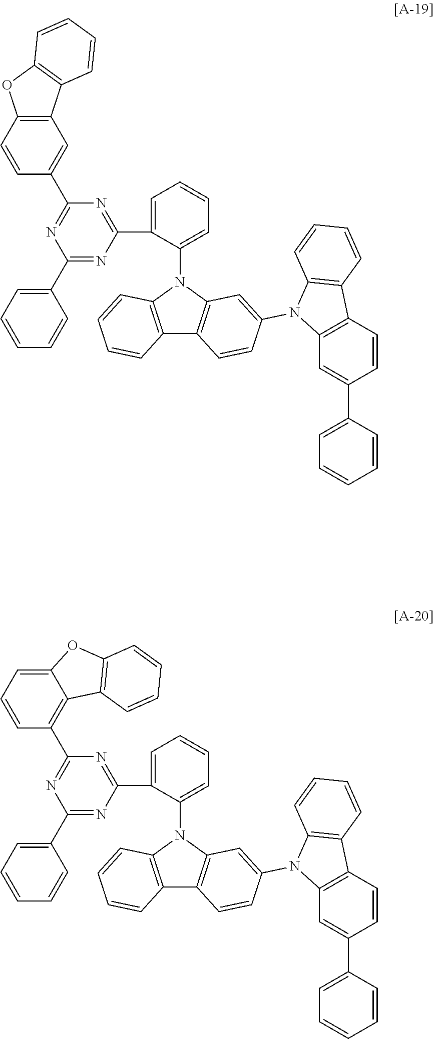 [SMILES: c1ccc(-c2ccc3c4ccccc4n(-c4ccc5c6ccccc6n(-c6ccccc6-c6nc(-c7ccccc7)nc(-c7ccc8oc9ccccc9c8c7)n6)c5c4)c3c2)cc1 ; c1ccc(-c2ccc3c4ccccc4n(-c4ccc5c6ccccc6n(-c6ccccc6-c6nc(-c7ccccc7)nc(-c7cccc8oc9ccccc9c78)n6)c5c4)c3c2)cc1]

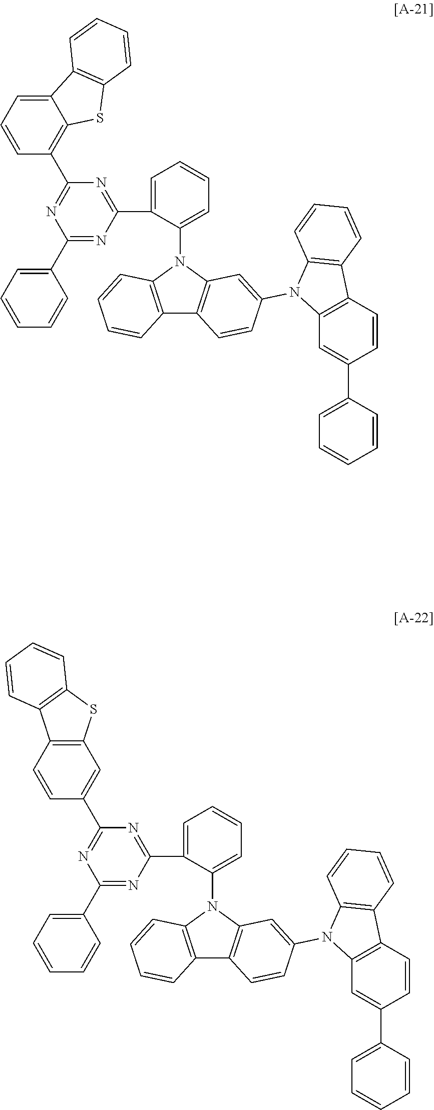 [SMILES: c1ccc(-c2ccc3c4ccccc4n(-c4ccc5c6ccccc6n(-c6ccccc6-c6nc(-c7ccccc7)nc(-c7cccc8c7sc7ccccc78)n6)c5c4)c3c2)cc1 ; c1ccc(-c2ccc3c4ccccc4n(-c4ccc5c6ccccc6n(-c6ccccc6-c6nc(-c7ccccc7)nc(-c7ccc8c(c7)sc7ccccc78)n6)c5c4)c3c2)cc1]

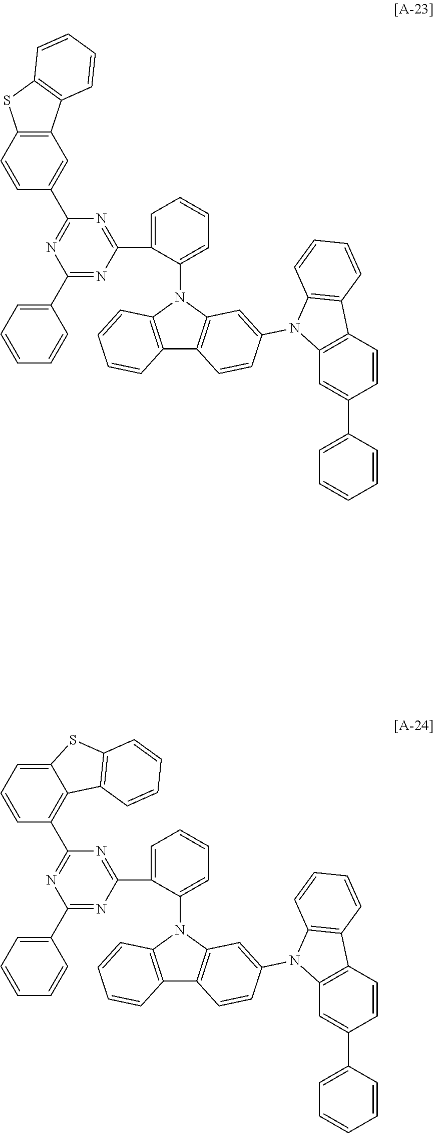 [SMILES: c1ccc(-c2ccc3c4ccccc4n(-c4ccc5c6ccccc6n(-c6ccccc6-c6nc(-c7ccccc7)nc(-c7ccc8sc9ccccc9c8c7)n6)c5c4)c3c2)cc1 ; c1ccc(-c2ccc3c4ccccc4n(-c4ccc5c6ccccc6n(-c6ccccc6-c6nc(-c7ccccc7)nc(-c7cccc8sc9ccccc9c78)n6)c5c4)c3c2)cc1]

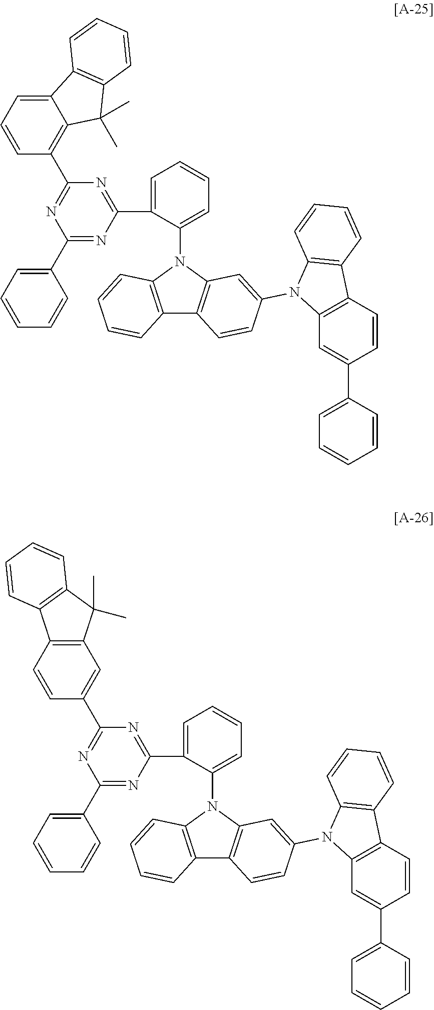 [SMILES: CC1(C)c2ccccc2-c2cccc(-c3nc(-c4ccccc4)nc(-c4ccccc4-n4c5ccccc5c5ccc(-n6c7ccccc7c7ccc(-c8ccccc8)cc76)cc54)n3)c21 ; CC1(C)c2ccccc2-c2ccc(-c3nc(-c4ccccc4)nc(-c4ccccc4-n4c5ccccc5c5ccc(-n6c7ccccc7c7ccc(-c8ccccc8)cc76)cc54)n3)cc21]

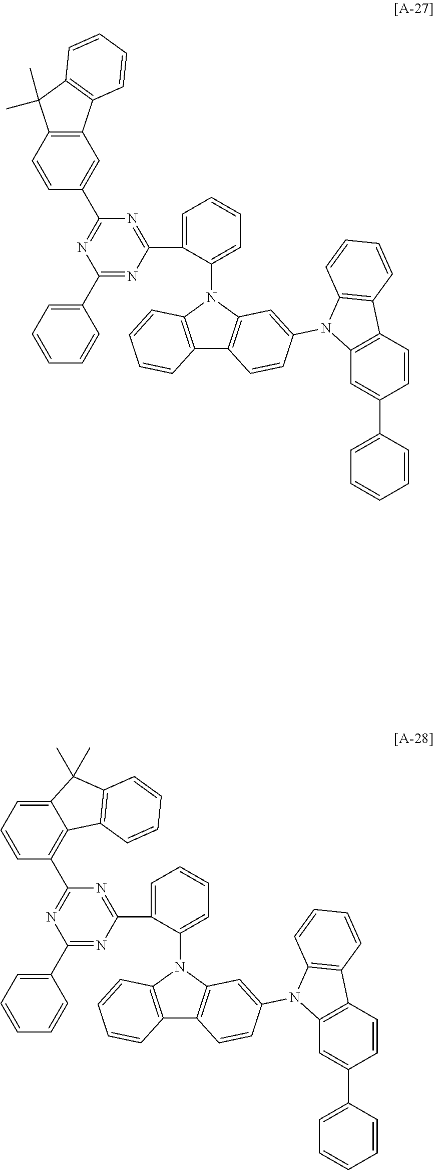 [SMILES: CC1(C)c2ccccc2-c2cc(-c3nc(-c4ccccc4)nc(-c4ccccc4-n4c5ccccc5c5ccc(-n6c7ccccc7c7ccc(-c8ccccc8)cc76)cc54)n3)ccc21 ; CC1(C)c2ccccc2-c2c(-c3nc(-c4ccccc4)nc(-c4ccccc4-n4c5ccccc5c5ccc(-n6c7ccccc7c7ccc(-c8ccccc8)cc76)cc54)n3)cccc21]

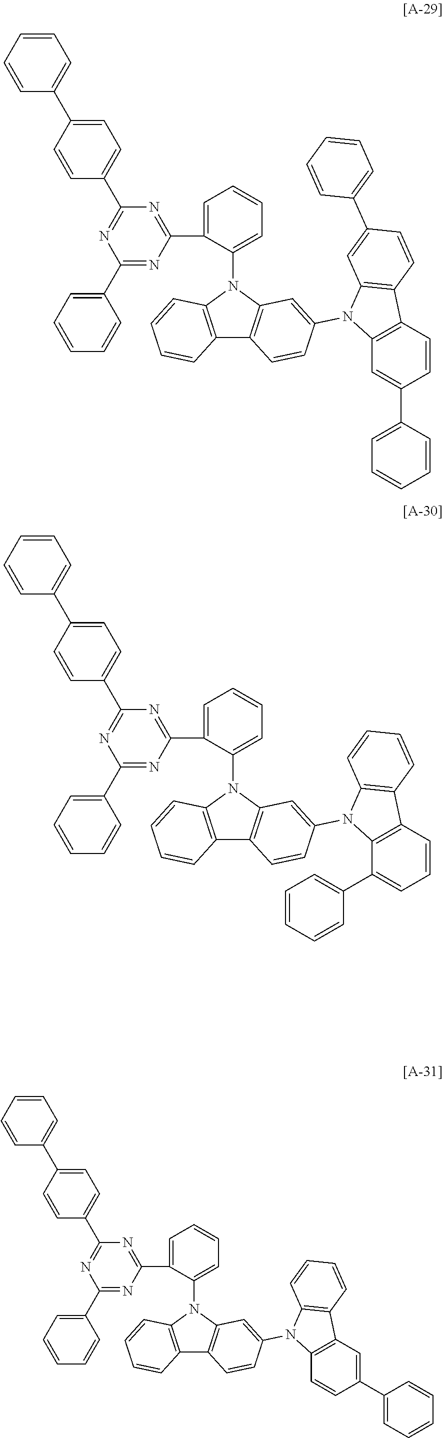 [SMILES: c1ccc(-c2ccc(-c3nc(-c4ccccc4)nc(-c4ccccc4-n4c5ccccc5c5ccc(-n6c7cc(-c8ccccc8)ccc7c7ccc(-c8ccccc8)cc76)cc54)n3)cc2)cc1 ; c1ccc(-c2ccc(-c3nc(-c4ccccc4)nc(-c4ccccc4-n4c5ccccc5c5ccc(-n6c7ccccc7c7cccc(-c8ccccc8)c76)cc54)n3)cc2)cc1 ; c1ccc(-c2ccc(-c3nc(-c4ccccc4)nc(-c4ccccc4-n4c5ccccc5c5ccc(-n6c7ccccc7c7cc(-c8ccccc8)ccc76)cc54)n3)cc2)cc1]

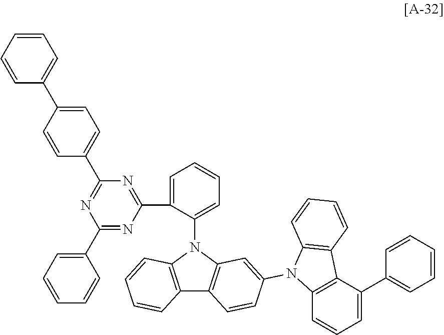 [SMILES: c1ccc(-c2ccc(-c3nc(-c4ccccc4)nc(-c4ccccc4-n4c5ccccc5c5ccc(-n6c7ccccc7c7c(-c8ccccc8)cccc76)cc54)n3)cc2)cc1]

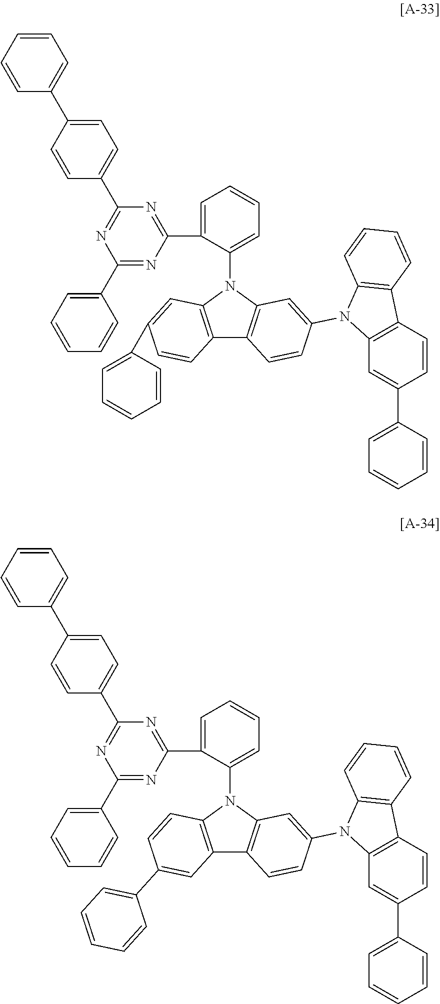 [SMILES: c1ccc(-c2ccc(-c3nc(-c4ccccc4)nc(-c4ccccc4-n4c5cc(-c6ccccc6)ccc5c5ccc(-n6c7ccccc7c7ccc(-c8ccccc8)cc76)cc54)n3)cc2)cc1 ; c1ccc(-c2ccc(-c3nc(-c4ccccc4)nc(-c4ccccc4-n4c5ccc(-c6ccccc6)cc5c5ccc(-n6c7ccccc7c7ccc(-c8ccccc8)cc76)cc54)n3)cc2)cc1]

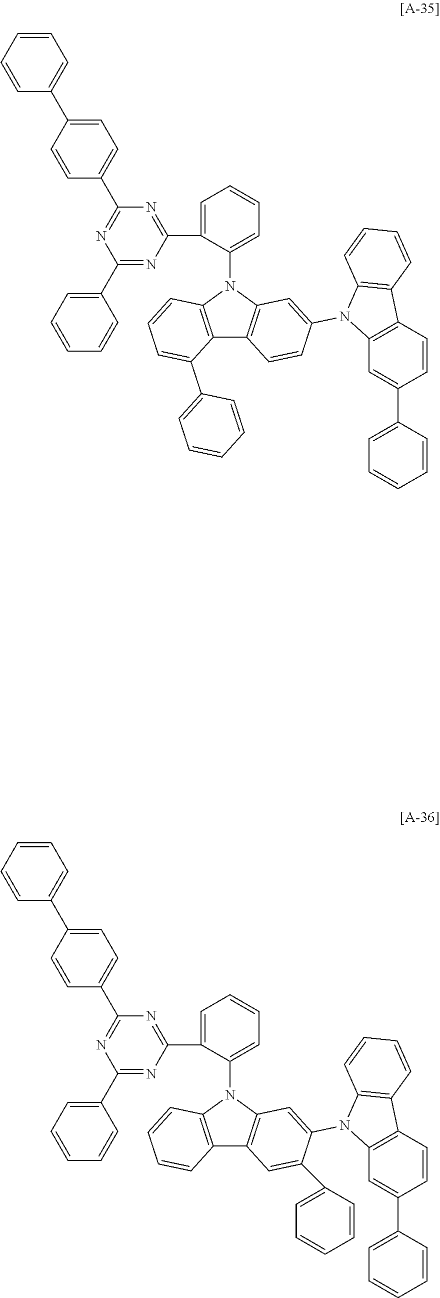 [SMILES: c1ccc(-c2ccc(-c3nc(-c4ccccc4)nc(-c4ccccc4-n4c5cc(-n6c7ccccc7c7ccc(-c8ccccc8)cc76)ccc5c5c(-c6ccccc6)cccc54)n3)cc2)cc1 ; c1ccc(-c2ccc(-c3nc(-c4ccccc4)nc(-c4ccccc4-n4c5ccccc5c5cc(-c6ccccc6)c(-n6c7ccccc7c7ccc(-c8ccccc8)cc76)cc54)n3)cc2)cc1]

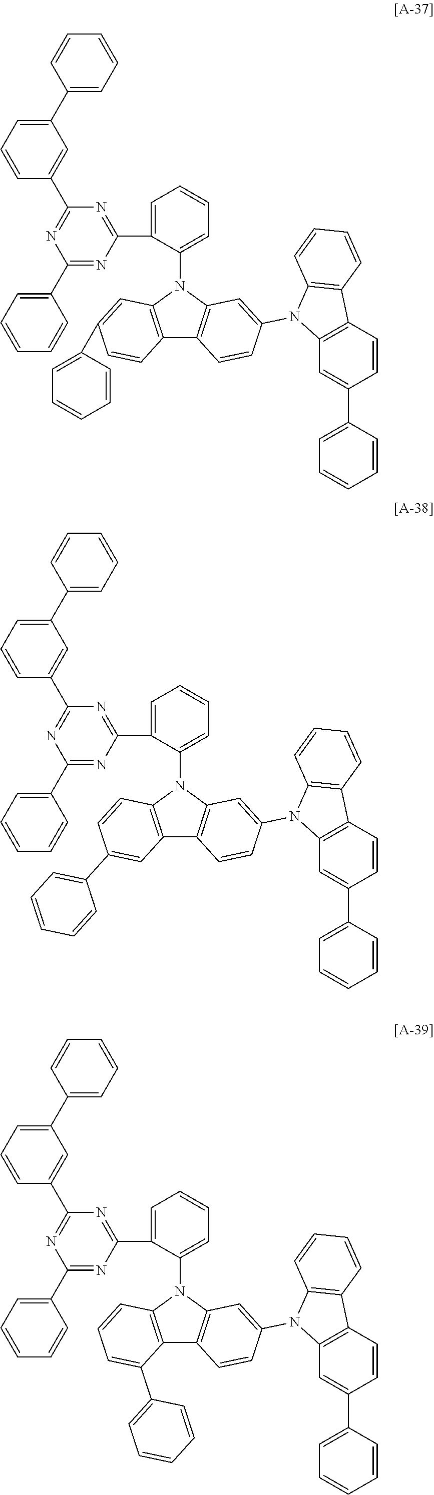 [SMILES: c1ccc(-c2cccc(-c3nc(-c4ccccc4)nc(-c4ccccc4-n4c5cc(-c6ccccc6)ccc5c5ccc(-n6c7ccccc7c7ccc(-c8ccccc8)cc76)cc54)n3)c2)cc1 ; c1ccc(-c2cccc(-c3nc(-c4ccccc4)nc(-c4ccccc4-n4c5ccc(-c6ccccc6)cc5c5ccc(-n6c7ccccc7c7ccc(-c8ccccc8)cc76)cc54)n3)c2)cc1 ; c1ccc(-c2cccc(-c3nc(-c4ccccc4)nc(-c4ccccc4-n4c5cc(-n6c7ccccc7c7ccc(-c8ccccc8)cc76)ccc5c5c(-c6ccccc6)cccc54)n3)c2)cc1]

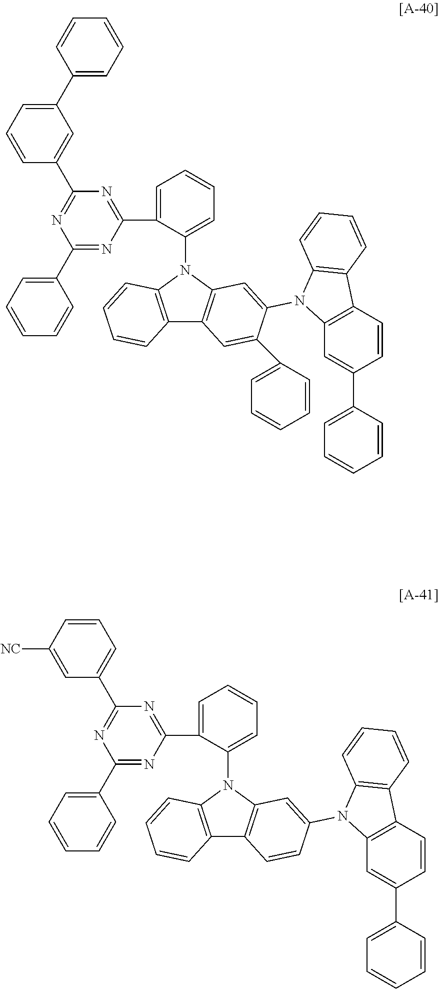 [SMILES: c1ccc(-c2cccc(-c3nc(-c4ccccc4)nc(-c4ccccc4-n4c5ccccc5c5cc(-c6ccccc6)c(-n6c7ccccc7c7ccc(-c8ccccc8)cc76)cc54)n3)c2)cc1 ; N#Cc1cccc(-c2nc(-c3ccccc3)nc(-c3ccccc3-n3c4ccccc4c4ccc(-n5c6ccccc6c6ccc(-c7ccccc7)cc65)cc43)n2)c1]

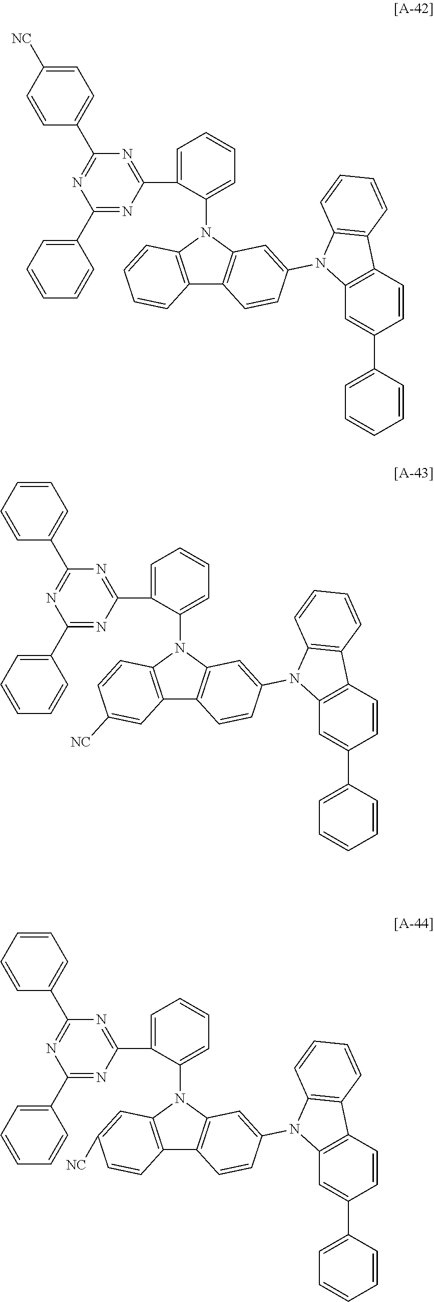 [SMILES: N#Cc1ccc(-c2nc(-c3ccccc3)nc(-c3ccccc3-n3c4ccccc4c4ccc(-n5c6ccccc6c6ccc(-c7ccccc7)cc65)cc43)n2)cc1 ; N#Cc1ccc2c(c1)c1ccc(-n3c4ccccc4c4ccc(-c5ccccc5)cc43)cc1n2-c1ccccc1-c1nc(-c2ccccc2)nc(-c2ccccc2)n1 ; N#Cc1ccc2c3ccc(-n4c5ccccc5c5ccc(-c6ccccc6)cc54)cc3n(-c3ccccc3-c3nc(-c4ccccc4)nc(-c4ccccc4)n3)c2c1]

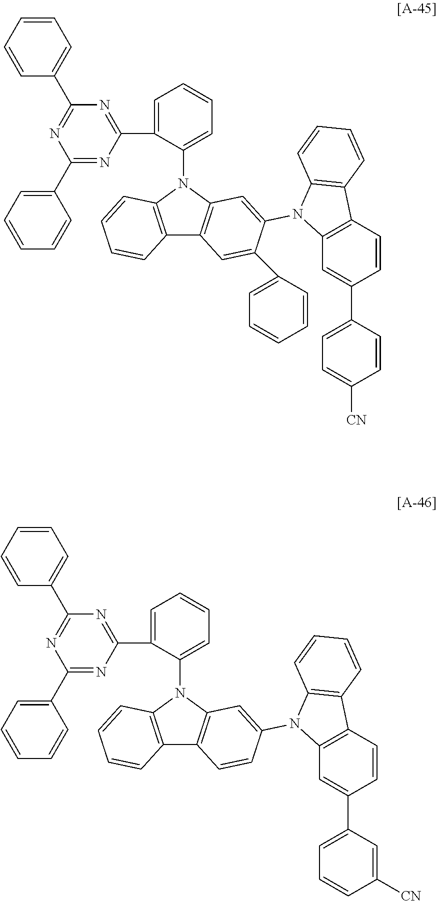 [SMILES: N#Cc1ccc(-c2ccc3c4ccccc4n(-c4cc5c(cc4-c4ccccc4)c4ccccc4n5-c4ccccc4-c4nc(-c5ccccc5)nc(-c5ccccc5)n4)c3c2)cc1 ; N#Cc1cccc(-c2ccc3c4ccccc4n(-c4ccc5c6ccccc6n(-c6ccccc6-c6nc(-c7ccccc7)nc(-c7ccccc7)n6)c5c4)c3c2)c1]

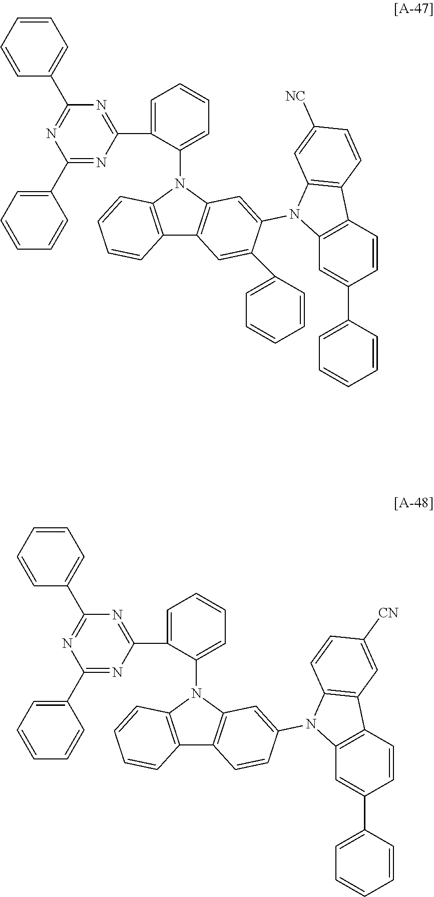 [SMILES: N#Cc1ccc2c3ccc(-c4ccccc4)cc3n(-c3cc4c(cc3-c3ccccc3)c3ccccc3n4-c3ccccc3-c3nc(-c4ccccc4)nc(-c4ccccc4)n3)c2c1 ; N#Cc1ccc2c(c1)c1ccc(-c3ccccc3)cc1n2-c1ccc2c3ccccc3n(-c3ccccc3-c3nc(-c4ccccc4)nc(-c4ccccc4)n3)c2c1]

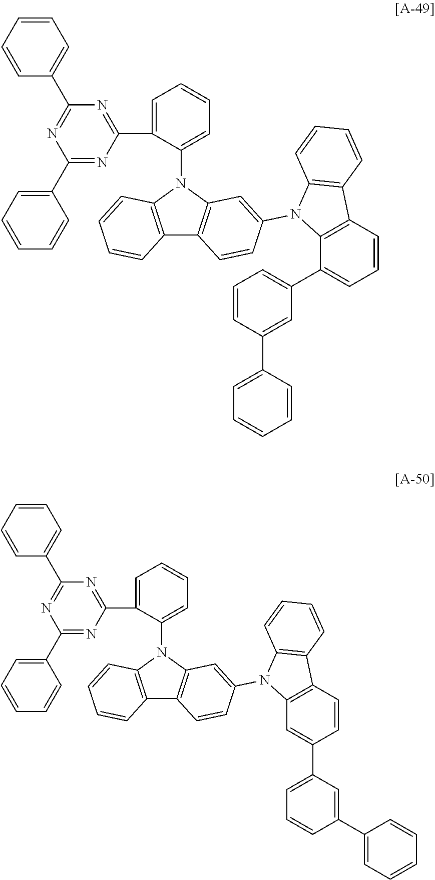 [SMILES: c1ccc(-c2cccc(-c3cccc4c5ccccc5n(-c5ccc6c7ccccc7n(-c7ccccc7-c7nc(-c8ccccc8)nc(-c8ccccc8)n7)c6c5)c34)c2)cc1 ; c1ccc(-c2cccc(-c3ccc4c5ccccc5n(-c5ccc6c7ccccc7n(-c7ccccc7-c7nc(-c8ccccc8)nc(-c8ccccc8)n7)c6c5)c4c3)c2)cc1]

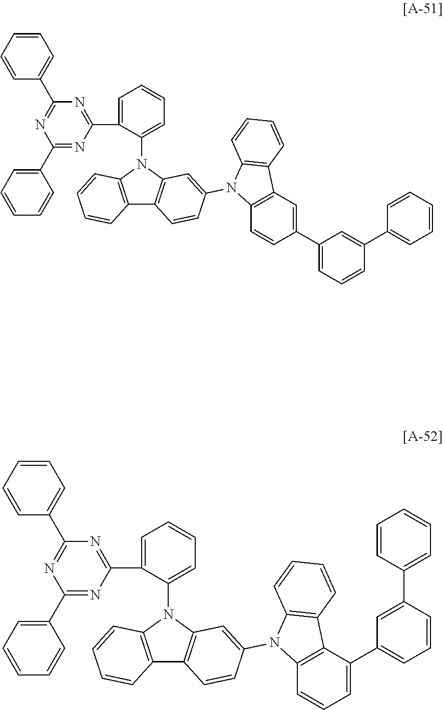 [SMILES: c1ccc(-c2cccc(-c3ccc4c(c3)c3ccccc3n4-c3ccc4c5ccccc5n(-c5ccccc5-c5nc(-c6ccccc6)nc(-c6ccccc6)n5)c4c3)c2)cc1 ; c1ccc(-c2cccc(-c3cccc4c3c3ccccc3n4-c3ccc4c5ccccc5n(-c5ccccc5-c5nc(-c6ccccc6)nc(-c6ccccc6)n5)c4c3)c2)cc1]

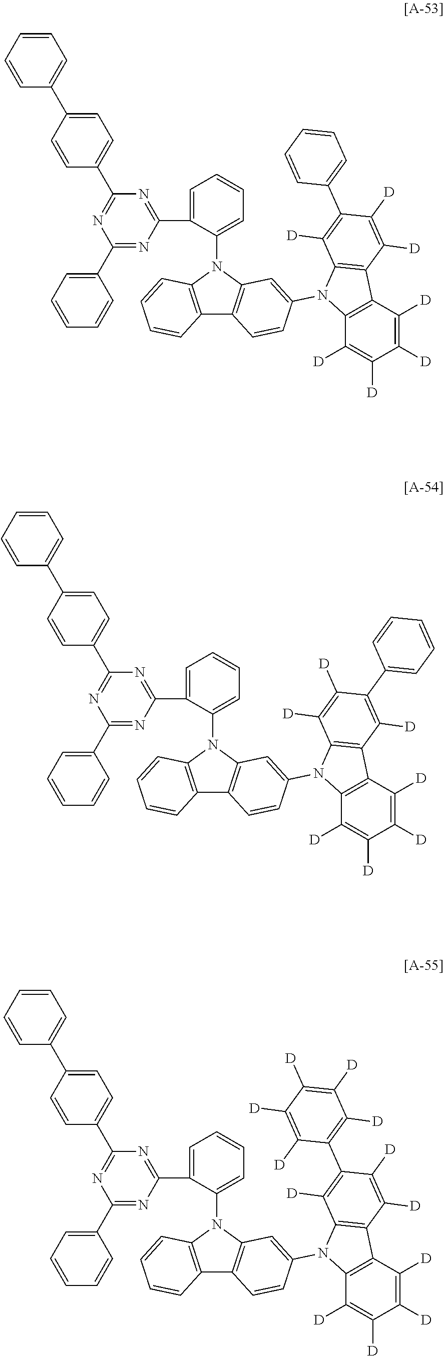 [SMILES: [2H]c1c([2H])c([2H])c2c(c1[2H])c1c([2H])c([2H])c(-c3ccccc3)c([2H])c1n2-c1ccc2c3ccccc3n(-c3ccccc3-c3nc(-c4ccccc4)nc(-c4ccc(-c5ccccc5)cc4)n3)c2c1 ; [2H]c1c([2H])c([2H])c2c(c1[2H])c1c([2H])c(-c3ccccc3)c([2H])c([2H])c1n2-c1ccc2c3ccccc3n(-c3ccccc3-c3nc(-c4ccccc4)nc(-c4ccc(-c5ccccc5)cc4)n3)c2c1 ; [2H]c1c([2H])c([2H])c(-c2c([2H])c([2H])c3c4c([2H])c([2H])c([2H])c([2H])c4n(-c4ccc5c6ccccc6n(-c6ccccc6-c6nc(-c7ccccc7)nc(-c7ccc(-c8ccccc8)cc7)n6)c5c4)c3c2[2H])c([2H])c1[2H]]

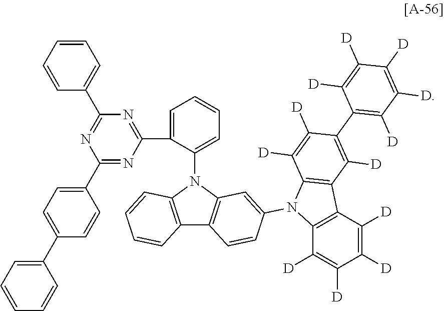

8. A composition for an organic optoelectronic device, the composition comprising:
a first compound; and
a second compound,
wherein:
the first compound is the compound for an organic optoelectronic device according to claim 1, and
the second compound is represented by:
Chemical Formula 2; or
a combination of Chemical Formula 3 and Chemical Formula 4: 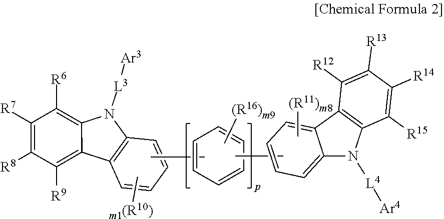
in Chemical Formula 2,
Ar³ and Ar⁴ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁶ to R¹⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m7 and m8 are each independently an integer of 1 to 3,
m9 is an integer of 1 to 4, and
p is an integer of 0 to 2; 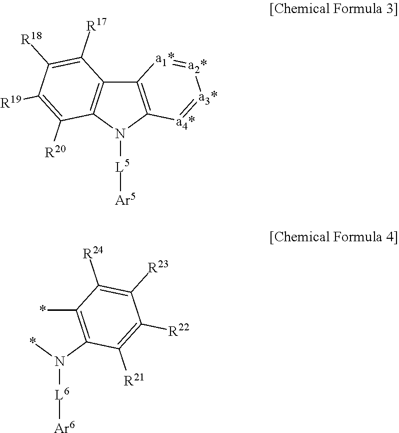
in Chemical Formulas 3 and 4,
Ar⁵ and Ar⁶ are each independently a substituted or unsubstituted C6 to C20 aryl group or a substituted or unsubstituted C2 to C30 heterocyclic group,
two adjacent ones of a₁* to a₄* of Chemical Formula 3 are linking carbons linked at * of Chemical Formula 4,
the remaining two of a₁* to a₄* of Chemical Formula 3, not linked at * of Chemical Formula 4, are each independently C-L^{a}-R^{a},
L^{a}, L⁵, and L⁶ are each independently a single bond or a substituted or unsubstituted C6 to C20 arylene group, and
R^{a} and R¹⁷ to R²⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

9. The composition for an organic optoelectronic device of claim 8, wherein;
the second compound is represented by Chemical Formula 2,
Chemical Formula 2 is represented by Chemical Formula 2-8: 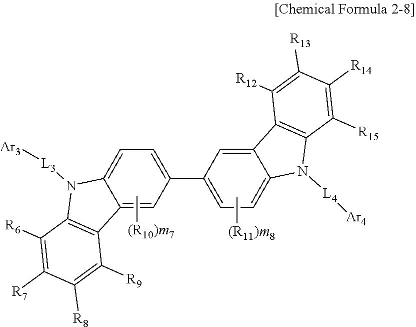
in Chemical Formula 2-8,
R⁶ to R¹⁵ are each independently hydrogen, deuterium, or a substituted or unsubstituted C6 to C12 aryl group,
moieties *-L³-Ar³ and *-L⁴-Ar⁴ are each independently a moiety of Group II, and
m7 and m8 are each independently an integer of 1 to 3, 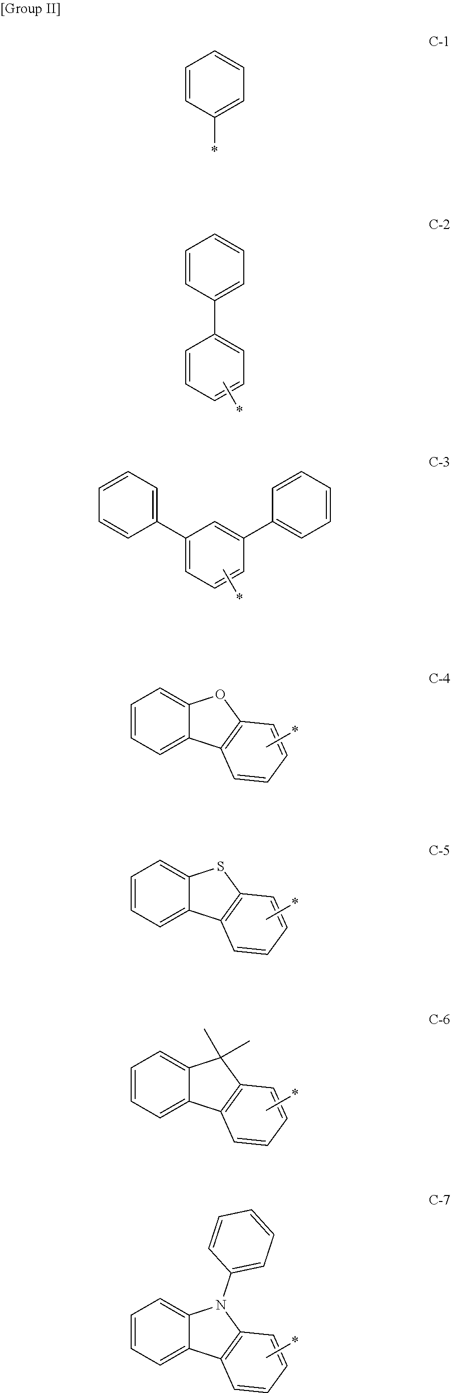 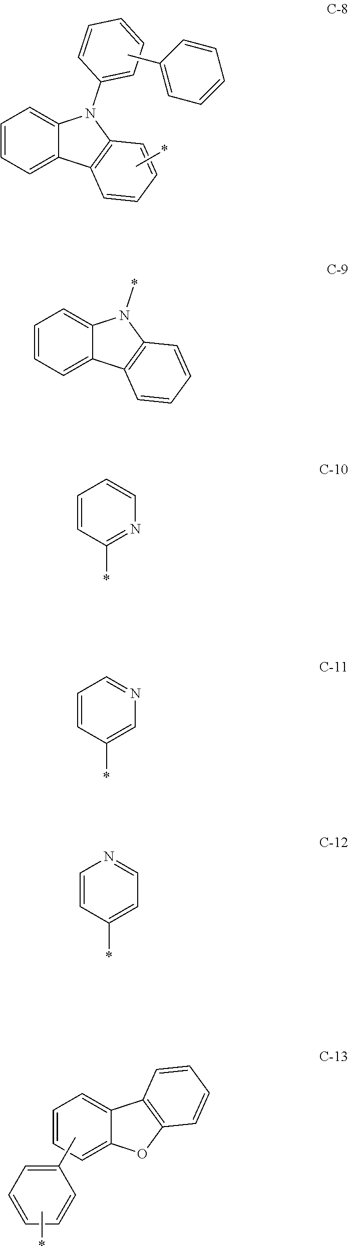 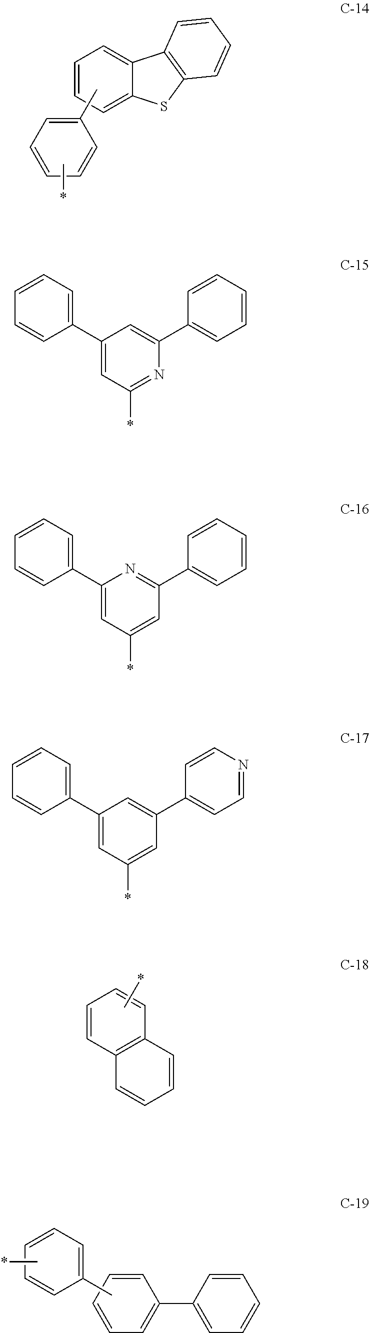 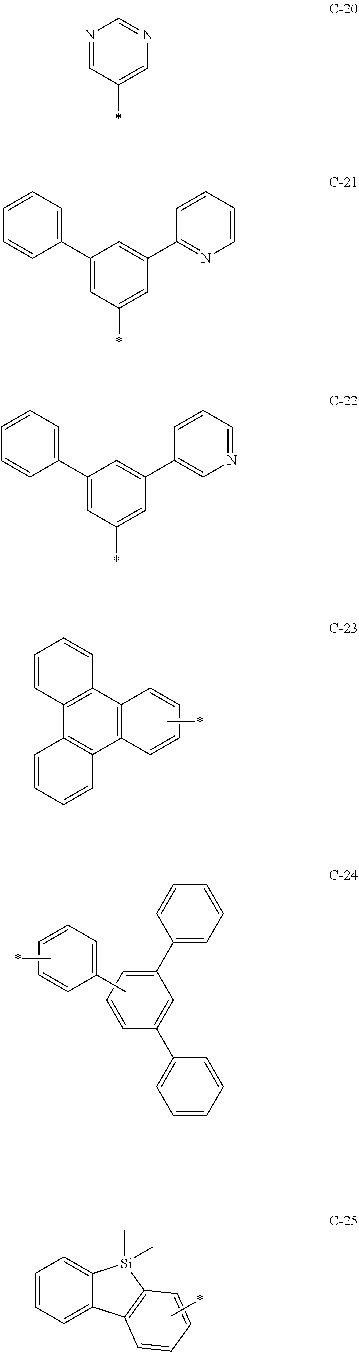 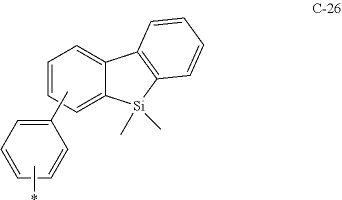
in Group II, * is a linking point.

10. The composition for an organic optoelectronic device of claim 8, wherein:
the second compound is represented by a combination of Chemical Formula 3 and Chemical Formula 4,
the combination of Chemical Formula 3 and Chemical Formula 4 is represented by Chemical Formula 3C: 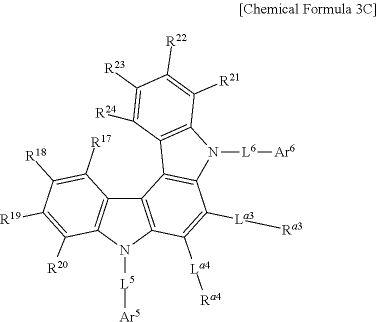
in Chemical Formula 3C,
L^{a3} and L^{a4} are each a single bond,
L⁵ and L⁶ are each independently a single bond or a substituted or unsubstituted C6 to C12 arylene group,
R¹⁷ to R²⁴, R^{a3}, and R^{a4} are each independently hydrogen or a substituted or unsubstituted C6 to C12 aryl group, and
Ar⁵ and Ar⁶ are each independently a substituted or unsubstituted phenyl group, a substituted or unsubstituted naphthyl group, or a substituted or unsubstituted biphenyl group.

11. An organic optoelectronic device, comprising:
an anode and a cathode facing each other,
at least one organic layer between the anode and the cathode,
wherein the at least one organic layer includes the compound for an organic optoelectronic device of claim 1.

12. The organic optoelectronic device of claim 11, wherein:
the at least one organic layer includes a light emitting layer, and
the light emitting layer includes the compound.

13. A display device comprising the organic optoelectronic device of claim 11.

14. An organic optoelectronic device, comprising:
an anode and a cathode facing each other,
at least one organic layer between the anode and the cathode,
wherein the at least one organic layer includes the composition for an organic optoelectronic device of claim 8.

15. The organic optoelectronic device of claim 14, wherein:
the at least one organic layer includes a light emitting layer, and
the light emitting layer includes the composition.

16. A display device comprising the organic optoelectronic device of claim 14.
